(19) United States
(12) Patent Application Publication
(10) Pub. No.: US 2007/0154561 A1
(43) Pub. Date: Jul. 5, 2007

(54) Metal oxide particle and its uses

(75) Inventors: Mitsuo Takeda, Hyogo (JP); Ryuji Aizawa, Osaka (JP); Yumiko Mori, Osaka (JP); Tomoyuki Kuwamoto, Nara (JP)

Correspondence Address: ROBERT J JACOBSON PA, 650 BRIMHALL STREET SOUTH, ST PAUL, MN, 551161511, US

(73) Assignee: Nippon Shokubai Co., Ltd.

(21) Appl. No.: 10/588,526

(22) PCT Filed: Feb. 17, 2005
(86) PCT No.: PCT/JP05/02956

§ 371 Date: Aug. 5, 2006

(30) Foreign Application Priority Data
Feb. 18, 2004 (JP) 2004-041983
Feb. 18, 2004 (JP) 2004-041984
Jan. 5, 2005 (JP) 2005-001034
Jan. 5, 2005 (JP) 2005-001035

(51) Int. Cl. A61K 9/14; A01N 25/00

(52) U.S. Cl. 424/489

(57) ABSTRACT

An object of the present invention is to provide a metal oxide particle which exercises more excellent ultraviolet absorbency as a matter of course and combines therewith merits of, for example, either being shifted in ultraviolet absorption edge toward the longer wavelength side and being excellent also in the absorption efficiency of a long-wavelength range of ultraviolet rays, or having good transparency and, for example, even in cases where added into or coated onto substrates, not damaging the transparency or hue of the substrates. As a means of achieving this object, a metal oxide particle according to the present invention is a metal oxide particle such that a hetero-element is contained in a particle comprising an oxide of a specific metal element (M), wherein the metal oxide particle is: 1) a metal oxide particle in the form of a fine particle wherein the hetero-element is at least one specific metal element (M′); 2) a metal oxide particle wherein the hetero-element includes at least two specific metal elements (M′); 3) a metal oxide particle wherein: the hetero-element is a more specified metal element (M′) and at least a part thereof is 2 in valence; or the metal element (M) is a more specified metal element and the metal oxide particle is in a specific range in crystal grain diameter in the vertical direction to each of the (002) plane and the (100) plane; or 4) a metal oxide particle wherein: the hetero-element is at least one specific nonmetal element and an acyl group is contained in the particle; or the hetero-element includes at least two specific nonmetal elements; or the hetero-element is at least one specific nonmetal element and a component derived from a metal element (M′) other than the metal element (M) is contained in the particle.

### TECHNICAL FIELD

The present invention relates to a metal oxide particle and its uses, wherein the metal oxide particle exercises excellent ultraviolet absorbency. In detail, the present invention relates to: a metal oxide particle which exercises more excellent ultraviolet absorbency and further, for example, even in cases where added into or coated onto substrates, does not damage the transparency or hue of the substrates; a metal oxide particle of which an ultraviolet absorption edge is shifted toward the longer wavelength side and which is excellent also in the absorption efficiency of a long-wavelength range of ultraviolet rays; a composition which comprises the above particle; and a membrane which comprises the above particle.

### BACKGROUND ART

Hitherto, for the purpose of providing the ultraviolet intercepting ability, there have been carried out methods in which ultraviolet absorbent materials are added into such as fibers, plates, plastic moldings (e.g. films), paints, and cosmetic materials or methods in which such as glass and plastic films are coated with coating agents comprising ultraviolet absorbent materials along with solvents and binder resins.

For example, ultraviolet absorbent materials which are used in various fields of such as cosmetics, building materials, window glass for automobiles or displays, and flat panel displays are, in recent years, required to have an excellent absorption performance not only for ultraviolet rays of not longer than 380 nm (particularly, ultraviolet rays of near 380 nm) which have hitherto been commonly said, but also for a longer-wavelength range of ultraviolet rays (short-wavelength visible rays). Its reason is that a short-wavelength range of visible rays among visible rays are so high energy as to give worry of bringing about deterioration of plastics and bad influences on human bodies. In addition, the aforementioned ultraviolet absorbent materials are required to exercise high visible-ray transmittability without scattering visible rays and have good transparency, and to cause no coloring (e.g. yellowing) and make no change of hue of substrates, and to be excellent in the durability and the heat resistance.

As the ultraviolet absorbent materials for providing the ultraviolet intercepting ability, inorganic materials are desirable in points of the durability and the heat resistance. Above all, for example, zinc oxide having a physical property of entire interception of ultraviolet rays having wavelengths of not longer than 370 nm is known to be effective and has been used generally in the form of particles. However, these materials do not satisfy the aforementioned requirements. For example, there has also been a problem of being lacking in the utility, such that: the ultraviolet absorption performance is so insufficient that, for example, in order to entirely intercept ultraviolet rays of not longer than 380 nm, a large amount of super fine particles per unit area must be used and therefore the membrane becomes too thick.

Thus, as a method for more improving the ultraviolet absorption performance of the ultraviolet absorbent materials, there are proposed combinations of the zinc oxide with such as hetero-metals. For example, there are proposed the following: i) a material such that the zinc oxide is caused to contain (is doped with) Fe and/or Co (e.g. refer to patent document 1 and non-patent document 1 below); ii) a compound oxide of at least one member selected from the group consisting of Ce, Ti, Al, Fe, Cr, and Zr with Zn (e.g. refer to patent document 2 below); and iii) a material such that the zinc oxide is caused to contain at least one member selected from the group consisting of Ce, Ti, Al, Fe, Co, La, and Ni (e.g. refer to patent document 3 below).

By the way, though not targeting the ultraviolet absorbent materials, yet, as an art of causing the zinc oxide to contain a hetero-metal, there is reported a zinc oxide in which Cu is held in solid solution in an amount of 10² ppm (e.g. refer to non-patent document 2 below). It seems that such a zinc oxide can also be expected to be improved in the ultraviolet absorption performance.

| | |
|---|---|
| [Patent Document 1] | JP-A-188517/1997 (Kokai) |
| [Patent Document 2] | JP-A-275182/1987 (Kokai) |
| [Patent Document 3] | JP-A-222317/1993 (Kokai) |

[Non-Patent Document 1] Jun OHTSUKA, “Inorganic Pigments Comprising ZnO as a Main Component”, Ceramics, published by Corporate Juridical Party: The Society of Ceramics, Japan, published in 1983, Vol. 18, No. 11, p. 958-964

[Non-Patent Document 2] Noboru SAI(AGAMI and another person, “Optical Properties of Impurities-doped Hydrothermally Grown Zinc Oxide”, The Journal of the Society of Ceramics, Japan, published by Corporate Juridical Party: The Society of Ceramics, Japan, published in 1969, Vol. 77 [9], p. 309-312

However, in recent years, under circumstances where uses which demandhigher ultraviolet intercepting abilities are increasing, prior metal oxides such as the aforementioned i) to iii) still cannot be said to be sufficient in the ultraviolet absorption ability. In detail, as to them, the absorption performance for light of longer than 380 nm in wavelength may be enhanced somewhat, but the effect of shifting an ultraviolet absorption edge toward the longer wavelength side is still insufficient. Also, in cases such as the aforementioned i) where Fe and/or Co is caused to be contained, the absorption performance at 380 nm on which the highest demand for interception is made results in rather deteriorating, and besides, because the absorption band exists in the visible-ray range, Fe and Co result in strongly coloring the materials yellow and blue respectively, so that there has occurred a problem that, if such a prior metal oxide is used as an ultraviolet absorbent material and added into or coated onto substrates, the transparency or hue of the substrates is damaged. Furthermore, as to prior arts, it is even substantially difficult to produce fine particles which are doped with the above metals and excellent in the isolating ability and the dispersibility.

The prior zinc oxide in which Cu is held in solid solution, as reported in non-patent document 2, is a product synthesized by the hydrothermal method and has a very large particle size of 10 to 25 mm and further is colored yellow. Accordingly, in cases where such a prior zinc oxide is used as an ultraviolet absorbent material, there cannot be obtained a property of exercising a high visible-ray transmittability and having good transparency, so that there occurs a problem that, if such a prior metal oxide is used as an ultraviolet absorbent material and added into or coated onto substrates, the transparency or hue of the substrates is damaged.

### DISCLOSURE OF THE INVENTION

### Object of the Invention

Thus, an object of the present invention is to provide: a metal oxide particle which exercises more excellent ultraviolet absorbency as a matter of course and combines therewith merits of, for example, either being shifted in ultraviolet absorption edge toward the longer wavelength side and being excellent also in the absorption efficiency of a long-wavelength range of ultraviolet rays, or having good transparency and, for example, even in cases where added into or coated onto substrates, not damaging the transparency or hue of the substrates; a composition which comprises the above particle; a membrane which comprises the above particle; a metal-oxide-containing article which comprises the above particle; and an ultraviolet absorbent material which comprises the above particle.

Incidentally, the ultraviolet wavelength range which is to be intercepted (cut oft) in the present invention is defined as including not only the range of not longer than 380 nm which has hitherto been commonly said, but also the short-wavelength range of visible rays (specifically, wavelength range of 380 to 450 nm). Hereinafter, the ultraviolet rays and those in the ultraviolet interception (cutting-off) and ultraviolet absorption are defined as referring to light of which the wavelength is in the above range (not longer than 450 nm).

### SUMMARY OF THE INVENTION

In order to solve the above problems, the present inventors diligently studied about causing an oxide of the metal element (M) to contain a hetero-element (e.g. hetero-metal element (M′)) different from the metal element (M). As a result, they have found out, to begin with, that: it is optimum, in point of enabling the exercise of the extremely excellent enhancing effects, to select, as the oxide of the metal element (M), a single or compound oxide comprising at least one metal element selected from the group consisting of Zn, Ti, Ce, In, Sn, Al, and Si and to select at least one of Cu, Ag, Mn, and Bi as the hetero-metal element (M′) being caused to be contained, and, if such a hetero-metal-containing metal oxide particle is in the form of a fine particle, then this particle exercises more excellent ultraviolet absorbency and further has good transparency and, for example, even in cases where added into or coated onto substrates, does not damage the transparency or hue of the substrates.

The present inventors have found out secondly that a metal oxide particle comprising a metal oxide, such that a single or compound oxide comprising at least one metal element selected from the group consisting of Zn, Ti, Ce, In, Sn, Al, and Si, as the oxide of the metal element (M), is caused to further contain at least two specific hetero-metal elements (metal elements (M′) selected from the group consisting of Co, Cu, Fe, Bi, In, Al, Ga, Ti, Sn, Ag, Mn, Ni, and Ce) in combination with each other, would exercise more excellent ultraviolet absorbency as a matter of course and shift its ultraviolet absorption edge toward the longer wavelength side to thus exercise an effect excellent also in the absorption efficiency of a long-wavelength range of ultraviolet rays.

Thirdly, the present inventors have found out that: if, in cases where a single or compound oxide comprising at least one metal element selected from the group consisting of Zn, Ti, Ce, In, Sn, Al, and Si is selected as the oxide of the metal element (M) and at least one of Co, Fe, and Ni (which can exercise effects particularly excellent in point of the ultraviolet intercepting ability (ultraviolet absorbency)) is selected as the hetero-metal element (M′) being caused to be contained, at least a part of these Co, Fe, and Ni is 2 in valence, then a very useful metal oxide particle is obtained. Specifically, as to Fe, if it is 3 in valence, then it strongly colors the particle yellow to brown and therefore may be unfavorable for uses which demand to be more colorless transparent. However, if Fe is 2 in valence or those of 2 and 3 in valence coexist, then the particle is colored green or greenish. Therefore, when a membrane is formed from such a particle, its coloring is inconspicuous. Even if the coloring occurs somewhat, it can be suppressed to such a soft hue as to be sufficiently usable in uses which demand to be more colorless transparent. As to Co, cases where it is 2 in valence or that of 2 in valence is included exercise more excellent ultraviolet absorption performance than cases where it is 3 alone in valence. As to Ni, if it is 2 in valence, then it gives a greenish powder, so that the same effects as of the aforementioned Fe can be expected, and further that the ultraviolet absorption performance is also excellent. In addition, the present inventors have noticed that, in cases where a single or compound oxide comprising Zn is selected as the oxide of the metal element (M) and at least one of Co, Fe, and Ni is selected as the hetero-metal element (M′) being caused to be contained, then the size of the crystal grain in a specific direction is important, and the present inventors have found it favorable that this size is in the nano-size level range which has never been seen in the prior metal oxide particles further containing Co, Fe, or Ni.

The present inventors have found out fourthly that: if a single or compound oxide comprising at least one metal element selected from the group consisting of Zn, Ti, Ce, In, Sn, Al, and Si is used as the oxide of the metal element (M) and if this oxide of the metal element (M) is caused to contain at least one member selected from the group consisting of N, S, and group-17 (group-7B) elements and if conditions such as composition (surface composition, internal composition) are optimized, then there is obtained a particle excellent in the ultraviolet absorption performance, the visible-ray transmission performance, and the hue.

Furthermore, the present inventors have found out that: if the aforementioned metal oxide particles are combined with a metal oxide particle including a specific metal element (metal element selected from the group consisting of Cu, Fe, Ag, and Bi) as a metal component and/or with a superfine metal particle including, as a metal element, at least one member selected from the group consisting of Ag, Cu, Au, and platinum group metal elements, then the effect of intercepting a short-wavelength range of visible rays can more be enhanced.

The present invention has been completed from these knowledge and findings.

Accordingly, the metal oxide particles according to the present invention are as follows.

That is to say, a first invention is a metal oxide particle in the form of a fine particle, which is a metal oxide particle such that a component derived from a metal element (M′) other than a metal element (M) is contained in a particle comprising an oxide of the metal element (M), with the metal oxide particle being characterized in that: the metal element (M) is at least one member selected from the group consisting of Zn, Ti, Ce, In, Sn, Al, and Si; and the metal element (M′) is at least one member selected from the group consisting of Cu, Ag, Mn, and Bi. Incidentally, in this first invention, the “fine particle” refers to a particle of which the primary particle diameter is not larger than 0.1 μm. In detail, the aforementioned primary particle diameter can be judged from the crystal grain diameter or the specific surface area diameter, and a particle of which either the crystal grain diameter or the specific surface area diameter is not larger than 0.1 μm is regarded as the “fine particle”.

A second invention is a metal oxide particle, which is a metal oxide particle such that a component derived from a metal element (M′) other than a metal element (M) is contained in a particle comprising an oxide of the metal element (M), with the metal oxide particle being characterized in that: the metal element (M) is at least one member selected from the group consisting of Zn, Ti, Ce, In, Sn, Al, and Si; and the metal element (M′) includes at least two members which are different from the metal element (M) and selected from the group consisting of Co, Cu, Fe, Bi, In, Al, Ga, Ti, Sn, Ce, Ni, Mn, and Ag.

A third invention is a metal oxide particle, which is a metal oxide particle such that a component derived from a metal element (M′) other than a metal element (M) is contained in a particle comprising an oxide of the metal element (M), with the metal oxide particle being characterized in that: the metal element (M′) is at least one member selected from the group consisting of Co, Fe, and Ni; and in either i) that: the metal element (M) is at least one member selected from the group consisting of Zn, Ti, Ce, In, Sn, Al, and Si; and at least a part of Co, Fe, and Ni as the metal element (M′) is 2 in valence; or ii) that: the metal element (M) is Zn; and the metal oxide particle is not larger than 30 nm in crystal grain diameter in the vertical direction to the (002) plane and not smaller than 8 nm in crystal grain diameter in the vertical direction to the (100) plane.

A fourth invention is a metal oxide particle, which is a metal oxide particle comprising an oxide of a metal element (M), with the metal oxide particle being characterized in that: the metal element (M) is at least one member selected from the group consisting of Zn, Ti, Ce, In, Sn, Al, and Si; and in: i) that: at least one member selected from the group consisting of N, S, and group-17 (group-7B) elements, and further an acyl group, are contained in the oxide of the metal element (M); or ii) that: at least two members selected from the group consisting of N, S, and group-17 (group-7B) elements are contained in the oxide of the metal element (M); or iii) that: at least one member selected from the group consisting of N, S, and group-17 (group-7B) elements is contained in the oxide of the metal element (M); and a component derived from a metal element (M′) other than the metal element (M) is contained in the particle.

A composition according to the present invention comprises a metal oxide particle and a medium, wherein the metal oxide particle is dispersed in the medium and includes, as an essential component, the aforementioned metal oxide particle according to the present invention.

In the aforementioned composition according to the present invention, a composition according to the present invention for membrane formation comprises the following essential constitutional components: the aforementioned metal oxide particle according to the present invention; and a dispersion solvent and/or a binder.

A membrane according to the present invention comprises a metal oxide as an essential constitutional component, wherein the metal oxide includes the following essential components: the aforementioned metal oxide particle according to the present invention; and/or a metal oxide crystal derived from this particle.

A metal-oxide-containing article according to the present invention is an article comprising a metal oxide particle and/or a metal oxide crystal derived from this particle, wherein the article includes, as essential components, a combination of the aforementioned metal oxide particle according to the present invention with: a metal oxide particle including, as a metal element, at least one member selected from the group consisting of Cu, Fe, Ag, and Bi; and/or a superfine metal particle including, as a metal element, at least one member selected from the group consisting of Cu, Ag, Au, and platinum group metal elements.

An ultraviolet absorbent material according to the present invention comprises the aforementioned metal oxide particle according to the present invention.

### EFFECTS OF THE INVENTION

The present invention can provide: a metal oxide particle which exercises more excellent ultraviolet absorbency as a matter of course and combines therewith merits of, for example, either being shifted in ultraviolet absorption edge toward the longer wavelength side and being excellent also in the absorption efficiency of a long-wavelength range of ultraviolet rays, or having good transparency and, for example, even in cases where added into or coated onto substrates, not damaging the transparency or hue of the substrates; a composition (e.g. composition for membrane formation) which comprises the above particle; a membrane which comprises the above particle; a metal-oxide-containing article which comprises the above particle; and an ultraviolet absorbent material which comprises the above particle.

### BRIEF DESCRIPTION OF THE DRAWINGS

FIG. 1 is a graph showing the results of the evaluation (4-1) of the absorption properties in the Example A1 series and the Comparative Example A1 series.

FIG. 2 is a graph showing the results (transmission spectra) of the evaluation of the absorption properties in Example A3-1.

FIG. 3 is a graph showing the results (transmission spectrum) of the evaluation of the absorption properties in Example A3-2.

FIG. 4 is a graph showing the results (transmission spectrum) of the evaluation of the absorption properties in Example A3-3.

FIG. 5 is a graph showing the results of diffused reflectance spectra as to the metal oxide particles having been obtained from Example A1-17 and Comparative Example A1-1.

FIG. 6 shows transmission spectra of dispersion-membrane-coated glasses having been obtained in the evaluations about the metal oxide particles in the reaction liquids having been obtained from Example B1-1 and Comparative Example B1-1.

FIG. 7 shows transmission spectra of dispersion-membrane-coated glasses having been obtained in the evaluations about the metal oxide particles in the reaction liquids having been obtained from Example B1-2 and Comparative Example B1-2.

FIG. 8 shows transmission spectra of dispersion-membrane-coated glasses (three kinds different in dry membrane thickness based on the difference in wet membrane thickness) having been obtained from Example B3-1.

### DETAILED DESCRIPTION OF THE INVENTION

Hereinafter, detailed descriptions are given about the present invention. However, the scope of the present invention is not bound to these descriptions. And other than the following illustrations can also be carried out in the form of appropriate modifications of the following illustrations within the scope not departing from the spirit of the present invention.

[Metal Oxide Particle]:

Any of the metal oxide particles according to the present invention is, as aforementioned, a metal oxide particle comprising an oxide of a metal element (M) wherein the metal oxide particle contains a specific hetero-element different from the metal element (M). In detail, the aforementioned metal oxide particles according to the first, second, and third inventions are such that a component derived from a metal element (M′) (which may hereinafter be referred to as “hetero-metal element”) other than the specific metal element (M) is contained in a particle comprising an oxide of the metal element (M), and the aforementioned metal oxide particle according to the fourth invention is such that at least one member (which may hereinafter be referred to as “hetero-nonmetal element”) selected from the group consisting of N, S, and group-17 (group-7B) elements is contained in the oxide of the specific metal element (M). In more detail, in the present invention, as to that the component derived from the aforementioned hetero-metal element, or the aforementioned hetero-nonmetal element, is contained, it will do if the metal oxide constituting the metal oxide particle according to the present invention is a metal oxide containing the aforementioned hetero-metal element or hetero-nonmetal element, and it doesn't matter in what existence form the aforementioned hetero-metal element or hetero-nonmetal element is contained. Incidentally, hereinafter, the aforementioned hetero-metal element and the aforementioned hetero-nonmetal element are referred to generically as “hetero-(metal/nonmetal) element”.

In the metal oxide constituting the metal oxide particle according to the present invention, specific examples of the existence form of the aforementioned hetero-(metal/nonmetal) element include: (I) a form in which the hetero-(metal/nonmetal) element exists in solid solution in a crystal of the oxide of the metal element (M); (II) a form in which the hetero-(metal/nonmetal) element exists in a state contained as a metal component of the oxide of the metal element (M) (favorably, compound oxide); (III) a form in which the hetero-(metal/nonmetal) element is adsorbed on surfaces of the crystal of the oxide of the metal element (M); and (IV) a form in which the hetero-(metal/nonmetal) element is attached to surfaces of the oxide of the metal element (M) in the form of a particle or membrane as a metal or a simple substance. Above all, a form of solid solution such that the aforementioned hetero-(metal/nonmetal) element is uniformly dispersed in an atomic state (including an ionic state) in the crystal of the oxide of the metal element (M) is favorable in point of the effects provided by the hetero-(metal/nonmetal) element, namely, the excellence in the ultraviolet absorbency and also the smallness of the degree of coloring caused by addition of the hetero-(metal/nonmetal) element and the enablement of the retention of good transparency.

As to the dispersed state of the aforementioned hetero-(metal/nonmetal) element in the metal oxide particle according to the present invention, (i) the aforementioned hetero-(metal/nonmetal) element may be contained in a state dispersed uniformly in the particle, or (ii) the aforementioned hetero-(metal/nonmetal) element may be contained partly in the particle (this case means not the segregation but the following: when one particle is taken notice of, the aforementioned hetero-(metal/nonmetal) element is contained locally in a high concentration). Examples of the above case (i) include a case where, in the aforementioned form (I), the aforementioned hetero-(metal/nonmetal) element is held in solid solution uniformly (from the surface layer up to the crystal grain inside) in the crystal of the oxide of the metal element (M). Examples of the above case (ii) include a case where a metal oxide solid solution phase (the aforementioned form (I)), in which the hetero-(metal/nonmetal) element is held in solid solution, or a phase (the aforementioned form (II)) of, if the hetero-(metal/nonmetal) element is the hetero-metal element (M′), then a compound oxide of the hetero-metal element (M′) and the metal element (M) or, if the hetero-(metal/nonmetal) element is the hetero-nonmetal element, then a metal oxide nitride (wherein the hetero-nonmetal element is N), a metal oxide sulfide (wherein the hetero-nonmetal element is S), or a metal oxide halide (wherein the hetero-nonmetal element is a group-17 element), is formed as a surface layer on surfaces of the crystal of the oxide of the metal element (M). These are also encompassed in what are referred to as “metal oxides” in the present invention.

Incidentally, metal oxides are generally classified into those which exhibit the crystallinity (crystal structures) and those which do not exhibit the crystallinity (noncrystal structures). The above crystal structure can be defined as a metal oxide comprising a crystal grain such that a regular atomic configuration is seen with periodicity. And the above crystal structure refers to such that the metal oxide can be identified from a lattice constant and/or a diffraction pattern by electron diffraction analysis and/or X-ray diffraction analysis. A metal oxide which does not correspond to this can be defined as the noncrystal structure. In point of the excellence in such as ultraviolet absorbency, it is favorable that the metal oxide is the crystal structure. The same as this applies also to the metal oxide constituting the metal oxide particle according to the present invention. Particularly, as to the third invention,, in cases where the aforementioned metal element (M) is Zn, then the metal oxide constituting the metal oxide particle is the crystal structure.

The above crystal structure may be either a single crystal structure or a polycrystalline structure. Examples of the shape of the crystal grain constituting these include a sphere, an oval sphere, a cube, a rectangular parallelepiped, a polyhedron, a pyramid, a pillar, a tube, a thin piece (e.g. a scale, a (hexagonal) plate), and tree branches and a skeleton crystal (formed by prior extension of edges and corners of crystals under high supersaturation degree conditions). The orientation of the crystal grain is not limited. The orientations of the crystal grains may either all align or be random. It is also permitted that: a part of them have the same orientation, and the rest is random. Thus there is no limitation.

The shape of the metal oxide particle according to the present invention is not limited. Specifically, in cases where the metal oxide particle according to the present invention is a particle comprising a single crystal structure of a metal oxide, then the shape of the particle is the same as the above crystal grain shape. However, in cases where the metal oxide particle according to the present invention is a particle comprising a polycrystalline structure or a particle such that crystal grains are fixed or aggregated together, then the shape of the particle is not always the same as the shape of the crystal grain, and it is exemplified by such as a sphere (true sphere), an oval sphere, a cube, a rectangular parallelepiped, a pyramid, a needle, a pillar, a bar, a tube, and a thin piece (e.g. a scale, a (hexagonal) plate).

The metal oxide constituting the metal oxide particle according to the present invention is favorably a solid solution metal oxide (solid solution oxide) formed by solid solution (doping) of the hetero-(metal/nonmetal) element into a single oxide (specifically, oxide of the metal element (M)) or compound oxide (oxide comprising at least two metal elements (M)). In addition, this metal oxide has either a stoichiometric or nonstoichiometric composition of the metal element and oxygen. Thus there is no limitation.

The above solid solution oxide may be what is called an interstitial solid solution oxide, or a substitutional solid solution oxide, or their combination. Thus there is no limitation.

In the present invention (first, second, third, and fourth inventions), the aforementioned metal element (M) is at least one member selected from the group consisting of Zn (zinc), Ti (titanium), Ce (cerium), In (indium), Sn (tin), Al (aluminum), and Si (silicon). Among these, Zn, Ti, Ce, In, and Sn are favorable in that the ultraviolet absorption performance is excellent and in that the. effects obtained by containing the metal atom (M′) are high when a semiconductor is formed from the particle. Particularly, Zn, Ti, and Ce are favorable in points of being able to exercise excellent ultraviolet absorption performance even in their single oxides and therefore having high intercepting effects on ultraviolet rays of not longer than 380 nm (which have hitherto been commonly said) and further being able to greatly exercise the effect provided by the hetero-nonmetal element, namely, the effect such that an ultraviolet absorption edge is shifted toward the longer wavelength side.

The aforementioned oxide of the metal element (M) may be either a single or compound oxide. Examples of the compound oxide include compound oxides comprising at least two kinds of M and compound oxides of which the metal components are the metal element (M) and a metal element other than this metal element (M), such as ZnIn₂O₄, Zn₂In₂O₅, Zn₃In₂O₆, GaInO₃, In₄Sn₃O₁₂, Zn₂SnO₄, and ZnSnO₃. In detail typically about cases where the metal element (M) is Zn, favorable examples include ZnAl₂O₄, Zn₂B₆O₁₁, ZnFe₂O₄, ZnMoO₄, ZnSeO₃, Zn₂SiO₄, and ZnWO₄. In addition, in the present invention (first to fourth inventions), a form such that the hetero-element (e.g. the aforementioned hetero-nonmetal element) is substituted for a part of oxygen in the metal oxide shall also be encompassed in the aforementioned oxide of the metal element (M). Particularly, in the fourth metal oxide particle (fourth invention), there are included forms such as: a solid solution such that the hetero-nonmetal element is held in solid solution in place of the oxygen in the aforementioned oxide; and besides, an oxide nitride, an oxide sulfide, and an oxide halide of the metal.

Particularly Zn is more favorable as the metal element (M) in point of the excellence also in the visible-ray transparency, and a zinc-oxide-containing particle is a favorable mode of the metal oxide particles according to the present invention (first, second, third, and fourth inventions).

(First Invention):

The first metal oxide particle according to the present invention (the first invention) is a metal oxide particle in the form of a fine particle (such a metal oxide particle may hereinafter be referred to as “fine particulate metal oxide”) such that: the metal element (M) is at least one member selected from the group consisting of Zn, Ti, Ce, In, Sn, Al, and Si; and the metal element (M′) is at least one member selected from the group consisting of Cu, Ag, Mn, and Bi. Incidentally, the aforementioned first metal oxide particle is a fine particle, namely, a particle of which the primary particle diameter is not larger than 0.1 μm. In other words, the primary particle diameter of the particle comprising the oxide of the aforementioned metal element (M) is not larger than 0.1 μm.

In the aforementioned first metal oxide particle, the aforementioned metal element (M) and its oxide are as aforementioned.

As to the aforementioned first metal oxide particle, favorable examples of the copper (Cu) to be caused to be contained in the oxide of the metal element (M) include Cu(0), Cu(I) and Cu(II). Above all, in cases of the solid solution, Cu(I) and Cu(II) are favorable, and particularly Cu(II) is favorable in point of the excellence in the ultraviolet absorbency.

As to the aforementioned first metal oxide particle, favorable examples of the silver (Ag) to be caused to be contained in the oxide of the metal element (M) include Ag(0) and Ag(I). Above all, in cases of the solid solution, Ag(I) is favorable in point of the excellence in the ultraviolet absorbency.

As to the aforementioned first metal oxide particle, examples of the manganese (Mn) to be caused to be contained in the oxide of the metal element (M) include those of 1 to 7 in valence. Above all, that of 2 or 3 in valence is favorable.

As to the aforementioned first metal oxide particle, examples of the bismuth (Bi) to be caused to be contained in the oxide of the metal element (M) include those of 0 to 3 in valence. Above all, that of 3 in valence is favorable.

Incidentally, in cases where these copper (Cu), silver (Ag), manganese (Mn), and bismuth (Bi) are held in solid solution, the form of their respective solid solutions may be either an interstitial or substitutional solid solution. Thus there is no limitation.

In the aforementioned first metal oxide particle, the content of the aforementioned hetero-metal element (M′) (Cu, Ag) to be caused to be contained in the oxide of the metal element (M) is favorably in the range of 0.01 to 10 atomic %, more favorably 0.1 to 10 atomic %, still more favorably 0.2 to 10 atomic %, yet still more favorably 0.7 to 10 atomic %, relative to the total number of atoms of the aforementioned metal element (M). In comparison, in cases where the hetero-metal element is Mn, its content is favorably in the range of 0.01 to 30 atomic %, particularly favorably 3 to 10 atomic %. In addition, in cases where the. hetero-metal element is Bi, its content is favorably in the range of 0.01 to 10 atomic %, particularly favorably 0.1 to 5 atomic %. If the above content of the hetero-metal element (M′) (Cu, Ag, Mn, Bi) is lower than 0.01 atomic %, then the ultraviolet absorption ability tends to be difficult to sufficiently exercise. On the other hand, if the above content of the hetero-metal element (M′) is higher than each upper limit value, then the visible-ray transmittance tends to be low.

As to the aforementioned first metal oxide particle, it is also possible to cause the aforementioned oxide of the metal element (M) to contain another metal element besides the aforementioned hetero-metal element (M′) (Cu, Ag, Mn, Bi) within the range not damaging the effects of the present invention. Though not limited, favorable examples of such a metal element other than the hetero-metal element (M′) (Cu, Ag, Mn, Bi) include Al, In, Sn, Fe, Co, Ce, alkaline metal elements, and alkaline earth metal elements.

(Second Invention):

The second metal oxide particle according to the present invention (the second invention) is a metal oxide particle such that: the metal element (M) is at least one member selected from the group consisting of Zn, Ti, Ce, In, Sn, Al, and Si; and the metal element (M′) includes at least two members which are different from the metal element (M) and selected from the group consisting of Co, Cu, Fe, Bi, In, Al, Ga, Ti, Sn, Ce, Ni, Mn, and Ag.

In the aforementioned second metal oxide particle, the aforementioned metal element (M) and its oxide are as aforementioned.

In the aforementioned second metal oxide particle, the aforementioned metal element (M′) includes at least two members selected from the group consisting of Co (cobalt), Cu (copper), Fe (iron), Bi (bismuth), In (indium), Al (aluminum), Ga (gallium), Ti (titanium), Sn (tin), Ag (silver), Mn (manganese), Ni (nickel), and Ce (cerium). However, as is aforementioned, the metal elements (M′) must be metal elements which are different from the aforementioned metal element (M). There is no limitation on the combination of the at least two metal elements (M′) if they are at least two members selected from among metal elements, other than the aforementioned metal element (M), of the aforementioned group. Incidentally, it is herein provided that In, Al, Ga, Ti, Ce, and Sn of the aforementioned group selectable as the metal elements (M′) shall generally be designated as n-type dopants.

As to the aforementioned second metal oxide particle, examples of Co of the group selectable as the aforementioned metal elements (M′) include Co(II) and Co(III). Examples of Fe include Fe(II) and Fe(III). In cases where Co and Fe are solid solution components, then those of 2 in valence exercise more excellent ultraviolet absorbency than those of 3 in valence. Therefore, the existence of those of 2 alone in valence or the coexistence of those of 2 and 3 in valence is more favorable than the existence of those of 3 alone in valence. Particularly, in cases of Fe, it is favorably 2 in valence, because the use of that of 3 in valence results in strong yellowing, but because the use of that of 2 in valence results in greenish coloring.

In addition, as to the aforementioned second metal oxide particle, examples of Cu of the group selectable as the aforementioned metal elements (M′) include Cu(0), Cu(I), and Cu(II). Examples of Bi include Bi(III). Examples of In include In(I) and In(III). Examples of Al include Al(III). Examples of Ga include Ga(III). Examples of Ti include Ti(IV) and Ti(III). Examples of Sn include Sn(II) and Sn(IV). Examples of Ag include Ag(0), Ag(I), and Ag(III). Examples of Mn include Mn(I), Mn(II), Mn(III), Mn(IV), Mn(V), Mn(VI), and Mn(VII). Examples of Ni include Ni(0), Ni(II), Ni(I), and Ni(III). Examples of Ce include Ce(IV) and Ce(III). Among these, Cu, Ag, and Ni can exercise their effects also by attaching (adhering) to surfaces of the metal oxide particle in the form of Cu(0), Ag(0), and Ni(0), in other words, metals. Among the above, Bi(III) as Bi, In(III) as In, Ag(I) as Ag, Mn(II) and Mn(III) as Mn, and Ni(II) as Ni are favorable in point of the excellence in such as the ultraviolet intercepting ability.

As to the aforementioned second metal oxide particle, modes favorable as the combination of the at least two being selected as the aforementioned metal elements (M′) are: a combination including the following essential components: at least one member selected from the group consisting of Co, Cu, and Fe; and at least one member selected from the group consisting of Bi, In, Al, Ga, Ti, Sn, and Ce (combination (i) mentioned below); and a combination including, as an essential component, one member selected from the group consisting of Co, Cu, Fe, Ag, Mn, Ni, and Bi (combinations (ii) to (v) mentioned below). Incidentally, it is a matter of course that a mode comprising a combination of at least two of the following (i) to (v) is more favorable.

(i) When compared with both cases where, among the above groups selectable as the aforementioned metal elements (M′), only the group consisting of In, Al, Ga, Ti, Sn, and Ce is caused to be contained in the oxide of the metal element (M) and where, among the above groups, only the group consisting of Co, Fe, and Cu is caused to be contained in the oxide of the metal element (M), then the case where both groups of metal elements (M′) are caused to be contained in combination in the oxide of the metal element (M) has an effect such that the absorption ability in the range of 370 to 450 nm is enhanced. It can be considered that: by causing the metal elements to be contained in such a combination, the functions and effects due to both groups of metal elements (M′) are synergistically exercised to thus enhance the absorption efficiency in the range of 370 to 450 nm. For example, in cases where the metal element (M) is Zn, the ability to absorb ultraviolet rays of 380 nm is greatly enhanced, so that ultraviolet rays can sufficiently be cut off with a smaller amount of material used.

(ii) A combination including Cu as an essential component. This inclusion of Cu as an essential component can greatly enhance the performance to absorb a higher-energy range (range of not longer than 380 nm which range is commonly called ultraviolet rays) of light and further can suppress the yellowing. In addition, for example, in cases where the metal oxide particle exhibits yellow hue (e.g. cases where Ti and/or Ce is contained as a metal element (M) and cases where Fe, Bi, and/or Mn is contained as a metal element (M′)), then the above combination is favorable also in such that: by causing Cu to coexist as an essential component, there can also be exercised the function of reducing the yellow or making it colorless. Cu can favorably be combined with any element of the group selectable as the aforementioned metal elements (M′). However, as metal elements (M′) to be partners of Cu in this combination, the aforementioned n-type dopants (above all, In, Al, Sn, Ce) are more favorable in point of being able to synergistically more enhance the effect of enhancing the performance to absorb light in the range of not longer than 380 nm which range is commonly called ultraviolet rays. In addition, as metal elements (M′) to be partners of Cu in the above combination, Co, Fe, Bi, Mn, Ag, and Ni are more favorable in point of being able to more enhance the performance to absorb light in the high-energy range (380 to 450 nm) of visible rays. As metal elements (M′) to be partners of Cu in the above combination, Fe, Mn, and Bi are particularly favorable in point of high safety of the metal oxide particle. As to their combinations, for example, a combination including Cu and any of Fe, Bi, and Mn as essential components, a combination comprising Cu and Fe and the aforementioned n-type dopant, a combination comprising Cu and Mn and the aforementioned n-type dopant, a combination comprising Cu and Bi and the aforementioned n-type dopant, a combination comprising Cu and Bi and either Fe or Mn, or a combination comprising Cu and Bi and either Fe or Mn and the aforementioned n-type dopant can be cited as a particularly favorable mode.

(iii) A combination including Fe, Co, Ni, and Mn as essential components. By this inclusion of Fe, Co, Ni, and Mn as essential components, these Fe, Co, Ni, and Mn can more enhance the performance to absorb light in the high-energy range (380 to 450 nm) of visible rays. Particularly, as is aforementioned, the cases where Fe(II) and/or Mn(II) is essential to at least a part of Fe, Co, Ni, and Mn (in other words, cases where Fe(II) exists alone or where Mn(II) exists alone, or cases where Fe(II) and Fe(III) coexist or cases where Mn(II) and Mn(III) coexist) are more favorable in that: the visible-ray absorption can be prevented, and ultraviolet rays can be absorbed up to a longer wavelength. As metal elements (M′) to be partners of Fe, Co, Ni, and Mn in the above combination, the aforementioned n-type dopants (above all, Ce, In, Al, Sn) and/or Cu are favorable (these enhance the performance to absorb light in the range of not longer than 380 nm which range is commonly called ultraviolet rays).

(iv) A combination including Bi as an essential component. This inclusion of Bi as an essential component can maintain the transmittance of visible rays of not shorter than 450 nm and at the same time enhance selectively the absorbance in the shorter-wavelength range (400 to 450 nm) than the above. As metal elements (M′) to be partners of Bi in the above combination, (1) the aforementioned n-type dopants (above all, In, Al, Ce, Sn), (2) Cu, (3) Co, Mn, Ni, and Fe, or (4) Ag are favorable.

(v) A combination including Ag as an essential component. This inclusion of Ag as an essential component can more enhance the performance to absorb light in the high-energy range (380 to 450 nm, particularly 410 to 440 nm) of visible rays. As metal elements (M′) to be partners of Ag in the above combination, the aforementioned n-type dopants (above all, In, Al, Sn, Ce) and Cu are favorable (these are effective to enhance the absorption ability at 380 nm).

As to the aforementioned second metal oxide particle, specific more favorable examples of the combination of the at least two being selected as the aforementioned metal elements (M′) include a combination of Co(II) and In(III), a combination of Co(II) and Bi(III), a combination of Fe(II) and In(III), a combination of Fe(II) and Bi(III), a combination of Co(III) and In(III), a combination of Co(III) and Bi(II), a combination of Co(II) and Al(III), a combination of Co(III) and Ga(III), a combination of Co(II) and Ti(IV), a combination of Fe(III) and In(III), a combination of Fe(III) and Bi(III), a combination of Fe(II) and Al(III), a combination of Fe(III) and Cu(II), a combination of Fe(II), Cu(I), and In(III), a combination of Fe(II), Cu(II), and Al(III), a combination of Fe(III), Cu(I), and In(III), a combination of Fe(III), Cu(II), and Al(III), a combination of Fe(II), Fe(III), and Al(III), a combination of Fe(II), Fe(III), and Cu(I), a combination of Cu(I) and In(III), a combination of Cu(II) and In(III), a combination of Cu(I) and Al(III), a combination of Cu(II) and Al(III), a combination of Cu(I) and Sn(IV), a combination of Cu(II) and Sn(IV), a combination of Cu(I) and Ti(IV), a combination of Cu(II) and Ti(III), a combination of Cu(I) and Ce(IV), a combination of Cu(I) and Co(III), a combination of Cu(II) and Co(III), a combination of Cu(I) and Bi(III), a combination of Cu(II) and Bi(III), a combination of Cu(I), Cu(II), and Bi(III), a combination of Cu(I), Cu(II), and In(III), a combination of Cu(I), Cu(II), and Al(III), a combination of Cu(I), Cu(II), and Sn(IV), a combination of Cu(I), Cu(II), and Sn(II), a combination of Cu(I), Cu(II), and Ti(IV), a combination of Cu(I), Cu(II), and Ti(III), a combination of Mn(II) and Bi(III), a combination of Ag(I) and Bi(III), a combination of Ni(II) and Bi(III), a combination of Cu(I), In(III), and Bi(III), a combination of Cu(I), Al(III), and Mn(II), a combination of Cu(I), Ga(III), and Fe(II), a combination of Cu(I), In(III), Bi(III), and Fe(II), a combination of Cu(I), In(III), Bi(III), and Mn(II), a combination of Sn(II) and In(III), a combination of Sn(IV) and Al(III), a combination of Ce(III) and Ga(III), a combination of Ce(IV) and Ti(III), a combination of Ag(I) and In(III), a combination of Ag(0) and In(III), a combination of Ag(I) and Cu(I), a combination of Sn(II) and Ce(III), a combination of Sn(IV) and Ti(IV), a combination of Mn(III) and In(III), a combination of Mn(II) and Al(III), and a combination of Mn(II), Cu(I), and In(III).

In the aforementioned second metal oxide particle, the total content of the aforementioned metal elements (M′) is favorably in the range of 0.02 to 20 atomic %, more favorably 0.2 to 10 atomic %, relative to the metal element (M). If the above content is lower than 0.02 atomic %, then there is a possibility that the ultraviolet interception performance may be insufficient. If the above content is higher than 20 atomic %, then there is a possibility that the visible-ray transmittability may be low.

As to the aforementioned second metal oxide particle, in cases where the aforementioned metal elements (M′) are selected in the aforementioned combination (i), the total content of the at least one metal element selected from the group consisting of Co, Cu, and Fe is favorably in the range of 0.01 to 20 atomic %, more favorably 0.1 to 5 atomic %, still more favorably 0.2 to 3 atomic %, relative to the metal element (M). If the above content is lower than 0.01 atomic %, then there is a possibility that the ultraviolet interception performance may be insufficient. If the above content is higher than 20 atomic %, then there is a possibility that the visible-ray transmittability may be low. On the other hand, in cases where the aforementioned metal elements (M′) are selected in the aforementioned combination (i), the total content of the at least one metal element selected from the group consisting of Bi, In, Al, Ga, Ti, Ce, and Sn is favorably in the range of 0.01 to 10 atomic %, more favorably 0.1 to 5 atomic %, still more favorably 0.2 to 3 atomic %, relative to the metal element (M). If the above content is lower than 0.01 atomic %, then there is a possibility that the synergistic effects due to the combination with the group consisting of Co, Cu, and Fe may not sufficiently be exercised. If the above content is higher than 10 atomic %, then there are cases where the effect of enhancing the ultraviolet intercepting ability is rather difficult to take and is rather low, and besides, in cases where Bi is contained, there is a possibility that there may occur problems of coloring (yellowing).

As to the aforementioned second metal oxide particle, in cases where the aforementioned metal elements (M′) are selected in the aforementioned combination (ii), the content of Cu is favorably in the range of 0.01 to 10 atomic %, more favorably 0.1 to 5 atomic %, still more favorably 0.1 to 1 atomic %, relative to the metal element (M). In addition, the content of the metal elements (M′) other than Cu (metal elements (M′) to be partners of Cu) in the aforementioned combination (ii) is favorably in the range of 0.01 to 10 atomic %, more favorably 0.1 to 5 atomic %, still more favorably 0.2 to 3 atomic %, relative to the metal element (M).

As to the aforementioned second metal oxide particle, in cases where the aforementioned metal elements (M′) are selected in the aforementioned combination (iii), the content of Fe, Co, and Ni is favorably in the range of 0.01 to 10 atomic %, more favorably 0.1 to 5 atomic %, relative to the metal element (M). In cases where the aforementioned metal elements (M′) are selected in the aforementioned combination (iii), the content of Mn is favorably in the range of 0.01 to 30 atomic %, more favorably 1 to 20 atomic %, still more favorably 1 to 10 atomic %, relative to the metal element (M).

In addition, the content of the metal elements (M′) other than Fe, Co, Ni, and Mn (metal elements (M′) to be partners of Fe, Co, Ni, and Mn) in the aforementioned combination (iii) is favorably in the range of 0.01 to 10 atomic %, more favorably 0.1 to 5 atomic %, still more favorably 0.2 to 3 atomic %, relative to the metal element (M).

In cases where the aforementioned metal elements (M′) are selected in the aforementioned combination (iv), the content of Bi is favorably in the range of 0.01 to 10 atomic %, more favorably 0.1 to 5 atomic %, relative to the metal element (M). In addition, the content of the metal elements (M′) other than Bi (metal elements (M′) to be partners of Bi) in the aforementioned combination (iv) is favorably in the range of 0.01 to 10 atomic %, more favorably 0.1 to 5 atomic %, still more favorably 0.2 to 3 atomic %, relative to the metal element (M).

In cases where the aforementioned metal elements (M′) are selected in the aforementioned combination (v), the content of Ag is favorably in the range of 0.01 to 10 atomic %, more favorably 0.05 to 2 atomic %, still more favorably 0.1 to 1 atomic %, relative to the metal element (M).

As to the aforementioned second metal oxide particle, it is permissible to cause another metal element besides the aforementioned metal elements (M′) to be contained in the oxide of the metal element (M) within the range not damaging the effects (provided by the metal elements (M′)) of the present invention. Examples of such a metal element other than the metal elements (M′) include B, Si, Ge, Sb, Hf, Y lanthanoid metal elements, alkaline metal elements, and alkaline earth metal elements.

(Third Invention):

The third metal oxide particle according to the present invention (the third invention) is a metal oxide particle which is characterized in that: the metal element (M′) is at least one member selected from the group consisting of Co, Fe, and Ni; and in either i) that: the metal element (M) is at least one member selected from the group consisting of Zn, Ti, Ce, In, Sn, Al, and Si; and at least a part of Co, Fe, and Ni as the metal element (M′) is 2 in valence (the particle according to such a mode i) may hereinafter be referred to as “third metal oxide particle A”); or ii) that: the metal element (M) is Zn; and the metal oxide particle is not larger than 30 nm in crystal grain diameter in the vertical direction to the (002) plane and not smaller than 8 nm in crystal grain diameter in the vertical direction to the (100) plane (the particle according to such a mode ii) may hereinafter be referred to as “third metal oxide particle B”).

In the aforementioned third metal oxide particle, the aforementioned metal element (M) and its oxide are as aforementioned.

As to Co, Fe, and Ni as the aforementioned metal element (M′) in the aforementioned third metal oxide particle, there can be applied thereto the same descriptions as aforementioned about the metal element (M′) in the second metal oxide particle in the section hereof headed “(Second invention)”.

As to the aforementioned third metal oxide particle A, at least a part of Co, Fe, and Ni as the metal element (M′) is 2 in valence. In other words, at least one of Co to which Co(II) is essential, Fe to which Fe(II) is essential, and Ni to which Ni(II) is essential is contained as the aforementioned metal element (M′).

Co to which Co(II) is essential may include Co(II) only or may include Co of another valence (e.g. Co(III)) besides Co(II), thus there being no limitation. However, favorable in point of the excellence in the ultraviolet intercepting ability (ultraviolet absorbency) is the former or the nearer to its composition (specifically, Co having a Co(II) content of not lower than 50 atomic %, favorably not lower than 70 atomic %, more favorably not lower than 90 atomic %, relative to the entire Co). Hereinafter, unless otherwise noted, the simple wording “Co(II)” is defined as referring to “Co to which Co(II) is essential”.

Similarly, also as to Fe to which Fe(II) is essential and Ni to which Ni(II) is essential, they may include Fe(II) only and Ni(II) only respectively, or may include Fe and Ni respectively of another valence (e.g. Fe(III) and Ni(III) respectively) besides Fe(II) and Ni(II) respectively, thus there being no limitation. However, favorable in point of the excellence in the ultraviolet intercepting ability (ultraviolet absorbency) is the former or the nearer to its composition (specifically, Fe or Ni having a Fe(II) or Ni(II) content of not lower than 50 atomic %, favorably not lower than 70 atomic %, more favorably not lower than 90 atomic %, relative to the entire Fe or Ni). Hereinafter, unless otherwise noted, the simple wording “Fe(II)” is defined as referring to “Fe to which Fe(II) is essential”, and the simple wording “Ni(II)” is defined as referring to “Ni to which Ni(II) is essential”.

Examples of favorable modes of the metal element (M′) in the aforementioned third metal oxide particle A include: Co(II) only; Ni(II) only; Fe(II) only; mingled states of at least two of Co(II), Ni(II), and Fe(II); mingled states of Co(II) and Co of a valence of other than 2 (favorably a valence of 3); mingled states of Fe(II) and Fe of a valence of other than 2 (favorably a valence of 3); mingled states of Ni(II) and Ni of a valence of other than 2 (favorably a valence of 3); mingled states of Co(II) and another metal element (Fe or Ni); mingled states of Fe(II) and another metal element (Co or Ni); mingled states of Ni(II) and another metal element (Co or Fe).

Among these, the combinations of Co(II) and Fe (its valence is favorably 2 though not especially limited) are particularly favorable modes. Prior Co-containing metal oxide particles are great in degree of coloring (bluing) due to Co. Therefore, in uses which demand to be more colorless transparent, those prior particles have a possibility of being difficult to use and lacking the utility. In addition, there is also a fact that the aforementioned prior metal oxide particles are lower in performance of absorbing ultraviolet rays of not longer than 370 nm in wavelength than the zinc oxide particle. Therefore, in uses which demand severer ultraviolet absorbency, the aforementioned prior metal oxide particles are lacking in the usefulness as the case may be. By using Co(II) and Fe in combination, these problems can be solved as follows. As to the aforementioned coloring due to Co, even if it occurs somewhat it can be suppressed to such a soft hue as to be sufficiently usable in uses which demand to be more colorless transparent, and further the performance of absorbing ultraviolet rays of not longer than 370 nm in wavelength can additionally be enhanced. On the other hand, hitherto, also in cases where, for example, only Fe of 3 in valence is caused to be contained as a hetero-metal element, then there has been the problem of coloring (dark-browning). However, by using Fe in combination with Co(II), there is also a side such that: the above problem of coloring can effectively be alleviated by the coloring (bluing) due to Co(II), thus resulting in obtaining a metal oxide particle which has sufficiently no problem also in point of the utility.

In addition, particularly in the fields where the safety is strongly demanded in use aspects, it is preferable for the metal element (M′) to include Fe(II) and/or Fe(III) as a main component rather than to include Co and/or Ni as a main component because the Co and Ni are strongly toxic. On the other hand, taking into consideration the present circumstances where compounds used as raw materials for the Fe(III) are generally inexpensive but where Fe(II) compounds (e.g. iron(II) acetate) which are compounds used as raw materials for the Fe(II) are expensive, then, as to Fe, the raw materials for Fe of 3 in valence are preferable in point of costs. Particles containing Fe(III) have a demerit of being colored yellow. However, in cases where the coloring is problematic, the yellowing caused by containing Fe(III) can be reduced if Fe(III) is made to be contained in joint use with at least one trace component selected from among Fe(II), Co(II), and Ni(II). In addition, the joint use of Fe(III) with the aforementioned trace component can also enhance the ultraviolet interception performance. In the fields where the safety is strongly demanded, the content (in terms of atomic ratio to the metal element (M)) of the metal element (M′) in the metal oxide particle is as follows: as to Fe, its content is favorably in the range of 1 to 10 atomic % in total of Fe of 2 and 3 in valence; and, as to Co and Ni, the content of each of them is favorably in the range of 0.01 to 1 atomic %, more favorably lower than 0.1 atomic %.

As to the aforementioned third metal oxide particle A, the total content of the metal element (M′) is favorably in the range of 0.1 to 10 atomic %, more favorably 0.2 to 5 atomic %, still more favorably 0.5 to 3 atomic %, relative to the metal element (M). If the above content is lower than 0.1 atomic %, then there is a possibility that the absorption performance in the ultraviolet long-wavelength range (which is an effect by containing Co(II), Fe(II), and/or Ni(II)) may be insufficient. If the above content is higher than 10 atomic %, then there is a possibility that: the absorption performance in the absorption wavelength range (of ultraviolet rays of not longer than 370 nm) inherently possessed by the oxide of the metal element (M) may be greatly low.

As to the aforementioned third metal oxide particle B, the aforementioned metal element (M) is Zn, and this metal oxide particle is a crystal structure comprising a zinc-oxide-containing metal oxide crystal. As to the sizes of the crystal grain, constituting this crystal structure, in specific directions (i.e. the size (Ds (002)) of the crystal grain diameter in the vertical direction to the (002) plane and the size (Ds (100)) of the crystal grain diameter in the vertical direction to the (100) plane), the Ds (002) is not larger than 30 μm, and the Ds (100) is not smaller than 8 nm. The above Ds (100) is favorably not smaller than 10 nm. If the above Ds (100) is smaller than 8 nm, then there is a possibility that there may occur a blue shift in ultraviolet absorption wavelength. If the above Ds (002) is larger than 30 nm, then there is a possibility that the transparency may be low. Incidentally, the above size of the crystal grain diameter is defined as values measured by methods as stated in the below-mentioned detailed description of Examples of some preferred embodiments.

As to the aforementioned third metal oxide particle (particularly, metal oxide particle B), its primary particle is favorably a single crystal structure (crystal structure comprising one crystal grain). Whether it is a single crystal structure or a polycrystalline structure can be confirmed by observation with a TEM.

Incidentally, also as to the third metal oxide particle B, at least a part of Co, Fe, and Ni as the metal element (M′) is favorably 2 in valence, and the aforementioned descriptions about the metal element (M′) can appropriately be applied thereto.

As to the aforementioned third metal oxide particle, it is also possible to cause the aforementioned oxide of the metal element (M) to contain another metal element besides the aforementioned hetero-metal element (M′) within the range not damaging the effects of the present invention. Such a metal element other than the hetero-metal element (M′) is not especially limited. However, for, example, it is favorable that such as is not contained as the aforementioned metal element (M) is selected from among such as Al, In, Sn, Mn, Ce, alkaline metal elements, and alkaline earth metal elements.

(Fourth Invention):

The fourth metal oxide particle according to the present invention (the fourth invention) is a metal oxide particle, which is a metal oxide particle comprising an oxide of a metal element (M), with the metal oxide particle being characterized in that: the metal element (M) is at least one member selected from the group consisting of Zn, Ti, Ce, In, Sn, Al, and Si; and in: i) that: at least one member selected from the group consisting of N, S, and group-17 (group-7B) elements, and further an acyl group, are contained in the oxide of the metal element (M) (the particle according to such a mode i) may hereinafter be referred to as “fourth metal oxide particle A”); or ii) that: at least two members selected from the group consisting of N, S, and group-17 (group-7B) elements are contained in the oxide of the metal element (M) (the particle according to such a mode ii) may hereinafter be referred to as “fourth metal oxide particle B”); or iii) that: a component derived from a metal element (M′) other than the metal element (M) is contained in the particle (the particle according to such a mode iii) may hereinafter be referred to as “fourth metal oxide particle C”). By causing the aforementioned hetero-nonmetal elements (N, S, and group-17 (group-7B) elements) to be contained, there can be expected the effect of improving the ultraviolet absorption performance, specifically, the effect such that there is provided the performance to absorb light of wavelengths longer than the light absorption edge inherently possessed by the oxide of the metal element (M).

In the aforementioned fourth metal oxide particle, the aforementioned metal element (M) and its oxide are as aforementioned. Particularly, a favorable mode of the fourth metal oxide particle is a solid solution in which the aforementioned element (N, S, a group-17 (group-7B) element) is substituted for a part of oxygen atoms in the oxide of the metal element (M). And besides, there can be taken forms of such as the aforementioned oxide nitride, oxide sulfide, and oxide halide.

As the aforementioned hetero-nonmetal elements (N, S, and group-17 (group-7B) elements) in the aforementioned fourth metal oxide particle, nitrogen, fluorine, and sulfur are favorable, and particularly the nitrogen is more favorable in point of being effective in shifting the light absorption edge toward the longer wavelength side.

In the aforementioned fourth metal oxide particle A, at least one of the aforementioned hetero-nonmetal elements, and further an acyl group, are contained in the oxide of the metal element (M). By causing the acyl group to also be contained along with the aforementioned hetero-nonmetal element, there can be obtained an effect such that: the dispersibility is enhanced to such a degree that a transparent membrane can be formed. For example, if nitrogen is caused to be contained, then the particle is colored yellow, so that this yellowing is conspicuous even when the particle is formed into a dispersion membrane. However, by causing the acyl group to be contained, the coloring degree is improved. Incidentally, as is mentioned below, also by bonding an alkoxide or its (partially) hydrolyzed product to surfaces of the aforementioned oxide of the metal element (M) (wherein the alkoxide includes at least one metal element (favorably, at least one member selected from the group consisting of Si, Ti, Al, and Zr) different from the metal element (M′) contained in the oxide), the same effect can be obtained. If such bonding of the alkoxide or its (partially) hydrolyzed product and the containing of the acyl group are combined, then it is possible to exercise a more excellent coloring-reduction effect.

In the aforementioned fourth metal oxide particle B, at least two of the aforementioned hetero-nonmetal elements are contained in the oxide of the metal element (M). The joint use of the at least two hetero-nonmetal elements more enhances the ultraviolet absorption effect. At lease one of the at least two hetero-nonmetal elements is favorably any of nitrogen, fluorine, and sulfur.

In the aforementioned fourth metal oxide particle C, a component derived from the metal element (M′) other than the metal element (M) is contained in the particle. Hereupon, the aforementioned metal element (M′) is not especially limited. This metal element (M′) can be selected at will from among the hetero-metal elements (M′) cited about the aforementioned first, second, and third metal oxide particles. However, the metal element (M′) is favorably at least one metal element which is different from the metal element (M) and selected from the group consisting of Co, Cu, Fe, Bi, In, Al, Ga, Ti, Sn, Ce, Ni, B, Mn, Ag, Au, platinum group metal elements, alkaline metal elements, and alkaline earth metal elements. The details of the metal elements of this group are as aforementioned as the metal elements (M′) in the second metal oxide particle. Particularly, Co, Fe, Bi, Ni, Mn, and Ag are effective in shifting the light absorption edge toward the longer wavelength side, and Co(II), Fe(II), Ni(II), Cu(I), and Cu(II) are effective in reducing the dispersion membrane coloring caused in cases such as where the aforementioned hetero-nonmetal element is N, and In, Al, Ga, Ti, Sn, Ce, and B are effective in enhancing the light absorption coefficient near the band absorption edge of the oxide of the metal element (M).

In the aforementioned fourth metal oxide particles A, B, and C, the content of the aforementioned hetero-nonmetal element (N, S, a group-17 (group-7B) element) is favorably in the range of 0.01 to 20 atomic %, more favorably 0.05 to 10 atomic %, relative to the metal element (M). If this content is lower than 0.01 atomic %, then there are cases where the effect of improving the ultraviolet absorption performance is insufficient. On the other hand, if the above content is higher than 20 atomic %, then there are cases where the absorption ability at shorter than 370 nm is low.

As to any of the first to fourth metal oxide particles according to the present invention, a mode in which, besides the aforementioned hetero-(metal/nonmetal) elements, a component derived from an alkaline metal element and/or an alkaline earth metal element is also contained in the range of 0.001 to 5 atomic % relative to the metal element (M) is favorable in point of more enhancing the ultraviolet absorption performance. The content of the alkaline metal element and/or alkaline earth metal element is more favorably in the range of 0.001 to 1 atomic % relative to the metal element (M).

As to any of the metal oxide particles according to the present invention, the existence of the aforementioned hetero-elements (i.e. the aforementioned hetero-(metal/nonmetal) elements and the other metal elements possible to cause besides the hetero-(metal/nonmetal) elements to be contained in the oxide of the metal element (M)) can be confirmed in the following way: as to primary particles of the metal oxide particles and assemblages of those primary particles, while their transmission images are observed with an FE-TEM (field emission transmission electron microscope), a place where no metal segregate but the particles is seen is sought, and then this place is subjected to elemental analysis with a high resolution XMA to thus detect a peak assigned to each element. As to the confirmation of the segregate, if the segregate is on a level usually impossible to confirm directly from the transmission images (observed with the FE-TEM) or in their combination with the XMA, then the segregate is regarded as absent.

It may be possible that the measurement of the content of the aforementioned hetero-elements (i.e. the aforementioned hetero-(metal/nonmetal) elements and the other metal elements possible to cause besides the hetero-(metal/nonmetal) elements to be contained in the oxide of the metal element (M)) is carried out by trace analysis methods such as fluorescent X-ray analysis, atomic absorption spectrometry, and ICP (Inductively Coupled Plasma Atomic Emission Spectroscopy). However, the above measurement is favorably carried out by a method in which: the above elemental analysis with the high resolution XMA is carried out with a desired space resolution (spot diameter) to thus measure the intensity of a peak assigned to each metal element, and then, from its result, the above content is calculated. As to the above spot diameter, its lower limit can be set to as low as 1 nmφ by narrowing the probe down, and also the above spot diameter can be freely and continuously enlarged. Specifically, in the transmission images observed with the FE-TEM, usually an assemblage of about 10 metal oxide particles such that there is seen no segregate is selected to carry out the elemental analysis with such a space resolution (spot diameter) as encompasses all these about 10 (fine) particles.

Incidentally, as to whether each particle contains the hetero-elements (i.e. the aforementioned hetero-(metal/nonmetal) elements and the other metal elements possible to cause besides the hetero-(metal/nonmetal) elements to be contained in the oxide of the metal element (M)) or not or as to whether the hetero-element is uniformly dispersed in each particle or not, it can be confirmed by narrowing the beam diameter down (e.g. to 1 nmφ) to thus carry out local elemental analysis.

As the aforementioned FE-TEM, for example, there can be used such as a field emission transmission electron microscope (HF-2000 model, acceleration voltage 200 kV) produced by Hitachi Co., Ltd. As the aforementioned high resolution XMA, for example, there can be used such as an X-ray microanalyzer (Sigma model, energy dispersion type, beam diameter: space resolution 10 Åφ) produced by Kevex.

The sizes of the first to fourth metal oxide particles according to the present invention are as follows. As is aforementioned, as to the first metal oxide particle, it is important that this particle is a fine particle, namely, a particle of which the average particle diameter in terms of primary particle diameter is not larger than 0.1 μm. Also as to the second to fourth metal oxide particles, they include all down to what are called superfine particles and fine particles, and thus there is no limitation. Usually, the average particle diameter of the primary particle is favorably in the range of 1 to 100 nm.

As to any of the first to fourth metal oxide particles according to the present invention, a favorable mode is that the primary particle diameter is in the range of 3 to 50 nm. In cases of the first metal oxide particle, the primary particle diameter is more favorably in the range of 3 to 30 nm, still more favorably 5 to 20 nm. In cases of the second to fourth metal oxide particles, the primary particle diameter is more favorably in the range of 5 to 30 nm, still more favorably 5 to 20 nm, particularly favorably 10 to 20 nm. If the average particle diameter of the primary particle is larger 100 nm, then there is a possibility that the transparency may be low. If the average particle diameter of the aforementioned primary particle is too small, then the ultraviolet absorption edge tends to shift toward the shorter wavelength side due to the quantum effect, so the metal oxide particle is unfavorable as an ultraviolet absorbent material. On the other hand, the average particle diameter of the aforementioned primary particle is too large, then there is a possibility that the transparency may be low.

Incidentally, in the present invention, the average particle diameter of the primary particle refers to the crystal grain diameter (Dw) or the specific surface area diameter (Ds). In detail, in cases where the aforementioned particle is the crystal, the crystal grain diameter (Dw) is referred to and, in cases where the aforementioned particle is the noncrystal, the specific surface area diameter (Ds) is referred to. Accordingly, it is favorable that either one of the crystal grain diameter (Dw) and the specific surface area diameter (Ds) is in the aforementioned range. In more detail, the crystal grain diameter (Dw) is applied to cases of X-ray-diffraction-crystallographically crystals and refers to the size of the crystal grain determined by Scherrer equation. As to this crystal grain diameter (Dw), usually, it is possible that: a powder X-ray diffraction pattern of the metal oxide particle is measured, and then, as to three intense rays thereof (the largest peak (1) of diffracted rays, the second largest peak (2) of diffracted rays, and the third largest peak (3) of diffracted rays), the crystal grain diameters D1, D2, and D3 in the vertical directions to the diffraction lattice planes assigned to the diffracted rays (1) to (3) respectively are determined from their respective full widths of half maximum intensity or integral widths in accordance with Scherrer equation, and then their average value ((D1+D2+D3)/3) is calculated as the crystal grain diameter (Dw). On the other hand, the specific surface area diameter (Ds) can be calculated in accordance with the following equation after the true specific gravity of a powder of the metal oxide particle and the specific surface area of this powder have been measured.
Ds(nm)=6000/(ρ×S)
wherein ρ: true specific gravity (no dimension) of particle
S: specific surface area (m²/g), measured by B.E.T. method, of particle

As to the first to fourth metal oxide particles according to the present invention, it is favorable that: the aforementioned oxide of the metal element (M) is a crystal; and the metal oxide particle is not larger than 30 nm in crystal grain diameter (Dw) (average value of values calculated in accordance with Scherrer equation as to three intense rays of XRD peaks).

Particularly, as to the first to fourth metal oxide particles according to the present invention, in cases where the oxide of the metal element (M) is a zinc oxide crystal, it is favorable in point of the excellence both in the transparency and the ultraviolet absorption performance that the metal oxide particle is not larger than 30 nm in crystal grain diameter in the vertical direction to the lattice plane (002) and not smaller than 8 nm in crystal grain diameter in the vertical direction to the lattice plane (100) and/or lattice plane (110) among crystal grain diameters by X-ray diffractometry. More favorably, the metal oxide particle is not larger than 20 nm in crystal grain diameter in the vertical direction to the lattice plane (002) and not smaller than 10 nm in crystal grain diameter in the vertical direction to the lattice plane (100) and/or lattice plane (110). Specifically, the crystal grain diameter in the vertical direction to the lattice plane (002) (optic axial direction) does not exercise a very great influence on the ultraviolet absorption performance and, as to this crystal grain diameter, the smaller the more favorable in point of enhancing the transparency. On the other hand, as to a crystal grain diameter in the direction of the lattice plane (002) (vertical direction to the optic axis), for example, the crystal grain diameter in the vertical direction to the lattice plane (100) and/or lattice plane (110), if it is too small, then it results in deteriorating the ultraviolet absorption performance. Incidentally, the crystal grain diameters in the vertical directions to the lattice planes can be determined by carrying out the powder X-ray diffractometry and then carrying out Scherrer analysis.

As to the first to fourth metal oxide particles (particularly, first metal oxide particle) according to the present invention, it is favorable, for enhancing the transparency of the resultant paint film or resin composite, that these metal oxide particles are not larger than 500 nm in dispersion particle diameter in a state dispersed in any solvent or resin. The dispersion particle diameter is more favorably not larger than 200 nm and still more favorably not larger than 100 nm, and particularly desirably such that the primary particle can be dispersed in a monodispersed state or a state near it, and most favorably not larger than 50 nm. Incidentally, the dispersion particle diameter can be measured, for example, with a dynamic light scattering type particle diameter distribution measurement device (e.g. “LB-500” produced by Horiba Seisakusho).

The optical performances of the metal oxide particle according to the present invention can be evaluated in a way that the performance of interception of light in the range of ultraviolet rays (ultraviolet rays of not longer than 380 nm and visible rays of not longer than 450 nm) (ultraviolet intercepting performance) and the performance of transmission of visible rays (450 to 780 nm) (visible-ray transmission performance) are used as indexes. It is favorable for the ultraviolet absorbing functional material to be high in the ultraviolet intercepting performance and the visible-ray transmission performance. Usually, the ultraviolet intercepting performance and the visible-ray transmission performance can be judged by evaluating the spectroscopic transmittance properties in a state of the particles alone, a state of a membrane formed from the below-mentioned composition for membrane formation, or a state where the particles are dispersed in a dispersion medium such as solvent. In detail, the ultraviolet intercepting performance is judged by evaluating the transmittance at any wavelength in the ultraviolet range (e.g. 380 nm, 400 nm, 420 nm) as a representative value or by evaluating the average transmittance at not longer than 450 nm or not longer than 380 nm. On the other hand, the visible-ray transmission performance is judged by evaluating the transmittance at any wavelength in the visible-ray range (e.g. 500 nm, 600 nm, 700 nm) as a representative value or by evaluating the average transmittance in the range of 450 to 780 nm or 380 to 780 nm. The values of these transmittances can be obtained by measurement of transmittances inclusive of parallel-ray transmission light and diffusion transmission light at each wavelength and, for example, can be measured with a spectrophotometer having an integrating sphere. However, in cases where the sample is so high in transparency that the diffusion transmission light can be ignored substantially (specifically, cases where the sample is less than 5% in haze), then only the parallel-ray transmittance may be used as the measured value.

In a favorable mode of the metal oxide particles according to the present invention, for example, when they are dispersed in an organic solvent in a particle concentration of 0.1 wt %, then the transmittance at 380 nm is favorably not more than 10%, more favorably not more than 5%, and the transmittance at 400 nm is favorably not more than 50%, more favorably not more than 20%, and the transmittance at 600 nm is favorably not less than 70%, more favorably not less than 80%, In addition, the transmittance at 420 nm is favorably not more than 50%.

In addition, the metal oxide particle according to the present invention is used without being limited to uses for the purpose of the ultraviolet interception, and is favorably high in transparency when having been formed into a membrane. Specifically, its haze is favorably not more than 10%, more favorably not more than 2%, still more favorably not more than 1%.

Incidentally, as a method for measuring the light absorption properties (including the ultraviolet interception property) of the particle, there can also be adopted a method in which the diffused reflectance of a powder of the particle is measured. In its case, if the reflectance is low, then the absorbance is high and, on the other hand, if the reflectance is high, then the absorbance is low. Therefore it is favorable that: the reflectance in the ultraviolet range as referred to in the present invention is low, and the reflectance in the visible-ray range of not shorter than 450 nm is high.

As to the metal oxide particle according to the present invention, favorably in point of the excellence in the performance to transmit visible rays and selectively absorb ultraviolet rays only, this particle is a particle such that, when this particle is formed into a membrane comprising this particle and/or a metal element (M) oxide crystal, derived from this particle, as an essential constitutional component, then the optical properties of the resultant membrane satisfy the following conditions. Hereupon, as to such as the form of the above membrane and its formation process, the below-mentioned description about the membrane according to the present invention is similarly applicable thereto. Incidentally, the following optical properties of the above membrane are defined as values measured and evaluated by methods as stated in the below-mentioned detailed description of Examples of some preferred embodiments. In addition, they are defined as physical properties of only the membrane portion (excluding the substrate) and as being evaluated with consideration given to optical properties of the membrane-coated substrate and optical properties of only the substrate. In cases where the metal oxide particle according to the present invention contains Co(II) as a metal element, then, furthermore, among the optical properties of the membrane, the transmittance (%) of light of 380 nm in wavelength which is an index of the ultraviolet absorption performance is defined as T³⁸⁰, and further, the transmittance (%) of light of 500 nm in wavelength which is an index of the visible-ray transmission performance is defined as T⁵⁰⁰, and the minimum value of the transmittances (%) of light of 550 to 700 nm in wavelength is defined as T¹, and the absolute value of the difference between T¹ and T⁵⁰⁰ (|T¹−T⁵⁰⁰|) is defined as ΔT.

As to the metal oxide particle according to the present invention, if its amount of coating (amount of use) to the substrate per unit area is varied in cases where this particle is formed into the membrane in the above way, then, accompanying this variation, the values of the T³⁸⁰, T⁵⁰⁰, and ΔT of the resultant membrane also vary. Thus, as to the optical properties of the resultant membrane, they are defined as being evaluated by the values of the T⁵⁰⁰ and ΔT when the value of the T³⁸⁰ is taken as the standard. Favorable modes of the above membrane are shown below in classification into the following cases: cases (a) of the Co(II)-containing metal oxide particle; and cases (b) of the other metal oxide particles.

Cases (a) of Co(II)-containing metal oxide particle:

(i) When the above membrane is formed in a way for the T³⁸⁰ not to be more than 40%, then the ΔT is favorably not more than 10%, and it is more favorable that: the ΔT is not more than 10%, and the T⁵⁰⁰ is not less than 90%; and it is still more favorable that: the ΔT is not more than 5%, and the T⁵⁰⁰ is not less than 95%.

Favorably, (ii) when the above membrane is formed in a way for the T³⁸⁰ not to be more than 30%, then the ΔT is favorably not more than 10%, and it is more favorable that: the ΔT is not more than 10%, and the T⁵⁰⁰ is not less than 90%; and it is still more favorable that: the ΔT is not more than 10%, and the T⁵⁰⁰ is not less than 95%; and it is particularly favorable that: the ΔT is not more than 5%, and the T⁵⁰⁰ is not less than 95%.

More favorably, (iii) when the above membrane is formed in a way for the T³⁸⁰ not to be more than 20%, then the ΔT is favorably less than 10%, and it is more favorable that: the ΔT is less than 10%, and the T⁵⁰⁰ is not less than 80%; and it is still more favorable that: the ΔT is not more than 5%, and the T⁵⁰⁰ is not less than 85%; and it is particularly favorable that: the ΔT is not more than 5%, and the T⁵⁰⁰ is not less than 90%.

Particularly favorably, (iv) when the above membrane is formed in a way for the T³⁸⁰ not to be more than 10%, then the ΔT is favorably less than 10%, and it is more favorable that: the ΔT is less than 10%, and the T⁵⁰⁰ is not less than 80%; and it is still more favorable that: the ΔT is not more than 5%, and the T⁵⁰⁰ is not less than 85%; and it is particularly favorable that: the ΔT is not more than 5%, and the T⁵⁰⁰ is not less than 90%.

Cases (b) of metal oxide particles other than aforementioned:

(i) When the above membrane is formed in a way for the T³⁸⁰ not to be more than 20%, then the T⁵⁰⁰ is favorably not less than 90%, more favorably not less than 90%.

Favorably, (ii) when the above membrane is formed in a way for the T³⁸⁰ not to be more than 10% (more favorably for the T³⁸⁰ not to be more than 5%), then the T⁵⁰⁰ is favorably not less than 70%, more favorably not less than 80%.

As to the first to fourth metal oxide particles according to the present invention, it is favorable that the particle comprising the oxide of the metal element (M) contains an acyl group of 0.1 to 14 mol % in molar ratio to the metal element (M). Its reason is that such a metal oxide particle is excellent in the dispersibility and gives a composition for formation of a membrane excellent in the transparency and such a membrane. Particularly, in cases where the metal element (M) is Zn, Ti, Ce, In, or Sn, then the refractive index of the crystal is so high that the scattering of visible rays tends to occur, and therefore, when the metal oxide particle is dispersed into a binder such as resin, a high-haze membrane tends to be formed. However, the aforementioned particle containing the acyl group would form a low-haze membrane excellent in the transparency. The aforementioned acyl group particularly favorably has 1 to 3 carbon atoms.

As to the first to fourth metal oxide particles according to the present invention, it is favorable that the oxide of the metal element (M) is an oxide to surfaces of which there is bonded an alkoxide or its (partially) hydrolyzed product wherein the alkoxide includes at least one metal element different from the metal element (M′) contained in the oxide. Its reason is the same as the above reason why it is favorable that the metal oxide particle is the particle in which the acyl group iscontained. Namely, such a particle is favorable in points of being excellent in the dispersibility and giving a composition for formation of a membrane excellent in the transparency and such a membrane. Particularly, in cases where the metal element (M) is Zn, Ti, Ce, In, or Sn, then this mode is effective. The aforementioned at least one metal element different from the metal element (M′) contained in the oxide of the metal element (M) is favorably selected from the group consisting of Si, Ti, Al, and Zr. For forming the metal oxide particle into the particle such that, to surfaces of the oxide of the metal element (M), there is bonded an alkoxide or its (partially) hydrolyzed product wherein the alkoxide includes at least one metal element (favorably, at least one metal element selected from the group consisting of Si, Ti, Al, and Zr) different from the metal element (M′) contained in the oxide, it will do to surface-treat the metal oxide particle with at least one of the following metal compounds (1) to (3).

Hereinafter, descriptions are given about the above metal compounds (1) to (3).

Metal compounds (1): metal alkoxides including the aforementioned at least one metal element different from the metal element (M′) contained in the oxide of the metal element (M), such as tetramethoxysilane and tetrabutoxysilane.

Metal compounds (2): organic-group-containing metal compounds shown by the following general formula (a). Incidentally, the kinds of the metal elements in these metal compounds are not limited.
Y¹ᵢM¹X¹ⱼ (a)
(wherein: Y¹ is an organic functional group; M¹ is a metal atom; X¹ is a hydrolyzable group; and i and j are integers of 1 to (s−1) and satisfy i+j=s (wherein s is the valence of M¹)).

Examples of the organic-group-containing metal compounds shown by the general formula (a) include the following.

Examples of the organic-group-containing metal compounds in which the M¹ is aluminum include various aluminum-containing coupling agents such as diisopropoxyaluminum ethyl acetoacetate, diisopropoxyaluminum alkyl acetoacetates, diisopropoxyaluminum monomethacrylate, aluminum stearate oxide trimer, and isopropoxyaluminum alkyl acetoacetate mono(dioctyl phosphates).

Examples of the organic-group-containing metal compounds in which the M¹ is silicon include various silane coupling agents such as: vinyl-containing silane coupling agents (e.g. vinyltrimethoxysilane, vinyltriethoxysilane, vinyltris(β-methoxyethoxy)silane, and vinyltriacetoxysilane); amino-containing silane coupling agents (e.g. N-(2-aminoethyl)-3-aminopropylmethyldimethoxysilane, 3-N-phenyl-γ-aminopropyltrimethoxysilane, and N,N′-bis[3-(trimethoxysilyl)propyl]ethylenediamine); epoxy-containing silane coupling agents (e.g. γ-glycidoxypropyltrimethoxysilane and 2-(3,4-epoxycyclohexyl)ethyltrimethoxysilane); chloro-containing silane coupling agents (e.g. 3-chloropropyltrimethoxysilane); acryloxy-containing silane coupling agents (e.g. acryloxypropyltrimethoxysilane and acryloxypropyltriethoxysilane); methacryloxy-containing silane coupling agents (e.g. 3-methacryloxypropyltrimethoxysilane and 3-methacryloxypropyltriethoxysilane); mercapto-containing silane coupling agents (e.g. 3-mercaptopropyltrimethoxysilane); ketimine type silane coupling agents (e.g. N-(1,3-dimethylbutylidene)-3-(triethoxysilyl)-1-propanamine); cationic silane coupling agents (e.g. N-[2-(vinylbenzylamino)ethyl]-3-aminopropyltrimethoxysilane hydrochloride); alkyl-containing silane coupling agents (e.g. methyltrimethoxysilane, trimethylmethoxysilane, decyltriethoxysilane, and hydroxyethyltrimethoxysilane); silicon compounds having a fluorine-containing organic group (e.g. (tridecafluoro-1,1,2,2-tetrahydrooctyl)triethoxysilane); silane coupling agents having an isocyanate-group-containing organic group (e.g. isocyanatopropyltrimethoxysilane); silane coupling agents shown by the following general formula (b):
R′O(C₂H₄O)ₙC₃H₆Si(OR″)₃ (b)
(wherein: R′ is hydrogen or at least one kind of group (which may have a substituent) selected from among alkyl groups (e.g. methyl group), cycloalkyl groups, aryl groups, acyl groups, and aralkyl groups; R″ is at least one kind of group (which may have a substituent) selected from among alkyl groups (e.g. methyl group), cycloalkyl groups, aryl groups, acyl groups, and aralkyl groups; and n is an integer of not smaller than 1); and
γ-ureidopropyltriethoxysilane and hexamethylenedisilazane.

Examples of the organic-group-containing metal compounds in which the M¹ is zirconium include various zirconium compounds such as zirconium di-n-butoxide (bis-2,4-pentanedionate), zirconium tri-n-butoxide pentanedionate, and zirconium dimethacrylate dibutoxide.

Metal compounds (3): (partially) hydrolyzed products or condensed productsof metal alkoxides (the metals may be any) and of the above (2), which are, for example, shown by the following general formula (c):
R¹—(O-M(-R²ₘ₁)(—R³ₘ₂))ₙ—R⁴ (c)
(wherein: each of R¹ and R² is a hydrogen atom or any one kind of group (which may have a substituent) selected from the group consisting of alkyl groups, cycloalkyl groups, aryl groups, aralkyl groups, and acyl groups; R³ is a hydrolyzable group (the same as X¹ in the above general formula (a)) or hydroxyl group; R⁴ is R² or R³; M is a metal atom; m1 and m2 are (valence of M−2); and n is an integer of 2 to 10,000; incidentally, as to the kinds and numbers (m1 and m2) of R² and R³ bonded to the metal atoms M, they may be the same between all the metal atoms M or may be different between at least a part of the metal atoms M).

For example, as to hydrolyzed-condensed products of the above metal compounds (2), examples thereof include: compounds obtained by a process in which a part or all of the hydrolyzable groups X¹ bonded to the metal atom M1 in the above general formula (a) are hydrolyzed to thus form OH groups; and compounds obtained by a process in which M¹-O-M¹ bonds are formed by condensation reactions (e.g. dehydration condensation) further between the resultant M¹-OH bonds. Specific examples thereof include linear (including those which contain a branch chain) or cyclic hydrolyzed-condensed products (from linear or cyclic trimers up) obtained by hydrolyzing-condensing and/or partially hydrolyzing-condensing the organic-group-containing compounds as herein previously enumerated as the metal compounds (2).

Examples of the metal compounds (3) include: titanium(IV) tetra-n-butoxide tetramer (C₄H₉O—[Ti(OC₄H₉)₂O]₄—C₄H₉, produced by Wako Pure Chemical Industries, Ltd.); silicon(IV) tetramethoxide tetramer; methyltrimethoxysilane tetramer; co-hydrolyzed-condensed products of tetramethoxysilane-methyltrimethoxysilane; and aluminum(III) tributoxide trimer.

The process for production of the metal oxide particle according to the present invention is not limited. Any process is adoptable if it is a publicly known process by which there can be obtained the particle such that the metal oxide particle is caused to contain (is doped with) the desired hetero-metal elements. For example, there is preferred a production process (hereinafter referred to as production process (A)) comprising a step in which: a metal element (M) compound and/or its hydrolyzed-condensed product, hetero-metal element (M′) compounds and/or hetero-nonmetal element compounds, and an alcohol are used as starting materials, and a mixed system of these is put in a high-temperature state to thus form (deposit) a metal oxide particle. In detail, the production process (A) is a process comprising a step in which: the metal element (M) compound and/or its hydrolyzed-condensed product, the hetero-metal element (M′) compounds and/or hetero-nonmetal element compounds, and the alcohol as the starting materials are mixed together and, at the same time as this mixing or thereafter, the resultant mixed system is put in a high-temperature state. By putting the above mixed system in a high-temperature state in this way, the metal oxide particle can be formed in the reaction system.

The metal element (M) compound usable in the production process (A) is not limited. However, a carboxylate of the metal element (M) is favorable. In addition, the hydrolyzed-condensed product of the metal element (M) compound is a hydrolyzed product and/or condensed product obtained by hydrolyzing and/or condensing the zinc compound and encompasses the range of from a monomer compound to a polymer compound (hereinafter the “metal element (M) compound” may refer to “metal element (M) compound and/or its hydrolyzed-condensed product”).

The above carboxylate of the metal element (M) is favorably a compound having in its molecule at least one substituent such that an atom of the metal element (M) is substituted for a hydrogen atom of a carboxyl group. Specific favorable examples thereof include compounds such that, into the aforementioned substituent, there is converted the carboxyl group in carboxylic acid compounds such as: chain carboxylic acids (e.g. saturated monocarboxylic acids, unsaturated monocarboxylic acids, saturated polycarboxylic acids, and unsaturated polycarboxylic acids); cyclic saturated carboxylic acids; aromatic carboxylic acids (e.g. aromatic monocarboxylic acids and aromatic unsaturated polycarboxylic acids); and, in these carboxylic acids, compounds further having, in their molecules, functional groups or atomic groups (e.g. a hydroxyl group, an amino group, a nitro group, alkoxy groups, a sulfone group, a cyano group, and halogen atoms).

More favorable among these carboxylates of the metal element (M) are compounds shown by the following general formula (I):
M(O)_{(m-x-y-z)/2}(OCOR¹)ₓ(OH)_{y}(OR²)_{z} (I)
(wherein: M is an atom of the metal element (M) (at least one member selected from the group consisting of Zn, Ti, Ce, In, Sn, Al, and Si); R¹ is at least one kind selected from among a hydrogen atom, alkyl groups, cycloalkyl groups, aryl groups, and aralkyl groups (wherein these groups may have a substituent); R² is at least one kind selected from among alkyl groups, cycloalkyl groups, aryl groups, and aralkyl groups (wherein these groups may have a substituent); and m, x, y, and z are numbers which satisfy x+y+z≦m, 0<x≦m, 0≦y<m, 0≦z<m (wherein m is the valence of the above M))
(e.g. compounds such that such as a hydroxyl group or an alkoxy group is substituted for a moiety of the above-exemplified carboxylate of the metal element (M)), saturated carboxylates, unsaturated carboxylates, and basic acetates. Still more favorable are the compounds shown by the above general formula (I), and the most favorable are a formate of the metal element (M), an acetate of the metal element (M), and a propionate of the metal element (M), and further, their basic salts.

Incidentally, the carboxylate of the metal element (M) may be a hydrate of a carboxylate containing crystal water, but is favorably an anhydrate.

More detailed descriptions are given below about the compounds shown by the above general formula (I).

As the R¹ and R² in the general formula (I), in point of being easy to obtain a high dispersible metal oxide particle from, hydrogen and alkyl groups having 1 to 4 carbon atoms (e.g. a methyl group) are favorable, and hydrogen, a methyl group, and an ethyl group are particularly favorable. In addition, by the same reason, in the general formula (I), the x favorably satisfies 1≦x≦M, and the y favorably satisfies 0≦y<m/2, and the z favorably satisfies 1≦z<m/2.

As the compounds shown by the general formula (I), those which are fast in dissolution rate are favorable in point of being easy to obtain a high dispersible metal oxide particle from. It may be possible that the dissolution rate is measured directly by the reaction. However, it is defined as a time t needed until a transparent solution is obtained by entire dissolution when, at 25° C., 2 weight parts of the compound shown by the general formula (I) is mixed into 200 weight parts of ion-exchanged water of 25±3° C. (pH 5 to 8) to stir them together. The dissolution rate of the compound shown by the general formula (I) is favorably not more than 2 minutes, more favorably not more than 1 minute, still more favorably not more than 30 seconds.

Among the hydrolyzed-condensed products of the compounds shown by the general formula (I), the condensed product is favorably a compound having a bond chain -(M-O), (wherein n is not smaller than 1) such that the metal element (M) and oxygen (O) are metaloxane-bonded. Though not limited, the condensation degree (on average) of the above condensed product is favorably not more than 100, more favorably not more than 10, in that there can be obtained a metal oxide particle which is uniform in crystal grain size and form.

As to the metal element (M) compounds, only one kind of the above may be used alone, or at least two kinds of the above may be used in combinations with each other.

Though not limited, examples of the hetero-metal element (M′) compounds usable in the production process (A) include metal carboxylates and metal alkoxides. As to the hetero-metal element (M′) compounds, only one kind of the above may be used alone, or at least two kinds of the above may be used in combinations with each other.

As the above hetero-metal element (M′) compounds, there is essentially used a compound of a metal element (M′) (however, in cases of the second metal oxide particle, at least two metal elements (M′)), which is to be caused to be contained as the metal element (M′) in the metal oxide.

The above hetero-nonmetal element compound is not limited. However, for example, in cases where the hetero-nonmetal element is N, favorable examples include nitrogen compounds such as ammonia, ammonia water, urea, and ammonium carbonate. In cases where the hetero-nonmetal element is S, favorable examples include hydrogen sulfide and metal sulfides such as sodium sulfide. In cases where the hetero-nonmetal element is a group-17 element, favorable examples include: acids (e.g. hydrofluoric acid, hydrochloric acid, hydrobromic acid, hydroiodic acid); and fluorides, chlorides, bromides, and iodides of metals (favorably, the metal element (M) or (M′)). As to the hetero-nonmetal element compounds, only one kind of the above may be used alone, or at least two kinds of the above may be used in combinations with each other.

Though not limited, examples of the alcohol usable in the production process (A) include: monohydric alcohols such as aliphatic monohydric alcohols (e.g. methanol, ethanol, isopropyl alcohol, n-butanol, t-butyl alcohol, stearyl alcohol), aliphatic unsaturated monohydric alcohols (e.g. allyl alcohol, crotyl alcohol, propargyl alcohol), alicyclic monohydric alcohols (e.g. cyclopentanol, cyclohexanol), aromatic monohydric alcohols (e.g. benzyl alcohol, cinnamyl alcohol, methylphenylcarbinol), phenols (e.g. ethylphenol, octylphenol, catechol, xylenol, guaiachol, p-cumylphenol, cresol, m-cresol, o-cresol, p-cresol, dodecylphenol, naphthol, nonylphenol, phenol, benzylphenol, p-methoxyethylphenol), and heterocyclic monohydric alcohols (e.g. furfuryl alcohol); glycols such as alkylene glycols (e.g. ethylene glycol, propylene glycol, trimethylene glycol, 1,4-butanediol, 1,5-pentanediol, 1,6-hexanediol, 1,8-octanediol, 1,10-decanediol, pinacol, diethylene glycol, triethylene glycol), alicyclic glycols (e.g. cyclopentane-1,2-diol, cyclohexane-1,2-diol, cyclohexane-1,4-diol), and polyoxyalkylene glycols (e.g. polyethylene glycol, polypropylene glycol); derivatives (e.g. monoethers or monoesters) from the above glycols, such as propylene glycol monomethyl ether, propylene glycol monoethyl ether, dipropylene glycol monomethyl ether, tripropylene glycol monomethyl ether, 3-methyl-3-methoxybutanol, ethylene glycol monoethyl ether, ethylene glycol monobutyl ether, triethylene glycol monomethyl ether, and ethylene glycol monoacetate; and polyhydric alcohols of not less than 3 in functionality such as trihydric alcohols (e.g. glycerol, trimethylolethane), tetrahydric alcohols (e.g. erythritol, pentaerythritol), pentahydric alcohols (e.g. ribitol, xylitol), and hexahydric alcohols (e.g. sorbitol), polyhydric aromatic alcohols such as hydrobenzoin, benzpinacol, and phthalyl alcohol, polyhydric phenols such as dihydric phenols (e.g. catechol, resorcin, hydroquinone) and trihydric phenols (e.g. pyrogallol, phloroglucin), and derivatives such that a part (1 to (n−1) (wherein n is the number of OH groups per molecule)) of OH groups in these polyhydric alcohols are converted into ester bonds or ether bonds. As to the alcohols, only one kind of the above may be used alone, or at least two kinds of the above may be used in combinations with each other.

As the above alcohol, favorable among the above ones are alcohols which easily form the metal oxide particle by reacting with the metal element (M) compound, the hetero-metal element (M′) compound, and/or the hetero-nonmetal element compound. Aliphatic alcohols and high water-soluble alcohols are favorable and, specifically, alcohols having a solubility of not less than 1 weight % relative to water are favorable, and alcohols having a solubility of not less than 10 weight % relative to water are more favorable.

The mutual ratio (formulation ratio) between the metal element (M) compound and the alcohol, which are used as the starting materials, is not limited. However, the ratio of the number of hydroxyl groups (derived from the alcohol) in the alcohol to the number of atoms in terms of metal in the metal element (M) compound is favorably in the range of 0.8 to 1,000, more favorably 0.8 to 100, still more favorably 1 to 50, particularly favorably 1 to 20.

In addition, the mutual ratio (formulation ratio) between the metal element (M) compound and the hetero-metal element (M′) compound or hetero-nonmetal element compound, being used, is not limited. However, this ratio will do if it is set soappropriately that the ratio between the number of atoms in terms of metal in the metal element (M) compound and the number of atoms in terms of metal in the hetero-metal element (M′) compound or number of atoms of the hetero-nonmetal element in the hetero-nonmetal element compound satisfies the aforementioned favorable range of the content of the hetero-metal element (M′) or content of the hetero-nonmetal element. Furthermore, in the second invention, basically, at least two kinds of hetero-metal element (M′) compounds are used. Similarly to the above, the mutual ratio (formulation ratio) between these hetero-metal compounds, being used, also will do if it is adjusted appropriately to such an value as satisfies the aforementioned favorable range.

The aforementioned mixed system of the starting materials is favorably in the form of a flowable liquid such as paste, emulsion, suspension, or solution. If necessary, it may be made the above liquid form by further mixing it with the below-mentioned reaction solvent. In this mixed system, usually, the metal element (M) compound and the hetero-metal element (M′) compound or hetero-nonmetal element compound exist in a state such as a state dispersed in a particulate form, a dissolved state, or a state where a part is dissolved and the rest is dispersed in a particulate form.

In the production process (A), a reaction solvent may further be used. Specifically, when the aforementioned starting materials are mixed together or when the aforementioned mixed system of the starting materials is put in a high-temperature state, such an operation may be carried out after further the reaction solvent has been added thereto.

The amount of the reaction solvent being used is not limited. However, the ratio of the amount of the aforementioned metal element (M) compound being used to the total amount of the aforementioned starting materials and reaction solvent being used is favorably in the range of 0.1 to 50 weight % in that the metal oxide particle can economically be obtained.

As the above reaction solvent, a solvent other than water, in other words, a nonaqueous solvent, is favorable. Examples of the nonaqueous solvent include: hydrocarbons; various halogenated hydrocarbons; alcohols (including also such as phenols, polyhydric alcohols, and hydroxyl-group-containing compounds which are their derivatives); ethers and acetals; ketones and aldehydes; esters such as carboxylate esters and phosphate esters; amides; derivative compounds such that alkyl groups and/or acyl groups are substituted for hydrogen atoms of all hydroxyl groups of polyhydric alcohols; carboxylic acids and their anhydrides; and silicone oils and mineral oils. As the reaction solvent, hydrophilic solvents are particularly favorable. Specifically, the reaction solvent is favorably a solvent which can, at normal temperature (25° C.), contain water in an amount of not smaller than 5 weight % and come in a solution state, more favorably, contain any amount of water and come in a uniform solution state. Favorable examples of the alcohol as the reaction solvent include the same as herein previously enumerated as alcohols which are used as starting materials. As to the reaction solvent, only one kind of the above may be used alone, or at least two kinds of the above may be used in combinations with each other.

In the production process (A), as to the water content of the mixed system of such as the aforementioned starting materials (including also the reaction solvent used if necessary), the lower the more favorable because the defect of the resultant metal oxide particle becomes less. Specifically, it is favorable that the above mixed system has only a slight water content of lower than 4, more favorably lower than 1, particular favorably lower than 0.5, most favorably lower than 0.1, in molar ratio to the metal atoms of the metal element (M) compound used as a starting material. If the above water content is too high, then there is a possibility that it may be so difficult for the hetero-metal elements (M′) or hetero-nonmetal element to be contained in the crystal of the metal oxide that there cannot be obtained the metal oxide particle which can sufficiently exercise the aforementioned effects of the present invention. The above water content can, for example, be measured by Karl Fischer method.

Incidentally, the above water content refers generally to a free-water content. However, in cases where the metal element (M) compound and/or the hetero-metal element (M′) compound or hetero-nonmetal element compound contain crystal water, the water content of this crystal water is also included. In addition, the above water content is a value relating to water contained in such as the starting materials (sum total of water such as: free water in the alcohol and the other reaction solvent component being used; and crystal water in the metal element (M) compound and/or the hetero-metal element (M′) compound or hetero-nonmetal element compound). Water which forms as a by-product in the reaction caused by putting the aforementioned mixed system in a high-temperature state is treated as not being taken into consideration.

In the production process (A), putting the aforementioned mixed system of the starting materials in a high-temperature state is to raise the temperature of this mixed system to not lower than a temperature which is higher than normal temperature and at which the metal oxide particle can be formed. Specifically, though depending on such as the kind of the metal oxide particle to be obtained (e.g. the kinds of the metal element (M) and hetero-metal elements (M′)), the above raised temperature is generally not lower than 50° C. and, in order to obtain a metal oxide particle of high crystallinity, the above, raised temperature is favorably not lower than 80° C., more favorably in the range of 100 to 300° C., still more favorably 100 to 200° C., particularly favorably 120 to 200° C. Incidentally, the above temperature of the mixed system is defined as the bottom temperature of the reactor.

The above high-temperature state of the mixed system is favorably retained at a predetermined temperature for not less than 30 minutes, more favorably not less than 2 hours, in that the crystallinity of the metal oxide particle can be enhanced to thus obtain a metal oxide particle excellent in the physical properties such as ultraviolet intercepting ability.

As to the formed metal oxide particle, if necessary for the purpose of such as removal of residual organic groups or still more promotion of crystal growth, this metal oxide particle may be heated in the range of 300 to 800° C.

When the above mixed system is put in a high-temperature state, a specific temperature-raising means is generally heating with a heater, warm air, or hot air. However, there is no limitation thereto. For example, it is also possible to adopt means such as ultraviolet irradiation.

When the above mixed system is put in a high-temperature state, the operation may be carried out under any pressure of normal pressure, increased pressure, and reduced pressure, thus there being no limitation. However, more favorably, the starting materials are put in a high-temperature state by such as heating under increased pressure. In addition, in cases where the boiling points of such as starting materials and/or reaction solvent (being used jointly therewith) are lower than the reaction temperature at which the metal oxide particle is formed, then it is also favorable to use a pressure-resistant reactor to carry out the reaction. Usually, as to the reaction temperature and as to the gas phase pressure during the reaction, the reaction is carried out at not higher than the critical point of the component used as the solvent. However, it is also possible that the reaction is carried out under supercritical conditions.

In cases where the starting materials are put in a high-temperature state under increased pressure, the pressure (pressure of the gas phase portion) during the heating is not limited. However, this pressure favorably satisfies P>1 kg/cm², more favorably 1.5 kg/cm²≦P≦100 kg/cm², when being shown by absolute pressure P wherein the normal pressure (atmospheric pressure) is defined as 1 kg/cm².

Furthermore, the above pressure particularly favorably satisfies 3 kg/cm²≦P≦20 kg/cm² in that the effects of increasing the pressure are high and that the reaction can be carried out with economical facilities. The method for increasing the pressure is not limited. However, adoptable examples thereof include: a method in which the materials are heated to a temperature higher than the boiling point of the alcohol; and a method in which the gas phase portion is put under increased pressure with an inert gas such as nitrogen gas or argon gas.

In the production process (A), the above high-temperature state of the mixed system will do if, as aforementioned, it is obtained at the same time as the mixing of such as the starting materials or thereafter. In other words, the mixing of such as the starting materials to obtain the above mixed system, and the temperature raising to put this mixed system in a high-temperature state, may be carried out either separately from or at the same time (including also the partly same time) as each other, thus there being no limitation. In detail, the specific means (e.g. heating) for the above temperature raising of the mixed system can be carried out by any method and in any timing regardless of the above mixing of such as the starting materials. For example, at least one of such as the starting materials may be subjected to such as heating in advance before the mixing, thereby raising the temperature of the mixed system at the same time as the mixing. Or the mixed system obtained by the mixing may be subjected to such as heating while this mixing is carried out or after this mixing has been finished, thereby raising the temperature of the mixed system. Thus, there is no limitation. Accordingly, favorable examples of modes for carrying out the present invention, relating to the timing of the above mixing with such as heating for the temperature raising, include: (i) a mode in which the metal element (M) compound, the hetero-metal element (M′) compound or hetero-nonmetal element compound, and the alcohol are mixed together, and then the temperature of the resultant mixed system is raised by such as heating to thus put it in a high-temperature state; (ii) a mode in which the alcohol is subjected to such as heating to a predetermined temperature and then mixed with the metal element (M) compound and the hetero-metal element (M′) compound or hetero-nonmetal element compound, thereby raising the temperature of the resultant mixed system to thus put it in a high-temperature state; (iii) a mode in which the reaction solvent, the metal element (M) compound, and the hetero-metal element (M′) compound or hetero-nonmetal element compound are mixed together and then subjected to such as heating to a predetermined temperature and then mixed with the alcohol, thereby raising the temperature of the resultant mixed system to thus put it in a high-temperature state; and (iv) a mode in which the components (the metal element (M) compound, the hetero-metal element (M′) compound or hetero-nonmetal element compound, and the alcohol, and further the reaction solvent being used if necessary) are subjected, separately from each other, to such as heating and then mixed together, thereby raising the temperature of the resultant mixed system to thus put it in a high-temperature state. Incidentally, in cases of the fourth metal oxide particle, a raw material gas of the hetero-nonmetal element (e.g. ammonia gas, hydrogen sulfide) may be supplied in any stage of such as: the stage of putting the system in a high-temperature state; a stage of this high-temperature state; and a stage after the formation of the metal oxide particle.

In the above modes (particularly, modes (ii) to (iv)) for carrying out the present invention, the method for mixing such as the starting materials is not limited. However, among such as the starting materials, those which are to be added may be added either in a lump (e.g. within 1 minute) or gradually (e.g. spending a time longer than 1 minute). The gradual addition may be continuous addition (continuous feed), or intermittent addition (pulsewise addition), or their combination, thus there being no limitation. In the intermittent addition (pulsewise, addition), each pulse may be ether continuous addition or lump addition, thus there being no limitation. Incidentally, in the above mixing method, as to the temperature variation (caused by the addition) of the mixed system, the less the more favorable in that it becomes easier to obtain metal oxide particles having a uniform primary particle diameter. Specifically, it is favorable to control such as addition rate so that the temperature variation of the mixed system will be suppressed within 10° C.

In the above modes (particularly, mode (ii)) for carrying out the present invention, the addition rate in cases of adding the metal element (M) compound to the alcohol to mix them together (ratio of the mols of the metal element (M) compound (being added) per minute to the mols of the alcohol to which the zinc compound is added) is favorably in the range of 0.0001 to 2, more favorably 0.0005 to 1.0. If the above addition rate is less than 0.0001, then there is a possibility that it may be difficult to obtain a product of not larger than 0.1 μm in average primary particle diameter. If the above addition rate is more than 2, then there is a possibility that the aforementioned temperature control of the high-temperature state may be difficult (particularly in cases where the reaction scale is large), thus resulting in difficulty in obtaining particles having a uniform particle diameter.

In the production process (A), at least while the aforementioned mixed system of the starting materials is put in a high-temperature state, this mixed system is favorably stirred by a motive power of not less than 0.0001 kw/m³, more favorably not less than 0.001 kw/m³, still more favorably in the range of 0.01 to 10 kw/m³, needed for the stirring.

In the production process (A), it is favorable that, for the purpose of enhancing the dispersibility of the formed metal oxide particle, aliphatic carboxylic acids or aliphatic amines or the aforementioned metal compounds (1) to (3) are added either in a process of until putting the mixed system comprising the starting materials in a high-temperature state to thus form the metal oxide particle, or in any stage after this formation of the metal oxide particle. By this addition, the metal oxide particle can effectively be prevented from aggregating secondarily, so that the particle excellent in the dispersibility can be obtained. Above all, the addition of the above metal compounds is particularly favorable in such that the crystal grain diameter of the metal oxide particle can be controlled, for example, to a very small particle diameter. The amount of the above aliphatic carboxylic acids and aliphatic amines being added (total amount of the addition) is favorably in the range of 0.1 to 10 mol % relative to the metal element (M) in the metal element (M) compound. The amount of the above metal compounds (1) to (3) being added (total amount of the addition) is favorably in the range of 0.1 to 10 atomic % in atomic ratio of the metal elements in these metal compounds to the metal element (M) in the metal element (M) compound.

The metal oxide particle according to the present invention is, as aforementioned, a metal oxide particle which exercises more excellent ultraviolet absorbency as a matter of course and combines therewith merits of, for example, either being shifted in ultraviolet absorption edge toward the longer wavelength side and being excellent also in the absorption efficiency of a long-wavelength range of ultraviolet rays, or having good transparency and, for example, even in cases where added into or coated onto substrates, not damaging the transparency or hue of the substrates.

The metal oxide particle according to the present invention is, for example, useful as a particle which is caused to be contained in: cosmetics; electronic materials for the purpose of ultraviolet interception; various films such as for packing materials; glass used for such as windows for built structures (e.g. buildings, houses), automobile windows, sunroofs, and windows for trains and airplanes; and transparent plastic sheets (e.g. polycarbonate); or useful as a membrane-formable raw material particle for ultraviolet absorbent paints.

In detail, in cases where the metal element (M) in the metal oxide particle according to the present invention is Zn, Ti, or Ce, then this metal oxide particle can simultaneously satisfy excellent ultraviolet absorption performance, excellent colorlessness, and excellent visible-ray transmission performance wherein such simultaneous satisfaction has never been obtained from prior metal oxide particles of the above metal elements (M) or prior particles such that oxides of the above metal elements (M) are caused to contain a hetero-metal element except the hetero-metal elements as specified in the present invention. Therefore, in the above cases, the metal oxide particle according to the present invention is, for example, useful as an ultraviolet absorbent material for interception of ultraviolet rays derived from excitation sources and light sources in display devices (e.g. LCD (liquid crystal displays), PDP (plasma displays), white LED, mercury lamps, fluorescent lamps) and illuminations, and also useful as an ultraviolet absorbent material for various glasses (e.g. inorganic glasses such as monoplate glass, multilayered glass, and laminated glass, and organic glasses such as polycarbonate resins) being used for such as various window materials and displays for such as built structures, cars (e.g. automobiles, electric trains), and air transportation machines (e.g. airplanes, helicopters), and also useful as an ultraviolet absorbent material for various films to which the ultraviolet intercepting ability is demanded (e.g. agricultural films, various packing films).

In cases where the metal element (M) in the metal oxide particle according to the present invention is Zn, Si, or Al, then this metal oxide particle is lower in whiteness degree and higher in transparent feeling than titanium oxide which has hitherto widely been used mainly as an ultraviolet absorbing agent for cosmetics. Therefore, in the above cases, the metal oxide particle according to the present invention is useful as an ultraviolet absorbing agent for cosmetics which agent can give a more excellent transparent feeling. Particularly, in cases where the metal element (M) in the metal oxide particle according to the present invention is Si or Al, then this metal oxide particle is a particle which is, above all, low in refractive index. Therefore, in the above cases, the metal oxide particle according to the present invention is an ultraviolet intercepting material of such a low refractive index as to have never been before and is therefore useful also as the aforementioned display device material and electronic material.

In cases where the metal element (M) in the metal oxide particle according to the present invention is Ti, Zn, Ce, In, or Sn, then this metal oxide particle is a particle which is high in refractive index. Therefore, in the above cases, it becomes possible to obtain a membrane having any refractive index by controlling the formulation ratio of the metal oxide particle to the resin or silicate as the binder component, so the metal oxide particle according to the present invention is useful as a raw material for an ultraviolet absorbent membrane which combines the antireflection property.

Furthermore, the metal oxide particle according to the present invention is applicable also to other uses besides the uses for the purpose of the ultraviolet interception. For example, in cases where the metal element (M) in the metal oxide particle according to the present invention is Ti, Zn, Ce, In, or Sn, then this metal oxide particle is a particle high in refractive index and is therefore favorably usable also as a high-refractive-index filler for enhancing the refractive index of such as resins, films, and membranes. Particularly, in cases where the metal oxide particle according to the present invention is a super fine particle, it is possible to obtain therefrom a transparent and high-refractive-index membrane or film favorable as an antireflective membrane.

In addition, in cases where the metal element (M) in the metal oxide particle according to the present invention is Zn, In, Sn, or Ti, then this metal oxide particle is useful also as an absorbent material for infrared rays (near-infrared to far-infrared rays). Particularly, a particle (first, second, or third metal oxide particle) in which the metal element (M) is Zn and which contains a metal element (e.g. In, Al, Ga, Bi, Fe, Co, Ni, Mn) of 3 in valence as a hetero-metal element (M′), and a particle (fourth metal oxide particle) in which the metal element (M) is Zn, In, Sn, or Ti and which contains fluorine as a hetero-nonmetal element, are useful as absorbent materials for infrared rays.

In addition, in cases where the metal element (M) in the metal oxide particle according to the present invention is Zn or Al, then this metal oxide particle is a particle excellent in the heat conductivity and is therefore useful as a heat-conductive filler and, for example, favorably usable when a high heat-conductive sheet, film, or membrane is obtained for white LED uses or electronic circuit substrate uses wherein these uses are required to have the heat radiation property.

In addition, in cases where the metal element (M) in the metal oxide particle according to the present invention is Zn, Ti, In, or Sn, then this metal oxide particle is a particle excellent in the electronic conductivity and is therefore useful as a semiconductor or dielectric. Particularly, in cases where the metal oxide particle according to the present invention is a super fine particle, this particle is favorably usable as a transparent antistatic membrane or transparent electrically conductive membrane for such as films by forming this particle into a paint.

In addition, the metal oxide particle according to the present invention contains one or at least two hetero-metal elements (M′) or hetero-nonmetal elements and is such that a new electronic level is formed in a band gap of the oxide of the metal element (M). Therefore, this particle is useful also as a photocatalyst material or fluorescent substance material. For example, in recent years, photocatalysts high in sunlight utilization efficiency (which are called visible-ray-working type photocatalysts) are demanded. In cases where the metal element (M) in the metal oxide particle according to the present invention is Zn or Ti, then this metal oxide particle is useful also as a raw material for the above photocatalysts.

In addition, as to the metal oxide particle according to the present invention, in cases where: the aforementioned metal element (M) is any of Zn, Ti, In, and Sn; and at least one member selected from the group consisting of Fe, Co, Ni, and Mn is contained as the aforementioned metal element (M′); or in cases where at least one member selected from the group consisting of Fe, Co, Ni, and Mn, and a metal element higher in valence than the metal element (M) (for example, if M is Zn, then a metal element of 3 or 4 in valence such as In, Al, B, Ga, or Sn), are contained as the aforementioned metal elements (M′); then this metal oxide particle is useful also as a particle which shows (ferro)magnetic (transparent) semiconductor properties.

The metal oxide particle according to the present invention can be adjusted to various colors according to the kinds and combinations of the hetero-metal elements (M′) or hetero-nonmetal elements being caused to be contained and is, for example, useful also as a color pigment. For example, the metal oxide particle tends to be colored as follows: vivid yellow in cases where the metal element (M′) is Bi or Ag; yellow in cases where the metal element (M′) is Fe(III); orange to beige in cases where the metal element (M′) is Mn; green in cases where the metal element (M′) is Fe(II) or Ni(II); blue to green in cases where the metal element (M′) is In or Co; and gray in cases where the metal element (M′) is Cu. By causing these metal elements to coexist with other metal elements (M′) or with the hetero-nonmetal elements, subtly or drastically color-adjusted particles can be formed.

[Composition]:

The composition according to the present invention comprises a metal oxide particle and a medium, wherein the metal oxide particle is dispersed in the medium and includes, as an essential component, the aforementioned metal oxide particle according to the present invention.

The metal oxide particle according to the present invention is applicable to the aforementioned various uses in the form of various liquid or solid compositions.

Examples of the liquid compositions include: a solvent dispersion such that the particles are dispersed in a dispersion solvent; a paint composition such that the particles are dispersed in a paint-film-formable binder; a dispersion such that the particles are dispersed in a plasticizer as a raw material for an intermediate membrane of laminated glass or for resin moldings; a dispersion such that the particles are dispersed in a liquid resin; and a polymerizable composition such that the particles are dispersed in a polymerizable compound such as acrylic monomer. Examples of the solid compositions include: membranes, membrane-coated substrates, and fibrous, filmy, or sheet-shaped resin moldings obtained by using the aforementioned liquid compositions as raw materials. For example, the aforementioned liquid compositions can easily be obtained by a process in which the metal oxide particle according to the present invention obtained in a powdery form, or a reaction liquid resultant from the production of the above metal oxide particle, is dispersed into various dispersion media by hitherto publicly known methods.

Hereinafter, descriptions are given about the composition for membrane formation, which is particularly useful in practical use (in the aforementioned classification, this composition corresponds to the solvent dispersion or paint composition).

As to the composition according to the present invention (including also the below-mentioned composition according to the present invention for membrane formation), it is favorable, in point of the excellence in the transparency of this composition or a membrane obtained therefrom, that the metal oxide particle is dispersed in the form of not larger than 1 μm in dispersion particle diameter. The dispersion particle diameter is more favorably not larger than 0.2 μm, still more favorably not larger than 0.1 μm, particularly favorably not larger than 0.07 μm.

[Composition for Membrane Formation]:

As is aforementioned, the composition according to the present invention for membrane formation is a composition comprising the following essential constitutional components: the aforementioned metal oxide particle according to the present invention; and a dispersion solvent and/or a binder. Incidentally, as to the metal oxide particle according to the present invention which is an essential constitutional component of the composition according to the present invention, the aforementioned description is similarly applicable thereto.

As to the dispersion solvent and/or binder which is an essential constitutional component of the composition according to the present invention, the mutual ratio (formulation ratio) between the amounts of these (dispersion solvent and binder) being used is not limited. This ratio can appropriately be set according to the kind (composition) and amount of the metal oxide particle being used as an essential constitutional component and according to the form of the membrane to be formed.

Examples of the above dispersion solvent include: water; organic solvents (e.g. (various halogenated) hydrocarbons, alcohols, ethers, acetals, ketones, aldehydes, carboxylate esters, amides, and carboxylic acids (anhydrides)); silicone oils; and mineral oils. Of these, only one kind may be used alone, or at least two kinds may be used in combinations with each other.

Examples of the above binder include: organic binders such as a variety of thermoplastic or thermosetting (including also such as thermosetting, ultraviolet-setting, electron-beam-setting, moisture-setting, and their combinations) synthetic resins and natural resins; and inorganic binders. Examples of the synthetic resins include polyester resins, fluororesins, alkyd resins, amino resins, vinyl resins, acrylic resins, epoxy resins, polyamide resins, polyurethane resins, thermosetting unsaturated polyester resins, phenol resins, chlorinated polyolefin resins, butyral resins, silicone resins, acrylic silicone resins, fluororesins, xylene resins, petroleum resins, ketone resins, rosin-modified maleic acid resins, liquid polybutadiene, and coumarone resins. Of these, only one kind may be used alone, or at least two kinds may be used in combinations with each other. Examples of the natural resins include shellac, rosin (pine resin), ester gum, hardened rosin, decolored shellac, and white shellac. Of these, only one kind may be used alone, or at least two kinds may be used in combinations with each other. As the synthetic resins, it is also possible to use such as natural or synthetic rubbers (e.g. ethylene-propylene copolymer rubber, polybutadiene rubber, styrene-butadiene rubber, acrylonitrile-butadiene copolymer rubber). Examples of components being used jointly with the synthetic resins include cellulose nitrate, cellulose acetate butyrate, cellulose acetate, ethyl cellulose, hydroxypropylmethyl cellulose, and hydroxyethyl cellulose.

The form of the binder component is not limited. Examples thereof include solvent-soluble types, water-soluble types, emulsion types, and dispersed types (any solvent such as water/organic solvents may be used).

Examples of the water-soluble type binder component include water-soluble alkyd resins, water-soluble acryl-modified alkyd resins, water-soluble oil-free alkyd resins (water-soluble polyester resins), water-soluble acrylic resins, water-soluble epoxyester resins, and water-soluble melamine resins.

Examples of the emulsion type binder component include alkyl (meth)acrylate copolymer dispersions, vinyl acetate resin emulsions, vinyl acetate copolymer resin emulsions, ethylene-vinyl acetate copolymer resin emulsions, acrylate ester (co)polymer resin emulsions, styrene-acrylate ester (co)polymer resin emulsions, epoxy resin emulsions, urethane resin emulsions, acryl-silicone emulsions, and fluororesin emulsions.

Examples of the inorganic binders include: metal oxide sols (e.g. silica sol, alumina sol, titania sol, zirconia sol, and ceria sol); alkali silicic acid; metal alkoxides (e.g. silicon alkoxides, zirconium alkoxides, and titanium alkoxides) and their (hydrolyzed-)condensed products; (poly)silazanes; and phosphate salts. In addition, as examples, it is possible to also cite metal compounds which can form metal oxides by pyrolysis, such as: metal carboxylates (e.g. metal formates, metal acetates, and metal oxalates) and their basic salts; and organometallic complexes (e.g. β-diketone complexes such as metal acetylacetonates).

These inorganic binders form metal oxides or metal hydroxides due to heat and/or moisture after coating. Among these inorganic binders, favorable in point of the excellence of the above metal oxides or metal hydroxides in the ultraviolet absorbency are inorganic binders containing Ti, Ce, or Zn as an metal element, and favorable in point of the excellence of the resultant membrane in the chemical durability are inorganic binders containing Si, Zr, Ti, or Al as an metal element, and favorable in point of the excellence in the dispersibility of the metal oxide particle are inorganic binders comprising the metal alkoxides, particularly favorably, metal alkoxides containing Si, Ti, or Al as an metal element and their (hydrolyzed-)condensed products.

The composition according to the present invention, favorably, further comprises: a metal oxide particle including, as a metal element, at least one member selected from the group consisting of Cu, Ag, Fe, and Bi (this metal oxide particle is added as a second component and may hereinafter be referred to as “added metal oxide particle”); and/or a superfine metal particle including, as a metal element, at least one member selected from the group consisting of Ag, Cu, Au, and platinum group metal elements (this superfine metal particle is added as a second component and may hereinafter be referred to as “added superfine metal particle”). Thereby the effect of intercepting a short-wavelength range of visible rays can more be enhanced.

Among the added metal oxide particles, examples of metal oxide particles including Cu as a metal element include particles comprising: single oxides or compound oxides such as cuprous oxide (Cu₂O), cupric oxide (CuO), copper ferrite (CuFe₂O₄), copper molybdate (CuMoO₄), copper tungstate (CuWO₄), copper titanate (CuTiO₃), copper selenate (CuSeO₄), and copper chromite (CuCr₂O₄); solid solution oxides such that hetero-metal elements are partly substituted for a part of metal elements of the single oxides or compound oxides; or solid solution oxides such that other elements (e.g. nitrogen element, sulfur element, halogen elements) are partly substituted for a part of oxygen of the single oxides or compound oxides. Incidentally, in the aforementioned oxides, there are also included nonstoichiometric compounds (e.g. Cu₁₋ᵢO). Above all, the copper oxide particles, or particles such that, to surfaces of these particles, there are bonded organic groups such as acyl groups (e.g. ethanoyl group) and alkoxy groups (e.g. ethoxy group), are favorable, and the cuprous oxide particles, or particles such that, to surfaces of these particles, there are bonded organic groups such as acyl groups (e.g. ethanoyl group) and alkoxy groups (e.g. ethoxy group), are particularly favorable.

Among the added metal oxide particles, examples of metal oxide particles including Fe as a metal element include particles comprising: single (hydr)oxides or compound oxides such as iron oxides (e.g. ferrous oxide (FeO), ferric oxides (α-Fe₂O₃, γ-Fe₂O₃), iron tritetraoxide (Fe₃O₄)), iron(III) hydroxides (e.g. α-FeO(OH), γ-FeO(OH)), various ferrite compounds shown by general formula M(II)Fe₂O₄ (wherein M is any one or more kinds of metal elements) (e.g. manganese ferrite, zinc ferrite, cobalt ferrite, nickel ferrite, barium ferrite, zinc nickel ferrite), iron titanate (FeTiO₃), iron molybdate (FeMoO₄), and iron tungstate (FeWO₄); solid solution oxides such that hetero-metal elements are partly substituted for a part of metal elements of the single (hydr)oxides or compound oxides; or solid solution oxides such that other elements (e.g. nitrogen element, sulfur element, halogen elements) are partly substituted for a part of oxygen of the single (hydr)oxides or compound oxides. Incidentally, in the aforementioned (hydr)oxides, there are also included nonstoichiometric compounds (e.g. Fe₁₋ᵢO). Above all, the iron (hydr)oxide particles, or particles such that, to surfaces of these particles, there are bonded organic groups such as acyl groups (e.g. ethanoyl group) and alkoxy groups (e.g. ethoxy group), are favorable, and the α-Fe₂O₃ particles, or particles such that, to surfaces of these particles, there are bonded organic groups such as acyl groups (e.g. ethanoyl group) and alkoxy groups (e.g. ethoxy group), are particularly favorable.

Among the added metal oxide particles, examples of metal oxide particles including Bi as a metal element include particles comprising: single oxides or compound oxides such as bismuth(III) trioxide (Bi₂O₃), bismuth titanate (Bi₄Ti₃O₁₂), bismuth molybdate (Bi₂MoO₆), bismuth tungstate (Bi₂WO₆), bismuth stannate (Bi₂Sn₂O₇), and bismuth zirconate (2Bi₂O₃.3ZrO₂); solid solution oxides such that hetero-metal elements are partly substituted for a part of metal elements of the single oxides or compound oxides; or solid solution oxides such that other elements (e.g. nitrogen element, sulfur element, halogen elements) are partly substituted for a part of oxygen of the single oxides or compound oxides. Incidentally, in the aforementioned oxides, there are also included nonstoichiometric compounds (e.g. Bi₂₋ᵢO₃). Above all, the oxide particles, or particles such that, to surfaces of these particles, there are bonded organic groups such as acyl groups (e.g. ethanoyl group) and alkoxy groups (e.g. ethoxy group), are favorable, and the particles comprising bismuth trioxide or bismuth titanate, or particles such that, to surfaces of these particles, there are bonded organic groups such as acyl groups (e.g. ethanoyl group) and alkoxy groups (e.g. ethoxy group), are particularly favorable.

Among the added metal oxide particles, examples of metal oxide particles including Ag as a metal element include particles comprising: single oxides or compound oxides such as silver oxide (Ag₂O); solid solution oxides such that hetero-metal elements are partly substituted for a part of metal elements of the single oxides or compound oxides; or solid solution oxides such that other elements (e.g. nitrogen element, sulfur element, halogen elements) are partly substituted for a part of oxygen of the single oxides or compound oxides. Incidentally, in the aforementioned oxides, there are also included nonstoichiometric compounds (e.g. Ag₂₋ᵢO). Above all, the oxide particles, or particles such that, to surfaces of these particles, there are bonded organic groups such as acyl groups (e.g. ethanoyl group) and alkoxy groups (e.g. ethoxy group), are favorable, and the particles comprising silver oxide (Ag₂O), or particles such that, to surfaces of these particles, there are bonded organic groups such as acyl groups (e.g. ethanoyl group) and alkoxy groups (e.g. ethoxy group), are particularly favorable.

Incidentally, the added metal oxide particle may be either a metal oxide particle including, as metal elements, at least two members selected from the group consisting of Cu, Fe, Ag, and Bi or a metal oxide particle further including a metal element other than Cu, Fe, Ag, and Bi.

Though not especially limited, the size of the added metal oxide particle is favorably in the range of 1 to 100 nm, more favorably 5 to 30 nm, still more favorably 5 to 20 nm, in average particle diameter of primary particles in point of exercising the excellent transparency.

The added superfine metal particle is a superfine metal particle including, as a metal element, at least one member selected from the group consisting of Cu, Ag, Au, and platinum group metal elements. In addition, as is aforementioned, there are also included cases where a part or the whole of this particle is oxidized to thus exist as an oxide. This added superfine metal particle favorably comprises a single metal or a particle for alloys and is recommended to be in the range of 1 to 100 nm, favorably 1 to 20 nm, in primary particle diameter. It is favorably strong in absorption by plasmon absorption at not longer than 450 nm, and examples of such include a superfine metal particle including Cu and/or Ag as a metal element.

The amounts of the metal oxide particle and the dispersion solvent and/or binder, which are used as the essential constitutional components of the composition according to the present invention, are not limited. However, specifically, the ratio of the amount of all metal oxide particles being used is favorably in the range of 10 to 90 weight %, more favorably 20 to 80 weight %, relative to the entire solid component content of the composition. If the above ratio of the amount being used is smaller than 10 weight %, then, for example, in cases where the composition is used for formation of an ultraviolet intercepting membrane, this membrane needs to be made so thick as to sufficiently exercise the UV intercepting ability and, particularly in cases such as where an inorganic binder or a setting resin is used as another membrane component besides the above particle, there is a possibility that the resultant membrane may tend to crack. If the above ratio of the amount being used is larger than 90 weight %, then, for example, in cases where the composition is used for formation of a membrane, there is a possibility that the physical strength of the resultant membrane may be insufficient. However, in cases where the composition according to the present invention is a composition comprising the dispersion solvent as an essential constitutional component and is coated to the substrate and then calcined (sintered) by heating to a high temperature, thus forming a membrane, then the ratio of the metal oxide particle in the composition may be more than 90 weight %, particularly be 100 weight %, relative to the entire solid component content of the composition.

The composition according to the present invention may further comprise another constitutional component and, for example, may further comprise such as dispersants, inorganic binders, and setting resins.

As the dispersants, for example, the aforementioned metal compounds (1) to (3) are favorable and, above all, the metal compounds (3) are particularly favorable. The amount of the above metal compounds (1) to (3) being added (total amount of the addition) is favorably in the range of 0.1 to 10 atomic % in atomic ratio of the metal elements in these metal compounds to the metal element (M) in the metal element (M) compound.

Uses of the composition according to the present invention are not limited. For example, this composition can be handled as a coating liquid for formation of an ultraviolet intercepting membrane or as an ultraviolet cutting paint. Specifically, this composition is, for example, useful as a coating liquid being used when an ultraviolet intercepting membrane is formed on a film or glass for interception of ultraviolet rays derived from excitation sources and light sources in display devices (e.g. LCD (liquid crystal displays), PDP (plasma displays), white LED, mercury lamps, fluorescent lamps) and illuminations, and also useful as a coating liquid being used when an ultraviolet intercepting membrane is formed on various glasses (e.g. inorganic glasses such as monoplate glass, multilayered glass, and laminated glass, and organic glasses such as polycarbonate resins) being used for such as various window materials and displays for such as built structures, cars (e.g. automobiles, electric trains), and air transportation machines (e.g. airplanes, helicopters), and also useful as a coating liquid being used when an ultraviolet intercepting membrane is formed on various films to which the ultraviolet intercepting ability is demanded (e.g. agricultural films, various packing films), and further useful as a coating liquid being used when an ultraviolet intercepting membrane is formed as an intermediate membrane of laminated glass being used for such as the aforementioned window materials. In addition, as is aforementioned in the section hereof headed “[Metal oxide particle]”, the composition according to the present invention is useful also as: a coating liquid being used for formation of various functional membranes such as infrared absorbent membranes, high-refractive-index membranes, low-refractive-index membranes, antireflective membranes, heat-conductive membranes, antistatic membranes, transparent electrically conductive membranes, photocatalyst membranes, fluorescent substance membranes, and magnetic substance membranes; or as ink-jet ink; in accordance with the kinds of the metal elements of the metal oxide particle contained in the above composition. Particularly, the composition according to the present invention containing the metal oxide particle which includes Zn as a main metal element (the aforementioned metal element (M)) can exercise especially good physical properties in the aforementioned various uses and is therefore favorable.

[Membrane]:

As is aforementioned, the membrane according to the present invention is a membrane obtained by comprising the aforementioned metal oxide particle according to the present invention and/or a metal oxide crystal, derived from this metal oxide particle, as an essential constitutional component. Specifically, the membrane according to the present invention encompasses all membranes obtained by using, as a raw material component, either a composition containing the metal oxide particle according to the present invention (e.g. an intermediate composition) or the composition according to the present invention for membrane formation, suchas: (1) a membrane such that the metal oxide particle according to the present invention is dispersed in the binder; (2) a membrane comprising only the above particle; (3) a membrane obtained by sintering the above particle; and (4) a membrane comprising a combination of these membrane forms (particularly a membrane comprising a combination of the above membrane (2) and the above membrane (3)).

The above membrane (1) is obtained by coating or molding the aforementioned composition containing the binder. The above membrane (2) is obtained by coating the aforementioned composition of the solvent dispersion type. The above membrane (3) is obtained as a membrane of the metal oxide crystal formed by such as calcining the above membrane (1) or (2) at a high temperature and thereby sintering the metal oxide particle. The above membrane (4) is, for example, obtained as a combined membrane of the metal oxide particle and the metal oxide crystal derived from this particle, wherein the combined membrane is formed by such as calcining the above membrane (2) at a high temperature and thereby sintering a part of the metal oxide particles. As a result, usually, in the above membranes (1) and (2), the metal oxide particle according to the present invention exists maintaining substantially its form. However, in the above membrane (3) and in a portion (which can be in the form of the above membrane (3)) of the above membrane (4), a structural variation (e.g. variation of the crystal grain diameter of the particle) is involved, so there may be cases where the resultant membrane is a polycrystal membrane or single-crystal membrane which is different from the crystal form of the original particle. Incidentally, as to the metal oxide particle according to the present invention which is an essential constitutional component (or essential raw material component) of the membrane according to the present invention, the aforementioned description is similarly applicable thereto.

A favorable mode of the membrane according to the present invention is that this membrane further comprises: a metal oxide particle including, as a metal element, at least one member selected from the group consisting of Cu, Fe, Ag, and Bi and/or a metal oxide crystal derived from this particle; and/or a superfine metal particle including, as a metal element, at least one member selected from the group consisting of Ag, Cu, Au, and platinum group metal elements (inclusive of a superfine metal oxide particle formed in a way that a metal including, as a metal element, at least one member selected from the group consisting of Ag, Cu, Au, and platinum group metal elements is oxidized in a membrane formation step or its subsequent step) and/or a crystal including a metal derived from this particle and/or a crystal including a metal oxide derived from this particle. Thereby the effect of intercepting a short-wavelength range of visible rays can more be enhanced. Incidentally, the metal oxide particle including, as a metal element, at least one member selected from the group consisting of Cu, Fe, Ag, and Bi, and the metal including, as a metal element, at least one member selected from the group consisting of Ag, Cu, Au, and platinum group metal elements, are the same as the added metal oxide particle and the added superfine metal particle respectively which are described in the aforementioned section hereof headed “[Composition for membrane formation]”.

Though not limited, the membrane according to the present invention is generally a membrane formable on a desired substrate surface, and its form may be either a form which exists spreading over a desired area portion of the substrate surface continuously without any gap (such a membrane may hereinafter be referred to as continuous membrane), or a form which exists discontinuously on a desired area portion of the substrate surface (such a membrane may hereinafter be referred to as discontinuous membrane), thus there being no limitation. As to the discontinuous membrane, the constitutional components of the membrane exist partly (are dotted) on the substrate surface. However, their sizes, areas, thicknesses, shapes, or the like are not limited. Examples of specific forms of the discontinuous membrane include: a form such that the constitutional components of the membrane exist in fine dots on the substrate surface; a form such that the constitutional components of the membrane exist like what is called sea-island structure on the substrate surface; a form such that the constitutional components of the membrane exist in a striped pattern on the substrate surface; and a form comprising a combination of these forms.

In cases where the above continuous membrane and discontinuous membrane comprise only the metal oxide particle as the constitutional component (i.e. comprise an assemblage of the metal oxide particles), the structures of these membranes are not limited. Specifically, they may be either porous structures having spaces of desired sizes or monolithic dense solid structures which are macroscopically not such porous structures (i.e. substantially dense structures). However, the denser structures the preferable in that it is possible to obtain membranes which are excellent in the UV intercepting ability and free from the visible-ray transparency deterioration caused by scattering. Incidentally, as to the discontinuous membrane, such a membrane structure as mentioned above may be provided either to all of individual membrane portions existing partly or to only a part of them.

The mode for carrying out the membrane according to the present invention is defined as encompassing both of a mode referring to a membrane itself formed on the substrate surface and a mode referring to a composition of: a membrane formed on the substrate; and this substrate.

As to the above substrate usable for the membrane according to the present invention, such as its material is not limited. Favorable examples thereof include: inorganic materials such as ceramics (e.g. oxides, nitrides, carbides) and glass; organic materials such as polyester resins (e.g. PET, PBT, PEN), polycarbonate resins, polyphenylene sulfide resins, polyether sulfone resins, polyether imide resins, polyimide resins, amorphous polyolefin resins, polyallylate resins, aramid resins, polyether ether ketone resins, resin films and sheets known as heat-resistant resin films (of such as liquid crystal polymers), and besides, hitherto publicly known films and sheets of various resin polymers including various resins (e.g. (meth)acrylic resins, PVC resins, PVDC resins, PVA resins, EVOH resins, polyimide resins, polyamideimide resins, fluororesins (e.g. PTFE, PVF, PGF, ETFE), epoxy resins, polyolefin resins), and processed products (e.g. films such that such as aluminum, alumina, and silica are vapor-deposited on these various resin polymers); and various metals.

Examples of the shape and form of the above substrate include films, sheets, plate, fibers, and laminates. However, they will do if they are selected according to such as usage and use purposes. Thus, there is no limitation. In addition, the above substrate is not limited in functional aspects, either. For example, the above substrate may be either optically transparent or opaque and will do if it is selected according to such as usage and use purposes.

The membrane according to the present invention is used without being limited to uses for the purpose of the ultraviolet interception, and is favorably high in transparency. Specifically, its haze is favorably not more than 10%, more favorably not more than 2%, still more favorably not more than 1%.

The optical performances of the membrane according to the present invention can be evaluated in a way that the performance of interception of light in the range of ultraviolet rays (ultraviolet rays of not longer than 380 nm and visible rays of not longer than 450 nm) (ultraviolet intercepting performance) and the performance of transmission of visible rays (450 to 780 nm) (visible-ray transmission performance) are used as indexes. It is favorable for the ultraviolet absorbing functional material to be high in the ultraviolet intercepting performance and the visible-ray transmission performance. As is aforementioned, usually, the ultraviolet intercepting performance and visible-ray transmission performance of the membrane can be judged by evaluating the spectroscopic transmittance properties in a state where the membrane is formed. The details of how to judge the ultraviolet intercepting performance and the visible-ray transmission performance are as aforementioned.

Incidentally, among the optical performances of the membrane according to the present invention, the following optical properties of the membrane are defined as values measured and evaluated by methods as stated in the below-mentioned detailed description of Examples of some preferred embodiments. In addition, they are defined as physical properties of only the membrane portion (excluding the substrate) and as being evaluated with consideration given to optical properties of the membrane-coated substrate and optical properties of only the substrate. In addition, among the optical properties of the membrane according to the present invention, the transmittance (%) of light of 380 nm in wavelength which is an index of the ultraviolet absorption performance is defined as T³⁸⁰, and further, the transmittance (%) of light of 500 nm in wavelength which is an index of the visible-ray transmission performance is defined as T⁵⁰⁰, and the minimum value of the transmittances (%) of light of 550 to 700 nm in wavelength is defined as T¹, and the absolute value of the difference between T¹ and T⁵⁰⁰ (|T¹−T⁵⁰⁰|) is defined as ΔT.

Particularly, as to the optical properties of the membrane according to the present invention in cases where the metal oxide particle according to the present invention is used as an ultraviolet absorbent material, the T³⁸⁰ is favorably not more than 40%, more favorably not more than 20%, still more favorably not more than 10%, particularly favorably not more than 5%. Similarly, the ΔT is favorably not more than 10%, more, favorably not more than 5%. Similarly, the T⁵⁰⁰ is favorably not less than 80%, more favorably not less than 85%, still more favorably not less than 90%, particularly favorably not less than 95%. Similarly, the haze value (value given by subtracting the haze value of the substrate) which is an index of the visible-ray transparency is favorably less than 3%, more favorably less than 1%, still more favorably less than 0.5%.

Furthermore, the membrane according to the present invention may be a membrane simultaneously satisfying the above ranges as to at least two kinds of the above various optical properties, and can be selected so as to meet the demands according to such as use purposes. For example, the following membranes can be cited (incidentally, the haze value is not more than 1% as to any membrane).

(i) A membrane such that: the T³⁸⁰ is not more than 40%, the ΔT is not more than 5%, and the T⁵⁰⁰ is not less than 95%.

(ii) A membrane such that: the T³⁸⁰ is not more than 20%, the ΔT is not more than 10%, and the T⁵⁰⁰ is not less than 90%.

(iii) A membrane such that: the T³⁸⁰ is not more than 10%, the ΔT is not more than 10%, and the T⁵⁰⁰ is not less than 80%.

The process for formation of the membrane according to the present invention is not limited. However, for example, favorable is a process in which the membrane is formed from the aforementioned composition according to the present invention for membrane formation. Incidentally, as to the composition according to the present invention usable for this formation process, the aforementioned description is similarly applicable thereto.

Hereinafter, descriptions are given about the process for membrane formation from the composition according to the present invention.

The process in which the membrane is formed from the composition according to the present invention is not limited. However, the following processes can be adopted: a process in which the membrane is formed by coating the composition onto the substrate surface by hitherto publicly known membrane formation processes such as coating processes (e.g. bar coater processes, roll coater processes, knife coater processes, die coater processes, and spin coating processes) and spray processes; and what is called dipping process in which the membrane is formed by carrying out the coating by dipping a part or the entirety of the substrate into the composition according to the present invention and then taking the substrate out of the composition. In addition, in cases such as where the dispersion solvent is used as an essential constitutional component of the composition according to the present invention, it is also possible to form the membrane by carrying out the coating followed by calcining at a high temperature. For example, there can be obtained a crystalline membrane such that at least a part of the metal oxide particles are fused together.

The membrane according to the present invention is, for example, useful as an ultraviolet intercepting membrane being used for such various uses as aforementioned in the sections hereof headed “[Metal oxide particle]” and “[Composition for membrane formation]”, and also useful as various functional membranes such as infrared absorbent membranes, high-refractive-index membranes, low-refractive-index membranes, antireflective membranes, heat-conductive membranes, antistatic membranes, transparent electrically conductive membranes, photocatalyst membranes, and fluorescent substance membranes. Furthermore, the membrane according to the present invention is useful also as a membrane having at least two functions which combines the ultraviolet intercepting membrane with any of the aforementioned various functional membranes (e.g. an ultraviolet intercepting membrane having a high refractive index, an ultraviolet intercepting membrane having transparency and electric conductivity).

[Metal-Oxide-Containing Article]:

The metal-oxide-containing article according to the present invention is an article comprising a metal oxide particle and/or a metal oxide crystal derived from this particle, wherein the article includes, as essential components, a combination of the aforementioned metal oxide particle according to the present invention with: a metal oxide particle including, as a metal element, at least one member selected from the group consisting of Cu, Ag, Fe, and Bi; and/or a superfine metal particle including, as a metal element, at least one member selected from the group consisting of Cu, Ag, Au, and platinum group metal elements. Due to such a combination, the metal-oxide-containing article according to the present invention is excellent not only in the effect of intercepting ultraviolet rays but also in the effect of intercepting a short-wavelength range of visible rays. Incidentally, the metal oxide particle including, as a metal element, at least one member selected from the group consisting of Cu, Ag, Fe, and Bi, and the superfine metal particle including, as a metal element, at least one member selected from the group consisting of Ag, Cu, Au, and platinum group metal elements, are the same as the added metal oxide particle and the added superfine metal particle respectively which are described in the aforementioned section hereof headed “[Composition for membrane formation]”.

In the metal-oxide-containing article according to the present invention, the ratio of the aforementioned added metal oxide particle and/or aforementioned added superfine metal particle to the aforementioned metal oxide particle according to the present invention is not limited. However, this ratio is favorably in the range of 0.1 to 50 weight parts, more favorably 1 to 10 weight parts, relative to 100 weight parts of the metal oxide particle according to the present invention. If the ratio of the aforementioned added metal oxide particle and/or aforementioned added superfine metal particle is smaller than the aforementioned ranges, then there are cases where the effects by the combination are insufficient. On the other hand, if this ratio is larger than the aforementioned ranges, then there are cases where there occur problems such that the visible-ray transmission property is reduced and that the coloring degree is increased.

[Ultraviolet Absorbent Material]:

The ultraviolet absorbent material according to the present invention comprises the metal oxide particle according to the present invention. Incidentally, as to the metal oxide particle according to the present invention which is an essential constitutional component of the ultraviolet absorbent material according to the present invention, the aforementioned description is similarly applicable thereto.

In addition, the ultraviolet absorbent material according to the present invention, favorably, further comprises: a metal oxide particle including, as a metal element, at least one member selected from the group consisting of Cu, Ag, Fe, and Bi; and/or a superfine metal particle including, as a metal element, at least one member selected from the group consisting of Ag, Cu, Au, and platinum group metal elements. Thereby the effect of intercepting a short-wavelength range of visible rays can more be enhanced. Incidentally, the metal oxide particle including, as a metal element, at least one member selected from the group consisting of Cu, Ag, Fe, and Bi, and the superfine metal particle including, as a metal element, at least one member selected from the group consisting of Ag, Cu, Au, and platinum group metal elements, are the same as the added metal oxide particle and the added superfine metal particle respectively which are described in the aforementioned section hereof headed “[Composition for membrane formation]”.

As to the ultraviolet absorbent material according to the present invention, in cases where this ultraviolet absorbent material further comprises the aforementioned added metal oxide particle and/or aforementioned added superfine metal particle, then the ratio of the aforementioned added metal oxide particle and/or aforementioned added superfine metal particle to the aforementioned metal oxide particle according to the present invention is not limited. However, this ratio is favorably in the range of 0.1 to 50 weight parts, more favorably 1 to 10 weight parts, relative to 100 weight parts of the metal oxide particle according to the present invention. If the ratio of the aforementioned added metal oxide particle and/or aforementioned added superfine metal particle is smaller than the aforementioned ranges, then there are cases where the effects by the combination are insufficient. On the other hand, if this ratio is larger than the aforementioned ranges, then there are cases where there occur problems such that the visible-ray transmission property is reduced and that the coloring degree is increased.

### DETAILED DESCRIPTION OF THE PREFERRED EMBODIMENTS

Hereinafter, the present invention is more specifically illustrated by the following Examples of some preferred embodiments in comparison with Comparative Examples not according to the present invention. However, the present invention is not limited to them. Hereinafter, for convenience, the unit “weight part(s)” may be referred to simply as “part(s)”, and the unit “weight %” may be referred to as “wt %”.

Incidentally, unless otherwise noted, the preparation of the powder sample being used in the evaluations of each Example and Comparative Example was carried out in the following way. That is to say, a reaction liquid resultant from a reaction to form metal oxide particles was subjected to centrifugal separation, and then washing of the resultant sediment with a reaction solvent (an operation of redispersing the sediment into the reaction solvent and then centrifugally separating the sediment) was thrice repeated, and then the resultant sediment was vacuum-dried at 60° C. with a vacuum drier for 12 hours, thus obtaining the powder sample of the metal oxide particles.

[First Metal Oxide Particle]:

The evaluation methods in the below-mentioned Examples and Comparative Examples are shown below.

(1) Crystal Identification of Metal Oxide Particles:

As to the above powder sample, the crystal system and crystal structure of the metal oxide particles were evaluated by powder X-ray diffractometry with a powder X-ray diffraction device (produced by Rigaku Denki K.K., product name: RINT 2400). Hereinafter the measurement conditions are shown.
X-rays: CuKα1 rays (wavelength: 1.54056 Å)/40 kV/200 mA
Scanning range: 2θ=20 to 80°
Scanning speed: 5°/min

Incidentally, in cases where the metal oxide particles contained Zn as a main metal component, then whether the metal oxide particles had the same crystal system and crystal structure as of ZnO or not was judged from whether three intense-ray peaks characteristic of ZnO of the hexagonal crystal system were seen or not. Specifically, if diffraction peaks existed in all positions of the following three diffraction angles (a) to (c), then it was judged that the metal oxide particles had the same crystal system and crystal structure as of ZnO.
(a) 2θ=31.65 to 31.95°
(b) 2θ=34.30 to 34.60°
(c) 20θ=36.10 to 36.40°

Incidentally, the diffraction peak existing in the position of the above (a) is judged to be based on diffracted rays to the (100) plane of the ZnO crystal, and thediffraction peak existing in the position of the above (b) is judged to be based on diffracted rays to the (002) plane of the ZnO crystal, and the diffraction peak existing in the position of the above (c) is judged to be based on diffracted rays to the (101) plane of the ZnO crystal.

Similarly also in cases where a metal element other than Zn was contained as a main metal component in the metal oxide particles, whether the metal oxide particles had the same crystal system and crystal structure as of an oxide of the above metal element or not was judged from whether three intense-ray peaks characteristic of an oxide crystal of the above metal element were seen or not.

(2) Particle diameter of metal oxide particles:

(2-1) Primary particle diameter:

The crystal grain diameter (Dw) of the metal oxide particles was measured and evaluated as the primary particle diameter.

The crystal grain diameter (Dw) was evaluated in the following way: as to the above powder sample, the crystal grain diameter (Dw) of the metal oxide particles was evaluated by powder X-ray diffractometry with a powder X-ray diffraction device (produced by Rigaku Denki K.K., product name: RINT 2400). Specifically, the crystal grain diameter Ds (hkl) (wherein the hkl denotes a Miller index: the Ds (hkl) is the size of the crystal grain in the vertical direction to the lattice plane of the Miller index (hkl)) was determined by Scherrer equation (analysis) from widths of diffracted rays in the resultant X-ray diffraction pattern, and the average value of three intense rays' respective Ds values was taken as the Dw. That is to say, unless otherwise noted, the crystal grain diameter (Dw) is usually calculated in the following way. A powder X-ray diffraction pattern of the metal oxide particle is measured, and then, as to three intense rays thereof (the largest peak (1) of diffracted rays, the second largest peak (2) of diffracted rays, and the third largest peak (3) of diffracted rays), the crystal grain diameters Ds1, Ds2, and Ds3 in the vertical directions to the diffraction planes assigned to the diffracted rays (1) to (3) respectively are determined from their respective full widths of half maximum intensity or integral widths in accordance with Scherrer equation, and then their average value ((Ds1+Ds2+Ds3)/3) is calculated as the crystal grain diameter (Dw).

(2-2) Dispersion Particle Diameter:

The resultant reaction liquid, or a solvent dispersion having been obtained from this reaction liquid by solvent displacement, was used as the sample, and its median diameter was measured with a dynamic light scattering type particle diameter distribution measurement device (“LB-500” produced by Horiba Seisakusho) and taken as the dispersion particle diameter. In cases where dilution was carried out in preparation for the measurement, then the solvent having been used in the reaction was used as a diluting solvent. The evaluation standards are as follows:
●: Dispersion particle diameter<0.05 μm
⊚: 0.05 μm≦Dispersion particle diameter<0.1 μm
◯: 0.1 μm≦Dispersion particle diameter<1 μm
×: 1 μm≦Dispersion particle diameter

Incidentally, also as to the dispersion particle diameter of fine particles in paints, the median diameter having been measured with the dynamic light scattering type particle diameter distribution measurement device (“LB-500” produced by Horiba Seisakusho) was taken as the dispersion particle diameter similarly to the above.

(2-3) Dispersed and Aggregated States:

The resultant reaction liquid was diluted with the reaction solvent to thus prepare a sample of 0.1 wt % in particle concentration, and then its dispersed state was examined with a transmission electron microscope. Its evaluation standards are as follows:
A: Primary particles are monodispersed or, even if they are aggregated, it is one-dimensional or two-dimensional aggregation.
B: Primary particles are three-dimensionally aggregated to thus form a granular material.

(3) Composition of Metal Oxide Particles:

(3-1) Content of Added Metal Element:

The reaction liquid resultant from the reaction to form the metal oxide particles was used as the sample, and this sample was subjected to quantitative analyses into metal elements by fluorescent X-ray analyses to thus determine the content of the added metal element (e.g. Cu, Ag) relative to the main metal element (e.g. Zn) and, in cases where the metal compound was used as an additive during the formation of the particles, the content of the metal element (Ms) of the above metal compound relative to the main metal element (e.g. Zn).

(3-2) Bonding Amount of Acyl Group:

An amount of 1 g of the above powder sample was added to a 0.1 N aqueous sodium hydroxide solution and then stirred for 24 hours. Thereafter, by ion chromatography, the acyl group was identified and quantified in bonding amount.

(3-3) Evaluation of Valence of Added Metal Element:

If necessary, the valence of the added metal element (e.g. Cu, Ag) in the metal oxide particles was evaluated in the following way. That is to say, as to the above powder sample, a 2p_{3/2} spectrum of the added metal element (e.g. Cu, Ag) contained in the metal oxide particles was measured by X-ray photoelectron spectroscopy (XPS) with a photoelectron spectroscope (produced by Nippon Denshi K.K., product name: JPS-90 model) and, from its peak position, the bond energy value was determined to thus judge the valence of the added metal element (e.g. Cu, Ag).

Incidentally, in order to reduce measured value errors caused by such as energy shift due to the electrification property, the determination of the bond energy value was put under corrections based on the C 1s peak position of the surface hydrocarbon.

In addition, as already known data for comparison, peak positions of 2p_{3/2} spectra of compounds of various metal elements as shown in “The Handbook of X-ray Photoelectron Spectroscopy” (1991) published by Nippon Denshi K.K. were referred to.

(4) Optical Properties of Metal Oxide Particles:

(4-1) Absorption Properties (1):

A dilution, having been prepared by diluting the resultant reaction liquid with 1-butanol as a diluting solvent so as to be 0.1 wt % in fine particle concentration, was used as the sample and, as to this sample, its transmission spectrum in the ultraviolet and visible regions was measured by use of an auto-recording spectrophotometer having an integrating sphere (“UV-3100” produced by Shimadzu Corporation).

Ultraviolet intercepting ability: evaluated by transmittances at 380 nm, 400 nm, 420 nm.

Visible-ray transmission property: evaluated by a transmittance at 600 nm.

Incidentally, also as to the transmission spectrum of a membrane-formed product, its transmission spectrum in the ultraviolet and visible regions was, similarly to the above, measured by use of the auto-recording spectrophotometer having an integrating sphere (“UV-3100” produced by Shimadzu Corporation).

(4-2) Absorption Properties (2):

A diffused reflectance spectrum of the powder sample was measured by use of the same auto-recording spectrophotometer having an integrating sphere as used in (4-1) above.

(4-3) Evaluation of Transparency and Hue:

A fine-particles-dispersed membrane was formed and evaluated. Specifically, a reaction liquid resultant from a reaction to form metal oxide particles was subjected to heating solvent displacement to thereby obtain a dispersion such that the metal oxide particles were dispersed in 1-butanol in a particle concentration of 20 wt %. An amount of 100 parts of the resultant dispersion was mixed with 20 parts of a silicate binder (solid component content in terms of SiO₂: 51 wt %) and 0.5 part of a catalyst. (n-butylamine) to thus prepare a paint. Incidentally, as to the above particle concentration, it was calculated in a way that the solid component amount as a result of vacuum drying of the resultant dispersion at 120° C. with a vacuum drier for 1 hour was taken as the particle weight.

The resultant paint was coated onto alkali-free glass (produced by Corning International Corporation, barium borosilicate glass, Glass Code No. 7059, thickness: 0.6 mm) by use of a bar coater in a way for the wet membrane thickness to be 24 μm. Thereafter, they were normally dried at 25° C. to thereby obtain glass, on the surface of which there was formed a metal-oxide-particles-dispersed membrane.

Then, the above dispersion-membrane-coated glass was used as the sample and evaluated by transparency and hue. The transparency was evaluated by a haze value measured with a turbidimeter (“NDH-1001 DP” produced by Nippon Denshoku Kogyo Co., Ltd.). As to the hue, the appearance was observed with the eye.

Incidentally, the haze value of the alkali-free glass as the substrate was 0%.

Incidentally, also as to the transparency and hue of a membrane-formed product, similarly to the above, the transparency was evaluated by the haze value measured with the turbidimeter (“NDH-1001 DP” produced by Nippon Denshoku Kogyo Co., Ltd.), and the hue was evaluated by observing the appearance with the eye.

(5) Hue of Powder Sample:

It was evaluated by observing the appearance of the powder sample with the eye.

(6) Fine Particle Concentration:

The fine particle concentration of the reaction liquid or of the dispersion was calculated by weighing out 0.5 g of the reaction liquid or of the dispersion in a melting pot and then vacuum-drying it at 120° C. for 1 hour and then measuring the weight of the resultant dry powder.

(7) Refractive Index of Membrane-Formed Product:

The refractive index at a wavelength of 550 nm was determined by measuring the reflectance of the membrane (having been formed on a film) in the range of 230 to 760 nm with a reflection-spectroscopic membrane thickness meter (“FE-3000” produced by Ohtsuka Electronics CO., Ltd.) and then citing nkCauchy's dispersion formula as a representative approximation formula of the wavelength dispersion of the refractive index to determine an unknown parameter by the nonlinear least-squares method from an actually measured value of a spectrum of the absolute reflectance.

### EXAMPLE A1-1

There was prepared a reaction apparatus comprising: a pressure-resistant glass reactor possible to externally heat and equipped with a stirrer, an addition inlet (connected directly to an addition tank), a thermometer, a distillate gas outlet, and a nitrogen-gas-introducing inlet; the addition tank connected to the above addition inlet; and a condenser (connected directly to a trap) connected to the above distillate gas outlet.

Into the above reactor, there was charged a mixture comprising 183 parts of zinc acetate anhydride powder, 0.13 part of copper(I) acetate anhydride powder, and 3,885 parts of 1-butanol, and then its gas phase portion was purged with nitrogen gas.

Thereafter, under stirring, the temperature of the mixture was raised from 20° C. and then heat-retained at 150° C.±1° C. for 10 hours to thus carry out a reaction to form metal oxide particles and then cooled, thereby obtaining a reaction liquid (1-1) containing light-grayish fine particles (metal oxide particles according to the present invention) in a concentration of 2 wt %.

The metal oxide particles in the reaction liquid (1-1) were subjected to the aforementioned various measurements and evaluations. Their results are shown in Table 2.

### EXAMPLES A1-2 TO A1-13 AND COMPARATIVE EXAMPLES A1-1 to A1-2

Reaction liquids (1-2) to (1-13), (c1-1), and (c1-2), containing fine particles (metal oxide particles) (as shown in Table 2) in concentrations as shown in Table 2, were obtained in the same way as of Example A1-1 except that such as the kinds and use amounts of the raw materials being charged and reaction conditions were changed as shown in Table 1.

The metal oxide particles in each of the reaction liquids (1-2) to (1-13), (c1-1), and (c1-2) were subjected to the aforementioned various measurements and evaluations. Their results are shown in Table 2.

Incidentally, as to the particles having been obtained from Examples A1-2, A1-5, A1-9, and A1-12, it was confirmed that the Si compound or Ti compound, having been added as the surface-treating metal compound, was bonded to particle surfaces.

TABLE 1

| | | | | | Reaction | |
|---|---|---|---|---|---|---|
| Raw material for | Compound of added | Raw material for | Surface-treating | | conditions | |
| metal oxide | metal element | another metal | metal compound | Solvent | Temper- | Reten- |

| | | Weight | | Weight | | Weight | | Weight | | Weight | ature | tion |
|---|---|---|---|---|---|---|---|---|---|---|---|---|
| | | parts | | parts | | parts | | parts | | parts | (° C.) | time (hr) |
| Example | Zinc | 183 | Copper (I) | 0.13 | — | 0 | — | 0 | 1-Butanol | 3885 | 150 | 10 |
| A1-1 | acetate | | acetate | | | | | | | | | |
| | anhydride | | anhydride | | | | | | | | | |
| Example | Zinc | 155 | Copper (II) | 3.7 | — | 0 | Tetra- | 1.5 | Methanol | 1875 | 120 | 5 |
| A1-2 | fonnate | | acetate | | | | methoxy- | | | | | |
| | anhydride | | anhydride | | | | silane | | | | | |
| Example | Zinc | 258 | Silver (I) | 1.7 | — | 0 | — | 0 | 1-Propanol | 1096 | 180 | 5 |
| A1-3 | propionate | | acetate | | | | | | | | | |
| | anhydride | | anhydride | | | | | | | | | |
| Example | Zinc | 183 | Copper (II) | 1.9 | — | 0 | — | 0 | 1-Propanol | 7670 | 180 | 5 |
| A1-4 | acetate | | acetate | | | | | | | | | |
| | anhydride | | anhydride | | | | | | | | | |
| | | | Silver (I) | 0.8 | | | | | | | | |
| | | | acetate | | | | | | | | | |
| | | | anhydride | | | | | | | | | |
| Example | Zinc | 183 | Copper (II) | 0.9 | Indium | 3 | Tetra- | 16 | 2-Propanol | 1847 | 150 | 6 |
| A1-5 | acetate | | acetate | | acetate | | butoxy- | | | | | |
| | anhydride | | anhydride | | anhydride | | silane | | | | | |
| Example | Zinc | 183 | Silver (I) | 0.9 | Tin (IV) | 4 | — | 0 | 1-Propanol | 3880 | 160 | 5 |
| A1-6 | acetate | | acetate | | acetate | | | | | | | |
| | anhydride | | anhydride | | anhydride | | | | | | | |
| Example | Zinc | 183 | Silver (I) | 0.9 | Bismuth | 3 | — | 0 | Ethanol | 3880 | 160 | 6 |
| A1-7 | acetate | | acetate | | acetate | | | | | | | |
| | anhydride | | anhydride | | oxide | | | | | | | |
| Example | Zinc | 183 | Manganese | 5 | Indium | 3 | — | 0 | 1-Butanol | 3900 | 180 | 5 |
| A1-8 | acetate | | (II) acetate | | acetate | | | | | | | |
| | anhydride | | tetrahydrate | | anhydride | | | | | | | |
| Example | Zinc | 183 | Manganese | 10 | Indium | 3 | Titanium | 19 | Methanol | 4000 | 180 | 5 |
| A1-9 | acetate | | (II) acetate | | acetate | | (IV) tetra- | | | | | |
| | anhydride | | tetrahydrate | | anhydride | | buloxide | | tetramer | | | |
| Example | Zinc | 183 | Manganese | 20 | Indium | 3 | — | 0 | 2-Propanol | 4150 | 180 | 5 |
| A1-10 | acetate | | (II) acetate | | acetate | | | | | | | |
| | anhydride | | tetrahydrate | | anhydride | | | | | | | |
| Example | Zinc | 183 | Manganese | 20 | — | 0 | — | 0 | 2-Propanol | 4150 | 180 | 5 |
| A1-11 | acetate | | (II) acetate | | | | | | | | | |
| | anhydride | | tetrahydrate | | | | | | | | | |
| Example | Zinc | 183 | Manganese | 40 | Indium | 3 | Tetra- | 1.6 | Methanol | 4420 | 180 | 5 |
| A1-12 | acetate | | (II) acetate | | acetate | | methoxy- | | | | | |
| | anhydride | | tetrahydrate | | anhydride | | silane | | | | | |
| Example | Zinc | 183 | Manganese | 40 | — | 0 | — | 0 | 2-Propanol | 4420 | 180 | 5 |
| A1-13 | acetate | | (II) acetate | | | | | | | | | |
| | anhydride | | tetrahydrate | | | | | | | | | |
| Compar- | Zinc | 183 | — | 0 | — | 0 | Tetra- | 6 | Methanol | 1850 | 150 | 5 |
| ative | acetate | | | | | | methoxy- | | | | | |
| Example | anhydride | | | | | | silane | | | | | |
| A1-1 | | | | | | | | | | | | |
| Compar- | Zinc | 183 | — | 0 | Indium | 3 | — | 0 | 2-Propanol | 3900 | 160 | 5 |
| ative | acetate | | | | acetate | | | | | | | |
| Example | anhydride | | | | anhydride | | | | | | | |
| A1-2 | | | | | | | | | | | | |

TABLE 2

| | | | Crystal | | Disper- | | Bonding amount | | Content of | |
|---|---|---|---|---|---|---|---|---|---|---|
| | Particle | X-ray | grain | Disper- | sed and | | of acyl group | | added metal | |
| | concen- | diffrac- | diameter | sion | and ag- | Color | | Bonding | element (M′) | |
| | tration | tion | Dw | particle | gregated | of | Identi- | amount | | Atomic |
| | (wt %) | pattern | (mm) | diameter | states | powder | fication | mol %/Zn | M′ | %/Zn |
| Example | 2 | Equal to | 18 | ◯ | B | Gray | Ethanoyl | 4 | Cu | 0.1 |
| A1-1 | | ZnO | | | | | group | | | |
| Example | 4 | Equal to | 11 | ● | A | Gray | Formyl | 8 | Cu | 2 |
| A1-2 | | ZnO | | | | | group | | | |
| Example | 6 | Equal to | 14 | ◯ | A | Yellow | Propanoyl | 0.8 | Ag | 1 |
| A1-3 | | ZnO | | | | | group | | | |
| Example | 1 | Equal to | 6 | ◯ | A | Brown | Ethanoyl | 0.5 | Cu | 0.9 |
| A1-4 | | ZnO | | | | | group | | Ag | 0.5 |
| Example | 4 | Equal to | 14 | ⊚ | A | Bluish | Ethanoyl | 3.1 | Cu | 0.4 |
| A1-5 | | ZnO | | | | gray | group | | In | 0.9 |
| Example | 2 | Equal to | 15 | ◯ | A | Yellow | Ethanoyl | 1.8 | Ag | 0.6 |
| A1-6 | | ZnO | | | | | group | | Su | 1.2 |
| Example | 2 | Equal to | 12 | ◯ | A | Yellow | Ethanoyl | 2.3 | Ag | 0.5 |
| A1-7 | | ZnO | | | | | group | | Bi | 1 |
| Example | 2 | Equal to | 14 | ◯ | A | Slight | Ethanoyl | 1.6 | Mn | 1.8 |
| A1-8 | | ZnO | | | | yellow | group | | In | 1 |
| Example | 2 | Equal to | 15 | ⊚ | A | Slight | Ethanoyl | 1.5 | Mn | 3.2 |
| A1-9 | | ZnO | | | | yellow | group | | In | 1 |
| Example | 2 | Equal to | 15 | ◯ | A | Light | Ethanoyl | 1.8 | Mn | 6.4 |
| A1-10 | | ZnO | | | | yellow | group | | In | 1 |
| Example | 2 | Equal to | 16 | ◯ | A | Dark | Ethanoyl | 1.5 | Mn | 6.3 |
| A1-11 | | ZnO | | | | yellow | group | | | |
| Example | 2 | Equal to | 13 | ● | A | Light | Ethanoyl | 3 | Mn | 13 |
| A1-12 | | ZnO | | | | yellow | group | | In | 1 |
| Example | 2 | Equal to | 15 | ◯ | B | Dark | Ethanoyl | 1.5 | Mn | 13 |
| A1-13 | | ZnO | | | | yellow | group | | | |
| Compar- | 4 | Equal to | 12 | ⊚ | A | White | Ethanoyl | 3 | — | 0 |
| ative | | ZnO | | | | | group | | | |
| Example | | | | | | | | | | |
| A1-1 | | | | | | | | | | |
| Compar- | 2 | Equal to | 15 | ◯ | A | Blue | Ethanoyl | 3 | In | 1 |
| ative | | ZnO | | | | | group | | | |
| Example | | | | | | | | | | |
| A1-2 | | | | | | | | | | |

| | Content of | | | | | | |
|---|---|---|---|---|---|---|---|
| | surface- | | | | Ultra- | | Visible- |
| | treating | | | | violet | | ray |
| | metal (Ms) | | Trans- | | transmit | | transmit |
| | | Atomic | parency | | tance % | | tance % |
| | Ms | %/Zn | Haze % | Hue | 380 nm | 420 nm | 600 nm |
| Example | — | 0 | 8 | Color- | 15 | 72 | 82 |
| A1-1 | | | | less | | | |
| Example | Si | 1 | 0.4 | Color- | 13 | 69 | 90 |
| A1-4 | | | | less | | | |
| Example | — | 0 | 0.8 | Yellow | 2 | 1 | 62 |
| A1-3 | | | | | | | |
| Example | — | 0 | 0.3 | Brown | 3 | 19 | 36 |
| A1-4 | | | | | | | |
| Example | Si | 5 | 0.2 | Yellow | 6 | 59 | 85 |
| A1-5 | | | | | | | |
| Example | — | 0 | 0.5 | Yellow | 3 | 1 | 77 |
| A1-6 | | | | | | | |
| Example | — | 0 | 0.4 | Yellow | 5 | 3 | 77 |
| A1-7 | | | | | | | |
| Example | — | 0 | 1.5 | Slight | 4 | 41 | 89 |
| A1-8 | | | | yellow | | | |
| Example | Ti | 2 | 0.3 | Slight | 4 | 33 | 85 |
| A1-9 | | | | yellow | | | |
| Example | — | 0 | 0.7 | Light | 3 | 19 | 77 |
| A1-10 | | | | yellow | | | |
| Example | — | 0 | 1.2 | Dark | 10 | 34 | 70 |
| A1-11 | | | | yellow | | | |
| Example | Si | 1 | 0.2 | Light | 3 | 17 | 75 |
| A1-12 | | | | yellow | | | |
| Example | — | 0 | 3 | Dark | 8 | 26 | 64 |
| A1-13 | | | | yellow | | | |
| Compar- | Si | 4 | 0.6 | Color- | 36 | 70 | 91 |
| ative | | | | less | | | |
| Example | | | | | | | |
| A1-1 | | | | | | | |
| Compar- | — | 0 | 1.2 | Color- | 20 | 70 | 87 |
| ative | | | | less | | | |
| Example | | | | | | | |
| A1-2 | | | | | | | |

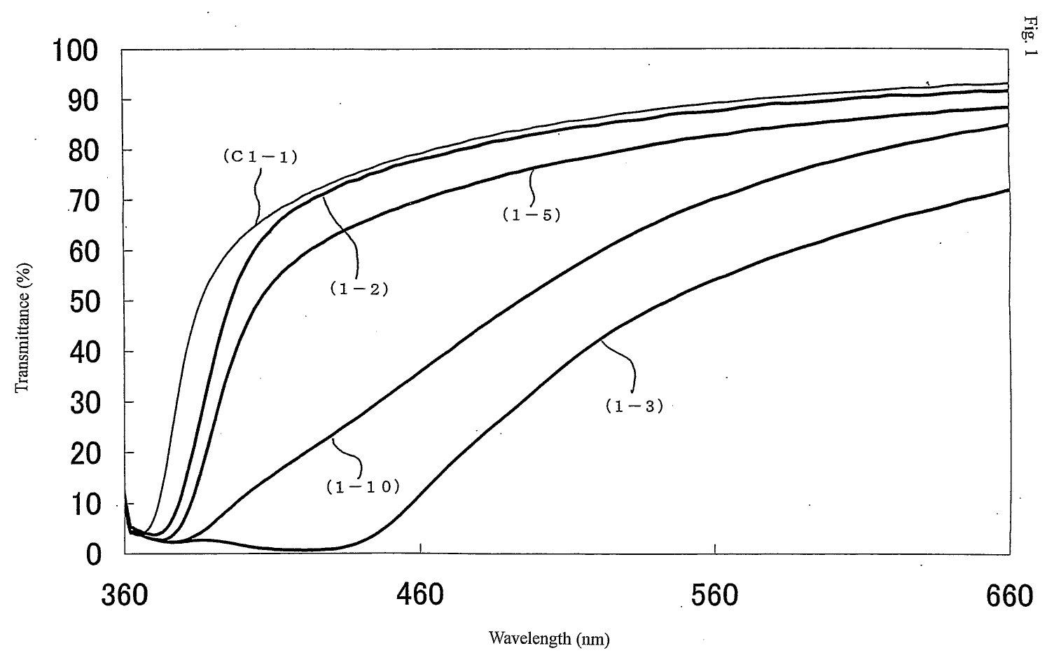

Shown in FIG. 1 are transmission spectra obtained by measuring the dilutions in accordance with the above evaluation method (4-1) wherein the dilutions were prepared in a way that the reaction liquids (1-2), (1-3), (1-5), (1-10), and (c1-1) having been obtained from Examples A1-2, A1-3, A1-5, A1-10, and Comparative Example A1-1 respectively were diluted to a particle concentration of 0.1 wt %.

From the results of Examples A1-8 to A1-13, it can be understood that, in the present invention, if the amount of Mn being added is increased, then the ultraviolet intercepting ability is enhanced, so that a transparent membrane excellent in the ultraviolet intercepting ability is obtained. Its reason is inferred as follows: the crystal grain diameter is not larger than 20 nm and therefore, even if the particles are secondarily aggregated in the stage of the reaction liquid, its aggregation force is so weak that the aggregated particles are easily dispersed due to the addition of the binder. On the other hand, if the amount of Mn is increased, then the ultraviolet intercepting ability is enhanced, but the visible-ray transmittance is deteriorated. Particularly, if the amount of Mn exceeds 10% (Example A1-13), then the yellowing tends to be strong, and the visible-ray transmittance is low. If this is considered, then it can be understood that: in the present invention, in order to achieve a higher ultraviolet intercepting ability, a higher visible-ray transmittance, and a low coloring degree, in cases of Mn, its content is favorably in the range of Mn/Zn=3 to 10 atomic % like in Examples A1-9 and A1-10. However, like in Example A1-12, it is also clear that: in the present invention, if the Si compound or Ti compound (as the surface-treating metal compound) is bonded to particle surfaces even in cases where the amount of Mn exceeds 10%, then it is possible to enhance not only the ultraviolet intercepting ability but also the visible-ray transmittance.

### EXAMPLE A1-14

The same reaction apparatus as of Example A1-1 was used and, into its reactor, there was charged a mixture comprising 3,000 parts of pure water, 50 parts of cerium(III) acetate monohydrate, and 0.6 part of copper(II) acetate anhydride, and then there was added 50 parts of a 30% aqueous hydrogen peroxide solution under stirring at room temperature. Next, under stirring, the temperature of the mixture was raised from the room temperature and then heat-retained at 90° C.±2° C. for 5 hours, and then 10 parts of a 30% aqueous hydrogen peroxide solution was added. Thereafter, the temperature was heat-retained at the same temperature as the above for another 1 hour to thus carry out a reaction to form metal oxide particles and then cooled, thereby obtaining a reaction liquid (1-14) containing slightly yellow and high-transparent-feeling fine particles (metal oxide particles according to the present invention) in a concentration of 0.8 wt %.

Next, the resultant reaction liquid (1-14) was subjected to filtration with an ultrafiltration membrane to thereby remove impurity ions and residual hydrogen peroxide and also make concentration, thus obtaining a water dispersion (1-14) of 7 wt % in fine particle concentration.

The metal oxide particles in the water dispersion (1-14) were subjected to the powder X-ray diffractometry. As a result, the peak was broad, but a pattern equal to CeO₂ was shown. In addition, as to the crystal grain diameter, the primary particle diameter was determined from a TEM image, because the peak was broad. The ultraviolet absorption property and the visible-ray transmission property were evaluated by the above evaluation method (4-1). Incidentally, in the measurement, ion-exchanged water was used as the diluting solvent. The results of the above are shown in Table 3.

### COMPARATIVE EXAMPLE A1-3

A water dispersion (c1-3) of 7 wt % in fine particle concentration was obtained in the same way as of Example A1-14 except that there was not used the copper(II) acetate anhydride.

The metal oxide particles in the water dispersion (c1-3) were evaluated in the same way as of Example A1-14. Their results are shown in Table 3.

TABLE 3

| | Content of | | | | Ultraviolet | | Visible- |
|---|---|---|---|---|---|---|---|
| | added metal | | X-ray | Primary | transmittance | | ray |
| | element (M′) | | diffrac- | particle | (%) | | transmit- |
| | | Atomic | tion | diameter | 380 | 400 | tance (%) |
| | M′ | %/Ce | pattern | (nm) | nm | nm | 600 nm |
| Example | Cu | 2 | Equal to | 2-4 | 4 | 70 | 99 |
| A1-14 | | | CeO₂ | | | | |
| Compar- | — | — | Equal to | 2-4 | 29 | 75 | 99 |
| ative | | | CeO₂ | | | | |
| Example | | | | | | | |
| A1-3 | | | | | | | |

### EXAMPLE A1-15

The same reaction apparatus as of Example A1-1 was used and, into its reactor, there was charged a mixture comprising 2,400 parts of ethylene glycol dimethyl ether (as a reaction solvent), 303 parts of titanium methoxypropoxide, 2.8 parts of silver(I) acetate, and 270 parts of acetic acid, and then its gas phase portion was purged with nitrogen gas. Thereafter, under stirring, the temperature of the mixture was raised from 20° C. and then heat-retained at 180° C.±1° C. for 5 hours to thus carry out a reaction to form metal oxide particles and then cooled, thereby obtaining a reaction liquid (1-15) containing fine particles (metal oxide particles according to the present invention) in a fine particle concentration of 2 wt %.

The metal oxide particles in the reaction liquid (1-15) were subjected to the powder X-ray diffractometry. As a result, a pattern equal to anatase type titanium oxide was shown, and the crystal grain diameter was 6 nm. In addition, the ultraviolet absorption property and the visible-ray transmission property were evaluated by the above evaluation method (4-1). The results of the above are shown in Table 4.

### EXAMPLE A1-16

A reaction liquid (1-16) containing fine particles (metal oxide particles according to the present invention) in a fine particle concentration of 2 wt % was obtained in the same way as of Example A1-15 except that the silver(I) acetate was replaced with 6.5 parts of manganese(II) acetate.

The metal oxide particles in the reaction liquid (1-16) were subjected to the powder X-ray diffractometry. As a result, a pattern equal to anatase type titanium oxide was shown, and the crystal grain diameter was 6 nm. In addition, the ultraviolet absorption property and the visible-ray transmission property were evaluated by the above evaluation method (4-1). The results of the above are shown in Table 4.

### [Comparative Example A1-4]

A reaction liquid (c1-4) containing fine particles in a fine particle concentration of 2 wt % was obtained in the same way as of Example A1-15 except that there was not used the silver(I) acetate.

The metal oxide particles in the reaction liquid (c1-4) were evaluated in the same way as of Example A1-15. Their results are shown in Table 4.

TABLE 4

| | Content of | | | | Ultraviolet | | Visible- |
|---|---|---|---|---|---|---|---|
| | added metal | | X-ray | Crystal | transmittance | | ray |
| | element (M′) | | diffrac- | grain | (%) | | transmit- |
| | | Atomic | tion | diameter | 380 | 400 | tance (%) |
| | M′ | %/Ti | pattern | Dw (nm) | nm | nm | 600 nm |
| Example | Ag | 2 | Equal to | 6 | 3 | 4 | 60 |
| A1-15 | | | TiO₂ | | | | |
| Example | Mn | 5 | Equal to | 6 | 30 | 20 | 75 |
| A1-16 | | | TiO₂ | | | | |
| Compar- | — | — | Equal to | 8 | 40 | 60 | 78 |
| ative | | | TiO₂ | | | | |
| Example | | | | | | | |
| A1-4 | | | | | | | |

### EXAMPLE A1-17

First, a powder of Bi-containing zinc acetate was synthesized. Specifically, a mixture of 250 parts of an 80 wt % aqueous acetic acid solution, 36.7 parts of zinc acetate, and 2.84 parts of bismuth(III) acetate oxide was charged into a glass reactor possible to externally heat and equipped with a stirrer, an addition inlet, and a thermometer. Thereafter, the mixture was heated under stirring to raise its temperature and then stirred at 100° C. for 5 hours, thus obtaining a homogeneous transparent solution. Thereafter, the internal temperature was raised to 120° C. and then cooled, thereby obtaining a white slurry. The solvent component was removed from the resultant slurry at a bath temperature of 50° C. under reduced pressure with an evaporator. Furthermore, the resultant white powder was heat-dried at 40° C. with a vacuum drier for 10 hours, thus obtaining a powder (1). The resultant powder (1) was subjected to elemental analysis by fluorescent X-ray analysis and to crystal analysis by powder XRD. As a result, it was found to be zinc acetate containing Bi in a ratio of 5 atomic % relative to Zn.

Next, the same reaction apparatus as of Example A1-1 was used and, into its reactor, there was charged a mixture comprising 18 parts of the above-obtained powder (1) and 180 parts of methanol, and then its gas phase portion was purged with nitrogen gas. Thereafter, under stirring, the temperature of the mixture was raised from 20° C. to 150° C. and then retained at 150° C.-±1° C. for 5 hours and then cooled, thereby obtaining a reaction liquid (1-17) containing yellow fine particles. This reaction liquid (1-17) was found to be a material in which fine particles (average particle diameter: 10 nm) comprising a ZnO crystal in which Bi(III) was held in solid solution in a ratio of 5 atomic % relative to Zn were contained and dispersed in a concentration of 4 wt %.

From the results of having evaluated a diffused reflectance spectrum of the fine particles in the reaction liquid (1-17), they were found to exercise: an absorption having an absorption maximum at 422 nm based on the containing of Bi; and a band edge absorption (absorption having its longer-wavelength-side absorption edge at 400 nm) based on the ZnO crystal. In addition, it was found such that: the above fine particles exercised an absorption (though it was weak as the absorption ability) from near 650 nm to the longer wavelength side (infrared wavelength range); and their ultraviolet absorption edge wavelength (on the shorter wavelength side) was blue-shifted (the absorption edge was on the shorter wavelength side) when compared with a diffused reflectance spectrum of Bi-free ZnO. Thereby it was indirectly supported that a Bi ion of 3 in valence was held in solid solution.

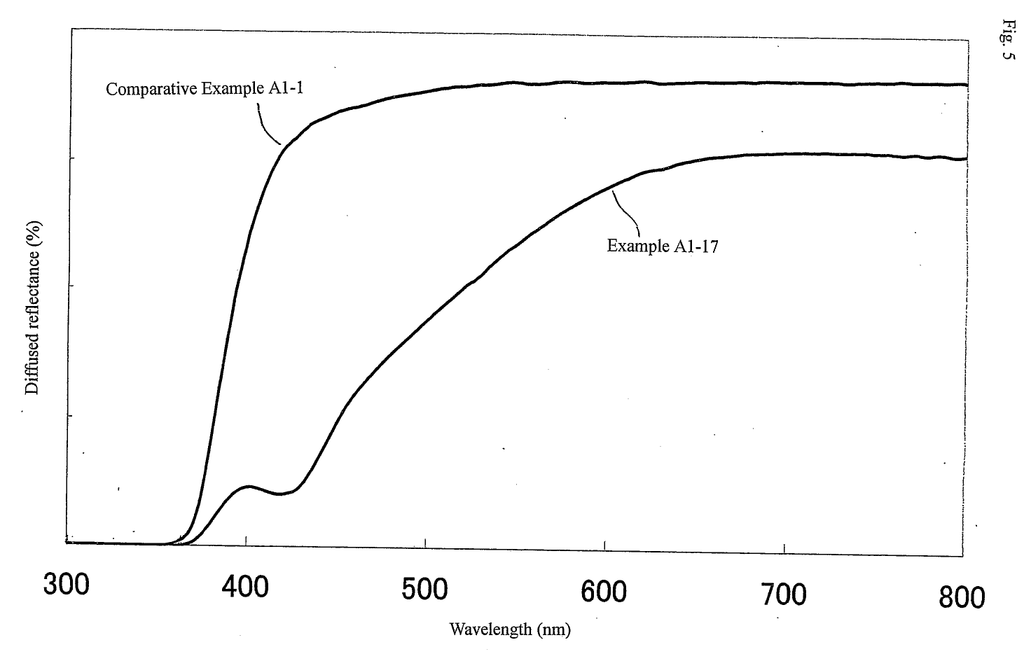

The diffused reflectance spectrum measurement result of the fine particles having been obtained from Example A1-17 is shown in FIG. 5 along with that of the fine particles having been obtained from Comparative Example A1-1.

The resultant reaction liquid (1-17) was subjected to heating solvent displacement to thereby obtain a dispersion (1-17) such that the above fine particles were contained and dispersed in 2-propanol in a concentration of 20 wt %.

### EXAMPLE A1-18

The same reaction apparatus as of Example A1-1 was used and, into its reactor, there was charged a dispersion such that 10 parts of superfine CeO₂ particles (average particle diameter: 8 nm) were dispersed in 190 parts of n-butanol, and then this dispersion was stirred. On the other hand, 44 parts of a solution was prepared by dissolving bismuth(III) acetate oxide into a mixed solvent of propionic acid and n-butanol so that the concentration would be 20 wt %, and then this prepared solution was charged into the addition tank. Under stirring, the temperature of the above dispersion of the superfine CeO₂ particles was raised and retained at 200° C. Thereto from the addition tank, there was added the bismuth(III) acetate oxide solution. Still after the end of this addition, the resultant mixture was retained at 200° C. for 5 hours and then cooled, thereby obtaining a reaction liquid (1-18) containing yellow fine particles.

The reaction liquid (1-18) was found to be such that fine particles (average particle diameter: 10 nm) comprising superfine CeO₂ particles of which the surfaces were coated with a Bi oxide (Bi₂O₃) layer of about 1 nm in thickness were dispersed and contained in a solvent (its main solvent: n-butanol) in a concentration of 7 wt %. In addition, the above fine particles were found to have an average metal composition of Bi/Ce=0.53/1 (atomic ratio).

The resultant reaction liquid (1-18) was subjected to heating solvent displacement to thereby obtain a dispersion (1-18) such that the above fine particles were contained and dispersed in butyl acetate in a concentration of 20 wt %.

### EXAMPLE A1-19

A reaction liquid (1-19) containing yellow fine particles was obtained in the same way as of Example A1-18 except that the superfine CeO₂ particles were replaced with superfine TiO₂ particles (average particle diameter: 12 nm) and that the n-butanol was all replaced with ethanol.

The reaction liquid (1-19) was found to be such that fine particles (average particle diameter: 14 nm) comprising superfine TiO₂ particles of which the surfaces were coated with a Bi oxide (Bi₂O₃) layer of about 1 nm in thickness were dispersed and contained in a solvent (its main solvent: ethanol) in a concentration of 7 wt %. In addition, the above fine particles were found to have an average metal composition of Bi/Ti=0.25/1 (atomic ratio).

The resultant reaction liquid (1-19) was subjected to heating solvent displacement to thereby obtain a dispersion (1-19) such that the above fine particles were contained and dispersed in water in a concentration of 20 wt %.

### EXAMPLE A1-20

Into the same reactor as of Example A1-17, there was charged a mixture comprising 18 parts of zinc acetate anhydride powder, 0.9 part of indium acetate anhydride powder, and 160 parts of 2-butoxyethanol, and then its gas phase portion was purged with nitrogen gas. Thereafter, under stirring, the temperature of the mixture was raised and then retained at 200° C. for 3 hours (at this point of time, superfine ZnO particles in which In was held in solid solution were formed). Thereafter, a suspension, such that 18 parts of the powder (1) having been obtained from Example A1-17 was dispersed in 18 parts of 2-butoxyethanol, was added from the addition tank. After the end of this addition, the resultant mixture was retained at 200° C. for 3 hours and then cooled, thereby obtaining a reaction liquid (1-20) containing fine particles.

This reaction liquid (1-20) was found to be a material in which fine particles (average particle diameter: 18 nm) comprising a ZnO crystal in which Bi(III) and In(III) were held in solid solution in ratios of 2.5 atomic % and 1.5 atomic % respectively relative to Zn (wherein Bi(III) was held in solid solution locally in a surface ZnO layer of the fine particles) were contained and dispersed in a concentration of 7.4 wt %.

From the results of having evaluated a diffused reflectance spectrum of the fine particles in the reaction liquid (1-20), they were found to exercise: an absorption having an absorption maximum at 422 nm based on the containing of Bi; and a band edge absorption (absorption having its longer-wavelength-side absorption edge at 400 nm) based on the ZnO crystal; and besides, a strong absorption in the near-infrared range which could be considered to be caused by plasma absorption due mainly to the solid solution of In.

The resultant reaction liquid (1-20) was heat-concentrated under reduced pressure with an evaporator, thereby obtaining a dispersion (1-20) of 20 wt % in concentration of the above fine particles.

### EXAMPLE A1-21

First, a powder of Bi-and-In-containing zinc acetate was synthesized. Specifically, a powder (2) was obtained in the same way as of the synthesis of the Bi-containing zinc acetate in Example A1-17 except that the amount of the bismuth(III) acetate oxide being used was changed to 1.7 parts and that 1.8 parts of indium acetate anhydride was further used. The resultant powder (2) was subjected to elemental analysis by fluorescent X-ray analysis and to crystal analysis by powder XRD. As a result, it was found to be zinc acetate containing Bi and In wherein their contents were both 3 atomic % relative to Zn.

Next, into the same reactor as of Example A1-17, there was charged a mixture comprising 18 parts of the above-obtained powder (2) and 160 parts of 2-butoxyethanol, and then its gas phase portion was purged with nitrogen gas. Thereafter, under stirring, the temperature of the mixture was raised and then retained at 200° C. for 3 hours and then cooled, thereby obtaining a reaction liquid (1-21) containing fine particles.

This reaction liquid (1-21) was found to be a material in which fine particles (average particle diameter: 8 nm) comprising a ZnO crystal in which Bi(III) and In(III) were held in solid solution (wherein their contents were both 3 atomic % relative to Zn) were contained and dispersed in a concentration of 4.5 wt %.

The resultant reaction liquid (1-21) was heat-concentrated under reduced pressure with an evaporator, thereby obtaining a dispersion (1-21) of 20 wt % in concentration of the above fine particles.

### EXAMPLE A1-22

A reaction liquid (1-22) was obtained in the same way as of Example A1-1 except that 0.1 part of lithium acetate dihydrate was used as an additional raw material.

The resultant reaction liquid (1-22) was evaluated in the same way as of Example A1-1. As a result, the metal oxide particles in the reaction liquid (1-22) were fine particles of 18 nm in crystal grain diameter comprising a ZnO crystal containing Cu and Li in ratios of 0.1 atomic % and 0.08 atomic % respectively relative to Zn and were 10% in ultraviolet transmittance at 380 nm and 82% in visible-ray transmittance.

### EXAMPLE A1-23

A reaction liquid (1-23) was obtained in the same way as of Example A1-11 except that 0.3 part of cesium hydroxide was used as an additional raw material.

The resultant reaction liquid (1-23) was evaluated in the same way as of Example A1-11. As a result, the metal oxide particles in the reaction liquid (1-23) were fine particles of 16 nm in crystal grain diameter comprising a ZnO crystal containing Mn and Cs in ratios of 6.3 atomic % and 0.2 atomic % respectively relative to Zn and were 7% in ultraviolet transmittance at 380 nm and 70% in visible-ray transmittance.

### EXAMPLE A1-24

A reaction liquid (1-24) was obtained in the same way as of Example A1-7 except that 2 parts of calcium acetate monohydrate was used as an additional raw material.

The resultant reaction liquid (1-24) was evaluated in the same way as of Example A1-7. As a result, the metal oxide particles in the reaction liquid (1-24) were fine particles of 12 nm in crystal grain diameter comprising a ZnO crystal containing Ag, Bi, and Ca in ratios of 0.5 atomic %, 1 atomic %, and 1.1 atomic % respectively relative to Zn and were 3% in ultraviolet transmittance at 380 nm and 78% in visible-ray transmittance.

### EXAMPLE A1-25

A reaction liquid (1-25) was obtained in the same way as of Example A1-15 except that 0.3 part of magnesium acetate tetrahydrate was used as an additional raw material.

The resultant reaction liquid (1-25) was evaluated in the same way as of Example A1-15. As a result, the metal oxide particles in the reaction liquid (1-25) were fine particles of 6 nm in crystal grain diameter comprising an anatase type TiO₂ crystal containing Ag and Mg in ratios of 2 atomic % and 0.1 atomic % respectively relative to Ti and were 1% in ultraviolet transmittance at 380 nm and 62% in visible-ray transmittance.

### EXAMPLE A2-1

An amount of 1,000 parts of the reaction liquid (1-1) having been obtained from Example A1-1 was heated with an evaporator and thereby concentrated to 100 parts. Thereafter, while propylene glycol methyl ether acetate was added thereto as a substitutional solvent, the solvent component was distilled off at the same time, thus obtaining a dispersion containing the propylene glycol methyl ether acetate as a solvent component. Next, this dispersion was subsequently heat-concentrated and then cooled, and then 0.2 part of titanium(IV) tetrabutoxide tetramer (produced by Wako Pure Chemical Industries, Ltd.) was added thereto as a dispersant, thus obtaining a dispersion (2-1). The particle concentration and dispersion particle diameter of the resultant dispersion are shown in Table 5.

### EXAMPLES A2-2 to A2-10

Dispersions (2-2) to (2-10) were obtained in the same way as of Example A2-1 except that the reaction liquids as shown in Table 5 were used and that the substitutional solvents and the dispersants, as shown in Table 5, were used. The particle concentrations and dispersion particle diameters of the resultant dispersions are shown in Table 5.

TABLE 5

| | | | Dispersant | | Particle | Dispersion |
|---|---|---|---|---|---|---|
| | | Substitu- | | Addition | concen- | particle |
| | Reaction | tional | | amount (wt | tration | diameter |
| | liquid | solvent | Kind | %/particles) | (wt %) | (μm) |
| Example | (1-1) | Propylene | Titanium (IV) | 1 | 20 | 0.08 |
| A2-1 | | glycol methyl | tetrabutoxide | | | |
| | | ether acetate | tetramer | | | |
| Example | (1-2) | 1-Propanol | — | 0 | 20 | 0.03 |
| A2-2 | | | | | | |
| Example | (1-3) | Methyl | Titanium (IV) | 10 | 20 | 0.04 |
| A2-3 | | isobutyl | tetrabutoxide | | | |
| | | ketone | tetramer | | | |
| Example | (1-4) | Xylene | — | 0 | 30 | 0.09 |
| A2-4 | | | | | | |
| Example | (1-5) | 1-Butanol | — | 0 | 25 | 0.04 |
| A2-5 | | | | | | |
| Example | (1-6) | Water | — | 0 | 16 | 0.05 |
| A2-6 | | | | | | |
| Example | (1-7) | Methyl ethyl | — | 0 | 22 | 0.21 |
| A2-7 | | ketone | | | | |
| Example | (1-9) | 1-Butanol | — | 0 | 20 | 0.05 |
| A2-8 | | | | | | |
| Example | (1-10) | Propylene | Partially | 5 | 30 | 0.03 |
| A2-9 | | glycol | hydrolyzed-condensed | | | |
| | | monomethyl | products (trimer to | | | |
| | | ether | pentamer) of | | | |
| | | | tetramethoxysilane | | | |
| Example | (1-12) | 1-Butanol | — | 0 | 30 | 0.03 |
| A2-10 | | | | | | |

### EXAMPLE A3-1

An amount of 36 parts of a silicate binder (“MKC Silicate MS56” produced by Mitsubishi Chemical Corporation) and 1 part of n-butylamine (as a catalyst) were added to 100 parts of the dispersion (2-1) (having been obtained from Example A2-1), and then they were stirred together, thus obtaining a paint (3-1). The dispersion particle diameter of the fine particles in this paint was 0.04 μm.

Next, alkali glass was used as the substrate to repeatedly carry out a plurality of times an operation of coating the paint (3-1) onto the above substrate by use of a bar coater and then drying them at normal temperature and then heating them at 200° C. for 60 minutes. Thereby there were obtained a fine-particles-dispersed-membrane-coated substrate (3-1-A) having a fine-particles-dispersed membrane of 2 μm in dry membrane thickness and a fine-particles-dispersed-membrane-coated substrate (3-1-B) having a fine-particles-dispersed membrane of 4 μm in dry membrane thickness.

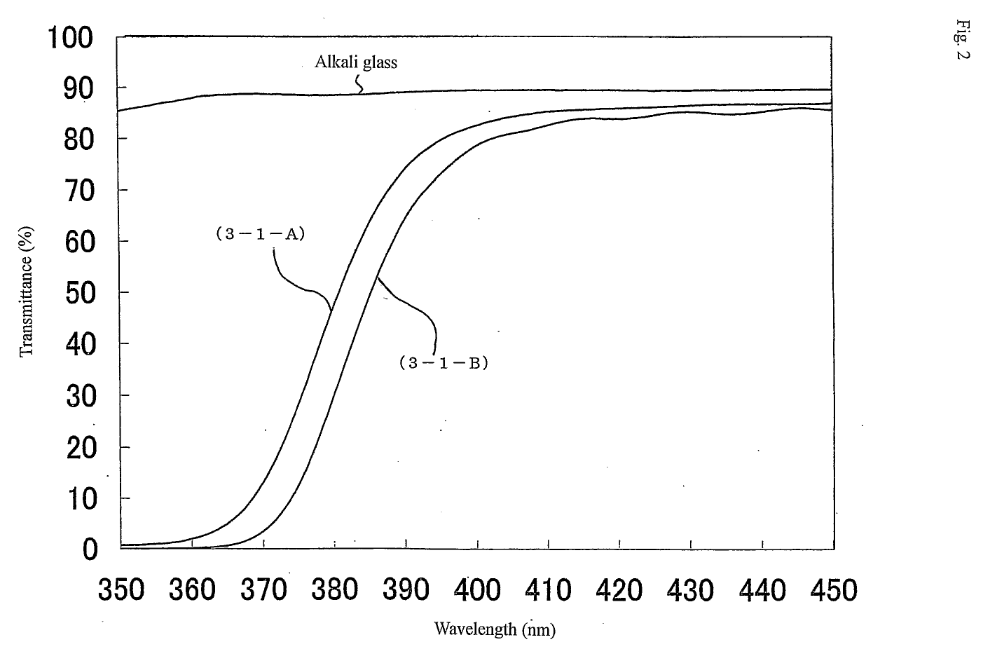

Transmission spectra of the resultant fine-particles-dispersed-membrane-coated substrates (3-1-A) and (3-1-B) are shown in FIG. 2 along with that of the alkali glass which is the raw substrate.

In addition, the transparency and hue of the resultant fine-particles-dispersed-membrane-coated substrates (3-1-A) and (3-1-B) were evaluated. As a result, as to both, the transparency was 0.3% in haze, and the hue was colorless.

### EXAMPLE A3-2

An amount of 36 parts of a silicate binder (“MICC Silicate MS56” produced by Mitsubishi Chemical Corporation) and 1 part of n-butylamine (as a catalyst) were added to 100 parts of the dispersion (2-3) (having been obtained from Example A2-3), and then they were stirred together, thus obtaining a paint (3-2). The dispersion particle diameter of the fine particles in this paint was 0.05 μm.

Next, alkali glass was used as the substrate to repeatedly carry out a plurality of times an operation of coating the paint (3-2) onto the above substrate by use of a bar coater and then drying them at normal temperature and then heating them at 200° C. for 60 minutes. Thereby there was obtained a fine-particles-dispersed-membrane-coated substrate (3-2) having a fine-particles-dispersed membrane of 3 μm in dry membrane thickness.

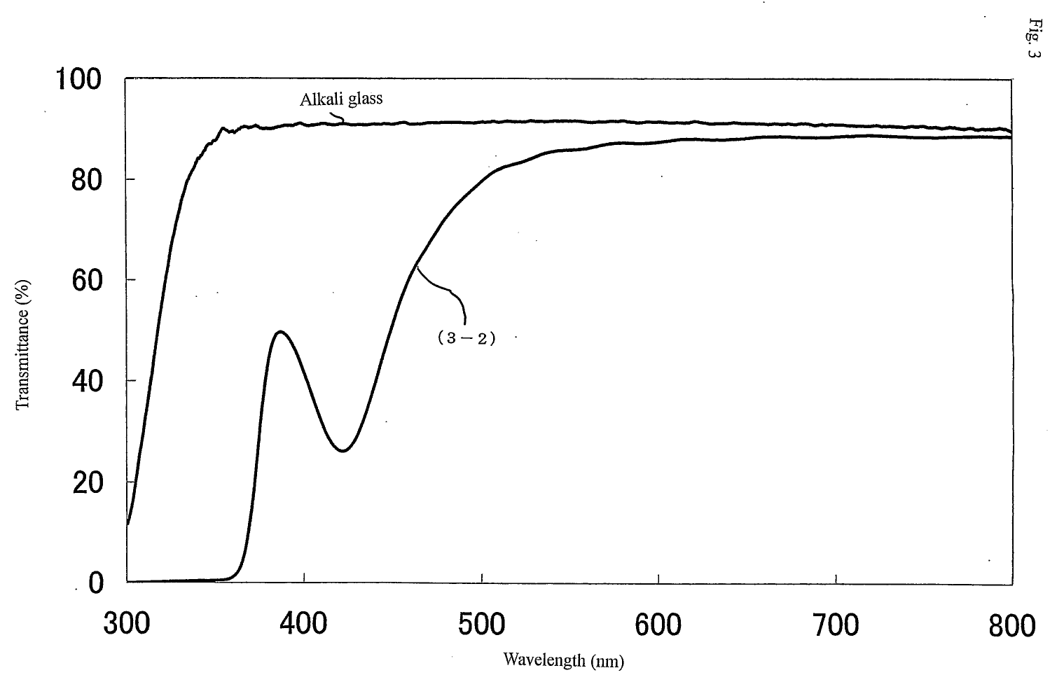

A transmission spectrum of the resultant fine-particles-dispersed-membrane-coated substrate (3-2) is shown in FIG. 3 along with that of the alkali glass which is the raw substrate.

In addition, the transparency and hue of the resultant fine-particles-dispersed-membrane-coated substrate (3-2) were evaluated. As a result, the transparency was 0.5% in haze, and the hue was yellow.

### EXAMPLE A3-3

An amount of 60 parts of an acrylic resin solution (solid component concentration: 50 wt %) and 20 parts of a mixed solvent of toluene/methyl ethyl ketone=1/1 (weight ratio) (as a diluting solvent) were added to 100 parts of the dispersion (2-9) (having been obtained from Example A2-9), and then they were stirred together, thus obtaining a paint (3-3). The dispersion particle diameter of the fine particles in this paint was 0.06 μm.

Next, a polyester film was used as the substrate and, onto this substrate, the paint (3-3) was coated by use of a bar coater in a way for the wet membrane thickness to be 24 μm. Thereafter, they were heated at 100° C. for 10 minutes to thereby obtain a fine-particles-dispersed-membrane-coated substrate (3-3) having a fine-particles-dispersed membrane.

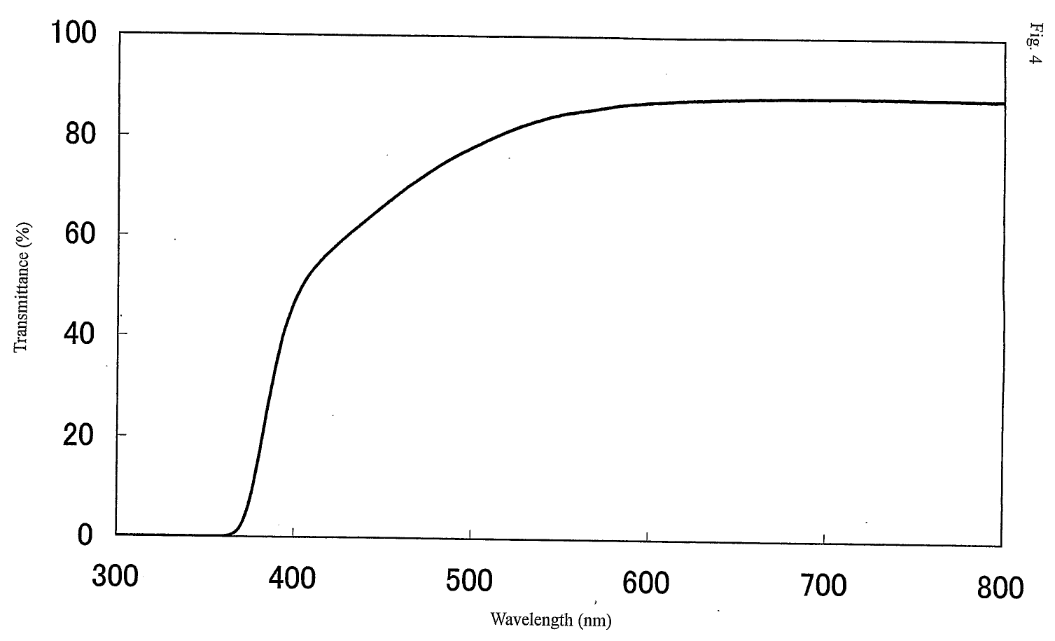

A transmission spectrum of the resultant fine-particles-dispersed-membrane-coated substrate (3-3) is shown in FIG. 4.

In addition, the transparency and hue of the resultant fine-particles-dispersed-membrane-coated substrate (3-3) were evaluated. As a result, the transparency was 0.5% in haze, and the hue was slightly yellow.

### EXAMPLE A3-4

An amount of 20 parts of an ultraviolet curing type coating agent (“HIC2000” produced by KYOEISHA CHEMICAL Co., LTD.; solid component content: 50 wt %; refractive index: 1.58) was added to 100 parts of the dispersion (2-2) (having been obtained from Example A2-2), and then they were stirred together, thus obtaining a paint (3-4).

Next, a high transparent polyethylene terephthalate film was used as the substrate and, onto this substrate, the paint (3-4) was coated by use of a bar coater. Thereafter, they were heated at 100° C. for 10 minutes and then irradiated with ultraviolet rays by use of a high-pressure mercury lamp for 10 minutes to thereby obtain a fine-particles-dispersed-membrane-coated substrate (3-4) having a fine-particles-dispersed membrane of 5 μm in dry membrane thickness.

The resultant fine-particles-dispersed-membrane-coated substrate (3-4) was, similarly to Example A3-1, a material which intercepted ultraviolet rays and had a high visible-ray transmission property.

In addition, the transparency and hue of the resultant fine-particles-dispersed-membrane-coated substrate (3-4) were evaluated. As a result, the transparency was 0.5% in haze, and the hue was colorless, and the refractive index of the membrane was 1.7.

### EXAMPLE A3-5

An amount of 100 parts of the dispersion (1-17) (having been obtained from Example A1-17), 40 parts of a silicate binder (hydrolyzed-condensed product of tetramethoxysilane; concentration in terms of silica=50 wt %), and 1 part of n-butylamine (as a catalyst) were mixed together, thereby obtaining a composition (3-5) for membrane formation.

The composition (3-5) was coated onto a glass plate (as a substrate) by use of a bar coater and then wet-cured at normal temperature and then, in a heating furnace, heated from the normal temperature at a temperature-raising rate of 2° C./min and then retained at 300° C. for 2 hours, thus forming a membrane of 4 μm in membrane thickness on a surface of the glass plate.

The formed membrane was a membrane such that superfine ZnO particles in which Bi was held in solid solution were dispersed and contained in a noncrystal silica membrane.

The spectroscopic properties of the glass plate (membrane-coated glass plate), on the surface of which there was formed the membrane, were evaluated. From their results, this glass plate was found to absorb ultraviolet rays over a wide range, specifically, to intercept ultraviolet rays of not longer than 370 nm by absorption based on a band gap of the ZnO (first absorption) and to further exercise an absorption (based on the containing of Bi) (second absorption) on the longer wavelength side (having its absorption edge at 416 nm). The transmittances of light of the designated wavelengths were 600 nm: 84%, 500 nm: 77%, 410 nm: 37%, and 370 nm: 1.5%.

The visible-ray transparency of the membrane-coated glass plate was “◯”, and its coloring was also in an inconspicuous degree.

### EXAMPLE A3-6

An amount of 24 parts of the dispersion (1-17) (having been obtained from Example A1-17), 16 parts of an acrylic resin binder (containing a polyisocyanurate curing agent), and 50 parts of butyl acetate-toluene (as a solvent) were mixed together, thereby obtaining a composition (3-6) for membrane formation.

The composition (3-6) was coated onto a PET film (as a substrate) by use of a bar coater in a way for the dry membrane thickness to be 8 μm and then retained at 100° C. for 5 minutes, thus forming a membrane of 8 μm in membrane thickness on a surface of the PET film.

The formed membrane was a membrane such that superfine ZnO particles in which Bi was held in solid solution were dispersed and contained in an acrylic resin membrane.

The spectroscopic properties of the PET film (membrane-coated PET film), on the surface of which there was formed the membrane, were evaluated. From their results, this PET film was found to be a film exercising the first and second absorptions similarly to the membrane-coated glass plate of Example A3-5 and therefore exercising an excellent ultraviolet-rays-cutting property.

The visible-ray transparency of the membrane-coated PET film was “◯”, and its coloring was also in an inconspicuous degree.

### EXAMPLE A3-7

An amount of 100 parts of the dispersion (1-18) (having been obtained from Example A1-18) and 100 parts of silica sol (solvent: IPA, silica concentration: 20 wt %) (as a binder) were mixed together, thereby obtaining a composition (3-7) for membrane formation.

The composition (3-7) was coated onto the same glass plate as of Example A3-5 by use of a bar coater and then heated at 300° C., thus forming a membrane of 2 μm in membrane thickness on a surface of the glass plate.

The membrane having been formed from the composition (3-7) was a membrane such that superfine CeO₂ particles as coated with Bi₂O₃ were dispersed and contained in a noncrystal silica membrane.

The spectroscopic properties of the glass plate (membrane-coated glass plate), on the surface of which there was formed the membrane, were evaluated. From their results, this glass plate was found to absorb ultraviolet rays of not longer than 360 nm in wavelength and to further exercise also an absorption at a wavelength of not longer than 420 nm based on the containing of Bi.

### EXAMPLE A3-8

An amount of 100 parts of the dispersion (1-19) (having been obtained from Example A1-19) and 100 parts of silica sol (solvent: IPA, silica concentration: 20 wt %) (as a binder) were mixed together, thereby obtaining a composition (3-8) for membrane formation.

The composition (3-8) was coated onto the same glass plate as of Example A3-5 by use of a bar coater and then heated at 300° C., thus forming a membrane of 2 μm in membrane thickness on a surface of the glass plate.

The membrane having been formed from the composition (3-8) was a membrane such that superfine TiO₂ particles as coated with Bi₂O₃ were dispersed and contained in a noncrystal silica membrane.

The spectroscopic properties of the glass plate (membrane-coated glass plate), on the surface of which there was formed the membrane, were evaluated. From their results, this glass plate was found to absorb ultraviolet rays of not longer than 360 nm in wavelength and to further exercise also an absorption at a wavelength of not longer than 420 nm based on the containing of Bi.

### EXAMPLE A3-9

The dispersion (1-17) (having been obtained from Example A1-17) was coated onto a glass plate (as a substrate) by use of a bar coater and then heat-dried at 100° C. in a heating furnace, of which the temperature was thereafter raised and then retained for 2 hours after having reached 350° C., thus forming a membrane of 2 μm in membrane thickness on a surface of the glass plate.

The formed membrane was a ZnO-crystal membrane in which Bi was contained in a ratio of 5 atomic % relative to Zn.

The spectroscopic properties of the glass plate (membrane-coated glass plate), on the surface of which there was formed the membrane, were evaluated. From their results, this glass plate was found to absorb ultraviolet rays over a wide range, specifically, to intercept ultraviolet rays of not longer than 370 nm by absorption based on a band gap of the ZnO (first absorption) and to further exercise an absorption (based on the containing of Bi) (second absorption) on the longer wavelength side (having its absorption edge at 416 nm).

The transparency of the membrane-coated glass plate was 0.6% in haze.

### EXAMPLE A3-10

A membrane of 2 μm in membrane thickness was formed on a surface of the glass plate in the same way as of Example A3-9 except that the dispersion (1-17) was replaced with the dispersion (1-21) (having been obtained from Example A1-21) and that the heat-drying temperature was changed to 200° C.

The formed membrane was a ZnO-crystal membrane containing Bi and In wherein their contents were both 3 atomic % relative to Zn.

The spectroscopic properties of the glass plate (membrane-coated glass plate), on the surface of which there was formed the membrane, were evaluated. From their results, this glass plate was found to absorb ultraviolet rays over a wide range, specifically, to exercise the first and second absorptions similarly to Example A3-9 and to further exercise an interception property against a near-infrared range of light.

The transparency of the membrane-coated glass plate was 1.2% in haze.

### EXAMPLE A3-11

A membrane of 2 μm in membrane thickness was formed on a surface of the glass plate in the same way as of Example A3-9 except that the dispersion (1-17) was replaced with the dispersion (1-19) (having been obtained from Example A1-19) and that the heat-drying temperature was changed to 500° C.

The formed membrane was a crystal membrane comprising a crystal in which Bi was contained in a ratio of 25 atomic % relative to Ti.

The spectroscopic properties of the glass plate (membrane-coated glass plate), on the surface of which there was formed the membrane, were evaluated. From their results, the membrane was found to be a membrane excellent in reflection properties.

### EXAMPLE A3-12

A membrane of 2 μm in membrane thickness was formed on a surface of the glass plate in the same way as of Example A3-9 except that the dispersion (1-17) was replaced with a 2-propanol dispersion (1-3) of 20 wt % in concentration having been obtained by subjecting the reaction liquid (1-3) (having been obtained from Example A1-3) to heating solvent displacement and that the heat-drying temperature was changed to 200° C.

The formed membrane was a ZnO-crystal membrane in which Ag was contained in a ratio of 1 atomic % relative to Zn.

The spectroscopic properties of the glass plate (membrane-coated glass plate), on the surface of which there was formed the membrane, were evaluated. From their results, this glass plate was found to absorb ultraviolet rays over a wide range, specifically, to exercise an interception property against ultraviolet rays of not longer than 430 nm.

The transparency of the membrane-coated glass plate was 0.8% in haze.

### EXAMPLE A3-13

A membrane of 2 μm in membrane thickness was formed on a surface of the glass plate in the same way as of Example A3-9 except that the dispersion (1-17) was replaced with a 2-propanol dispersion (1-2) of 20 wt % in concentration having been obtained by subjecting the reaction liquid (1-2) (having been obtained from Example A1-2) to heating solvent displacement and that the heat-drying temperature was changed to 350° C.

The formed membrane was a ZnO-crystal membrane in which Cu was contained in a ratio of 2 atomic % relative to Zn.

The spectroscopic properties of the glass plate (membrane-coated glass plate), on the surface of which there was formed the membrane, were evaluated. From their results, this glass plate was found to absorb ultraviolet rays over a wide range, specifically, to exercise an interception property against ultraviolet rays of not longer than 380 nm.

The transparency of the membrane-coated glass plate was 0.2% in haze.

[Second Metal Oxide Particle]:

The measurements and evaluations in the below-mentioned Examples and Comparative Examples were carried out by the following methods unless otherwise noted in each of these Examples and Comparative Examples.

<Evaluation of Metal Oxide Particles>:

(1) Crystal Identification of Metal Oxide Particles:

As to the above powder sample, the crystal system and crystal structure of the metal oxide particles were evaluated by powder X-ray diffractometry with a powder X-ray diffraction device (produced by Rigaku Denki K.K., product name: RINT 2400). Hereinafter the measurement conditions are shown.
X-rays: CuKα1 rays (wavelength: 1.54056 Å)/40 kV/200 mA
Scanning range: 2θ=20 to 80°
Scanning speed: 5°/min

For example, in cases where the metal oxide particles contained Zn as a main metal component, then whether the metal oxide particles had the same crystal system and crystal structure as of ZnO or not was judged from whether three intense-ray peaks characteristic of ZnO of the hexagonal crystal system were seen or not. Specifically, if diffraction peaks existed in all positions of the following three diffraction angles (a) to (c), then it was judged that the metal, oxide particles had the same crystal system and crystal structure as of ZnO.
(a) 2θ=31.65 to 31.95°
(b) 2θ=34.30 to 34.60°
(c) 2θ=36.10 to 36.40°

Incidentally, the diffraction peak existing in the position of the above (a) is judged to be based on diffracted rays to the (100) plane of the ZnO crystal, and the diffraction peak existing in the position of the above (b) is judged to be based on diffracted rays to the (002) plane of the ZnO crystal, and the diffraction peak existing in the position of the above (c) is judged to be based on diffracted rays to the (101) plane of the ZnO crystal.

Similarly also in cases where a metal element other than Zn was contained as a main metal component in the metal oxide particles, whether the metal oxide particles had the same crystal system and crystal structure as of an oxide of the above metal element or not was judged from whether three intense-ray peaks characteristic of an oxide crystal of the above metal element were seen or not.

(2) Crystal Grain Diameters (Ds) and (Dw) of Metal Oxide Particles:

As to the above powder sample, the crystal grain diameter (Ds) of the metal oxide particles was evaluated by powder X-ray diffractometry with a powder X-ray diffraction device (produced by Rigaku Denki K.K., product name: RINT 2400).

Specifically, as to the crystal grain diameter (Ds), the crystal grain diameter Ds (hkl) (wherein the hkl denotes a Miller index: the Ds (hkl) is the size of the crystal grain in the vertical direction to the lattice plane of the Miller index (hkl) (incidentally, the Miller index (hkl) differs according to the Examples and Comparative Examples and is shown in tables which show their respective results)) was determined by Scherrer equation (analysis) from widths of diffracted rays in the resultant X-ray diffraction pattern.

As to the crystal grain diameter (Dw), the average value of three intense rays' respective Ds values having been determined in the above way was taken as the Dw. That is to say, unless otherwise noted, the crystal grain diameter (Dw) is usually calculated in the following way. A powder X-ray diffraction pattern of the metal oxide particle is measured, and then, as to three intense rays thereof (the largest peak (1) of diffracted rays, the second largest peak (2) of diffracted rays, and the'third largest peak (3) of diffracted rays), the crystal grain diameters Ds1, Ds2, and Ds3 in the vertical directions to the diffraction planes assigned to the diffracted rays (1) to (3) respectively are determined from their respective full widths of half maximum intensity or integral widths in accordance with Scherrer equation, and then their average value ((Ds1+Ds2+Ds3)/3) is calculated as the crystal grain diameter (Dw).

(3) Composition of Metal Oxide Particles (Average Composition of Metal Elements):

The above powder sample was subjected to quantitative analyses into metal elements by fluorescent X-ray analyses to thus determine the contents of the at least two hetero-metal elements (M′) (hereinafter referred to as M1, M2) relative to the main metal element (M) and, in cases where the metal compound was used as an additive during the formation of the particles, the content of the metal element (Ms) of the above metal compound relative to the main metal element (M).

In addition, while each particle in the above powder sample was observed with an FE-TEM (field emission transmission electron microscope) as equipped with an XMA device (X-ray microanalyzer) of 1 nmφ in resolution, any portion of from the surface layer of the particle up to its central portion was subjected to local elemental analysis, and the deflection of the intensity ratio of a peak intensity assigned to each metal element to a peak intensity assigned to the main metal element (M) was evaluated to thus judge whether each metal element contained in the particle was uniformly distributed or not (i.e. judge the uniformity of the distribution). In addition, when the local elemental analysis into each metal element was carried out, whether or not there was any segregate of the hetero-metal elements (M1, M2) or of the metal element (Ms) of the metal compound was also evaluated.
◯: The metal elements (M1, M2, Ms) other than the main metal element (M) are uniformly contained.
×: The metal elements (M1, M2, Ms) other than the main metal element (M) are not uniformly contained and/or segregates of their metals or compounds were seen.

(4) Evaluation of Valences of Hetero-Metal Elements Contained in Metal Oxide Particles:

As to the above powder sample, 2p_{3/2} spectra of the hetero-metal elements (particularly, Co and Fe) contained in the metal oxide particles were measured by X-ray photoelectron spectroscopy (XPS) with a photoelectron spectroscope (produced by Nippon Denshi K.K., product name: JPS-90 model) and, from their peak positions, the bond energy values were determined to thus judge the valences of the hetero-metal elements.

Incidentally, in order to reduce measured value errors caused by such as energy shift due to the electrification property, the determination of the bond energy values was put under corrections based on the C 1s peak position of the surface hydrocarbon.

In addition, as already known data for comparison, peak positions of 2p_{3/2} spectra of compounds of various metal elements as shown in “The Handbook of X-ray Photoelectron Spectroscopy” (1991) published by Nippon Denshi K.K. were referred to.

(5) Performances of Metal Oxide Particles: (5-1):

Performances in a metal-oxide-particles-dispersed membrane state were evaluated in the following way. That is to say, a reaction liquid (dispersion) resultant from a reaction to form metal oxide particles was subjected to heating solvent displacement to thereby obtain a dispersion such that the metal oxide particles were dispersed in 1-butanol in a particle concentration of 20 wt % (in cases where the boiling point of the solvent in the reaction liquid was higher than that of 1-butanol, then it was arranged to obtain the 20 wt % dispersion by heat-concentrating the reaction liquid (dispersion) and to use this dispersion). An amount of 100 parts of the resultant dispersion was mixed with 20 parts of a silicate binder (solid component content in terms of SiO₂: 51 wt %) and 0.5 part of a catalyst (n-butylamine) to thus prepare a paint. Incidentally, as to the above particle concentration, it was calculated in a way that the solid component amount as a result of vacuum drying of the resultant dispersion at 120° C. with a vacuum drier for 1 hour was taken as the particle weight.

The resultant paint was coated onto alkali-free glass (produced by Corning International Corporation, barium borosilicate glass, Glass Code No. 7059, thickness: 0.6 mm) by use of a bar coater in a way for the wet membrane thickness to be 24 μm. Thereafter, they were normally dried at 25° C. to thereby obtain glass, on the surface of which there was formed the metal-oxide-particles-dispersed membrane. Then, only the membrane portion of this dispersion-membrane-coated glass was evaluated by visible-ray transmission property, ultraviolet absorption property, and visible long-wavelength absorption property based on transmission spectra. Incidentally, because the transmission spectra of the above dispersion membrane are influenced also by the dispersed state of the particles, it is herein provided that only a dispersion membrane satisfying the haze <3% in transparency shall be evaluated. Therefore, when the paint was prepared, in cases of particles difficult to disperse, their dispersion treatment is carried out for a long time (refer to the results of the below-mentioned evaluation (6)) and, if necessary, another dispersion method is also used.

The transmission spectrum of each of the dispersion-membrane-coated glass and the above alkali-free glass (substrate only) was measured by use of an auto-recording spectrophotometer having an integrating sphere (produced by Shimadzu Corporation, product name: UV-3100).

From the resultant transmission spectrum, as to each of the dispersion-membrane-coated glass and the alkali-free glass, the visible-ray transmission property was evaluated by a visible-ray transmittance (transmittance (%/) of light of 500 or 600 nm in wavelength) (transmittance (%) at 600 nm in Examples B4-1, B4-2, and Comparative Examples B4-1 to B4-3 and transmittance (%) at 500 nm in the other Examples and Comparative Examples), and the ultravioletabsorption property was evaluated by transmittances in the range of from visible rays to visible-ray short wavelengths (transmittances (%) at 380 nm, 400 nm, 420 nm in wavelength) (however, in some of the Examples and Comparative Examples, the ultraviolet absorption property was evaluated by a transmittance at 380 nm only, or 380 nm and 400 nm only, among the above wavelengths). In addition, the visible long-wavelength absorption property was evaluated in the following way: as to absorption performances in the range of 550 to 700 nm, Δ (%) was determined by the following equation:
Δ(%)=[|T⁵⁰⁰−T¹|/T⁵⁰⁰]×100
(wherein: T¹ is the minimum value of transmittances (%) in the range of 550 to 700 nm; and T⁵⁰⁰ is a transmittance (%) at 500 nm), and its value was evaluated on the following standards:
A: Δ(%)<10%
B: 10%≦Δ(%)

Incidentally, the above transmittance at each wavelength of only the membrane portion is determined by the following equation:
Transmittance (%) at each wavelength of only the membrane portion=[transmittance (%) at each wavelength of dispersion-membrane-coated glass/transmittance (%) at each wavelength of alkali-free glass]×100
(wherein: as to the transmittance (%) at each wavelength of the alkali-free glass determined by the above evaluation method, any of the transmittances at 380 nm, 400 nm, 420 nm and 500 nm and the transmittances in the range of 550 to 700 nm is 91%)

(5-2):

The same evaluation was carried out in the same way as of the above evaluation method (5-1) except that a dispersion-membrane-coated glass was obtained in a way for the wet membrane thickness of the paint by the bar coater to be 66 μm. Incidentally, this evaluation was carried out only about the metal oxide particles as specified-in the Examples.

(5-3):

A dilution, having been prepared by diluting the resultant reaction liquid (dispersion) with 1-butanol as a diluting solvent so as to be 0.1 wt % in fine particle concentration, was used as the sample and, as to this sample, its transmission spectrum in the ultraviolet and visible ranges was measured by use of an auto-recording spectrophotometer having an integrating sphere (“UV-3100” produced by Shimadzu Corporation).

Ultraviolet intercepting ability: evaluated by transmittances at 380 nm, 400 nm, 420 nm.

Visible-ray transmission property: evaluated by a transmittance at 600 nm.

Incidentally, also as to the transmission spectrum of a membrane-formed product, its transmission spectrum in the ultraviolet and visible ranges was, similarly to the above, measured by use of the auto-recording spectrophotometer having an integrating sphere (“UV-3100” produced by Shimadzu Corporation).

(5-4) Evaluation of Transparency and Hue:

A fine-particles-dispersed membrane was formed and evaluated.

The dispersion-membrane-coated glass having been obtained from (5-1) above was used as the sample and evaluated by transparency and hue. That is to say, the transparency was evaluated by a haze value measured with a third diameter (“NDH-1001 DP” produced by Nippon Denshoku Kogyo Co., Ltd.). As to the hue, the appearance was observed with the eye. Incidentally, the haze value of the alkali-free glass as the substrate was 0%.

Incidentally, also as to the transparency and hue of a membrane-formed product, similarly to the above, the transparency was evaluated by the haze value measured with the turbidimeter (“NDH-1001 DP” produced by Nippon Denshoku Kogyo Co., Ltd.), and the hue was evaluated by observing the appearance with the eye.

(6) Dispersing-Ease (Dispersibility) of Metal Oxide Particles:

The dispersing-ease of the metal oxide particles is evaluated by dispersing-conditions for the haze value of the dispersion-membrane-coated glass to be less than 3% when, in the same way as of the above (5), the paint containing the metal oxide particles is prepared and the glass, on the surface of which there is formed the metal-oxide-particles-dispersed membrane, is obtained. Specifically, on the following standards, there was evaluated how long dispersing-treatment time (ultrasonic wave irradiation time) was needed for the dispersion-membrane-coated glass satisfying the above haze value (less than 3%) to be obtained when the uncoated paint was subjected to a dispersing-treatment with an ultrasonic homogenizer.
◯: Shorter than 5 minutes
Δ: 5 to 10 minutes (but not including 10 minutes)
×: Not shorter than 10 minutes

(7) Identification and Bonding Amount of Acyl Group:

An amount of 1 g of the powder sample was added to a 0.1 N aqueous sodium hydroxide solution and then stirred for 24 hours. Thereafter, by ion chromatography, the acyl group was identified and quantified in bonding amount.

<Evaluation of Composition for Membrane Formation>:

(1) Dispersing-Stability:

The dispersing-stability of the resultant dispersion was evaluated on the following standards:
A: There is seen no occurrence of separation into two layers or of sediment even if 1 week has passed since leaving the dispersion undisturbed.
B: A slight amount of sediment occurred in 1 week after having left the dispersion undisturbed.
C: A large amount of sediment occurred in 1 week after having left the dispersion undisturbed.

(2) Transparency:

The resultant dispersion was coated in the form of a membrane on a transparent glass plate and then observed with the eye in a wet state to evaluate it on the following standards:
A: The feeling of the transparency is extremely high.
B: The feeling of the transparency is high.
C: The feeling of the transparency is low.

(3) Dispersion Particle Diameter:

The median diameter was measured with a dynamic light. scattering'type particle diameter distribution measurement device (“LB-500” produced by Horiba Seisakusho) and taken as the dispersion particle diameter.

<Evaluation of Membrane (or Membrane-Coated Substrate)>:

(1) Visible-Ray Transmission Property and Ultraviolet Absorption Property-Based on Transmission Spectrum:

The resultant membrane-coated substrate was evaluated by visible-ray transmission property and ultraviolet absorption property based on its transmission spectrum.

The transmission spectrum of the membrane-coated substrate was measured by use of an auto-recording spectrophotometer having an integrating sphere (produced by Shimadzu Corporation, product name: UV-3 100).

From the resultant transmission spectrum, as to the membrane-coated substrate, the visible-ray transmission property was evaluated by a visible-ray transmittance (transmittance (%) at 500 nm), and the ultraviolet absorption property was evaluated by transmittances in the range of from visible rays to visible-ray short wavelengths (transmittances (%/) at 380 nm, 400 nm, and 420 nm).

Incidentally, also as to only the substrate used for the membrane-coated substrate, the transmittance at each wavelength was determined by the same method as the above.

(2) Visible-Ray Transparency:

Each of the membrane-coated substrate and only the substrate was measured by the total ray transmittance, the diffused-ray transmittance, the parallel-ray transmittance, and the haze value with a turbidimeter (produced by Nippon Denshoku Kogyo Co., Ltd., product name: NDH-1001 DP) to evaluate the transparency of the membrane from the measured haze value on the following standards. Incidentally, the haze value of the membrane is given by subtracting a haze value of only the substrate from that of the membrane-coated substrate.
◯: Haze <3%
×: Haze ≧3%

(3) Coloring Degree:

As to the resultant membrane (membrane-coated substrate), its appearance was observed with the eye to thereby evaluate the coloring degree on the following standards:
×: Coloring is noticeable.
◯: There is no coloring or, even if any, coloring is not noticeable.

(4) Refractive Index:

The refractive index at a wavelength of 550 nm was determined by measuring the reflectance of the membrane (having been formed on the substrate) in the range of 230 to 760 nm with a reflection-spectroscopic membrane thickness meter (“FE-3000” produced by Ohtsuka Electronics CO., Ltd.) and then citing nkCauchy's dispersion formula as a representative approximation formula of the refractive index to determine an unknown parameter by the nonlinear least-squares method from an actually measured value of a spectrum of the absolute reflectance.

(5) Dry Membrane Thickness:

This was measured with a reflection-spectroscopic membrane thickness meter (“FE-3000” produced by Ohtsuka Electronics CO., Ltd.).

### EXAMPLE B1-1

There was prepared a reaction apparatus comprising: a pressure-resistant glass reactor possible to externally heat and equipped with a stirrer, an addition inlet (connected directly to an addition tank), a thermometer, a distillate gas outlet, and a nitrogen-gas-introducing inlet; the addition tank connected to the above addition inlet; and a condenser (connected directly to a trap) connected to the above distillate gas outlet.

Into the above reactor, there was charged a mixture comprising 183 parts of zinc acetate anhydride, 3.8 parts of iron(III) acetate hydroxide powder, 7.1 parts of tin(IV) acetate, and 1,700 parts of methanol, and then its gas phase portion was purged with nitrogen gas. Thereafter, under stirring, the temperature of the mixture was raised (from 20° C.) to 150° C. and then heat-retained at 150° C.±1° C. for 5 hours to thus carry out a reaction to form metal oxide particles and then cooled, thereby obtaining a reaction liquid (11) containing yellow fine particles (metal oxide particles). Furthermore, the resultant reaction liquid was subjected to heating solvent displacement to thereby obtain a dispersion (11) such that the above metal oxide particles were dispersed in 1-butanol in a particle concentration of 20 wt %.

The metal oxide particles in the reaction liquid (11) were subjected to the aforementioned various measurements and evaluations. Their results are shown in Table 7. Incidentally, as to the valence of Fe contained in the metal oxide particles in the reaction liquid (11), it was evaluated by measuring a 2p_{3/2} spectrum of Fe in the aforementioned way. As a result, from its peak position, it was judged that Fe of 3 in valence (Fe(III)) was contained.

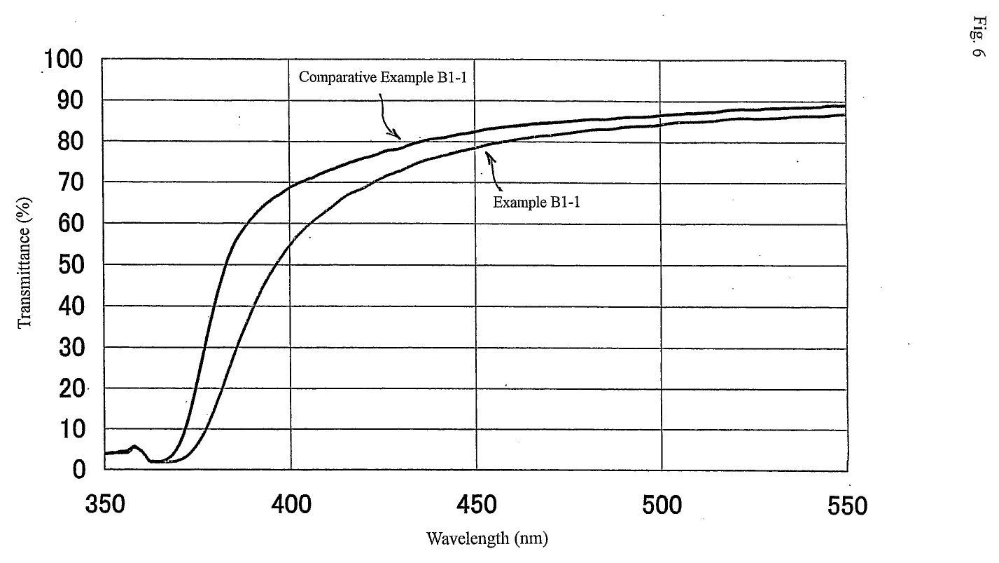

Furthermore, a transmission spectrum of dispersion-membrane-coated glass, having been obtained in the aforementioned various measurements and evaluations, is shown in FIG. 6 along with that in the below-mentioned Comparative Example B1-1.

### COMPARATIVE EXAMPLE B1-1

The same reaction apparatus as of Example B1-1, comprising such as the pressure-resistant glass reactor, was prepared.

Into the above reactor, there was charged a mixture comprising 183 parts of zinc acetate anhydride powder, 3.8 parts of iron(III) acetate hydroxide powder, and 1,700 parts of methanol, and then its gas phase portion was purged with nitrogen gas. Thereafter, under stirring, the temperature of the mixture was raised (from 20° C.) to 180° C. and then heat-retained at 180° C.±1° C. for 5 hours to thus carry out a reaction to form metal oxide particles and then cooled, thereby obtaining a reaction liquid (c11) containing yellow fine particles (metal oxide particles). Furthermore, the resultant reaction liquid was subjected to heating solvent displacement to thereby obtain a dispersion (c11) such that the above metal oxide particles were dispersed in 1-butanol in a particle concentration of 20 wt %.

The metal oxide particles in the reaction liquid (c11) were subjected to the aforementioned various measurements and evaluations. Their results are shown in Table 7.

Furthermore, a transmission spectrum of dispersion-membrane-coated glass, having been obtained in the aforementioned various measurements and evaluations, is shown in FIG. 6 along with that in the aforementioned Example B 1- 1.

### EXAMPLES B1-2 to B1-4

Reaction liquids (12) to (14), containing yellow fine particles (metal oxide particles), and dispersions (12) to (14) were obtained in the same way as of Example B1-1 except that such as the kinds and use amounts of the raw materials being charged and reaction conditions were changed as shown in Table 6.

The metal oxide particles in each of the reaction liquids (12) to (14) were subjected to the aforementioned various measurements and evaluations. Their results are shown in Table 7. Incidentally, as to the valence of Co contained in the metal oxide particles in the reaction liquids (12) to (14), it was evaluated by measuring a 2p_{3/2} spectrum of Co in the aforementioned way. As a result, from its peak position, it was judged that Co of 2 in valence (Co(II)) was contained.

As to the metal oxide particles in the reaction liquid (12), the evaluation (5-2) among the aforementioned evaluations of metal oxide particles was also carried out. As a result, the transmittance at 380 nm was less than 1%, and the transmittance at 500 nm was 80%, and the A (%) was 4%.

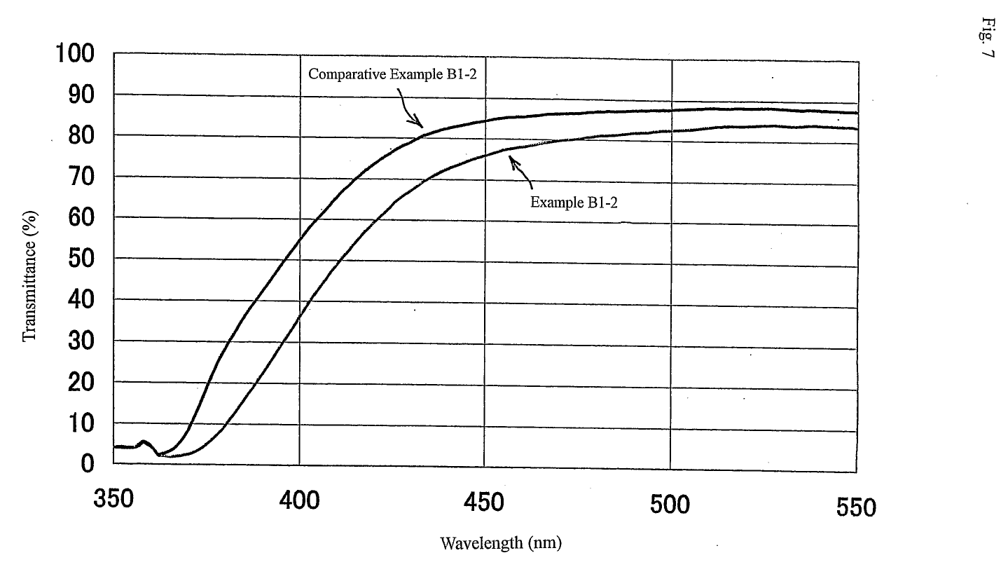

Furthermore, as to the metal oxide particles in the reaction liquid (12), a transmission spectrum of dispersion-membrane-coated glass, having been obtained in the aforementioned various measurements and evaluations, is shown in FIG. 7 along with that in the below-mentioned Comparative Example B 1-2.

Incidentally, as to the particles in the dispersion having been obtained from Example B1-3, the valences of the trace metal elements were measured by XPS. As a result, it was shown that Cu(II) and Cu(I) were mingled.

### EXAMPLES B1-5 to B1-8

Reaction liquids (15) to (18), containing yellow fine particles (metal oxide particles), and dispersions (15) to (18) were obtained in the same way as of Example B1-1 except that such as the kinds and use amounts of the raw materials being charged and reaction conditions were changed as shown in Table 6 (as the raw materials being charged, the additives as shown in Table 6 are also included therein).

The metal oxide particles in each of the reaction liquids (15) to (18) were subjected to the aforementioned various measurements and evaluations. Their results are shown in Table 7. Incidentally, as to the valence of Co contained in the metal oxide particles in the reaction liquid (17), it was evaluated by measuring a 2p_{3/2} spectrum of Co in the aforementioned way. As a result, from its peak position, it was judged that Co of 2 in valence (Co(II)) was contained.

### COMPARATIVE EXAMPLES B1-2 to B1-3

Reaction liquids (c12) to (c13), containing yellow fine particles (metal oxide particles), and dispersions (c12) to (c13) were obtained in the same way as of Example B1-1 except that such as the kinds and use amounts of the raw materials being charged and reaction conditions were changed as shown in Table 6.

The metal oxide particles in each of the reaction liquids (c12) to (c13) were subjected to the aforementioned various measurements and evaluations. Their results are shown in Table 7. Furthermore, as to the metal oxide particles in the reaction liquid (c12), a transmission spectrum of dispersion-membrane-coated glass, having been obtained in the aforementioned various measurements and evaluations, is shown in FIG. 7 along with that in the aforementioned Example B 1-2.

TABLE 6

| | | | | | Reaction | |
|---|---|---|---|---|---|---|
| | | | | | conditions | |
| Zn | M1 | M2 | | | | reten- |
| compound | compound | compound | Additive | Solvent | Temper- | tion |

| | | Weight | | Weight | | Weight | | Weight | | Weight | ature | time |
|---|---|---|---|---|---|---|---|---|---|---|---|---|
| | | parts | | parts | | parts | | parts | | parts | (° C.) | (hr) |
| Example | Zinc | 183 | Iron (III) | 3.8 | Tin (IV) | 7.1 | — | 0 | Methanol | 1700 | 150 | 5 |
| B1-1 | acetate | | acetate | | acetate | | | | | | | |
| | | | hydroxide | | | | | | | | | |
| Example | Zinc | 183 | Cobalt (II) | 3.5 | Indium (III) | 2.9 | — | 0 | 1-Propanol | 1700 | 150 | 5 |
| B1-2 | acetate | | acetate | | anhydride | | | | | | | |
| | | | anhydride | | | | | | | | | |
| Example | Zinc | 183 | Copper | 3.6 | Aluninum (III) | 0.49 | — | 0 | Ethanol | 1700 | 150 | 5 |
| B1-3 | acetate | | (II) | | s-butoxide | | | | | | | |
| | | | acetate | | | | | | | | | |
| | | | anhydride | | | | | | | | | |
| Example | Zinc | 183 | Cobalt (II) | 7.1 | Titanium (IV) | 8.1 | — | 0 | 1-Propanol | 1700 | 180 | 5 |
| B1-4 | acetate | | acetate | | methoxy- | | | | | | | |
| | | | anhydride | | propoxide | | | | | | | |
| Example | Zinc | 183 | Cobalt (II) | 1.8 | Gallium (III) | 2.0 | Methyl- | 2.7 | Ethanol | 1700 | 160 | 5 |
| B1-5 | acetate | | acetate | | ethoxide | | trimethoxy- | | | | | |
| | | | anhydride | | | | silane | | | | | |
| Example | Zinc | 183 | Iron (III) | 1.2 | Bismuth (III) | 9.4 | Partially | 11.0 | 1-Propanol | 1700 | 150 | 5 |
| B1-6 | acetate | | isoprop- | | t-pentoxide | | hydrolyzed- | | | | | |
| | | | oxide | | | | condensed | | | | | |
| | | | | | | | products | | | | | |
| | | | | | | | (trimer to | | | | | |
| | | | | | | | pentamer) of | | | | | |
| | | | | | | | tetramethoxy- | | | | | |
| | | | | | | | silane | | | | | |
| Example | Zinc | 183 | Cobalt (II) | 3.5 | Bismuth (III) | 0.77 | Aluminum | 1.0 | Ethanol | 1700 | 160 | 5 |
| B1-7 | acetate | | acetate | | acetate | | (III) | | | | | |
| | | | anhydride | | | | n-butoxide | | | | | |
| | | | | | | | trimer | | | | | |
| Example | Zinc | 183 | Cobalt (II) | 0.88 | Indium (III) | 2.9 | Titanium (IV) | 12.1 | 1-Propanol | 1700 | 180 | 5 |
| B1-8 | acetate | | acetate | | acetate | | tetra-n- | | | | | |
| | | | anhydride | | anhydride | | butoxide | | | | | |
| | | | | | Bismuth (III) | 5.7 | tetramer | | | | | |
| | | | | | acetate | | | | | | | |
| Compar- | Zinc | 183 | Iron (llI) | 3.8 | — | 0 | — | 0 | Methanol | 1700 | 180 | 5 |
| ative | acetate | | acetate | | | | | | | | | |
| Example | | | hydroxide | | | | | | | | | |
| B1-1 | | | | | | | | | | | | |
| Compar- | Zinc | 183 | Cobalt (II) | 3.5 | — | 0 | — | 0 | 1-Propanol | 1700 | 150 | 5 |
| ative | acetate | | acetate | | | | | | | | | |
| Example | | | anhydride | | | | | | | | | |
| B1-2 | | | | | | | | | | | | |
| Compar- | Zinc | 183 | — | 0 | Indium (III) | 5.8 | — | 0 | Ethanol | 1700 | 180 | 5 |
| ative | acetate | | | | acetate | | | | | | | |
| Example | | | | | | | | | | | | |
| B1-3 | | | | | | | | | | | | |

TABLE 7

| | | | | | | | | | | | Visible- |
|---|---|---|---|---|---|---|---|---|---|---|---|
| | | | | | | | | | | | ray |
| | | | | | | | | Uni- | | | trans- |
| | X-ray | Metal composition of fine | | | | | | formity | | | mission |
| | diffrac- | particles (fluorescent X-ray analysis) | | | | | | of | Crystal grain | | property |
| | tion | | Atomic | | Atomic | | Atomic | distri- | diameter (nm) | | (%) |
| | pattern | M1 | %/Zn | M2 | %/Zn | Ms | %/Zn | bution | Ds (100) | Ds (002) | 500 nm |
| Example | Equal to | Fe | 1.9 | Sn | 1.9 | — | <0.01 | ◯ | 18 | 17 | 92 |
| B1-1 | ZnO | | | | | | | | | | |
| Example | Equal to | Co | 1.9 | In | 1 | — | <0.01 | ◯ | 24 | 21 | 91 |
| B1-2 | ZnO | | | | | | | | | | |
| Example | Equal to | Cu | 1.5 | Al | 0.2 | — | <0.01 | ◯ | 25 | 24 | 93 |
| B1-3 | ZnO | | | | | | | | | | |
| Example | Equal to | Co | 3.8 | Ti | 1.9 | — | <0.01 | ◯ | 15 | 15 | 93 |
| B1-4 | ZnO | | | | | | | | | | |
| Example | Equal to | Co | 0.9 | Ga | 1 | Si | 2 | ◯ | 8 | 18 | 90 |
| B1-5 | ZnO | | | | | | | | | | |
| Example | Equal to | Fe | 0.5 | Bi | 1.3 | Si | 8.8 | ◯ | 11 | 16 | 90 |
| B1-6 | ZnO | | | | | | | | | | |
| Example | Equal to | Co | 2 | Bi | 0.2 | Al | 0.6 | ◯ | 8 | 20 | 89 |
| B1-7 | ZnO | | | | | | | | | | |
| Example | Equal to | Co | 0.5 | In | 0.5 | Ti | 4.9 | ◯ | 10 | 12 | 88 |
| B1-8 | ZnO | | | Bi | 1.5 | | | | | | |
| Compar- | Equal to | Fe | 1.8 | — | 0 | — | <0.01 | ◯ | 21 | 21 | 96 |
| ative | ZnO | | | | | | | | | | |
| Example | | | | | | | | | | | |
| B1-1 | | | | | | | | | | | |
| Compar- | Equal to | Co | 1.7 | — | 0 | — | <0.01 | ◯ | 26 | 23 | 97 |
| ative | ZnO | | | | | | | | | | |
| Example | | | | | | | | | | | |
| B1-2 | | | | | | | | | | | |
| Compar- | Equal to | — | 0 | In | 1.9 | — | <0.01 | ◯ | 20 | 18 | 98 |
| ative | ZnO | | | | | | | | | | |
| Example | | | | | | | | | | | |
| B1-3 | | | | | | | | | | | |

| | | | | Visible | |
|---|---|---|---|---|---|
| | | | | long- | |
| | Ultraviolet absorption | | | wavelength | |
| | property (%) | | | absorption | Dispersi- |
| | 380 nm | 400 nm | 420 nm | property | bility |
| Example | 18 | 60 | 75 | A | Δ |
| B1-1 | | | | | |
| Example | 10 | 39 | 65 | A | Δ |
| B1-2 | | | | | |
| Example | 19 | 61 | 75 | A | X |
| B1-3 | | | | | |
| Example | 14 | 44 | 69 | A | Δ |
| B1-4 | | | | | |
| Example | 9 | 35 | 58 | A | ◯ |
| B1-5 | | | | | |
| Example | 43 | 57 | 52 | A | ◯ |
| B1-6 | | | | | |
| Example | 29 | 48 | 60 | A | ◯ |
| B1-7 | | | | | |
| Example | 10 | 27 | 27 | A | ◯ |
| B1-8 | | | | | |
| Compar- | 46 | 76 | 84 | A | X |
| ative | | | | | |
| Example | | | | | |
| B1-1 | | | | | |
| Compar- | 30 | 60 | 81 | A | X |
| ative | | | | | |
| Example | | | | | |
| B1-2 | | | | | |
| Compar- | 36 | 87 | 73 | A | Δ |
| ative | | | | | |
| Example | | | | | |
| B1-3 | | | | | |

### EXAMPLE B 1-9

The same reaction apparatus as of Example B1-1 was used and, into its reactor, there was charged a mixture comprising 183 parts of zinc acetate anhydride, 0.9 part of copper(II) acetate anhydride, 3 parts of indium acetate anhydride, 16 parts of tetrabutoxysilane, and 1,847 parts of 2-propanol, and then its gas phase portion was purged with nitrogen gas. Thereafter, under stirring, the temperature of the mixture was raised from 20° C. and then heat-retained at 150° C.±1° C. for 5 hours to thus carry out a reaction to form metal oxide particles and then cooled, thereby obtaining a dispersion (19) comprising a reaction liquid containing metal oxide particles in a concentration of 4 wt %.

It was confirmed that the fine particles in the resultant dispersion were those which comprised a ZnO crystal and were 14 nm in crystal grain diameter and contained copper and indium in ratios of 0.4 and 0.9 atomic % respectively relative to Zn, and to surfaces of which there was bonded the ethanoyl group in a ratio of 3.1 mol % relative to Zn and the Si compound in a ratio of Si/Zn=5 atomic %.

The metal oxide particles in the dispersion (19) were evaluated in the ways of (5-3) and (5-4) above. That is to say, their ultraviolet absorption property and visible-ray absorption property were evaluated in the following way: a dilution, having been prepared by diluting the resultant dispersion with 1-butanol as a diluting solvent so as to be 0.1 wt % in fine particle concentration, was used as the sample, and this sample was filled into a quartz cell of 1 cm in thickness to measure its transmission spectrum in the ultraviolet and visible ranges by use of an auto-recording spectrophotometer having an integrating sphere (“UV-3100” produced by Shimadzu Corporation). The ultraviolet absorption property was evaluated by transmittances at 380 nm, 400 nm, 420 nm, and the visible-ray transmission property was evaluated by a transmittance at 600 nm. Their results are shown in Table 9.

### EXAMPLES B1-10 to B1-15 and Comparative Examples B1-4 to B1-6

Dispersions (110) to (115) and (c14) to (c16) comprising reaction liquids, in which metal oxide particles were dispersed in a particle concentration of 4 wt %, were obtained by heating the mixture with the alcohol in the range of 120 to 200° C. in the same way as of Example B 1-9 except that the kinds of the raw materials being 5 charged were changed as shown in Table 8 (as the raw materials being charged, the additives as shown in Table 8 are also included therein).

The resultant dispersions were used to carry out the evaluations in the same way as of Example B1-9. Their results are shown in Table 9.

The fine particles in the dispersions having been obtained from Examples B1-9 to B1-15 were evaluated by the uniformity of the distribution. As a result, there was seen no segregate comprising (super)fine single metal particles or metal oxide particles containing the added metal element (M1, M2, M3 of Table 4) as a main component, and any case was evaluated as “◯”.

As to the materials of which the valences of the contained metals could be judged from the bond energy values as measured with the photoelectron spectroscope, those valences are shown in the table.

TABLE 8

| | | | | | Surface- |
|---|---|---|---|---|---|
| | | | | | treating |
| | | | | | metal |
| | Zn | M1 | M2 | M3 | (Ms) |
| | compound | compound | compound | compound | compound |
| Example | Zinc | Copper (II) | Indium | — | Tetra- |
| B1-9 | acetate | acetate | acetate | | butoxy- |
| | anhydride | anhydride | anhydride | | silane |
| Example | Zinc | Copper (II) | Cerium | — | Tetra- |
| B1-10 | acetate | acetate | (III) | | methoxy- |
| | anhydride | anhydride | acetate | | silane |
| | | | hydrate | | |
| Example | Zinc | Copper (II) | Titanium | — | Methyl- |
| B1-11 | acetate | acetate | tetra-n- | | tri- |
| | anhydride | anhydride | butoxide | | methoxy- |
| | | | | | silane |
| Example | Zinc | Copper (II) | Tin (II) | — | — |
| B1-12 | formate | acetate | acetate | | |
| | anhydride | anhydride | | | |
| Example | Zinc | Copper (II) | Bismuth | — | — |
| B1-13 | propionate | acetate | (III) | | |
| | anhydride | anhydride | acetate | | |
| | | | oxide | | |
| Example | Zinc | Copper (II) | Iron (III) | — | — |
| B1-14 | acetate | acetate | acetate | | |
| | anhydride | anhydride | hydroxide | | |
| Example | Zinc | Copper (II) | Nickel (II) | Indium | — |
| B1-15 | acetate | acetate | acetate | acetate | |
| | anhydride | anhydride | tetra- | anhydride | |
| | | | hydrate | | |
| Compar- | Zinc | Copper (II) | — | — | Tetra- |
| ative | acetate | acetate | | | methoxy- |
| Example | anhydride | anhydride | | | silane |
| B1-4 | | | | | |
| Compar- | Zinc | Indium | — | — | Tetra- |
| ative | acetate | acetate | | | methoxy- |
| Example | anhydride | anhydride | | | silane |
| B1-5 | | | | | |
| Compar- | Zinc | — | — | — | Tetra- |
| ative | acetate | | | | methoxy- |
| Example | anhydride | | | | silane |
| B1-6 | | | | | |

TABLE 9

| | | | | | | | | | | | Bonding amount | |
|---|---|---|---|---|---|---|---|---|---|---|---|---|
| | | | | | | | | | | | of acyl group | |
| | X-ray | Metal composition of fine | | | | | | | | Crystal | | Bonding |
| | diffrac- | particles (fluorescent X-ray analysis) | | | | | | | | grain | | amount |
| | tion | | Atomic | | Atomic | | Atomic | | Atomic | diameter | Identi- | mol |
| | pattern | M1 | %/Zn | M2 | %/Zn | M3 | %/Zn | | %/Zn | Dw (nm) | fication | %/Zn |
| Example | Equal to | Cu | 0.4 | In | 0.8 | — | — | Si | 5 | 14 | Ethanoyl | 3.1 |
| B1-9 | ZnO | | | | | | | | | | group | |
| Example | Equal to | Cu | 0.1 | Ce | 0.2 | — | — | Si | 1 | 13 | Ethanoyl | 2 |
| B1-10 | ZnO | | | (III) | | | | | | | group | |
| Example | Equal to | Cu | 0.5 | Ti | 0.05 | — | — | Si | 2 | 14 | Ethanoyl | 1.5 |
| B1-11 | ZnO | | | | | | | | | | group | |
| Example | Equal to | Cu | 1 | Sn | 0.5 | — | — | — | — | 16 | Formyl | 1.1 |
| B1-12 | ZnO | | | (II) | | | | | | | group | |
| Example | Equal to | Cu | 1 | Bi | 0.9 | — | — | — | — | 15 | Propanoyl | 1.4 |
| B1-13 | ZnO | | | (III) | | | | | | | group | |
| Example | Equal to | Cu | 1.3 | Fe | 1.5 | — | — | — | — | 13 | Ethanoyl | 2 |
| B1-14 | ZnO | | | (II), | | | | | | | group | |
| | | | | Fe | | | | | | | | |
| | | | | (III) | | | | | | | | |
| Example | Equal to | Cu | 3 | Ni | 2.5 | In | 0.1 | — | — | 16 | Ethanoyl | 10 |
| B1-15 | ZnO | | | (II) | | | | | | | group | |
| Compar- | Equal to | Cu | 2 | — | — | — | — | Si | 1 | 11 | Ethanoyl | 8 |
| ative | ZnO | | | | | | | | | | group | |
| Example | | | | | | | | | | | | |
| B1-4 | | | | | | | | | | | | |
| Compar- | Equal to | In | 1.5 | — | — | — | — | Si | 1 | 15 | Ethanoyl | 3 |
| ative | ZnO | | | | | | | | | | group | |
| Example | | | | | | | | | | | | |
| B1-5 | | | | | | | | | | | | |
| Compar- | Equal to | — | — | — | — | — | — | Si | 4 | 12 | Ethanoyl | 3 |
| ative | ZnO | | | | | | | | | | group | |
| Example | | | | | | | | | | | | |
| B1-6 | | | | | | | | | | | | |

| | | | | | | Visible- |
|---|---|---|---|---|---|---|
| | | | | | | ray |
| | | | Ultraviolet | | | trans- |
| | Trans- | | absorption | | | mission |
| | parency | | property | | | property |
| | Haze | | | (%) | | (%) |
| | (%) | Hue | 380 nm | 400 nm | 420 nm | 600 nm |
| Example | 0.2 | Colorless | 6 | 43 | 59 | 85 |
| B1-9 | | | | | | |
| Example | 0.3 | Colorless | 2 | 16 | 31 | 83 |
| B1-10 | | | | | | |
| Example | 0.1 | Colorless | 3 | 19 | 30 | 83 |
| B1-11 | | | | | | |
| Example | 0.5 | Colorless | 3 | 18 | 29 | 87 |
| B1-12 | | | | | | |
| Example | 0.6 | Colorless | 7 | 11 | 13 | 88 |
| B1-13 | | | | | | |
| Example | 0.9 | Slight | 5 | 18 | 28 | 78 |
| B1-14 | | yellow | | | | |
| Example | 0.6 | Slight | 2 | 16 | 29 | 76 |
| B1-15 | | yellow | | | | |
| Compar- | 0.4 | Colorless | 13 | 55 | 69 | 90 |
| ative | | | | | | |
| Example | | | | | | |
| B1-4 | | | | | | |
| Compar- | 1.2 | Colorless | 20 | 57 | 70 | 87 |
| ative | | | | | | |
| Example | | | | | | |
| B1-5 | | | | | | |
| Compar- | 0.6 | Colorless | 36 | 62 | 70 | 91 |
| ative | | | | | | |
| Example | | | | | | |
| B1-6 | | | | | | |

From Table 9, it can be understood that: if In, Sn, Ti, or Ce (which are n-type dopants) besides Cu is caused to be contained, then the absorbance of ultraviolet rays of 380 nm is enhanced more than in cases where a large amount of Cu is caused to be contained; and, if Bi besides Cu is caused to be contained, then the absorbance of light in the range of 400 to 420 nm is enhanced; and, if, above all, Sn or Bi is caused to be contained, then the visible-ray transmission property (transmittance at 600 nm) is enhanced.

EXAMPLE B1-16

The same reaction apparatus as of Example B1-1 was used and, into its reactor, there was charged a mixture comprising 183 parts of zinc acetate anhydride, 5.8 parts of iron(III) acetate anhydride, 4.6 parts of gallium(III) acetate nonahydrate, 20 parts of titanium tetrabutoxide tetramer, and 3,940 parts of 1-propanol, and then its gas phase portion was purged with nitrogen gas. Thereafter, under stirring, the temperature of the mixture was raised from 20° C. and then heat-retained at 140° C.±1° C. for 5 hours to thus carry out a reaction to form metal oxide particles and then cooled, thereby obtaining a dispersion (116) comprising a reaction liquid containing metal oxide particles in a concentration of 2 wt %.

The resultant dispersion was used to carry out the evaluations in the same way as of Example B 1-9. Their results are shown in Table 11.

### EXAMPLES B1-17 to B1-23 and Comparative Example B1-7

Dispersions (117) to (123) and (c17) comprising reaction liquids, in which metal oxide particles were dispersed in a particle concentration of 2 wt %, were obtained by heating the mixture with the alcohol in the range of 120 to 200° C. in the same way as of Example B1-16 except that the kinds of the raw materials being charged were changed as shown in Table 10 (as the raw materials being charged, the additives as shown in Table 10 are also included therein).

The resultant dispersions were used to carry out the evaluations in the same way as of Example B 1-9. Their results are shown in Table 11.

The fine particles in the dispersions having been obtained from Examples B1-16 to B1-23 were evaluated by the uniformity of the distribution. As a result, any case was evaluated as “◯”.

As to the materials of which the valences of the contained metals could be judged from the bond energy values as measured with the photoelectron spectroscope, those valences are shown in the table.

TABLE 10

| | | | | | Surface- |
|---|---|---|---|---|---|
| | | | | | treating |
| | | | | | metal |
| | Zn | M1 | M2 | M3 | (Ms) |
| | compound | compound | compound | compound | compound |
| Example | Zinc | Iron (III) | Gallium | — | Titanium |
| B1-16 | acetate | acetate | acetate | | tetra- |
| | anhydride | anhydride | hydrate | | butoxide |
| | | | | | tetramer |
| Example | Zinc | Iron (III) | Silver (I) | Indium | — |
| B1-17 | acetate | acetate | acetate | acetate | |
| | anhydride | hydroxide | anhydride | anhydride | |
| Example | Zinc | Iron (III) | Cerium | — | — |
| B1-18 | acetate | acetate | (III) | | |
| | anhydride | hydroxide | acetate | | |
| | | | hydrate | | |
| Example | Zinc | Iron (II) | Tin (IV) | — | — |
| B1-19 | acetate | acetate | acetate | | |
| | anhydride | | | | |
| Example | Zinc | Iron(II) | Copper | — | Tetra- |
| B1-20 | acetate | acetate | (II) | | methoxy- |
| | anhydride | | acetate | | silane |
| | | | anhydride | | |
| Example | Zinc | Cobalt (II) | Indium | — | Zirconium |
| B1-21 | formate | acetate | acetate | | tetra- |
| | anhydride | tetra- | anhydride | | butoxide |
| | | hydrate | | | oligomer |
| | | | | | (condensa- |
| | | | | | tion |
| | | | | | degree: |
| | | | | | 6-10) |
| Example | Zinc | Nickel (II) | Copper | Aluminum | — |
| B1-22 | propionate | acetate | (II) | (III) | |
| | anhydride | tetrahydrate | acetate | s-butoxide | |
| | | | anhydride | | |
| Example | Zinc | Nickel (II) | Titanium | — | — |
| B1-23 | propionate | acetate | tetra-n- | | |
| | anhydride | tetrahydrate | butoxide | | |
| Compar- | Zinc | Iron (III) | — | — | — |
| ative | acetate | acetate | | | |
| Example | anhydride | | | | |
| B1-7 | | | | | |

TABLE 11

| | | | | | | | | | | | Bonding amount | |
|---|---|---|---|---|---|---|---|---|---|---|---|---|
| | | | | | | | | | | | of acyl group | |
| | X-ray | Metal composition of fine particles | | | | | | | | Crystal | | Bonding |
| | dif- | (fluorescent X-ray analysis) | | | | | | | | grain | | amount |
| | fraction | | Atomic | | Atomic | | Atomic | | Atomic | diameter | Identi- | mol |
| | pattern | M1 | %/Zn | M2 | %/Zn | M3 | %/Zn | Ms | %/Zn | Dw (nm) | fication | %/Zn |
| Example | Equal to | Fe | 2.3 | Ga | 1 | — | — | Ti | 8 | 15 | Ethanoyl | 4 |
| B1-16 | ZnO | (III) | | | | | | | | | group | |
| Example | Equal to | Fe | 1.1 | Ag | 0.05 | In | 1 | — | — | 18 | Ethanoyl | 3.3 |
| B1-17 | ZnO | (II), | | | | | | | | | group | |
| | | Fe | | | | | | | | | | |
| | | (III) | | | | | | | | | | |
| Example | Equal to | Fe | 0.2 | Ce | 0.5 | — | — | — | — | 18 | Ethanoyl | 0.9 |
| B1-18 | ZnO | (II), | | | | | | | | | group | |
| | | Fe | | | | | | | | | | |
| | | (III) | | | | | | | | | | |
| Example | Equal to | Fe | 0.6 | Sn | 0.5 | — | — | — | — | 15 | Ethanoyl | 1.7 |
| B1-19 | ZnO | (II) | | | | | | | | | group | |
| Example | Equal to | Fe | 5.2 | Cu | 0.1 | — | — | Si | 5 | 7 | Ethanoyl | 6 |
| B1-20 | ZnO | (II) | | | | | | | | | group | |
| Example | Equal to | Co | 1.9 | In | 1 | — | — | Zr | 0.4 | 15 | Formyl | 2 |
| B1-21 | ZnO | (II) | | | | | | | | | group | |
| Example | Equal to | Ni | 3 | Cu | 0.5 | Al | 1 | — | — | 20 | Propanoyl | 0.6 |
| B1-22 | ZnO | (II) | | | | | | | | | group | |
| Example | Equal to | Ni | 0.5 | Ti | 0.5 | — | — | — | — | 22 | Propanoyl | 0.5 |
| B1-23 | ZnO | (II) | | | | | | | | | group | |
| Compar- | Equal to | Fe | 2.2 | — | — | — | — | — | — | 16 | Ethanoyl | 2 |
| ative | ZnO | (III) | | | | | | | | | group | |
| Example | | | | | | | | | | | | |
| B1-7 | | | | | | | | | | | | |

| | | | | | | Visible- |
|---|---|---|---|---|---|---|
| | | | | | | ray |
| | | | Ultraviolet | | | trans- |
| | Trans- | | absorption | | | mission |
| | parency | | property | | | property |
| | Haze | | | (%) | | (%) |
| | (%) | Hue | 380 nm | 400 nm | 420 nm | 600 nm |
| Example | 0.2 | Slight | 3 | 22 | 37 | 86 |
| B1-16 | | yellow | | | | |
| Example | 0.6 | Slight | 4 | 16 | 14 | 79 |
| B1-17 | | yellow | | | | |
| Example | 0.8 | Slight | 5 | 16 | 28 | 76 |
| B1-18 | | yellow | | | | |
| Example | 0.5 | Slight | 3 | 13 | 29 | 88 |
| B1-19 | | green | | | | |
| Example | 0.2 | Slight | 2 | 10 | 23 | 82 |
| B1-20 | | green | | | | |
| Example | 0.2 | Slight | 2 | 11 | 27 | 72 |
| B1-21 | | blue | | | | |
| Example | 0.9 | Slight | 2 | 16 | 29 | 76 |
| B1-22 | | green | | | | |
| Example | 1.2 | Slight | 6 | 20 | 31 | 76 |
| B1-23 | | green | | | | |
| Compar- | 3 | Yellow | 15 | 24 | 40 | 70 |
| ative | | | | | | |
| Example | | | | | | |
| B1-7 | | | | | | |

### EXAMPLE B1-24

The same reaction apparatus as of Example B1-1 was used and, into its reactor, there was charged a mixture comprising 193 parts of zinc acetate anhydride, 20 parts of manganese(II) acetate tetrahydrate, 3 parts of indium acetate anhydride, and 4,150 parts of 2-propanol, and then its gas phase portion was purged with nitrogen gas. Thereafter, under stirring, the temperature of the mixture was raised from 20° C. and then heat-retained at 180° C.±1° C. for 5 hours to thus carry out a reaction to form metal oxide particles and then cooled, thereby obtaining a dispersion (124) comprising a reaction liquid containing metal oxide particles in a concentration of 2 wt %.

The resultant dispersion was used to carry out the evaluations in the same way as of Example B 1-9. Their results are shown in Table 13.

### EXAMPLES B1-25 to B1-33 and Comparative Examples B1-8 to B1-9

Dispersions (125) to (133) and (c18) to (cl9) comprising reaction liquids, in which metal oxide particles were dispersed in a particle concentration of 2 wt %, were obtained by heating the mixture with the alcohol in the range of 120 to 200° C. in the same way as of Example B1-24 except that the kinds and use amounts of the raw materials being charged were changed as shown in Table 12 (as the raw materials being charged, the additives as shown in Table 12 are also included therein).

The resultant dispersions were used to carry out the evaluations in the same way as of Example B 1-9. Their results are shown in Table 13.

The fine particles in the dispersions having been obtained from Examples B1-24 to B1-33 were evaluated by the uniformity of the distribution. As a result, any case was evaluated as “◯”.

As to the materials of which the valences of the contained metals could be judged from the bond energy values as measured with the photoelectron spectroscope, those valences are shown in the table.

TABLE 12

| | | | | | Surface- |
|---|---|---|---|---|---|
| | | | | | treating |
| | | | | | metal |
| | Zn | M1 | M2 | M3 | (Ms) |
| | compound | compound | compound | compound | compound |
| Example | Zinc | Manganese | Indium | — | — |
| B1-24 | acetate | (II) | acetate | | |
| | anhydride | acetate | anhydride | | |
| | | tetra- | | | |
| | | hydrate | | | |
| Example | Zinc | Manganese | Indium | — | — |
| B1-25 | acetate | (II) | acetate | | |
| | anhydride | acetate | anhydride | | |
| | | tetra- | | | |
| | | hydrate | | | |
| Example | Zinc | Manganese | Aluminum | Iron | — |
| B1-26 | acetate | (III) | (III) | (II) | |
| | anhydride | acetate | s-butoxide | acetate | |
| | | dihydrate | | | |
| Example | Zinc | Manganese | Indium | Bismuth | — |
| B1-27 | acetate | (II) | acetate | (III) | |
| | anhydride | acetate | anhydride | acetate | |
| | | tetra- | | oxide | |
| | | hydrate | | | |
| Example | Zinc | Manganese | Tin (IV) | Indium | Tetra- |
| B1-28 | acetate | (II) | acetate | acetate | methoxy- |
| | anhydride | acetate | | anhydride | silane |
| | | tetra- | | | |
| | | hydrate | | | |
| Example | Zinc | Manganese | Tin (II) | Indium | Zirconium |
| B1-29 | acetate | (II) | acetate | acetate | tetra- |
| | anhydride | acetate | | anhydride | butoxide |
| | | tetra- | | | oligomer |
| | | hydrate | | | (conden- |
| | | | | | sation |
| | | | | | degree: |
| | | | | | 6-10) |
| Example | Zinc | Manganese | Cerium | — | Titanium |
| B1-30 | acetate | (II) | (III) | | tetra- |
| | anhydride | acetate | acetate | | butoxide |
| | | tetra- | hydrate | | tetramer |
| | | hydrate | | | |
| Example | Zinc | Manganese | Titanium | Indium | — |
| B1-31 | acetate | (II) | tetra-n- | acetate | |
| | anhydride | acetate | butoxide | anhydride | |
| | | anhydride | | | |
| Example | Zinc | Manganese | Bismuth | — | — |
| B1-32 | acetate | (II) | (III) | | |
| | anhydride | acetate | acetate | | |
| | | tetra- | | | |
| | | hydrate | | | |
| Example | Zinc | Manganese | Copper | — | — |
| B1-33 | acetate | (II) | (II) | | |
| | anhydride | acetate | acetate | | |
| | | tetra- | anhydride | | |
| | | hydrate | | | |
| Compar- | Zinc | Manganese | — | — | — |
| ative | acetate | (II) | | | |
| Example | anhydride | acetate | | | |
| B1-8 | | tetra- | | | |
| | | hydrate | | | |
| Compar- | Zinc | Indium | — | — | — |
| ative | acetate | acetate | | | |
| Example | anhydride | anhydride | | | |
| B1-9 | | | | | |

TABLE 13

| | | | | | | | | | | | Bonding amount | |
|---|---|---|---|---|---|---|---|---|---|---|---|---|
| | | | | | | | | | | | of acyl group | |
| | X-ray | Metal composition of fine particles | | | | | | | | Crystal | | Bonding |
| | dif- | (fluorescent X-ray analysis) | | | | | | | | grain | | amount |
| | fraction | | Atomic | | Atomic | | Atomic | | Atomic | diameter | Identi- | mol |
| | pattern | M1 | %/Zn | M2 | %/Zn | M3 | %/Zn | Ms | %/Zn | Dw (nm) | fication | %/Zn |
| Example | Equal to | Mn | 6.4 | In | 1 | — | — | — | — | 15 | Ethanoyl | 1.6 |
| B1-24 | ZnO | (II) | | | | | | | | | group | |
| Example | Equal to | Mn | 1.8 | In | 1 | — | — | — | — | 14 | Ethanoyl | 1.6 |
| B1-25 | ZnO | (II) | | | | | | | | | group | |
| Example | Equal to | Mn | 4 | Al | 0.2 | Fe | 1.2 | — | — | 15 | Ethanoyl | 1.8 |
| B1-26 | ZnO | (III) | | | | (II) | | | | | group | |
| Example | Equal to | Mn | 6.2 | In | 0.8 | Bi | 1.7 | — | — | 15 | Ethanoyl | 1.6 |
| B1-27 | ZnO | (II) | | | | | | | | | group | |
| Example | Equal to | Mn | 3.3 | Sn | 0.2 | In | 1 | Si | 2 | 10 | Ethanoyl | 2.3 |
| B1-28 | ZnO | (II) | | | | | | | | | group | |
| Example | Equal to | Mn | 6.3 | Sn | 0.4 | In | 1 | Zr | 0.5 | 14 | Ethanoyl | 3 |
| B1-29 | ZnO | (II) | | (II) | | | | | | | group | |
| Example | Equal to | Mn | 6.2 | Ce | 1.8 | — | — | Ti | 1 | 14 | Ethanoyl | 3.2 |
| B1-30 | ZnO | (II) | | (III) | | | | | | | group | |
| Example | Equal to | Mn | 6.2 | Ti | 1 | In | 1 | — | — | 15 | Ethanoyl | 4 |
| B1-31 | ZnO | (II) | | | | | | | | | group | |
| Example | Equal to | Mn | 6.5 | Bi | 2 | — | — | — | — | 15 | Ethanoyl | 1.9 |
| B1-32 | ZnO | (II) | | | | | | | | | group | |
| Example | Equal to | Mn | 5.8 | Cu | 0.5 | — | — | — | — | 15 | Ethanoyl | 1.7 |
| B1-33 | ZnO | (II) | | | | | | | | | group | |
| Compar- | Equal to | Mn | 6.3 | — | — | — | — | — | — | 16 | Ethanoyl | 1.5 |
| ative | ZnO | (II) | | | | | | | | | group | |
| Example | | | | | | | | | | | | |
| B1-8 | | | | | | | | | | | | |
| Compar- | Equal to | In | 1 | — | — | — | — | — | — | 15 | Ethanoyl | 3 |
| ative | ZnO | | | | | | | | | | group | |
| Example | | | | | | | | | | | | |
| B1-9 | | | | | | | | | | | | |

| | | | | | | Visible- |
|---|---|---|---|---|---|---|
| | | | | | | ray |
| | | | Ultraviolet | | | trans- |
| | Trans- | | absorption | | | mission |
| | parency | | property | | | property |
| | Haze | | | (%) | | (%) |
| | (%) | Hue | 380 nm | 400 nm | 420 nm | 600 nm |
| Example | 0.7 | Light | 3 | 11 | 19 | 77 |
| B1-24 | | yellow | | | | |
| Example | 1.5 | Light | 4 | 26 | 41 | 89 |
| B1-25 | | yellow | | | | |
| Example | 0.7 | Light | 3 | 20 | 36 | 82 |
| B1-26 | | yellow | | | | |
| Example | 0.8 | Light | 2 | 8 | 15 | 80 |
| B1-27 | | yellow | | | | |
| Example | 0.3 | Light | 3 | 17 | 29 | 80 |
| B1-28 | | yellow | | | | |
| Example | 0.2 | Slight | 3 | 18 | 29 | 87 |
| B1-29 | | yellow | | | | |
| Example | 0.2 | Yellow | 4 | 11 | 17 | 72 |
| B1-30 | | | | | | |
| Example | 0.5 | Light | 2 | 9 | 17 | 75 |
| B1-31 | | yellow | | | | |
| Example | 0.8 | Yellow | 7 | 10 | 17 | 80 |
| B1-32 | | | | | | |
| Example | 0.6 | Slight | 2 | 12 | 20 | 75 |
| B1-33 | | yellow | | | | |
| Compar- | 1.2 | Yellow | 10 | 20 | 34 | 70 |
| ative | | | | | | |
| Example | | | | | | |
| B1-8 | | | | | | |
| Compar- | 1.2 | Color- | 20 | 57 | 70 | 87 |
| ative | | less | | | | |
| Example | | | | | | |
| B1-9 | | | | | | |

### EXAMPLES B1-34 to BI-40 and Comparative Examples B1-10 to B1-14

Dispersions (134) to (140) and (c110) to (c114) comprising reaction liquids, in which metal oxide particles were dispersed in a particle concentration of 2 wt %, were obtained in the same way as of Example B 1-1 except that the kinds of the raw materials being charged were changed as shown in Table 14.

The resultant dispersions were used to carry out the evaluations in the same way as of Example B1-9. Their results are shown in Table 15.

The fine particles in the dispersions having been obtained from Examples B1-34 to B1-40 were evaluated by the uniformity of the distribution. As a result, there was seen no segregate comprising (super)fine single metal particles or metal oxide particles containing the added metal element (M1, M2, M3 of Table 10) as a main component, and any case was evaluated as “◯”.

As to the materials of which the valences of the contained metals could be judged from the bond energy values as measured with the photoelectron spectroscope, those valences are shown in the table.

TABLE 14

| | Zn compound | M1 compound | M2 compound | M3 compound |
|---|---|---|---|---|
| Example B1-34 | Zinc acetate | Cerium (III) | Indium acetate | — |
| | anhydride | acetate hydrate | anhydride | |
| Example B1-35 | Zinc propionate | Cerium (III) | Bismuth (III) | — |
| | anhydride | acetate hydrate | acetate | |
| Example B1-36 | Zinc acetate | Tin (II) acetate | Aluminum (III) | — |
| | anhydride | | s-butoxide | |
| Example B1-37 | Zinc formate | Tin (IV) acetate | Titanium | — |
| | anhydride | | tetra-n-butoxide | |
| Example B1-38 | Zinc acetate | Silver (I) acetate | Tin (IV) acetate | — |
| | anhydride | anhydride | | |
| Example B1-39 | Zinc acetate | Silver (I) acetate | Bismuth (III) | — |
| | anhydride | anhydride | acetate oxide | |
| Example B1-40 | Zinc acetate | Silver (I) acetate | Indium acetate | Bismuth (III) |
| | anhydride | anhydride | anhydride | acetate oxide |
| Comparative | Zinc acetate | Aluminum (III) | — | — |
| Example B1-10 | anhydride | s-butoxide | | |
| Comparative | Zinc acetate | Titanium | — | — |
| Example B1-11 | anhydride | tetra-n-butoxide | | |
| Comparative | Zinc acetate | Tin (IV) acetate | — | — |
| Example B1-12 | anhydride | | | |
| Comparative | Zinc acetate | Cerium (III) | — | — |
| Example B1-13 | anhydride | acetate hydrate | | |
| Comparative | Zinc acetate | Silver (I) acetate | — | — |
| Example B1-14 | anhydride | anhydride | | |

TABLE 15

| | | | | | | | | | Bonding amount | |
|---|---|---|---|---|---|---|---|---|---|---|
| | | | | | | | | | of acyl group | |
| | X-ray | Metal composition of fine particles | | | | | | Crystal | | Bonding |
| | dif- | (fluorescent X-ray analysis) | | | | | | grain | | amount |
| | fraction | | Atomic | | Atomic | | Atomic | diameter | Identi- | mol |
| | pattern | M1 | %/Zn | M2 | %/Zn | M3 | %/Zn | Dw (nm) | fication | %/Zn |
| Example | Equal to | Ce | 1 | In | 1 | — | — | 13 | Ethanoyl | 3 |
| B1-34 | ZnO | (III) | | | | | | | group | |
| Example | Equal to | Ce | 2.5 | Bi | 0.5 | — | — | 15 | Propanoyl | 1.7 |
| B1-35 | ZnO | (III) | | | | | | | group | |
| Example | Equal to | Sn | 1 | Al | 1 | — | — | 14 | Ethanoyl | 2 |
| B1-36 | ZnO | (II) | | | | | | | group | |
| Example | Equal to | Sn | 1 | Ti | 1 | — | — | 13 | Formyl | 2.3 |
| B1-37 | ZnO | | | | | | | | group | |
| Example | Equal to | Ag | 0.6 | Sn | 1.2 | — | — | 15 | Ethanoyl | 1.8 |
| B1-38 | ZnO | (I) | | (IV) | | | | | group | |
| Example | Equal to | Ag | 0.5 | Bi | 1 | — | — | 12 | Ethanoyl | 3.1 |
| B1-39 | ZnO | | | | | | | | group | |
| Example | Equal to | Ag | 0.1 | In | 0.9 | Bi | 0.5 | 12 | Ethanoyl | 2.9 |
| B1-40 | ZnO | | | | | | | | group | |
| Compar- | Equal to | Al | 1.5 | — | — | — | — | 15 | Ethanoyl | 3 |
| ative | ZnO | | | | | | | | group | |
| Example | | | | | | | | | | |
| B1-10 | | | | | | | | | | |
| Compar- | Equal to | Ti | 1.5 | — | — | — | — | 15 | Ethanoyl | 3 |
| ative | ZnO | | | | | | | | group | |
| Example | | | | | | | | | | |
| B1-11 | | | | | | | | | | |
| Compar- | Equal to | Sn | 1.5 | — | — | — | — | 15 | Ethanoyl | 3 |
| ative | ZnO | | | | | | | | group | |
| Example | | | | | | | | | | |
| B1-12 | | | | | | | | | | |
| Compar- | Equal to | Ce | 1.5 | — | — | — | — | 15 | Ethanoyl | 3 |
| ative | ZnO | (III) | | | | | | | group | |
| Example | | | | | | | | | | |
| B1-13 | | | | | | | | | | |
| Compar- | Equal to | Ag | 1 | — | — | — | — | 14 | Ethanoyl | 0.8 |
| ative | ZnO | | | | | | | | group | |
| Example | | | | | | | | | | |
| B1-14 | | | | | | | | | | |

| | | | | | | Visible- |
|---|---|---|---|---|---|---|
| | | | | | | ray |
| | | | Ultraviolet | | | trans- |
| | Trans- | | absorption | | | mission |
| | parency | | property | | | property |
| | Haze | | | (%) | | (%) |
| | (%) | Hue | 380 nm | 400 nm | 420 nm | 600 nm |
| Example | 0.2 | Color- | 4 | 28 | 41 | 81 |
| B1-34 | | less | | | | |
| Example | 0.9 | Slight | 4 | 10 | 12 | 83 |
| B1-35 | | yellow | | | | |
| Example | 0.3 | Color- | 6 | 18 | 27 | 83 |
| B1-36 | | less | | | | |
| Example | 0.5 | Color- | 4 | 23 | 35 | 80 |
| B1-37 | | less | | | | |
| Example | 0.5 | Yellow | 3 | 4 | 1 | 77 |
| B1-38 | | | | | | |
| Example | 0.4 | Yellow | 5 | 6 | 3 | 77 |
| B1-39 | | | | | | |
| Example | 0.6 | Yellow | 4 | 10 | 12 | 80 |
| B1-40 | | | | | | |
| Compar- | 0.8 | Color- | 22 | 58 | 70 | 77 |
| ative | | less | | | | |
| Example | | | | | | |
| B1-10 | | | | | | |
| Compar- | 1 | Color- | 21 | 58 | 68 | 76 |
| ative | | less | | | | |
| Example | | | | | | |
| B1-11 | | | | | | |
| Compar- | 0.9 | Color- | 26 | 58 | 70 | 77 |
| ative | | less | | | | |
| Example | | | | | | |
| B1-12 | | | | | | |
| Compar- | 0.9 | Color- | 23 | 48 | 60 | 77 |
| ative | | less | | | | |
| Example | | | | | | |
| B1-13 | | | | | | |
| Compar- | 0.8 | Color- | 2 | 2 | 1 | 62 |
| ative | | less | | | | |
| Example | | | | | | |
| B1-14 | | | | | | |

From Table 15, the following can be understood. Like in Comparative Example B1-14, if ZnO is caused to contain silver, then it comes to absorb visible rays of short wavelengths, but its visible-ray transmittance also becomes low. In comparison, like in Examples B1-38 to B1-40, if, besides silver, another hetero-metal element is caused to be contained, then, while the absorption ability of visible rays of short wavelengths is retained, the visible-ray transmittance also becomes high.

As to the metal oxide particles having been obtained from the above Examples B1-9 to B1-40, the crystal grain diameter in the vertical direction to the lattice plane (002) was not larger than 30 nm. In addition, as to the metal oxide particles having been obtained from the above Examples B 1-9 to B 1-15, the crystal grain diameter in the vertical direction to the lattice plane (002) was not larger than 20 nm in any case, and the crystal grain diameter in the vertical direction to the lattice plane (110) was not smaller than 10 nm in any case.

### EXAMPLE B1-41

A dispersion comprising a reaction liquid of 4 wt % in particle concentration was obtained in the same way as of Example B1-10 except that the reaction solvent was changed to methanol. As to the crystal grain diameters of the resultant fine particles, the crystal grain diameter in the vertical direction to the lattice plane (110) was 8 nm, and the crystal grain diameter in the vertical direction to the lattice plane (002) was 16 nm. In addition, as to the membrane formation evaluations, the transparency was 0.3% in haze, and the hue was colorless. In the particles-dispersed state, the ultraviolet transmittances were 8% at 380 nm and 45% at 420 nm, and the visible-ray transmittance was 86%. From these results, it can be understood that the resultant fine particles are lower in ultraviolet absorption performance than Example B1-10.

### EXAMPLE B1-42

Into the same reactor as of Example B1-1, there was charged a mixture comprising 183 parts of zinc acetate anhydride powder, 12 parts of bismuth(III) acetate oxide, 0.4 part of copper(JI) acetate anhydride powder, 24 parts of indium acetate anhydride powder, and 3,850 parts of 1-propanol, and then its gas phase portion was purged with nitrogen gas. Thereafter, under stirring, the temperature of the mixture was raised and then heat-retained at 180° C.±1° C. for 10 hours to thus carry out a reaction to form metal oxide particles and then cooled, thereby obtaining a dispersion (142) comprising a reaction liquid containing gray fine particles (metal oxide particles) in a concentration of 2 wt %.

It was confirmed that the fine particles in the resultant dispersion (142) were those which comprised a ZnO crystal and were 14 nm in crystal grain diameter and contained copper, indium, and bismuth in ratios of 0.2 atomic %, 4.8 atomic %, and 3 atomic % respectively relative to Zn, and to surfaces of which there was bonded the ethanoyl group in a ratio of 2.5 mol % relative to Zn.

The fine metal oxide particles in the dispersion (142). were evaluated in the ways of (5-3) and (5-4) above. As a result, the ultraviolet absorption property was 3% in transmittance at 380 nm and 35% in transmittance at 400 nm, and the visible-ray transmission property was 85% in transmittance at 600 nm, and the transparency was 1.2% in haze, and the hue was colorless.

### EXAMPLE B1-43

A dispersion (143) was obtained in the same way as of Example B1-42 except that 0.5 part of lithium acetate dihydrate was used as an additional raw material.

It was confirmed that the fine particles in the resultant dispersion (143) were those which comprised a ZnO crystal and were 12 nm in crystal grain diameter and contained copper, indium, bismuth, and lithium in ratios of 0.2 atomic %, 7.8 atomic %, 3.8 atomic %, and 0.5 atomic % respectively relative to Zn, and to surfaces of which there was bonded the ethanoyl group in a ratio of 2.4 mol % relative to Zn.

The fine metal oxide particles in the dispersion (143) were evaluated in the same way as of Example B 1-42. As a result, the ultraviolet absorption property was not more than 1% in transmittance at 380 nm and 20% in transmittance at 400 nm, and the visible-ray transmission property was 87% in transmittance at 600 nm, and the transparency was 0.3% in haze, and the hue was colorless.

### EXAMPLE B1-44

A dispersion (144) was obtained in the same way as of Example B1-9 except that 0.07 part of sodium acetate anhydride was used as an additional raw material.

The resultant dispersion (144) was evaluated in the same way as of Example B1-9. As a result, the metal oxide particles in the dispersion (144) were fine particles of 14 nm in crystal grain diameter comprising a ZnO crystal containing Cu, In, and Na in ratios of 0.4 atomic %, 0.8 atomic %, and 0.08 atomic % respectively relative to Zn. The ultraviolet absorption property was 3% in transmittance at 380 nm and 48% in transmittance at 420 nm, and the visible-ray transmission property was 85% in transmittance at 600 nm.

### EXAMPLE B1-45

A dispersion (145) was obtained in the same way as of Example B1-18 except that 0.3 part of cesium acetate was used as an additional raw material.

The resultant dispersion (145) was evaluated in the same way as of Example B1-18. As a result, the metal oxide particles in the dispersion (145) were fine particles of 18 nm in crystal grain diameter comprising a ZnO crystal containing Fe, Ce, and Cs in ratios of 0.2 atomic %, 0.5 atomic %, and 0.2 atomic % respectively relative to Zn. The ultraviolet absorption property was 2% in transmittance at 380 nm and 22% in transmittance at 420 nm, and the visible-ray transmission property was 78% in transmittance at 600 nm.

### EXAMPLE B1-46

A dispersion (146) was obtained in the same way as of Example B 1-24 except that 0.1 part of magnesium acetate tetrahydrate was used as an additional raw material.

The resultant dispersion (146) was evaluated in the same way as of Example B1-24. As a result, the metal oxide particles in the dispersion (146) were fine particles of 15 nm in crystal grain diameter comprising a ZnO crystal containing Mn, In, and Mg in ratios of 6.6 atomic 1 atomic %, and 0.04 atomic % respectively relative to Zn. The ultraviolet absorption property was not more than 1% in transmittance at 380 nm and 15% in transmittance at 420 nm, and the visible-ray transmission property was 78% in transmittance at 600 nm.

### EXAMPLE B2-1

An amount of 1,000 parts of the reaction liquid (11) (particle concentration: 4.4 wt %), having been obtained from Example B 1-1, was heated under normal pressure to distill off 710 parts of solvent components such as methanol, thereby concentrating the reaction liquid (11). While this concentrated reaction liquid was further heated, ethanol was continuously dropwise added thereto, thereby carrying out solvent displacement with ethanol at the same time as distilling off residual solvent components from the reaction liquid, so that a dispersion (21) such that the metal oxide particles were dispersed in ethanol in a particle concentration of 25 wt % was obtained as a composition for membrane formation.

The dispersing-stability and transparency of the resultant dispersion (21) were evaluated by the aforementioned methods. Their results are shown in Table 16 along with the dispersion particle diameter.

### EXAMPLES B2-2 to B2-5

Dispersions (22) to (25) were obtained as compositions for membrane formation in the same way as of Example B2-1 except that the reaction liquid being used, the solvent being used for the solvent displacement, and the particle concentration were changed as shown in Table 16.

The dispersing-stability and transparency of the resultant dispersions (22) to (25) were evaluated by the aforementioned methods. Their results are shown in Table 16 along with the dispersion particle diameters.

### EXAMPLE B2-6

An amount of 1,000 parts of the reaction liquid (12) (particle concentration: 4.4 wt %), having been obtained from Example B 1-2, was heated under normal pressure to distill off 710 parts of solvent components such as 1-propanol, thereby concentrating the reaction liquid (12). While this concentrated reaction liquid was further heated, t-butanol was continuously dropwise added thereto, thereby carrying out solvent displacement with t-butanol at the same time as distilling off residual solvent components from the reaction liquid. Furthermore, partially hydrolyzed-condensed products (trimer to pentamer) of tetramethoxysilane were added as an additive to the dispersion (resultant from the above solvent displacement) in a formulation ratio of 6 wt % relative to the metal oxide particles in the above dispersion, and then the resultant mixture was subjected to an ultrasonic homogenizer treatment and then, if necessary, its particle concentration was adjusted. As a result, a dispersion (26) such that the metal oxide particles were dispersed in t-butanol in a particle concentration of 20 wt % was obtained as a composition for membrane formation.

The dispersing-stability and transparency of the resultant dispersion (26) were evaluated by the aforementioned methods. Their results are shown in Table 16 along with the dispersion particle diameter.

### EXAMPLES B2-7 to B2-91

Dispersions (27) to (29) were obtained as compositions for membrane formation in the same way as of Example B2-6 except that the reaction liquid being used, the solvent being used for the solvent displacement, the kind and formulation ratio of the additive, and the particle concentration were changed as shown in Table 16.

The dispersing-stability and transparency of the resultant dispersions (27) to (29) were evaluated by the aforementioned methods. Their results are shown in Table 16 along with the dispersion particle diameters.

TABLE 16

| | | Dispersion obtained | | | | | | | |
|---|---|---|---|---|---|---|---|---|---|
| | Re- | | | Particle | Dispersion | Additive | | | |
| | action | | | concen- | particle | | Addition | Evaluation results | |
| | liquid | | | tration | diameter | | amount wt | Dispersing- | trans- |
| | used | No. | Solvent | wt % | μm | Kind | %/particles | stability | parency |
| Example | 11 | 21 | Ethanol | 25 | 0.34 | — | 0 | C | C |
| B2-1 | | | | | | | | | |
| Example | 12 | 22 | 1-Butanol | 26 | 0.31 | — | 0 | C | C |
| B2-2 | | | | | | | | | |
| Example | 13 | 23 | MIBK | 25 | 0.50 | — | 0 | C | C |
| B2-3 | | | | | | | | | |
| Example | 15 | 24 | Butyl | 20 | 0.14 | — | 0 | B | B |
| B2-4 | | | acetate | | | | | | |
| Example | 18 | 25 | PGM | 20 | 0.09 | — | 0 | A | A |
| B2-5 | | | | | | | | | |
| Example | 12 | 26 | t-Butanol | 20 | 0.08 | *1 | 6 | A | A |
| B2-6 | | | | | | | | | |
| Example | 12 | 27 | 1-Butanol | 20 | 0.07 | *2 | 0.8 | A | A |
| B2-7 | | | | | | | | | |
| Example | 12 | 28 | PGMAC | 20 | 0.05 | *3 | 2 | A | A |
| B2-8 | | | | | | | | | |
| Example | 11 | 29 | Ethanol | 20 | 0.10 | *4 | 4 | B | B |
| B2-9 | | | | | | | | | |

(Notes):
*1 Partially hydrolyzed-condensed products (trimer to pentamer) of tetramethoxysilane
*2 Titanium (IV) tetra-n-butoxide tetramer
*3 Aluminum (III) n-butoxide trimer
*4 Titanium (IV) tetra-n-butoxide (monomer)
PGM: Propylene glycol monomethyl ether
PGMAC: Propylene glycol monomethyl ether acetate
MIBK: Methyl isobutyl ketone

### EXAMPLES B2-10 to B2-15

Dispersions (210) to (215) were obtained as compositions for membrane formation in the same way as of Example B 2-1 except that the dispersions as shown in Table 17 were used as the reaction liquids being used, and that the solvent being used for the solvent displacement, and the kind and formulation ratio of the additive, were changed as shown in Table 17.

The particle concentrations and dispersion particle diameters of the resultant dispersions (210) to (215) are shown in Table 17.

TABLE 17

| | | Dispersion obtained | | | | | |
|---|---|---|---|---|---|---|---|
| | | | | Particle | Dispersion | Additive | |
| | Reaction | | | concen- | particle | | Addition |
| | liquid | | | tration | diameter | | amount wt |
| | used | No. | Solvent | wt % | μm | Kind | %/particles |
| Example | Dispersion | 210 | Dipropylene | 20 | 0.04 | — | 0 |
| B2-10 | of Example | | glycol | | | | |
| | B1-10 | | | | | | |
| Example | Dispersion | 211 | 1-Butanol | 20 | 0.04 | — | 0 |
| B2-11 | of Example | | | | | | |
| | B1-16 | | | | | | |
| Example | Dispersion | 212 | MIBK | 25 | 0.04 | *1 | 5 |
| B2-12 | of Example | | | | | | |
| | B1-24 | | | | | | |
| Example | Dispersion | 213 | PGMAC | 25 | 0.05 | *2 | 0.5 |
| B2-13 | of Example | | | | | | |
| | B1-34 | | | | | | |
| Example | Dispersion | 214 | Toluene | 30 | 0.02 | *2 | 12 |
| B2-14 | of Example | | | | | | |
| | B1-36 | | | | | | |
| Example | Dispersion | 215 | Xylene | 20 | 0.09 | — | 0 |
| B2-15 | of Example | | | | | | |
| | B1-38 | | | | | | |

(Notes):
*1 Partially hydrolyzed-condensed products (trimer to pentamer) of tetrametlioxysilane
*2 Titanium (IV) tetra-n-butoxide tetramer
PGMAC: Propylene glycol monomethyl ether acetate
MIBK: Methyl isobutyl ketone

### EXAMPLE B2-16

An amount of 100 parts of the dispersion (215) (having been obtained from Example B2-15), 50 parts of a fluororesin (resin concentration: 40 wt %, solvent: xylene), and 50 parts of xylene (as a diluting solvent) were mixed together and then subjected to a dispersing-treatment with a homogenizer, thereby obtaining a paint.

The resin concentration, particle - concentration, and dispersion particle diameter of the resultant paint are shown in Table 18.

### EXAMPLES B2-17 to B2-20

Paints were obtained in the same way as of Example B2-16 except that the dispersions as shown in Table 18 were used and that the fluororesin was replaced with the resins as shown in Table 18.

The resin concentrations, particle concentration, and dispersion particle diameters of the resultant paints are shown in Table 18.

TABLE 18

| | | | Particle | Resin | Dispersion |
|---|---|---|---|---|---|
| | | | concen- | concen- | particle |
| | Dispersion | Resin | tration | tration | diameter |
| | No. Used | used | wt % | wt % | μm |
| Example | 215 | Fluoro- | 10 | 10 | 0.03 |
| B2-16 | | resin | | | |
| Example | 212 | Polyester | 10 | 10 | 0.07 |
| B2-17 | | resin | | | |
| Example | 213 | Butyral | 1 | 20 | 0.05 |
| B2-18 | | resin | | | |
| Example | 211 | Acrylic | 20 | 10 | 0.03 |
| B2-19 | | polyol | | | |
| Example | 210 | Silica | 10 | 10 | 0.03 |
| B2-20 | | sol | | | |

### EXAMPLE B3-1

An amount of 100 parts of the dispersion (27), having been obtained from Example B2-7, was mixed with 20 parts of a silicate binder (solid component content in terms of SiO₂: 51 wt %) and 0.2 part of a catalyst (n-butylamine) to thus prepare a paint. The dispersion particle diameter of the resultant paint was 0.048 μm.

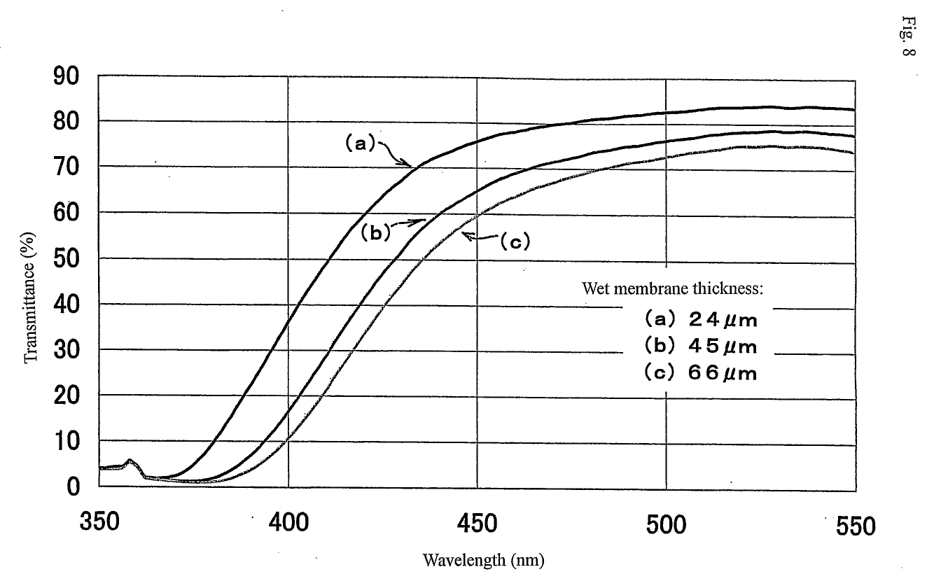

The resultant paint was coated onto alkali-free glass (produced by Corning International Corporation, barium borosilicate glass, Glass Code No. 7059, thickness: 0.6 mm) by use of a bar coater in a way for the wet membrane thickness to be predetermined ones (24 μm, 45 μm, 66 μm). Thereafter, they were normally dried at 25° C. to thereby obtain glasses (three kinds different in dry membrane thickness based on the difference in wet membrane thickness), on the surfaces of which there was formed a metal-oxide-particles-dispersed membrane. These dispersion-membrane-coated glasses were excellent ultraviolet cutting glasses. Transmission spectra of these dispersion-membrane-coated glasses are shown as FIG. 8.

The resultant dispersion-membrane-coated glasses were subjected to the aforementioned evaluations. As a result, the visible-ray transparency was “◯ (haze: not more than 1%)”, and the coloring degree was “◯”. Incidentally, as to the transmittance (%) at each wavelength of only the alkali-free glass used as the substrate, any of the transmittances at 380 nm, 400 nm, 420 nm and 500 nm was 91% (the same also in the below-mentioned Examples B).

### EXAMPLE B3-2

An amount of 100 parts of the dispersion (27), having been obtained from Example B2-7, was mixed with 50 parts of an acrylic resin binder (containing a polyisocyanurate curing agent; entire solid component content: 50 wt %) and 50 parts of a solvent (butyl acetate-toluene) to thus prepare a paint. The dispersion particle diameter of the resultant paint was 0.015 μm.

The resultant paint was coated onto a PET film by use of a bar coater in a way for the wet membrane thickness to be predetermined ones (24 μm, 45 μm, 66 μm). Thereafter, they were heated at 100° C. for 5 minutes to thereby obtain PET films (three kinds different in membrane thickness), on the surfaces of which there was formed a membrane of a dispersion such that Co-In-codoped ZnO particles were dispersed in the acrylic resin. These dispersion-membrane-coated PET films were excellent ultraviolet cutting films similarly to the dispersion-membrane-coated glasses of Example B3-1.

The resultant dispersion-membrane-coated PET films were subjected to the aforementioned evaluations. As a result, the visible-ray transparency was “◯”, and the coloring degree was “◯”. Incidentally, as to the transmittance (%) at each wavelength of only the PET film used as the substrate, any of the transmittances at 380 nm, 400 nm, 420 nm and 500 nm was 85±11%.

### EXAMPLE B3-3

The dispersion (27), having been obtained from Example B2-7, was coated onto the same alkali-free glass as of Example B3-1 by use of a bar coater, and then its temperature was raised from normal temperature in a calcination furnace and then retained at 400° C. for 1 hour and then cooled, thus obtaining glass, on the surface of which there was formed a thin membrane of Co-In-codoped ZnO particles.

The resultant thin-membrane-coated glass was subjected to the aforementioned evaluations. As a result, the visible-ray transparency was “◯ (haze: not more than 1%)”, and the coloring degree was “◯”. As to the visible-ray transmission property, the transmittance at 500 nm was 88%. As to the ultraviolet absorption property, the transmittance at 400 nm was 60%.

### EXAMPLE B3-4

An amount of 100 parts of the dispersion (27), having been obtained from Example B2-7, was mixed with 10 parts of an ultraviolet curing type coating agent (“HIC2000” produced by KYOEISHA CHEMICAL Co., LTD.; entire solid component content: 50 wt %; refractive index: 1.576) (as a binder solution) and 15 parts of a solvent (methyl ethyl ketone) to thus prepare a paint of 20 wt % in total solid component content.

The resultant paint was coated onto a PET film by use of a bar coater and then set for 10 minutes and then heat-dried at 100° C. for 1 minute and then irradiated with ultraviolet rays by use of a high-pressure mercury lamp (ultraviolet exposure dose: 600 mJ/cm²) to thereby obtain a membrane-coated PET film, on the surface of which there was formed a membrane of 5 μm in dry membrane thickness.

The resultant membrane-coated PET film was evaluated by the refractive index and the visible-ray transparency (haze). As a result, the refractive index was not less than 1.7, and the haze was less than 1%. In addition, the resultant membrane-coated PET film was less than 20% in transmittance at 380 nm and was 80% in transmittance at 500 nm and was a film excellent in the ultraviolet intercepting ability.

### EXAMPLE B3-5

The dispersion (19), having been obtained from Example B1-9, was coated onto an alkali glass as a substrate by use of a bar coater and then dried at normal temperature and then heated at 400° C. under a nitrogen atmosphere in a heating furnace for 1 hour, thus obtaining a membrane-coated substrate, on the surface of which there was formed a membrane of 0.5 μm in membrane thickness.

The resultant membrane-coated substrate was evaluated. As a result, this membrane-coated substrate was provided with a ZnO crystal membrane containing Cu, In, and Si in ratios of 0.4 atomic %, 0.8 atomic %, and 5 atomic % respectively relative to Zn and was a colorless ultraviolet-interception glass of which: the ultraviolet absorption property was 50% in transmittance at 380 nm, and the visible-ray transmission property was 88% in transmittance at 600 nm, and the transparency was 0.8% in haze.

### EXAMPLE B3-6

The dispersion (130), having been obtained from Example B1-30, was coated onto an alkali glass as a substrate by use of a bar coater and then dried at normal temperature and then heated at 400° C. under a nitrogen atmosphere in a heating furnace for 1 hour, thus obtaining a membrane-coated substrate, on the surface of which there was formed a membrane of 1.2 μm in membrane thickness.

The resultant membrane-coated substrate was evaluated. As a result, this membrane-coated substrate was provided with a ZnO crystal membrane containing Mn, Ce, and Ti in ratios of 6.2 atomic %, 1.8 atomic %, and 1 atomic % respectively relative to Zn and was an ultraviolet-interception glass of which: the ultraviolet absorption property was 20% in transmittance at 380 nm, and the visible-ray transmission property was 88% in transmittance at 600 nm, and the transparency was 0.3% in haze.

### EXAMPLE B4-1

Into the same reactor as of Example B1-1, there was charged a mixture comprising 303 parts of titanium methoxypropoxide, 3.5 parts of a 21% ethanol solution of iron(III)ethoxide, 22 parts of a 15% diethylene glycol monoethyl ether solution of aluminum(III)ethoxyethoxyethoxide, 4 parts of copper(II) 2-(2-butoxyethoxy)ethoxide, 2,400 parts of ethylene glycol dimethyl ether, and 270 parts of acetic acid, and then its gas phase portion was purged with nitrogen gas. Thereafter, under stirring, the temperature of the mixture was raised (from 20° C.) to 180° C. and then heat-retained at 180° C.±1° C. for 5 hours to thus carry out a reaction to form metal oxide particles and then cooled, thereby obtaining a reaction liquid (41) containing fine particles (metal oxide particles) in a fine particle concentration of 2 wt %. Furthermore, the resultant reaction liquid was subjected to heating solvent displacement in the same way as of Example B1-1 to thereby obtain a dispersion (41) such that the above metal oxide particles were dispersed in 1-butanol in a particle concentration of 20 wt %.

The metal oxide particles in the dispersion (41) were subjected to the aforementioned various measurements and evaluations. Their results are shown in Table 19. Incidentally, as to the ultraviolet absorption property and visible-ray absorption property, they were evaluated in the following way according to the aforementioned (5-3): the dispersion was diluted with 1-butanol to thus prepare a sample liquid of 0.1 wt % in particle concentration, and then this sample liquid was filled into a quartz cell of 1 cm in thickness, and then this filled cell was used to measure a transmission spectrum with the auto-recording spectrophotometer and, from this transmission spectrum, the ultraviolet absorption property and the visible-ray absorption property were evaluated by transmittances at 380 nm and 600 nm respectively.

### COMPARATIVE EXAMPLE B4-1

A dispersion (c41) such that metal oxide particles were dispersed in 1-butanol in a particle concentration of 20 wt % was obtained in the same way as of Example B4-1 except that there was used neither 22 parts of the 15% diethylene glycol monoethyl ether solution of aluminum(III) ethoxyethoxyethoxide nor 4 parts of copper(II) 2-(2-butoxyethoxy)ethoxide.

The metal oxide particles in the dispersion (c41) were subjected to the aforementioned various measurements and evaluations. Their results are shown in Table 19. Incidentally, as to the ultraviolet absorption property and visible-ray absorption property, they were evaluated in the same way as of Example B4-1 according to the aforementioned (5-3).

TABLE 19

| | | | | | | | Visible- | |
|---|---|---|---|---|---|---|---|---|
| | | | | | | | ray | |
| | | Metal composition of | | | | Crystal | trans- | Ultraviolet |
| | X-ray | fine particles (fluorescent | | | | grain | mission | absorption |
| | dif- | x-ray analysis) | | | | diameter | property | property |
| | fraction | | Atomic | | Atomic | (nm) | (%) | (%) |
| | pattern | M1 | %/Ti | M2 | %/Ti | Ds (101) | 600 nm | 380 nm |
| Example | Equal to | Fe | 0.5 | Al | 1 | 6 | 95 | 2 |
| B4-1 | TiO₂ | | | Cu | 1.2 | | | |
| Compar- | Equal to | Fe | 0.5 | — | — | 7 | 95 | 15 |
| ative | TiO₂ | | | | | | | |
| Example | | | | | | | | |
| B4-1 | | | | | | | | |

### EXAMPLE B4-2

Into the same reactor as of Example B1-1, there was charged a mixture comprising 50 parts of cerium(III) acetate monohydrate, 0.6 part of iron(III) acetate hydroxide, 0.14 part of copper(II) acetate, and 3,000 parts of pure water, and then there was added 50 parts of a 30% aqueous hydrogen peroxide solution under stirring at room temperature. Next, under stirring, the temperature of the mixture was raised (from the room temperature) to 90° C. and then heat-retained at 90° C.±2° C. for 5 hours, and then 10 parts of a 30% aqueous hydrogen peroxide solution was added. Thereafter, the temperature was heat-retained for another 1 hour to thus carry out a reaction to form metal oxide particles and then cooled, thereby obtaining a reaction liquid (42) containing slightly yellow and high-transparent-feeling fine particles (metal oxide particles) in a fine particle concentration of 0.8 wt % Next, the resultant reaction liquid was subjected to filtration with an ultrafiltration membrane to thereby remove impurity ions and residual hydrogen peroxide and also make concentration, thus obtaining a dispersion (42) such that the above metal oxide particles were dispersed in water in a particle concentration of 7 wt %.

The metal oxide particles in the dispersion (42) were subjected to the aforementioned various measurements and evaluations. Their results are shown in Table 20. Incidentally, as to the crystal system and crystal structure, the particles were so fine as to give a broad peak by the powder X-ray diffractometry. Thus, the crystal system and crystal structure was judged by measuring the lattice constant by electron diffractometry and then comparing its results with data of a standard powder. In addition, as to the primary particle diameter, it was judged with a transmission electron microscope. In addition, as to the ultraviolet absorption property and visible-ray absorption property, they were evaluated in the following way according to the aforementioned (5-3): the dispersion was diluted with pure water to thus prepare a sample liquid of 0.1 wt % in particle concentration, and then this sample liquid was filled into a quartz cell of 1 cm in thickness, and then this filled cell was used to measure a transmission spectrum with the auto-recording spectrophotometer and, from this transmission spectrum, the ultraviolet absorption property was evaluated by transmittances at 380 nm and 400 nm, and the visible-ray absorption property was evaluated by a transmittance at 600 nm.

### COMPARATIVE EXAMPLE B4-2

A dispersion (c42) such that metal oxide particles were dispersed in water in a particle concentration of 7 wt % was obtained in the same way as of Example B4-2 except that there was used neither 0.6 part of iron(III) acetate hydroxide nor 0.14 part of copper(II) acetate.

The metal oxide particles in the dispersion (c42) were subjected to the aforementioned various measurements and evaluations. Their results are shown in Table 20. Incidentally, as to the crystal system and crystal structure, the primary particle diameter, the ultraviolet absorption property, and the visible-ray absorption property, they were evaluated in the same way as of Example B4-2.

### COMPARATIVE EXAMPLE B4-3

A dispersion (c43) such that metal oxide particles were dispersed in water in a particle concentration of 7 wt % was obtained in the same way as of Example B4-2 except that there was not used the 0.14 part of copper(II) acetate.

The metal oxide particles in the dispersion (c43) were subjected to the aforementioned various measurements and evaluations. Their results are shown in Table 20. Incidentally, as to the crystal system and crystal structure, the primary particle diameter, the ultraviolet absorption property, and the visible-ray absorption property, they were evaluated in the same way as of Example B4-2.

TABLE 20

| | | | | | | | Visible- | | |
|---|---|---|---|---|---|---|---|---|---|
| | | | | | | | ray | | |
| | | Metal composition of | | | | | trans- | Ultraviolet | |
| | X-ray | fine particles (fluorescent | | | | Primary | mission | absorption | |
| | dif- | X-ray analysis) | | | | particle | property | property | |
| | fraction | | Atomic | | Atomic | diameter | (%) | (%) | |
| | pattern | M1 | %/Ce | M2 | %/Ce | (nm) | 600 nm | 380 nm | 400 nm |
| Example | Equal to | Fe | 2 | Cu | 0.5 | 2-4 | 99 | <1 | 18 |
| B4-2 | CeO₂ | | | | | | | | |
| Compar- | Equal to | — | — | — | — | 2-4 | 99 | 29 | 75 |
| ative | CeO₂ | | | | | | | | |
| Example | | | | | | | | | |
| B4-2 | | | | | | | | | |
| Compar- | Equal to | Fe | 2 | — | — | 2-4 | 99 | 5 | 24 |
| ative | CeO₂ | | | | | | | | |
| Example | | | | | | | | | |
| B4-3 | | | | | | | | | |

### EXAMPLE B4-3

Into the same reactor as of Example B1-1, there was charged a mixture comprising 146 parts of indium acetate anhydride, 1.9 parts of iron(III) acetate hydroxide, 4.25 parts of titanium tetra-n-butoxide, and 3,322 parts of I1-propanol, and then its gas phase portion was purged with nitrogen gas. Thereafter, under stirring, the temperature of the mixture was raised (from 20° C.) to 180° C. and then heat-retained at 180° C.±1° C. for 5 hours to thus carry out a reaction to form metal oxide particles and then cooled, thereby obtaining a reaction liquid (43) containing yellow fine particles (metal oxide particles) in a fine particle concentration of 2 wt % Furthermore, the resultant. reaction liquid was subjected to heating solvent displacement in the same way as of Example B1-1 to thereby obtain a dispersion (43) such that the above metal oxide particles were dispersed in 1-butanol in a particle concentration of 20 wt %.

The metal oxide particles in the dispersion (43) were subjected to the aforementioned various measurements and evaluations. Their results are shown in Table 21. Incidentally, as to the ultraviolet absorption property and visible-ray absorption property, they are mentioned below.

### EXAMPLE B4-4

A reaction liquid (44) containing yellow fine particles (metal oxide particles) in a fine particle concentration of 2 wt % was obtained in the same way as of Example B4-3 except that the 1.9 parts of iron(III) acetate hydroxide and the 4.25 parts of titanium tetra-n-butoxide were replaced with 0.2 part of silver acetate and 3.6 parts of tin(IV) acetate. Furthermore, the resultant reaction liquid was subjected to heating solvent displacement in the same way as of Example B1-1 to thereby obtain a dispersion (44) such that the above metal oxide particles were dispersed in 1-butanol in a particle concentration of 20 wt %.

The metal oxide particles in the dispersion (44) were subjected to the aforementioned various measurements and evaluations. Their results are shown in Table 21. Incidentally, as to the ultraviolet absorption property and visible-ray absorption property, they are mentioned below.

### COMPARATIVE EXAMPLE B4-4

A reaction liquid (c44) containing yellow fine particles (metal oxide particles) in a fine particle concentration of 2 wt % was obtained in the same way as ofExample B4-3 except that there was used neither 1.9 parts of iron(III) acetate hydroxide nor 4.25 parts of titanium tetra-n-butoxide. Furthermore, the resultant reaction liquid was subjected to heating solvent displacement in the same way as of Example B1-1 to thereby obtain a dispersion (c44) such that the above metal oxide particles were dispersed in 1-butanol in a particle concentration of 20 wt %.

The metal oxide particles in the dispersion (c44) were subjected to the aforementioned various measurements and evaluations. Their results are shown in Table 21. Incidentally, as to the ultraviolet absorption property and visible-ray absorption property, they are mentioned below.

As to the ultraviolet absorption property and visible-ray absorption property of the metal oxide particles in each dispersion having been obtained from Example B4-3, Example B4-4, and Comparative Example B4-4, they were evaluated in the following way according to the aforementioned (5-1): the dispersion was diluted with 1-butanol to thus prepare a sample liquid of 0.5 wt %. in particle concentration, and then this sample liquid was filled into a quartz cell of 1 cm in thickness, and then this filled cell was used to measure a transmission spectrum with the auto-recording spectrophotometer and, from this transmission spectrum, the ultraviolet absorption property was evaluated by transmittances at 380 nm and 400 nm, and the visible-ray absorption property was evaluated by a transmittance at 600 nm. As a result, the transmittance at 600 nm, namely, the visible-ray absorption property, was equal in any case. However, as to the transmittances at 380 nm and 400 nm, Example B4-3 and Example B4-4 were lower than Comparative Example B4-4. Therefore, Example B4-3 and Example B4-4 were more excellent than Comparative Example B4-4 in the ultraviolet absorption property.

TABLE 21

| | Metal composition of fine | | | | Crystal |
|---|---|---|---|---|---|
| X-ray | fine particles (fluorescent | | | | grain |
| dif- | X-ray analysis | | | | diameter |
| fraction | | Atomic | | Atomic | (nm) |
| pattern | M1 | %/In | M2 | %/In | Ds (222) |

| | | | | | | |
|---|---|---|---|---|---|---|
| Example | Equal to | Fe | 2 | Ti | 2.5 | 4.2 |
| B4-3 | In₂O₃ | | | | | |
| Example | Equal to | Ag | 0.2 | Sn | 2 | 4.5 |
| B4-4 | In₂O₃ | | | | | |
| Compar- | Equal to | — | — | — | — | 4.8 |
| ative | In₂O₃ | | | | | |
| Example | | | | | | |
| B4-4 | | | | | | |

### EXAMPLE B4-5

A dispersion (45) was obtained in the same way as of Example B4-1 except that 2 parts of strontium acetate 0.5-hydrate was used as an additional raw material.

The resultant dispersion (45) was evaluated in the same way as of Example B4-1. As a result, the metal oxide particles in the dispersion (45) were fine particles of 5 nm in crystal grain diameter comprising an anatase type TiO₂ crystal containing Fe, Al, Cu, and Sr in ratios of 0.5 atomic %, 1 atomic %, 1.2 atomic %, and 1.2 atomic % respectively relative to Ti. The ultraviolet absorption property was not more than 1% in transmittance at 380 nm, and the visible-ray transmission property was 96% in transmittance at 600 nm.

### EXAMPLE B5-1

Into the same reactor as of Example B1-1, there was charged a mixture comprising 183 parts of zinc acetate anhydride, 2 parts of silver acetate, 3.5 parts of indium acetate, and 3,900 parts of 1-propanol, and then its gas phase portion was purged with nitrogen gas. Thereafter, under stirring, the temperature of the mixture was raised (from 20° C.) to 250° C. and then heat-retained at 250° C.±1° C. for 5 hours to thus carry out a reaction to form metal oxide particles and then cooled, thereby obtaining a reaction liquid (51) containing fine particles.

The fines particles in the reaction liquid (51) were analyzed and, as a result, found to comprise: superfine zinc oxide particles of 15 nm in crystal grain diameter containing Ag and In in a ratio of 0.2 atomic % relative to Zn; and superfine Ag particles of 18 nm in crystal grain diameter. Element mapping was carried out by the same method as the XMA analysis method with the transmission electron microscope in the aforementioned evaluation method (3). From its results, the content of the segregated superfine Ag particles was found to be about 1% in number ratio relative to the superfine zinc oxide particles.

From the reaction liquid (51), a 1-butanol dispersion of 20 wt % in particle concentration was obtained in the same way as of Example B1-1. The resultant dispersion was evaluated in the same way as of Example B1-9. From their results, the following were found. As to the membrane formation evaluations, the transparency was 0.9% in haze, and the hue was yellow. In the particles-dispersed state (fine particle concentration: 0.1 wt %), the ultraviolet transmittances were 2% at 380 nm and 1% at 420 nm, and the visible-ray transmittance was 65%.

### EXAMPLE B5-2

An amount of 6 parts of an Ag nano-particle powder of 8 nm in crystal grain diameter having been prepared by a separate process was added to 1,000 parts of the toluene dispersion (214) of 30 wt % in particle concentration (having been obtained from Example B2-14) to mix them together, and then the resultant mixture was subjected to a dispersing-treatment with a homogenizer, thereby obtaining a dispersion in which: superfine zinc oxide particles containing Sn and Al were dispersed and contained in a ratio of 30 wt %; and superfine Ag particles were dispersed and contained in a ratio of 0.6 wt %.

The resultant dispersion was evaluated in the same way as of Example B1-9. From their results, the following were found. As to the membrane formation evaluations, the transparency was 2% in haze, and the hue was yellow. In the particles-dispersed state, the ultraviolet transmittances were 5% at 380 nm and 8% at 420 nm, and the visible-ray transmittance was 60%.

The resultant dispersion was diluted with toluene to vary the particle concentration down to 0.1-0.01 wt % and then evaluated by the spectroscopic properties. As a result, it was confirmed that the absorption at 420 nm was due to absorption by the added superfine Ag particles having an absorption maximum at about 430 nm.

### EXAMPLE B5-3

In the same reactor as of Example B1-1, a mixture of a bismuth(III) acetate oxide powder and 1-propanol was heated at 200° C. to thereby obtain a reaction liquid in which superfine bismuth oxide (Bi₂O₃) particles of 20 nm in crystal grain diameter were dispersed and contained in a ratio of 2 wt %. From this reaction liquid, a 1-butanol dispersion of 20 wt % in fine particle concentration was obtained in the same way as of Example B1-1.

An amount of 100 parts of the resultant dispersion and 1,000 parts of the dipropylene glycol dispersion (having been obtained from Example B2-10) were mixed together and then subjected to a dispersing-treatment with a homogenizer, thereby obtaining a dispersion in which: superfine zinc oxide particles containing Cu and Ce were dispersed and contained in a ratio of 18 wt %; and superfine Bi₂O₃ particles were dispersed and contained in a ratio of 1.8 wt %.

The resultant dispersion was evaluated in the same way as of Example B1-9. From their results, the following were found. As to the membrane formation evaluations, the transparency was 0.9% in haze, and the hue was yellow. In the particles-dispersed state, the ultraviolet transmittances were 2% at 380 nm and 20% at 420 nm, and the visible-ray transmittance was 80%.

### EXAMPLE B5-4

In the same reactor as of Example B1-1, a mixture of a iron(III) acetate hydroxide powder and 1-propanol was heated at 160° C. to thereby obtain a reaction liquid in which superfine a-ferric oxide (α-Fe₂O₃) particles of 15 nm in crystal grain diameter (to which the ethanoyl group was bonded in a ratio of 5 mol % relative to iron) were dispersed and contained in a ratio of 2 wt %. From this reaction liquid, a 1-butanol dispersion of 20 wt % in fine particle concentration was obtained in the same way as of Example B1-1.

An amount of 50 parts of the resultant dispersion and 1,000 parts of the dipropylene glycol dispersion (having been obtained from Example B2-10) were mixed together and then subjected to a dispersing-treatment with a homogenizer, thereby obtaining a dispersion in which: superfine zinc oxide particles containing Cu and Ce were dispersed and contained in a ratio of 19 wt %; and superfine α-Fe₂O₃ particles were dispersed and contained in a ratio of 0.95 wt %.

The resultant dispersion was evaluated in the same way as of Example B1-9.

From their results, the following were found. As to the membrane formation evaluations, the transparency was 0.9% in haze, and the hue was yellow. In the particles-dispersed state (fine particle concentration: 0.1 wt %), the ultraviolet transmittances were 2% at 380 nm and 24% at 420 nm, and the visible-ray transmittance was 77%.

[Third Metal Oxide Particle]:

The measurement and evaluation methods in the below-mentioned Examples and Comparative Examples are shown below.

<Evaluation of Metal Oxide Particles>:

(1) Crystal Identification of Metal Oxide Particles:

As to the above powder sample, the crystal system and crystal structure of the metal oxide particles were evaluated by powder X-ray diffractometry with a powder X-ray diffraction device (produced by Rigaku Denki K.K., product name: RINT 2400). Hereinafter the measurement conditions are shown.
X-rays: CuKα1 rays (wavelength: 1.54056 Å)/40 kV/200 mA
Scanning range: 2θ=20 to 80°
Scanning speed: 5°/min

Whether the metal oxide particles had the same crystal system and crystal structure as of ZnO or not was judged from whether three intense-ray peaks characteristic of ZnO of the hexagonal crystal system were seen or not. Specifically, if diffraction peaks existed in all positions of the following three diffraction angles (a) to (c), then it was judged that the metal oxide particles had the same crystal system and crystal structure as of ZnO.
(a) 2θ=31.65 to 31.95°
(b) 2θ=34.30 to 34.60°
(c) 2θ=36.10 to 36.40°

Incidentally, the diffraction peak existing in the position of the above (a) is judged to be based on diffracted rays to the (100) plane of the ZnO crystal, and the diffraction peak existing in the position of the above (b) is judged to be based on diffracted rays to the (002) plane of the ZnO crystal, and the diffraction peak existing in the position of the above (c) is judged to be based on diffracted rays to the (101) plane of the ZnO crystal.

(2) Particle Diameter of Metal Oxide Particles:

(2-1) Crystal Grain Diameter (Ds):

As to the above powder sample, the crystal grain diameter (Ds) of the metal oxide particles was evaluated by powder X-ray diffractometry with a powder X-ray diffraction device (produced by Rigaku Denki K.K., product name: RINT 2400). Specifically, the crystal grain diameter Ds (hkl) (wherein the hkl denotes a Miller index: the Ds (hkl) is the size of the crystal grain in the vertical direction to the lattice plane of the Miller index (hkl)) was determined by Scherrer equation (analysis) from widths of diffracted rays in the resultant X-ray diffraction pattern.

(2-2) Primary particle diameter: crystal grain diameter (Dw):

The crystal grain diameter (Dw) of the metal oxide particles was measured and evaluated as the primary particle diameter.

The crystal grain diameter (Dw) was evaluated in the following way: as to the above powder sample, the crystal grain diameter (Dw) of the metal oxide particles was evaluated by powder X-ray diffractometry with a powder X-ray diffraction device (produced by Rigaku Denki K.K., product name: RINT 2400). Specifically, the crystal grain diameter Ds (hkl) (wherein the hlcl denotes a Miller index: the Ds (hkl) is the size of the crystal grain in the vertical direction to the lattice plane of the Miller index (hkl)) was determined by Scherrer equation (analysis) from widths of diffracted rays in the resultant X-ray diffraction pattern, and the average value of three intense rays' respective Ds values was taken as the Dw. That is to say, unless otherwise noted, the crystal grain diameter (Dw) is usually calculated in the following way. A powder X-ray diffraction pattern of the metal oxide particle is measured, and then, as to three intense rays thereof (the largest peak (1) of diffracted rays, the second largest peak (2) of diffracted rays, and the third largest peak (3) of diffracted rays), the crystal grain diameters Ds1, Ds2, and Ds3 in the vertical directions to the diffraction planes assigned to the diffracted rays (1) to (3) respectively are determined from their respective fall widths of half maximum intensity or integral widths in accordance with Scherrer equation, and then their average value ((Ds1+Ds2+Ds3)/3) is calculated as the crystal grain diameter (Dw).

(2-3) Dispersion Particle Diameter:

The resultant reaction liquid, or a solvent dispersion having been obtained from this reaction liquid by solvent displacement, was used as the sample, and its median diameter was measured with a dynamic light scattering type particle diameter distribution measurement device (“LB-500” produced by Horiba Seisakusho) and taken as the dispersion particle diameter.

(3) Composition of Metal Oxide Particles (Average Composition of Metal Elements):

The above powder sample was subjected to quantitative analyses into metal elements by fluorescent X-ray analyses or ICP analyses to thus determine the contents of the hetero-metal elements (Co, Fe, Ni) relative to the main metal element (M) and, in cases where the metal compound was used as an additive during the formation of the particles, the content of the metal element (Ms) of the above metal compound relative to the main metal element (M).

In addition, while each particle in the above powder sample was observed with an FE-TEM (field emission transmission electron microscope) as equipped with an XMA device (X-ray microanalyzer) of 1 nmφ in resolution, any portion of from the surface layer of the particle up to its central portion was subjected to local elemental analysis, and the deflection of the intensity ratio of a peak intensity assigned to each metal element to a peak intensity assigned to the main metal element (M) was evaluated to thus judge whether each metal element contained in the particle was uniformly distributed or not (i.e. judge the uniformity of the distribution). In addition, when the local elemental analysis into each metal element was carried out, whether or not there was any segregate of the hetero-metal elements (Co, Fe, Ni) or of the metal element (Ms) of the metal compound was also evaluated.
◯: The metal elements (Co, Fe, Ni, Ms) other than the main metal element (M) are uniformly contained.
×: The metal elements (Co, Fe, Ni, Ms) other than the main metal element (M) are not uniformly contained and/or segregates of their metals or compounds were seen.

(4) Evaluation of Valence of Hetero-Metal Element (Co, Fe, Ni) Contained in Metal Oxide Particles:

As to the above powder sample, a 2p_{3/2} spectrum of the hetero-metal element (Co, Fe, Ni) contained in the metal oxide particles was measured by X-ray photoelectron spectroscopy (XPS) with a photoelectron spectroscope (produced by Nippon Denshi K.K., product name: JPS-90 model) and, from its peak position, the bond energy value was determined to thus judge the valence of the hetero-metal element (Co, Fe, Ni).

Incidentally, in order to reduce measured value errors caused by such as energy shift due to the electrification property, the determination of the bond energy value was put under corrections based on the C 1s peak position of the surface hydrocarbon.

In addition, as already known data for comparison, peak positions of 2p_{3/2} spectra of compounds of the hetero-metal elements (Co, Fe, Ni) as shown in “The Handbook of X-ray Photoelectron Spectroscopy” (1991) published by Nippon Denshi K.K. were referred to.

(5) Optical Properties of Metal Oxide Particles:

(5-1) Evaluation in Form of Dispersion Membrane:

A reaction liquid resultant from a reaction to form metal oxide particles was subjected to heating solvent displacement to thereby obtain a dispersion such that the metal oxide particles were dispersed in 1-butanol in a particle concentration of 20 wt %. An amount of 100 parts of the resultant dispersion was mixed with 20 parts of a silicate binder (solid component content in terms of SiO₂: 51 wt %) and 0.5 part of a catalyst (n-butylamine), and then the resultant mixture was subjected to a dispersing-treatment with an ultrasonic homogenizer for 10 minutes and then stirred for 3 hours thus preparing a paint. Incidentally, as to the above particle concentration, it was calculated in a way that the solid component amount as a result of vacuum drying of the resultant dispersion at 120° C. with a vacuum drier for 1 hour was taken as the particle weight.

The resultant paint was coated onto alkali-free glass (produced by Corning International Corporation, barium borosilicate glass, Glass Code No. 7059, thickness: 0.6 mm) by use of a bar coater in a way for the wet membrane thickness to be 24 μm.

Thereafter, they were normally dried at 25° C. to thereby obtain glass, on the surface of which there was formed the metal-oxide-particles-dispersed membrane. Then, this dispersion-membrane-coated glass was evaluated by: (i) visible-ray transmission property, ultraviolet absorption property, and visible long-wavelength absorption property based on transmission spectra; (ii) visible-ray transparency; and (iii) coloring degree.
—About evaluation of (i) above—

(i-a):

This evaluation was carried out about only the membrane portion of the dispersion-membrane-coated glass.

The transmission spectrum of each of the dispersion-membrane-coated glass and the above alkali-free glass (substrate only) was measured by use of an auto-recording spectrophotometer having an integrating sphere (produced by Shimadzu Corporation, product name: UV-3100).

From the resultant transmission spectrum, as to each of the dispersion-membrane-coated glass and the alkali-free glass, the visible-ray transmission property was evaluated by a visible-ray transmittance (transmittance (%) of light of 500 nm in wavelength (transmittance at 500 nm; it is hereinafter provided that the wording portions analogous thereto shall also refer to the same meaning)), and the ultraviolet absorption property was evaluated by a transmittance (%) at 380 nm. In addition, the visible long-wavelength absorption property was evaluated in the following way: as to whether or not there was any absorption band in the range of 550 to 700 nm that could be caused by containing the ion of the hetero-metal element (Co, Fe, Ni) and as to the degree of such an absorption band, A (%) was determined by the following equation:
Δ(%)=[|T⁵⁰⁰−T¹|/T⁵⁰⁰]×100
(wherein: T¹ is the minimum value of transmittances (%) in the range of 550 to 700 nm; and T⁵⁰⁰ is a transmittance (%) at 500 nm), and its value was evaluated on the following standards:
A: Δ(%) <5%
B: 5%≦Δ(%)<10%
C: 10%≦Δ(%)

Incidentally, the above transmittance at each wavelength of only the membrane portion is determined by the following equation:
Transmittance (%) at each wavelength of only the membrane portion=[transmittance (%) at each wavelength of dispersion-membrane-coated glass/transmittance (%) at each wavelength of alkali-free glass]×100
(wherein: as to the transmittance (%) at each wavelength of the alkali-free glass determined by the above evaluation method, any of the transmittance at 500 nm, the transmittance at 380 nm, and the transmittances in the range of 550 to 700 nm is 91%)
(i-b):

The same evaluation was carried out in the same way as of the above evaluation method (i-a) except that a dispersion-membrane-coated glass was obtained in a way for the wet membrane thickness of the paint by the bar coater to be 66 μm. Incidentally, this evaluation was carried out only about the metal oxide particles as specified in the Examples.
—About evaluation of (ii) above—

The dispersion-membrane-coated glass was measured by the total ray transmittance, the diffused-ray transmittance, the parallel-ray transmittance, and the haze value (H (%)) with a turbidimeter (produced by Nippon Denshoku Kogyo Co., Ltd., product name: NDH-1001 DP)

The transparency of the dispersion-membrane-coated glass was evaluated on the following standards.
A: H(%) <1%
B: 1%≦H(%)<3%
C: 3%≦H (%)
—About evaluation of (iii) above—

As to the resultant dispersion-membrane-coated glass, its appearance was observed with the eye to thereby evaluate the coloring degree on the following standards:
×: Coloring is remarkable.
Δ: Coloring is seen a little.
◯: Colorless, or coloring is not noticeable.

(5-2) Evaluation in State Dispersed in Solvent:

A dilution, having been prepared by diluting the resultant reaction liquid with 1-butanol as a diluting solvent so as to be 0.1 wt % in fine particle concentration, was used as the sample and, as to this sample, its transmission spectrum in the ultraviolet and visible ranges was measured by use of an auto-recording spectrophotometer having an integrating sphere (“UV-3100” produced by Shimadzu Corporation).

Ultraviolet intercepting ability: evaluated by transmittances at 380 nm, 400 nm, 420 nm.

Visible-ray transmission property: evaluated by a transmittance at 600 nm.

Incidentally, also as to the transmission spectrum of a membrane-formed product, its transmission spectrum in the ultraviolet and visible ranges was, similarly to the above, measured by use of the auto-recording spectrophotometer having an integrating sphere (“UV-3100” produced by Shimadzu Corporation).

(6) Hue of Metal Oxide Particles:

It was evaluated by observing the appearance of the powder sample with the eye.

<Evaluation of Membrane (or Membrane-Coated Substrate)>:

(1) Visible-ray transmission property, ultraviolet absorption property, and visible long-wavelength absorption property based on transmission spectrum:

This evaluation was carried out about the membrane-coated substrate.

The transmission spectrum of the membrane-coated substrate was measured by use of an auto-recording spectrophotometer having an integrating sphere (produced by Shimadzu Corporation, product name: UV-3100).

From the resultant transmission spectrum, as to the membrane-coated substrate, the visible-ray transmission property was evaluated by a visible-ray transmittance (transmittance (%) at 500 nm), and the ultraviolet absorption property was evaluated by a transmittance (%) at 380 nm. In addition, the visible long-wavelength absorption property was evaluated in the following way: as to whether or not there was any absorption band in the range of 550 to 700 nm that could be caused by containing the ion of the hetero-metal element (Co, Fe, Ni) and as to the degree of such an absorption band, Δ(%) was determined by the following equation:
Δ(%)=[|T⁵⁰⁰−T¹|/T⁵⁰⁰]×100
(wherein: T¹ is the minimum value of transmittances (%) in the range of 550 to 700 nm; and T⁵⁰⁰ is a transmittance (%) at 500 nm), andits value was evaluated on the following standards:
A: Δ(%) <5%
B: 5%≦Δ(%) <10%
C: 10%≦Δ(%)

Incidentally, also as to only the substrate used for the membrane-coated substrate, the transmittance at each wavelength was determined by the same method as the above.

(2) Visible-Ray Transparency:

Each of the membrane-coated substrate and only the substrate was measured by the total ray transmittance, the diffused-ray transmittance, the parallel-ray transmittance, and the haze value (H (%)) with a turbidimeter (produced by Nippon Denshoku Kogyo Co., Ltd., product name: NDH-1001 DP) to evaluate the transparency of the membrane from the measured haze value on the following standards. Incidentally, the haze value of the membrane is given by subtracting a haze value of only the substrate from that of the membrane-coated substrate.
A: H(%)<1%
B: 1%≦H (%)<3%
C: 3%≦H (%)

(3) Coloring Degree:

As to the resultant membrane (membrane-coated substrate), its appearance was observed with the eye to thereby evaluate the coloring degree on the following standards:
×: Coloring is remarkable.
Δ: Coloring is seen a little.
◯: Colorless, or coloring is not noticeable.

### EXAMPLE C1-1

There was prepared a reaction apparatus comprising: a pressure-resistant glass reactor possible to externally heat and equipped with a stirrer, an addition inlet (connected directly to an addition tank), a thermometer, a distillate gas outlet, and a nitrogen-gas-introducing inlet; the addition tank connected to the above addition inlet; and a condenser (connected directly to a trap) connected to the above distillate gas outlet.

Into the above reactor, there was charged a mixture comprising 183 parts of zinc acetate anhydride powder, 3.5 parts of cobalt(II) acetate anhydride powder, 2 parts of methyltrimethoxysilane, and 1,700 parts of methanol, and then its gas phase portion was purged with nitrogen gas. Thereafter, under stirring, the temperature of the mixture was raised (from 20° C.) to 160° C. and then heat-retained at 160° C.±1° C. for 5 hours to thus carry out a reaction to form metal oxide particles and then cooled, thereby obtaining a reaction liquid (11) containing blue fine particles (metal oxide particles).

The metal oxide particles in the reaction liquid (11) were subjected to the aforementioned various measurements and evaluations. Their results are shown in Table 23. Incidentally, as to the valence of Co contained in the metal oxide particles in the reaction liquid (11), it was evaluated by measuring a 2p_{3/2} spectrum of Co in the aforementioned way. As a result, from its peak position being 780.3 eV, it was judged that Co of 2 in valence (Co(II)) was contained.

As to the metal oxide particles in the reaction liquid (11), the evaluation (5) (i-b) among the aforementioned evaluations of metal oxide particles was also carried out. As a result, the transmittance at 380 nm was less than 1%, and the transmittance at 500 nm was 80%, and the Δ(%) was rank A.

### EXAMPLES C1-2 to C1-4

Reaction liquids (12) to (14), containing blue fine particles (metal oxide particles), were obtained in the same way as of Example C1-1 except that the kinds and use amounts of the raw materials being charged were changed as shown in Table 22.

The metal oxide particles in each of the reaction liquids (12) to (14) were subjected to the aforementioned various measurements and evaluations. Their results are shown in Table 23. Incidentally, as to the valence of Co contained in the metal oxide particles in the reaction liquids (12) to (14), it was evaluated by measuring a 2p_{3/2} spectrum of Co in the aforementioned way. As a result, from its peak position, it was judged that Co of 2 in valence (Co(II)) was contained similarly to Example C1-1.

### EXAMPLE C1-5

There was prepared the same reaction apparatus as of Example C1-1 and, into the pressure-resistant glass reactor as equipped to this apparatus, there was charged a mixture comprising 183 parts of zinc acetate anhydride powder, 1.8 parts of cobalt(II) acetate anhydride powder, 1.9 parts of iron(III) acetate hydroxide powder, 3 parts of tetramethoxysilane, and 1,700 parts of 1-propanol, and then its gas phase portion was purged with nitrogen gas. Thereafter, under stirring, the temperature of the mixture was raised (from 20° C.) to 160° C. and then heat-retained at 160° C.±1° C. for 2 hours to thus carry out a reaction to form metal oxide particles and then cooled, thereby obtaining a reaction liquid (15) containing blue fine particles (metal oxide particles). Incidentally, the above color of the metal oxide particles was more alleviated and therefore favorable for uses which demanded to be more colorless transparent, when compared with the metal oxide particles having been obtained from Examples C1-1 to C1-4.

The metal oxide particles in the reaction liquid (15) were subjected to the aforementioned various measurements and evaluations. Their results are shown in Table 23. Incidentally, as to the valence of Co contained in the metal oxide particles in the reaction liquid (15), it was evaluated by measuring a 2p_{3/2} spectrum of Co in the aforementioned way. As a result, from its peak position, it was judged that Co of 2 in valence (Co(II)) was contained similarly to Example C1-1.

### COMPARATIVE EXAMPLE C1-1

A reaction liquid (c11), containing white fine particles (metal oxide particles), was obtained in the same way as of Example C1-1 except that the cobalt(II) acetate anhydride powder was not used as a raw material being charged.

The metal oxide particles in the reaction liquid (c11) were subjected to the aforementioned various measurements and evaluations. Their results are shown in Table 23.

### EXAMPLES C1-6 to C1-8

Reaction liquids (16) to (18), containing blue fine particles (metal oxide particles), were obtained in the same way as of Example C1-1 except that the kinds and use amounts of the raw materials being charged were changed as shown in Table 22.

The metal oxide particles in each of the reaction liquids (16) to (18) were subjected to the aforementioned various measurements and evaluations. Their results are shown in Table 23. Incidentally, as to the valence of Co contained in the metal oxide particles in the reaction liquids (16) to (18), it was evaluated by measuring a 2p_{3/2} spectrum of Co in the aforementioned way. As a result, from its peak position, it was judged that Co of 2 in valence (Co(II)) was contained similarly to Example C1-1.

TABLE 22

| | Zn compound | | Co compound | | Fe compound | | Additive | | Solvent | |
|---|---|---|---|---|---|---|---|---|---|---|
| | | Weight | | Weight | | Weight | | Weight | | Weight |
| | | parts | | parts | | parts | | parts | | parts |
| Example | Zinc acetate | 183 | Cobalt (II) | 3.5 | — | — | Methyltrimethoxysilane | 2 | Methanol | 1700 |
| C1-1 | | | acetate | | | | | | | |
| | | | anhydride | | | | | | | |
| Example | Zinc acetate | 183 | Cobalt (II) | 7.0 | — | — | Tetramethoxysilane | 8 | 1-Propanol | 1700 |
| C1-2 | | | acetate | | | | | | | |
| | | | anhydride | | | | | | | |
| Example | Zinc acetate | 183 | Cobalt (II) | 1.5 | — | — | Decryltrimethoxysilane | 10 | Ethanol | 1700 |
| C1-3 | | | acetate | | | | | | | |
| | | | anhydride | | | | | | | |
| Example | Zinc acetate | 183 | Cobalt (II) | 5.0 | — | — | Titanium (IV) | 16 | Methanol | 1700 |
| C1-4 | | | acetate | | | | tetra-n-butoxide | | | |
| | | | anhydride | | | | tetramer | | | |
| Example | Zinc acetate | 183 | Cobalt (II) | 1.8 | Iron (III) acetate | 1.9 | Tetramethoxysilane | 3.0 | 1-Propanol | 1700 |
| C1-5 | | | acetate | | hydroxide | | | | | |
| | | | anhydride | | | | | | | |
| Comparative | Zinc acetate | 183 | — | — | — | — | Methyltrimethoxysilane | 2 | Methanol | 1700 |
| Example | | | | | | | | | | |
| C1-1 | | | | | | | | | | |
| Example | Zinc acetate | 183 | Cobalt (II) | 3.5 | — | — | Methyltrimethoxysilane | 15 | Methanol | 1700 |
| C1-6 | | | acetate | | | | | | | |
| | | | anhydride | | | | | | | |
| Example | Zinc acetate | 183 | Cobalt (II) | 3.5 | — | — | — | — | Ethanol | 1700 |
| C1-7 | | | acetate | | | | | | | |
| | | | anhydride | | | | | | | |
| Example | Zinc acetate | 183 | Cobalt (II) | 3.5 | — | — | Tetramethoxysilane | 2 | 1-Propanol | 1700 |
| C1-8 | | | acetate | | | | | | | |
| | | | anhydride | | | | | | | |

TABLE 23

| | | | | | | | | Dispersion membrane evaluations | | | |
|---|---|---|---|---|---|---|---|---|---|---|---|
| | | | | | | | | | | Ultraviolet | |
| | | | | | | Crystal grain | | | Visible-ray | absorption | Visible long- |
| | Metal | X-ray | Co content | Fe content | Uniformity | diameter (nm) | | | transmission | property | wavelength |
| | oxide | diffraction | Atomic %/ | Atomic %/ | of | Ds | Ds | Trans- | property (%) | (%) | absorption |
| | particle | pattern | Zn | Zn | distribution | (100) | (002) | parency | 500 nm | 380 nm | property |
| Example C1-1 | 11 | Equal to ZnO | 2 | — | ◯ | 9 | 15 | A | 98 | 36 | A |
| Example C1-2 | 12 | Equal to ZnO | 3.8 | — | ◯ | 8 | 12 | A | 97 | 30 | A |
| Example C1-3 | 13 | Equal to ZnO | 0.8 | — | ◯ | 9 | 21 | A | 98 | 49 | A |
| Example C1-4 | 14 | Equal to ZnO | 2.8 | — | ◯ | 12 | 12 | A | 97 | 33 | A |
| Example C1-5 | 15 | Equal to ZnO | 1 | 1 | ◯ | 8 | 17 | A | 98 | 29 | A |
| Comparative | c11 | Equal to ZnO | — | — | — | 9 | 22 | A | 98 | 76 | A |
| Example C1-1 | | | | | | | | | | | |
| Example C1-6 | 16 | Equal to ZnO | 2 | — | ◯ | 6 | 14 | A | 98 | 55 | A |
| Example C1-7 | 17 | Equal to ZnO | 2 | — | ◯ | 16 | 33 | B | 94 | 32 | A |
| Example C1-8 | 18 | Equal to ZnO | 2 | — | ◯ | 15 | 15 | A | 97 | 28 | A |

### Comparative Example C1-2

There was prepared the same reaction apparatus as of Example C1-1 and, into the pressure-resistant glass reactor as equipped to this apparatus, there was charged a mixture comprising 300 parts of zinc acetate hexahydrate powder, 3 parts of cobalt(II) acetate anhydride powder, and 2,000 parts of ion-exchanged water. Thereafter, under stirring, the mixture was retained at 20° C. To this mixture, there was dropwise added 1,000 parts of ion-exchanged water in which 200 parts of sodium carbonate was dissolved. Thereafter, the stirring was carried out for 2 hours to thereby obtain a slurry. This slurry was subjected to centrifugal separation, and then the resultant sediment was washed with ion-exchanged water and then filtered off. The resultant cake was dried at 100° C. and then calcined at 500° C. under an air atmosphere in a calcination furnace for 2 hours and then cooled. The resultant powder was pulverized and then dispersed into 1-butanol with a sand mill so as to be 20 wt % in concentration, thus obtaining a dispersion (c12).

The evaluation results of the metal oxide particles in the resultant dispersion (c12) were as follows.

The Co contents in individual particles, as described in the above evaluation method (3), were non-uniform and immeasurable. However, the average content of Co, as determined by elemental analysis of the powder of the metal oxide particles, was 1.3 atomic % in terms of atomic ratio relative to Zn. The valence of Co as contained in the particles was 3 as a result of the judgment in the same way as of Example C1-1. The crystal grain diameter in the vertical direction to the (002) plane was 38 nm. As a result of having evaluated the optical performances of the metal oxide particles in accordance with the above evaluation method (5-1), the transparency was rank C, and the visible-ray transmission property was 78% in transmittance at 500 nm.

### EXAMPLES C1-9 to C1-16 and Comparative Examples C1-3 to C1-4

Reaction liquids (19) to (116), (c13), and (c14), containing metal oxide particles, were obtained in the same way as of Example C1-1 except that the kinds and use amounts of the raw materials being charged were changed as shown in Table 24.

The metal oxide particles in each of the reaction liquids (19) to (116), (c13), and (c14) were subjected to the aforementioned various measurements and evaluations. Their results are shown in Table 25. Incidentally, in this table, as comparison, there are also shown the results of the same evaluations about Comparative Example C1-1.

TABLE 24

| | Zn compound | | Fe compound | | Ni compound | | Co compound | |
|---|---|---|---|---|---|---|---|---|
| | | Weight | | Weight | | Weight | | Weight |
| | | parts | | parts | | parts | | parts |
| Example | Zinc | 183 | Iron (III) | 2.0 | — | — | — | — |
| C1-9 | acetate | | acetate | | | | | |
| | | | hydroxide | | | | | |
| Example | Zinc | 183 | Iron (II) | 3.5 | — | — | — | — |
| C1-10 | acetate | | acetate | | | | | |
| Example | Zinc | 183 | Iron (II) | 5.2 | — | — | — | — |
| C1-11 | acetate | | acetate | | | | | |
| Example | Zinc | 183 | Iron (II) | 3.5 | — | — | — | — |
| C1-12 | acetate | | acetate | | | | | |
| Example | Zinc | 183 | Iron (III) | 2.0 | — | — | — | — |
| C1-13 | acetate | | acetate | | | | | |
| | | | hydroxide | | | | | |
| | | | Iron (II) | 1.8 | | | | |
| | | | acetate | | | | | |
| Example | Zinc | 183 | — | — | Nickel (II) | 5 | — | — |
| C1-14 | acetate | | | | acetate | | | |
| | | | | | tetrahydrate | | | |
| Example | Zinc | 183 | Iron (III) | 2.0 | — | — | Cobalt (II) | 0.13 |
| C1-15 | acetate | | acetate | | | | acetate | |
| | | | hydroxide | | | | tetrahydrate | |
| Example | Zinc | 183 | Iron (III) | 2.0 | Nickel (II) | 0.3 | — | — |
| C1-16 | acetate | | acetate | | acetate | | | |
| | | | hydroxide | | tetrahydrate | | | |
| Comparative | Zinc | 183 | Iron (III) | 2.0 | — | — | — | — |
| Example | acetate | | acetate | | | | | |
| C1-3 | | | hydroxide | | | | | |
| Comparative | Zinc | 183 | Iron (III) | 2.0 | — | — | — | — |
| Example | acetate | | acetate | | | | | |
| C1-4 | | | hydroxide | | | | | |

| | Additive | | Solvent | | | |
|---|---|---|---|---|---|---|
| | | Weight | | Weight | Heating | Heating |
| | | parts | | parts | temperature (° C.) | time (hr) |
| Example | Tetramethoxysilane | 1 | 2-Propanol | 1700 | 120 | 12 |
| C1-9 | | | | | | |
| Example | Tetramethoxysilane | 2 | Ethanol | 1700 | 120 | 12 |
| C1-10 | | | | | | |
| Example | Tetramethoxysilane | 9 | Methanol | 1700 | 160 | 2 |
| C1-11 | | | | | | |
| Example | — | — | Ethanol | 1700 | 120 | 12 |
| C1-12 | | | | | | |
| Example | Tetramethoxysilane | 2 | 1-Propanol | 1700 | 120 | 12 |
| C1-13 | | | | | | |
| Example | — | — | Ethanol | 1700 | 200 | 12 |
| C1-14 | | | | | | |
| Example | Tetramethoxysilane | 1 | 2-Propanol | 1700 | 120 | 12 |
| C1-15 | | | | | | |
| Example | Tetramethoxysilane | 1 | 2-Propanol | 1700 | 120 | 12 |
| C1-16 | | | | | | |
| Comparative | Tetramethoxysilane | 9 | Methanol | 1700 | 120 | 12 |
| Example | | | | | | |
| C1-3 | | | | | | |
| Comparative | — | — | 1-Propanol | 1700 | 120 | 12 |
| Example | | | | | | |
| C1-4 | | | | | | |

TABLE 25

| | Metal | X-ray | Fe content | Ni content | Co content | Valence | |
|---|---|---|---|---|---|---|---|
| | oxide | diffraction | Atomic %/ | Atomic %/ | Atomic %/ | of metal | Uniformity of |
| | particle | pattern | Zn | Zn | Zn | element (M′) | distribution |
| Example | 19 | Equal to | 1 | — | — | Fe (III) | A |
| C1-9 | | ZnO | | | | | |
| Example | 110 | Equal to | 2 | — | — | Fe (II) | A |
| C1-10 | | ZnO | | | | | |
| Example | 111 | Equal to | 3 | — | — | Fe (II) | A |
| C1-11 | | ZnO | | | | | |
| Example | 112 | Equal to | 2 | — | — | Fe (II) | A |
| C1-12 | | ZnO | | | | | |
| Example | 113 | Equal to | 2 | — | — | Fe (II) | A |
| C1-13 | | ZnO | | | | Fe (III) | |
| Example | 114 | Equal to | — | 2 | — | Ni (II) | A |
| C1-14 | | ZnO | | | | | |
| Example | 115 | Equal to | 1 | — | 0.05 | Fe (III) | A |
| C1-15 | | ZnO | | | | Co (II) | |
| Example | 116 | Equal to | 1 | 0.12 | — | Fe (III) | A |
| C1-16 | | ZnO | | | | Ni (II) | |
| Comparative | c13 | Equal to | 1 | — | — | Fe (III) | A |
| Example | | ZnO | | | | | |
| C1-3 | | | | | | | |
| Comparative | c14 | Equal to | 1 | — | — | Fe (III) | A |
| Example | | ZnO | | | | | |
| C1-4 | | | | | | | |
| Comparative | c11 | Equal to | — | — | — | — | — |
| Example | | ZnO | | | | | |
| C1-1 | | | | | | | |

| | | | | Dispersion membrane evaluations | | | |
|---|---|---|---|---|---|---|---|
| | Crystal grain | | | | | Visible-ray | Ultraviolet |
| | diameter (nm) | | Hue of | | | transmission | absorption |
| | Ds | Ds | particle | Trans- | Coloring | property (%) | property (%) |
| | (100) | (002) | powder | parency | degree | 500 nm | 380 nm |
| Example | 14 | 15 | Yellow | A | Δ | 93 | 47 |
| C1-9 | | | | | | | |
| Example | 12 | 15 | Green | A | ◯ | 92 | 36 |
| C1-10 | | | | | | | |
| Example | 7 | 12 | Green | A | ◯ | 95 | 45 |
| C1-11 | | | | | | | |
| Example | 16 | 33 | Green | B | ◯ | 86 | 38 |
| C1-12 | | | | | | | |
| Example | 15 | 15 | Yellowish | A | ◯ | 90 | 40 |
| C1-13 | | | green | | | | |
| Example | 20 | 18 | Light | A | ◯ | 89 | 38 |
| C1-14 | | | green | | | | |
| Example | 14 | 14 | Yellowish | A | ◯ | 93 | 40 |
| C1-15 | | | green | | | | |
| Example | 12 | 14 | Yellowish | A | Δ | 92 | 41 |
| C1-16 | | | green | | | | |
| Comparative | 6 | 15 | Yellow | A | Δ | 94 | 58 |
| Example | | | | | | | |
| C1-3 | | | | | | | |
| Comparative | 16 | 33 | Yellow | C | X | 75 | 48 |
| Example | | | | | | | |
| C1-4 | | | | | | | |
| Comparative | 9 | 22 | White | A | ◯ | 98 | 76 |
| Example | | | | | | | |
| C1-1 | | | | | | | |

### EXAMPLE C1-17

A reaction liquid (117), containing metal oxide particles, was obtained in the same way as of Example C1-10 except that 0.18 part of lithium acetate dihydrate powder was used as an additional raw material.

The reaction liquid (117) was subjected to the same evaluations as of Example C1-10. As a result, the ultraviolet absorption property (transmittance at 380 nm) was 32%, which was an enhanced one when compared with the reaction liquid (110) having been obtained from Example C1-10. In addition, the lithium ion was contained in a ratio of 0.18 atomic % relative to Zn. The other evaluation results were the same as of the reaction liquid (110).

### EXAMPLE C1-18

A reaction liquid (118), containing metal oxide particles, was obtained in the same way as of Example C1-10 except that 0.14 part of calcium acetate monohydrate powder was used as an additional raw material.

The reaction liquid (118) was subjected to the same evaluations as of Example C1-10. As a result, the ultraviolet absorption property (transmittance at 380 nm) was 33%, which was an enhanced one when compared with the reaction liquid (110) having been obtained from Example C1-10. In addition, the calcium ion was contained in a ratio of 0.08 atomic % relative to Zn. The other evaluation results were the same as of the reaction liquid (110).

### EXAMPLE C1-19

A reaction liquid (119), containing metal oxide particles, was obtained in the same way as of Example C1-9 except that 0.05 part of magnesium acetate tetrahydrate powder was used as an additional raw material.

The reaction liquid (119) was subjected to the same evaluations as of Example C1-9. As a result, the ultraviolet absorption property (transmittance at 380 nm) was 43%, which was an enhanced one when compared with the reaction liquid (19) having been obtained from Example C1-9. In addition, the magnesium ion was contained in a ratio of 0.02 atomic % relative to Zn. The other evaluation results were the same as of the reaction liquid (19).

### EXAMPLE C1-20

A reaction liquid (120), containing metal oxide particles, was obtained in the same way as of Example C1-9 except that 0.78 part of cesium acetate anhydride powder was used as an additional raw material.

The reaction liquid (120) was subjected to the same evaluations as of Example C1-9. As a result, the ultraviolet absorption property (transmittance at 380 nm) was 44%, which was an enhanced one when compared with the reaction liquid (19) having been obtained from Example C1-9. In addition, the cesium ion was contained in a ratio of 0.4 atomic % relative to Zn. The other evaluation results were the same as of the reaction liquid (19).

### EXAMPLE C1-21

A reaction liquid (121), containing metal oxide particles, was obtained in the same way as of Example C1-14 except that 0.4 part of sodium acetate anhydride powder was used as an additional raw material.

The reaction liquid (121) was subjected to the same evaluations as of Example C1-14. As a result, the ultraviolet absorption property (transmittance at 380 nm) was 35%, which was an enhanced one when compared with the reaction liquid (114) having been obtained from Example C1-14. In addition, the sodium ion was contained in a ratio of 0.4 atomic % relative to Zn. The other evaluation results were the same as of the reaction liquid (114).

### EXAMPLE C1-22

A reaction liquid (122), containing metal oxide particles, was obtained in the same way as of Example C1-1 except that 0.51 part of barium acetate anhydride powder was used as an additional raw material.

The reaction liquid (122) was subjected to the same evaluations as of Example C1-1. As a result, the ultraviolet absorption property (transmittance at 380 nm) was 34%, which was an enhanced one when compared with the reaction liquid (11) having been obtained from Example C1-1. In addition, the barium ion was contained in a ratio of 0.2 atomic % relative to Zn. The other evaluation results were the same as of the reaction liquid (11).

### EXAMPLE C1-23

Into the same reactor as of Example C1-1, there was charged a mixture comprising 2,400 parts of ethylene glycol dimethyl ether (as a reaction solvent), 303 parts of titanium methoxypropoxide, 2.8 parts of iron(II) acetate powder, and 270 parts of acetic acid (as an additive), and then its gas phase portion was purged with nitrogen gas. Thereafter, under stirring, the temperature of the mixture was raised from 20° C. to 160° C. and then heated at 160° C.±1° C. for 5 hours and then cooled, thereby obtaining a reaction liquid (123) of 2 wt % in fine particle concentration.

The crystal system and crystal structure of the fine particles in the reaction liquid (123) were judged equal to anatase type titanium oxide from the results of the powder X-ray diffractometry.

The fine particles in the reaction liquid (123) were subjected to the aforementioned various evaluations. Their results are shown in Table 26. Incidentally, as to the evaluation of the optical performances, the visible-ray transmission property and the ultraviolet absorption property were evaluated in accordance with the above evaluation method (5-2) using the reaction liquid as the sample (however, the ultraviolet absorption property was evaluated by the transmittance at 400 nm), and the coloring degree was evaluated in accordance with the above evaluation method (5-1) in which the 1-butanol dispersion was used as the sample to carry out the evaluation in the form of the dispersion membrane.

### EXAMPLE C1-24

A reaction liquid (124), containing metal oxide particles, was obtained in the same way as of Example C1-23 except that the 2.8 parts of iron(II) acetate powder was replaced with 8 parts of cobalt(II) acetate anhydride powder.

The crystal system and crystal structure of the fine particles in the reaction liquid (124) were judged equal to anatase type titanium oxide from the results of the powder X-ray diffractometry.

The fine particles in the reaction liquid (124) were subjected to the same evaluations as of Example C1-23. Their results are shown in Table 26.

### EXAMPLE C1-25

A reaction liquid (125), containing metal oxide particles, was obtained in the same way as of Example C1-23 except that the 2.8 parts of iron(II) acetate powder was replaced with 1 part of nickel(II) acetate tetrahydrate powder.

The crystal system and crystal structure of the fine particles in the reaction liquid (125) were judged equal to anatase type titanium oxide from the results of the powder X-ray diffractometry.

The fine particles in the reaction liquid (125) were subjected to the same evaluations as of Example C1-23. Their results are shown in Table 26.

### EXAMPLE C1-26

A reaction liquid (126), containing metal oxide particles, was obtained in the same way as of Example C1-23 except that the 2.8 parts of iron(II) acetate powder was replaced with 0.65 part of iron(II) acetate powder and 3 parts of iron(III) acetate hydroxide powder.

The crystal system and crystal structure of the fine particles in the reaction liquid (126) were judged equal to anatase type titanium oxide from the results of the powder X-ray diffractometry.

The fine particles in the reaction liquid (126) were subjected to the same evaluations as of Example C1-23. Their results are shown in Table 26.

### EXAMPLE C1-27

A reaction liquid (127), containing metal oxide particles, was obtained in the same way as of Example C1-23 except that the 2.8 parts of iron(II) acetate powder was replaced with 0.1 part of cobalt(II) acetate anhydride powder and 1.5 parts of iron(III) acetate hydroxide powder.

The crystal system and crystal structure of the fine particles in the reaction liquid (127) were judged equal to anatase type titanium oxide from the results of the powder X-ray diffractometry.

The fine particles in the reaction liquid (127) were subjected to the same evaluations as of Example C1-23. Their results are shown in Table 26.

### COMPARATIVE EXAMPLE C1-5

A reaction liquid (c15), containing metal oxide particles, was obtained in the same way as of Example C1-23 except that there was not used the 2.8 parts of iron(II) acetate powder.

The crystal system and crystal structure of the fine particles in the reaction liquid (c15) were judged equal to anatase type titanium oxide from the results of the powder X-ray diffractometry.

The fine particles in the reaction liquid (c15) were subjected to the same evaluations as of Example C1-23. Their results are shown in Table 26.

### COMPARATIVE EXAMPLE C1-6

A reaction liquid (c16), containing metal oxide particles, was obtained in the same way as of Example C1-23 except that the 2.8 parts of iron(II) acetate powder was replaced with 3 parts of iron(III) acetate hydroxide powder.

The crystal system and crystal structure of the fine particles in the reaction liquid (c16) were judged equal to anatase type titanium oxide from the results of the powder X-ray diffractometry.

The fine particles in the reaction liquid (c16) were subjected to the same evaluations as of Example C1-23. Their results are shown in Table 26.

TABLE 26

| | | | | | | Crystal | Visible-ray | Ultraviolet | | Coloring degree |
|---|---|---|---|---|---|---|---|---|---|---|
| | X-ray | Metal composition | | | | grain | transmission | absorption | Hue of | in form of |
| | diffraction | | Atomic %/ | | Atomic %/ | diameter | property (%) | property (%) | particle | dispersion |
| | pattern | M′ (1) | Ti | M′ (2) | Ti | Dw (nm) | 600 nm | 400 nm | powder | membrane |
| Example C1-23 | Equal to anatase | Fe (II) | 2 | — | — | 7 | 72 | 15 | Green | ◯ |
| | type TiO₂ | | | | | | | | | |
| Example C1-24 | Equal to anatase | Co (II) | 6 | — | — | 6 | 77 | 15 | Blue | ◯ |
| | type TiO₂ | | | | | | | | | |
| Example C1-25 | Equal to anatase | Ni (II) | 0.5 | — | — | 8 | 73 | 15 | Green | ◯ |
| | type TiO₂ | | | | | | | | | |
| Example C1-26 | Equal to anatase | Fe (II) | 0.5 | Fe (III) | 2 | 5 | 74 | 11 | Yellowish | ◯ |
| | type TiO₂ | | | | | | | | green | |
| Example C1-27 | Equal to anatase | Co (II) | 0.07 | Fe (III) | 1 | 7 | 72 | 9 | Blue | ◯ |
| | type TiO₂ | | | | | | | | | |
| Comparative | Equal to anatase | — | — | — | — | 8 | 78 | 60 | Slight | ◯ |
| Example C1-5 | type TiO₂ | | | | | | | | yellow | |
| Comparative | Equal to anatase | Fe (III) | 2 | — | — | 8 | 72 | 20 | Yellow | X |
| Example C1-6 | type TiO₂ | | | | | | | | | |

### EXAMPLE C1-28

Into the same reactor as of Example C1-1, there was charged a mixture comprising 3,000 parts of pure water, 50 parts of cerium(III) acetate monohydrate powder, 1.5 parts of iron(III) acetate hydroxide powder, and 0.19 part of nickel(II) acetate tetrahydrate powder, and then there was added 50 parts of a 30% aqueous hydrogen peroxide solution under stirring at room temperature. Next, under stirring, the temperature of the mixture was raised from the room temperature to 90° C. and then heated at 90° C.±2° C. for 5 hours, and then 10 parts of a 30% aqueous hydrogen peroxide solution was added. Thereafter, the temperature was heat-retained for another 1 hour and then cooled, thereby obtaining a reaction liquid which was slightly yellow and gave a high-transparent feeling and had a fine particle concentration of 0.8 wt %. Next, the resultant reaction liquid was subjected to filtration with an ultrafiltration membrane to thereby remove impurity ions and residual hydrogen peroxide and also make concentration, thus obtaining a water dispersion (128) having a fine particle concentration of 7 wt %.

The fine particles in the water dispersion (128) were subjected to the aforementioned various evaluations. Their results are shown in Table 27. Incidentally, as to the evaluation of the optical performances, the visible-ray transmission property and the ultraviolet absorption property were evaluated in accordance with the above evaluation method (5-2) using the water dispersion as the sample (however, the ultraviolet absorption property was evaluated by the transmittances at 380 nm and 400 nm), and the coloring degree was evaluated in accordance with the above evaluation method (5-1) to carry out the evaluation in the form of the dispersion membrane. As to the crystal system and crystal structure, the particles were so fine as to give a broad peak by the powder X-ray diffractometry. Thus, the crystal system and crystal structure were judged by measuring the lattice constant by electron diffractometry and then comparing its results with data of a standard powder. As to the primary particle diameter, it was judged with a transmission electron microscope.

### COMPARATIVE EXAMPLE C1-7

A water dispersion (c17) having a fine particle concentration of 7 wt % was obtained in the same way as of Example C1-28 except that there was not used the 0.19 part of nickel(II) acetate tetrahydrate powder.

The fine particles in the water dispersion (c17) were subjected to the same evaluations as of Example C1-28. Their results are shown in Table 27.

### COMPARATIVE EXAMPLE C1-8

A water dispersion (c18) having a fine particle concentration of 7 wt % was obtained in the same way as of Example C1-28 except that there was used neither the 1.5 parts of iron(III) acetate hydroxide powder nor the 0.19 part of nickel(II) acetate tetrahydrate powder.

The fine particles in the water dispersion (c18) were subjected to the same evaluations as of Example C1-28. Their results are shown in Table 27.

TABLE 27

| | | | | | | | | | | | Coloring |
|---|---|---|---|---|---|---|---|---|---|---|---|
| | | | | | | Crystal | Visible-ray | Ultraviolet | | | degree |
| | X-ray | Metal composition | | | | grain | transmission | absorption | | | in form of |
| | diffraction | | Atomic %/ | | Atomic %/ | diameter | property (%) | property (%) | | Hue of particle | dispersion |
| | pattern | M′ (1) | Ce | M′ (2) | Ce | Dw (nm) | 600 nm | 380 nm | 400 nm | powder | membrane |
| Example C1-28 | Equal to CeO₂ | Ni (II) | 0.5 | Fe (III) | 5 | 2-4 | 98 | 10 | 40 | Yellowish | ◯ |
| | | | | | | | | | | green | |
| Comparative | Equal to CeO₂ | Fe (III) | 5 | — | — | 2-4 | 98 | 30 | 60 | Yellow | X |
| Example C1-7 | | | | | | | | | | | |
| Comparative | Equal to CeO₂ | — | — | — | — | 2-4 | 99 | 29 | 75 | Yellow | Δ |
| Example C1-8 | | | | | | | | | | | |

### EXAMPLE C1-29

Into the same reactor as of Example C1-1, there was charged a mixture comprising 146 parts of indium acetate anhydride powder, 1.72 parts of iron(III) acetate hydroxide powder, 0.18 part of iron(II) acetate powder, and 3,322 parts of methanol, and then its gas phase portion was purged with nitrogen gas. Thereafter, under stirring, the temperature of the mixture was raised from room temperature to 180° C. and then heated at 180° C. A 1° C. for 5 hours to thus carry out a reaction to form metal oxide particles and then cooled, thereby obtaining a reaction liquid (129) containing fine particles in a fine particle concentration of 2 wt %.

The fine particles in the reaction liquid (129) were subjected to the aforementioned various evaluations. Their results are shown in Table 28. Incidentally, the evaluation of the dispersion particle diameter was carried out by using as the sample a dispersion such that the metal oxide particles were dispersed in 1-butanol in a particle concentration of 20 wt % wherein the dispersion was obtained by subjecting the resultant reaction liquid to heating solvent displacement. The coloring degree was evaluated in accordance with the above evaluation method (5-1) in which the 1-butanol dispersion was used as the sample to carry out the evaluation in the form of the dispersion membrane.

### EXAMPLES C1-30 to C1-32

Reaction liquids (130) to (132) were obtained in the same way as of Example C1-29 except that the ratio between the iron(III) acetate hydroxide powder and the iron(II) acetate powder being charged was changed so that the ratio between Fe of 2 in valence and Fe of 3 in valence would be such a metal composition as shown in Table 28.

The fine particles in the reaction liquids (130) to (132) were subjected to the same evaluations as of Example C1-29. Their results are shown in Table 28.

### COMPARATIVE EXAMPLE C1-9

A reaction liquid (c19) was obtained in the same way as of Example C1-29 except that there was not used the 0.18 part of iron(II) acetate powder but that the iron(III) acetate hydroxide powder was used in an amount of 1.9 parts.

The fine particles in the reaction liquid (c19) were subjected to the same evaluations as of Example C1-29. Their results are shown in Table 28.

TABLE 28

| | | | | | | Coloring | |
|---|---|---|---|---|---|---|---|
| | | Metal composition | | Crystal | | degree in | |
| | X-ray | Fe (II) | Fe (III) | grain | Hue of | form of | Dispersion |
| | diffraction | Atomic %/ | Atomic %/ | diameter | particle | dispersion | particle |
| | pattern | In | In | Dw (nm) | powder | membrane | diameter (nm) |
| Example | Equal to In₂O₃ | 0.2 | 1.8 | 5 | Yellowish | Δ | <50 |
| C1-29 | | | | | green | | |
| Example | Equal to In₂O₃ | 1 | 1 | 5 | Green | ◯ | <50 |
| C1-30 | | | | | | | |
| Example | Equal to In₂O₃ | 1.8 | 0.2 | 5 | Green | ◯ | <50 |
| C1-31 | | | | | | | |
| Example | Equal to In₂O₃ | 2 | 0 | 5 | Green | ◯ | <50 |
| C1-32 | | | | | | | |
| Comparative | Equal to In₂O₃ | 0 | 2 | 5 | Yellow | X | <50 |
| Example | | | | | | | |
| C1-9 | | | | | | | |

### EXAMPLE C2-1

An amount of 1,000 parts of the reaction liquid (11) (particle concentration: 4.4 wt %), having been obtained from Example C1-1, was heated under normal pressure to distill off 710 parts of solvent components such as methanol, thereby concentrating the reaction liquid (11). While this concentrated reaction liquid was further heated, 1-butanol was continuously dropwise added thereto, thereby carrying out solvent displacement with 1-butanol at the same time as distilling off residual solvent components from the reaction liquid, so that a dispersion (21) such that the metal oxide particles were dispersed in 1-butanol in a particle concentration of 20 wt % was obtained as a composition for membrane formation.

The dispersion particle diameters of the resultant dispersion (21) were measured with a dynamic light scattering type particle diameter distribution measurement device (produced by Horiba Seisakusho, product name: LB-500). As a result, the average dispersion particle diameter was not larger than 100 nm.

### EXAMPLES C2-2 to C2-5

Dispersions (22) to (25) were obtained as compositions for membrane formation in the same way as of Example C2-1 except that the reaction liquid being used, the solvent being used for the solvent displacement, and the particle concentration were changed as shown in Table 29.

The dispersion particle diameters of each of the resultant dispersions (22) to (25) were measured with the same device as of Example C2-1. As a result, in any case, the average dispersion particle diameter was not larger than 100 nm.

### COMPARATIVE EXAMPLE C2-1

A dispersion (c21) was obtained as a composition for membrane formation in the same way as of Example C2-1 except that the reaction liquid being used, the solvent being used for the solvent displacement, and the particle concentration were changed as shown in Table 29.

The dispersion particle diameters of the resultant dispersion (c21) were measured with the same device as of Example C2-1. As a result, the average dispersion particle diameter was not larger than 100 nm.

TABLE 29

| | | Dispersion obtained | | | |
|---|---|---|---|---|---|
| | | | | Particle | |
| | Reaction | | | concen- | Dispersion |
| | liquid | | | tration | particle |
| | used | No. | Solvent | wt % | diameter |
| Example C2-1 | 11 | 21 | 1-Butanol | 20 | <100 nm |
| Example C2-2 | 12 | 22 | 1-Butanol | 20 | <100 nm |
| Example C2-3 | 13 | 23 | Toluene | 20 | <100 nm |
| Example C2-4 | 14 | 24 | Butyl | 30 | <100 nm |
| | | | acetate | | |
| Example C2-5 | 15 | 25 | 1-Butanol | 20 | <100 nm |
| Comparative | c11 | c21 | 1-Butanol | 20 | <100 nm |
| Example C2-1 | | | | | |

### EXAMPLE C3-1

An amount of 100 parts of the dispersion (21), having been obtained from Example C2-1, was mixed with 20 parts of a silicate binder (solid component content in terms of SiO₂: 51 wt %) and 0.2 part of a catalyst (n-butylamine) to thus prepare a paint.

The resultant paint was coated onto alkali-free glass (produced by Corning International Corporation, barium borosilicate glass, Glass Code No. 7059, thickness: 0.6 mm) by use of a bar coater in a way for the wet membrane thickness to be 45 μm. Thereafter, they were normally dried at 25° C. and then heat-treated at 250° C. to thereby obtain glass, on the surface of which there was formed a metal-oxide-particles-dispersed membrane.

The resultant dispersion-membrane-coated glass was subjected to the aforementioned evaluations. As a result, the visible-ray transparency was “A”, and the coloring degree was “◯”. As to the visible-ray transmission property, the transmittance at 500 nm was 76%. As to the ultraviolet absorption property, the transmittance at 380 nm was less than 2%, and the ultraviolet absorption property was a property of being able to almost entirely absorb light of not longer than 380 nm. The visible long-wavelength absorption property was “A”. Incidentally, as to the transmittance (%) at each wavelength of only the alkali-free glass used as the substrate, any of the transmittance at 500 nm, the transmittance at 380 nm, and the transmittances in the range of 550 to 700 nm was 91% (the same also in the below-mentioned Examples (C)).

### EXAMPLE C3-2

The dispersion (21), having been obtained from Example C2-1, was coated onto the same alkali-free glass as of Example C3-1 by use of a bar coater, and then its temperature was raised from normal temperature in a calcination furnace and then retained at 500° C. for 1 hour and then cooled, thus obtaining glass, on the surface of which there was formed a thin membrane comprising Co-doped ZnO particles.

The resultant thin-membrane-coated glass was a material which exercised the absorption from near 450 nm to the ultraviolet range (shorter wavelength side) and was able to almost entirely absorb and intercept light of not longer than 370 nm. In addition, this thin-membrane-coated glass was subjected to the aforementioned evaluations. As a result, the visible-ray transparency was “A”, and the coloring degree was “◯”. As to the visible-ray transmission property, the transmittance at 500 nm was 88%. The visible long-wavelength absorption property was “A”.

### EXAMPLE C3-3

A paint was prepared in the same way as of Example C3-1 except that 100 parts of the dispersion (21) was replaced with 100 parts of the dispersion (25) having been obtained from Example C2-5.

The resultant paint was coated onto the same alkali-free glass as of Example C3-1 by use of a bar coater in a way for the wet membrane thickness to be 42 μm

Thereafter, they were normally dried at 25° C. and then heat-treated at 250° C. to thereby obtain glass, on the surface of which there was formed a metal-oxide-particles-dispersed membrane.

The resultant dispersion-membrane-coated glass was subjected to the aforementioned evaluations. As a result, the visible-ray transparency was “A”, and the coloring degree was “◯”. As to the visible-ray transmission property, the transmittance at 500 nm was 78%. As to the ultraviolet absorption property, the transmittance at 380 nm was less than 1%, and the ultraviolet absorption property was a property of being able to almost entirely absorb light of not longer than 380 nm. The visible long-wavelength absorption property was “A”. Incidentally, the coloring degree was more alleviated and therefore favorable for uses which demanded to be more colorless transparent, when compared with the dispersion-membrane-coated glass of Example C3-1 and the thin-membrane-coated glass of Example C3-2.

### EXAMPLE C3-4

The reaction liquid (110), having been obtained from Example C1-10, was coated onto the same alkali-free glass as of Example C3-1 as a substrate by use of a bar coater and then dried at normal temperature and then heated at 400° C. under a nitrogen atmosphere in a heating furnace for 1 hour, thus obtaining a membrane-coated substrate, on the surface of which there was formed a membrane of 0.6 μm in membrane thickness.

The resultant membrane-coated substrate was evaluated. As a result, this membrane-coated substrate was provided with a ZnO crystal membrane containing Fe(II) in a ratio of 2 atomic % relative to Zn and was an ultraviolet-interception glass which was slightly-greenish and excellent in the colorlessness and of which: the ultraviolet absorption property was 30% in transmittance at 380 nm, and the visible-ray transmission property was 90% in transmittance at 500 nm, and the transparency was 0.3% in haze.

### COMPARATIVE EXAMPLE C3-1

A membrane-coated substrate, on the surface of which there was formed a membrane of 0.6 μm in membrane thickness, was obtained in the same way as of Example C3-4 except that the reaction liquid (110) was replaced with the reaction liquid (c13) having been obtained from Comparative Example C1-3.

The resultant membrane-coated substrate was evaluated. As a result, this membrane-coated substrate was provided with a ZnO crystal membrane containing Fe(III) in a ratio of 2 atomic % relative to Zn and was an ultraviolet-interception glass which was conspicuous in yellowing and of which: the ultraviolet absorption property was 50% in transmittance at 380 nm, and the visible-ray transmission property was 88% in transmittance at 500 nm, and the transparency was 0.6% in haze.

[Fourth Metal Oxide Particle]:

The measurement and evaluation methods in the below-mentioned Examples and Comparative Examples are shown below.

<Evaluation of Metal Oxide Particles>:

(1) Crystal Identification of Metal Oxide Particles:

As to the above powder sample, the crystal system and crystal structure of the metal oxide particles were evaluated by powder X-ray diffractometry with a powder X-ray diffraction device (produced by Rigaku Denki K.K., product name: RINT 2400). Hereinafter the measurement conditions are shown.
X-rays: CuKα1 rays (wavelength: 1.54056 Å)/40 kV/200 mA
Scanning range: 2θ=20 to 80°
Scanning speed: 5°/min

Incidentally, in cases where the metal oxide particles contained Zn as a main metal component, then whether the metal oxide particles had the same crystal system and crystal structure as of ZnO or not was judged from whether three intense-ray peaks characteristic of ZnO of the hexagonal crystal system were seen or not. Specifically, if diffraction peaks existed in all positions of the following three diffraction angles (a) to (c), then it was judged that the metal oxide particles had the same crystal system and crystal structure as of ZnO.
(a) 2θ=31.65 to 31.95°
(b) 2θ=34.30 to 34.60°
(c) 2θ=36.10 to 36.40°

Incidentally, the diffraction peak existing in the position of the above (a) is judged to be based on diffracted rays to the (100) plane of the ZnO crystal, and the diffraction peak existing in the position of the above (b) is judged to be based on diffracted rays to the (002) plane of the ZnO crystal, and the diffraction peak existing in the position of the above (c) is judged to be based on diffracted rays to the (101) plane of the ZnO crystal.

Similarly also in cases where a metal element other than Zn was contained as a main metal component in the metal oxide particles, whether the metal oxide particles had the same crystal system and crystal structure as of an oxide of the above metal element or not was judged from whether three intense-ray peaks characteristic of an oxide crystal of the above metal element were seen or not.

(2) Particle Diameter of Metal Oxide Particles:

(2-1) Primary Particle Diameter:

The crystal grain diameter (Dw) of the metal oxide particles was measured and evaluated as the primary particle diameter.

The crystal grain diameter (Dw) was evaluated in the following way: as to the above powder sample, the crystal grain diameter (Dw) of the metal oxide particles was evaluated by powder X-ray diffractometry with a powder X-ray diffraction device (produced by Rigaku Denki K.K., product name: RINT 2400). Specifically, the crystal grain diameter Ds (hkl) (wherein the hkl denotes a Miller index: the Ds (hkl) is the size of the crystal grain in the vertical direction to the lattice plane of theMiller index (hkl)) was determined by Scherrer equation (analysis) from widths of diffracted rays in the resultant X-ray diffraction pattern, and the average value of three intense rays' respective Ds values was taken as the Dw. That is to say, unless otherwise noted, the crystal grain diameter (Dw) is usually calculated in the following way. A powder X-ray diffraction pattern of the metal oxide particle is measured, and then, as to three intense rays thereof (the largest peak (1) of diffracted rays, the second largest peak (2) of diffracted rays, and the third largest peak (3) of diffracted rays), the crystal grain diameters Ds1, Ds2, and Ds3 in the vertical directions to the diffraction planes assigned to the diffracted rays (1) to (3) respectively are determined from their respective full widths of half maximum intensity or integral widths in accordance with Scherrer equation, and then their average value ((Ds1+Ds2+Ds3)/3) is calculated as the crystal grain diameter (Dw).

(2-2) Dispersion Particle Diameter:

The resultant reaction liquid, or a solvent dispersion having been obtained fromthis reaction liquid by solvent displacement, was used as the sample, and its median diameter was measured with a dynamic light scattering type particle diameter distribution measurement device (“LB-500” produced by Horiba Seisakusho) and taken as the dispersion particle diameter. In cases where dilution was carried out in preparation for the measurement, then the solvent having been used in the reaction was used as a diluting solvent. The evaluation standards are as follows:
A: Dispersion particle diameter<0.1 μm
B: 0.1 μm≦Dispersion particle diameter<0.5 μm
C: 0.5 μm≦Dispersion particle diameter

(3) Composition of Metal Oxide Particles:

(3-1) Contents of N, S, and Group-17 Elements:

They were determined by subjecting the above powder sample to elemental analysis.

(3-2) Content of Added Metal Element (M′):

It was determined by dissolving the above powder sample into an aqueous strong-acid solution and then subjecting the resultant solution to ICP analysis.

(3-3) Bonding Amount of Acyl Group:

An amount of 1 g of the above powder sample was added to a 0.1 N aqueous sodium hydroxide solution and then stirred for 24 hours. Thereafter, by ion chromatography, the acyl group was identified and quantified in bonding amount.

(3-4) Evaluation of Valence of Added Metal Element (M′):

If necessary, the valence of the added metal element (M′) in the metal oxide particles was evaluated in the following way. That is to say, as to the above powder sample, a 2p_{3/2} spectrum of the added metal element (M′) contained in the metal oxide particles was measured by X-ray photoelectron spectroscopy (XPS) with a photoelectron spectroscope (produced by Nippon Denshi K.K., product name: JPS-90 model) and, from its peak position, the bond energy value was determined to thus judge the valence of the added metal element (M′).

Incidentally, in order to reduce measured value errors caused by such as energy shift due to the electrification property, the determination of the bond energy value was put under corrections based on the C 1s peak position of the surface hydrocarbon.

In addition, as already known data for comparison, peak positions of 2p_{3/2} spectra of compounds of various metal elements as shown in “The Handbook of X-ray Photoelectron Spectroscopy” (1991) published by Nippon Denshi K.I(. were referred to.

(4) Optical Properties of Metal Oxide Particles:

(4-1) Absorption Properties:

A dilution, having been prepared by diluting the resultant dispersion with 1-butanol as a diluting solvent so as to be 0.1 wt % in fine particle concentration, was used as the sample and, as to this sample, its transmission spectrum in the ultraviolet and visible ranges was measured by use of an auto-recording spectrophotometer having an integrating sphere (“UV-3100” produced by Shimadzu Corporation). The ultraviolet absorption property was evaluated by a transmittance (T1) at 400 nm, and the visible-ray transmission property was evaluated by a transmittance (T2) at 600 nm, and they were judged on the following standards:
Ultraviolet absorption property: A: T1<30%
   B: 30%≦T1<60%
   C: 60%≦T1
Visible-ray transmission property: A: T2≧80%
   B: 80%>T2≧60%
   C: 60%>T2

(4-2) Evaluations of Coloring Degree and Transparency Degree:

A fine-particles-dispersed membrane was formed and evaluated. Specifically, 100 parts of the resultant dispersion was mixed with 20 parts of a silicate binder (solid component content in terms of SiO₂: 51 wt %) and 0.5 part of a catalyst (n-butylamine) to thus prepare a paint.

The resultant paint was coated onto alkali-free glass (produced by Corning International Corporation, barium borosilicate glass, Glass Code No. 7059, thickness: 0.6 mm) by use of a bar coater in a way for the wet membrane thickness to be 24 μm.Thereafter, they were normally dried at 25° C. to thereby obtain glass, on the surface of which there was formed a metal-oxide-particles-dispersed membrane.

Then, as to the coloring degree, the appearance of the above dispersion-membrane-coated glass was observed with the eye to evaluate the coloring degree on the following standards:
◯: Coloring is not seen.
Δ: Coloring is seen a little.
×: Coloring is seen remarkably.

As to the transparency degree, the appearance of the above dispersion-membrane-coated glass was observed with the eye to evaluate the transparency degree on the following standards:
◯: The transparency is high.
Δ: Transparent, but turbidity is slightly seen.
×: Turbid.

(5) Hue of Powder Sample:

It was evaluated by observing the appearance of the powder sample with the eye.

(6) Fine Particle Concentration:

The fine particle concentration of the reaction liquid or of the dispersion was calculated by weighing out 0.5 g of the reaction liquid or of the dispersion in a melting pot and then vacuum-drying it at 120° C. for 1 hour and then measuring the weight of the resultant dry powder.

### EXAMPLE D1-1

There was prepared a reaction apparatus comprising: a pressure-resistant glass reactor possible to externally heat and equipped with a stirrer, an addition inlet (connected directly to an addition tank), a thermometer, a distillate gas outlet, and a nitrogen-gas-introducing inlet; the addition tank connected to the above additioninlet; and a condenser (connected directly to a trap) connected to the above distillate gas outlet.

Into the above reactor, there was charged a mixture comprising 156 parts of zinc formate anhydride powder, 3,912 parts of 1-butanol, and 0.1 part of urea, and then its gas phase portion was purged with nitrogen gas. Thereafter, under stirring, the temperature of the mixture was raised from 20° C. to 150° C. and then heat-retained at 150° C.±1° C. for 10 hours to thus carry out a reaction to form metal oxide particles and then cooled, thereby obtaining a reaction liquid (11) containing fine particles in a concentration of 2 wt %. The resultant reaction liquid (11) was heated under reduced pressure to thus partially distill off the solvent component from the reaction liquid, thereby obtaining a dispersion (11) in which the fine particles were contained in a concentration of 20 wt %.

The resultant dispersion and the metal oxide particles in this dispersion were evaluated in accordance with the aforementioned various evaluation methods. Their results are shown in Table 31.

EXAMPLES D1-2 to D1-5

Reaction liquids (12) to (15) were obtained in the same way as of Example D1-1 except that the kinds and use amounts of the raw materials being charged were changed as shown in Table 30. Further in the same way, there were obtained dispersions (12) to (15) in which the fine particles were contained in a concentration of 20wt %.

The resultant dispersions and the metal oxide particles in these dispersions were evaluated in accordance with the aforementioned various evaluation methods. Their results are shown in Table 31.

### EXAMPLE D1-6

After the reaction liquid (12) had been obtained in the same way as of Example D1-2, the resultant reaction liquid (12) was subjected to centrifugal separation, and then the resultant sediment was vacuum-dried at 80° C. and then pulverized, thus obtaining a fine particle powder. This powder was heat-treated at 300° C. in a hydrogen-sulfide-containing nitrogen gas, and then the resultant powder was dispersed into 1-butanol with a beads mill so as to be 20 wt % in fine particle concentration, thus obtaining a dispersion (16).

The resultant dispersion and the metal oxide particles in this dispersion were evaluated in accordance with the aforementioned various evaluation methods. Their results are shown in Table 31 .

### COMPARATIVE EXAMPLE D1-1

A dispersion (c11) was obtained in the same way as of Example D1-6 except that the hydrogen-sulfide-containing nitrogen gas was replaced with nitrogen gas and that the heat treatment temperature was changed from 300° C. to 400° C.

The resultant dispersion and the metal oxide particles in this dispersion were evaluated in accordance with the aforementioned various evaluation methods. Their results are shown in Table 31.

### COMPARATIVE EXAMPLE D1-2

After the reaction liquid (13) had been obtained in the same way as of Example D1-3, the resultant reaction liquid (13) was subjected to centrifugal separation, and then the resultant sediment was vacuum-dried at 80° C. and then pulverized, thus obtaining a fine particle powder. This powder was exposed to a ammonia-containing nitrogen gas and then heat-treated at 350° C. in nitrogen, and then the resultant powder was dispersed into 1-butanol with a beads mill so as to be 20 wt % in fine particle concentration, thus obtaining a dispersion (c12).

The resultant dispersion and the metal oxide particles in this dispersion were evaluated in accordance with the aforementioned various evaluation methods. Their results are shown in Table 31.

### EXAMPLE D1-7

After the reaction liquid (12) had been obtained in the same way as of Example D1-2, the resultant reaction liquid (12) was subjected to centrifugal separation, and then the resultant sediment was vacuum-dried at 80° C. and then pulverized, thus obtaining a fine particle powder. This powder was dispersed into 1-butanol with a beads mill so as to be 20 wt % in fine particle concentration, thus obtaining a dispersion (17).

The resultant dispersion and the metal oxide particles in this dispersion were evaluated in accordance with the aforementioned various evaluation methods. Their results are shown in Table 31.

As to the fine particles in the dispersions having been obtained from Examples D1-1 to D1-5 and D1-7, their transparency degree and coloring degree in the dispersion membrane state were alleviated in yellowing when compared with the fine particles in the dispersion having been obtained from Comparative Example D1-2. Incidentally, the dispersion membrane having been obtained from the dispersion having been obtained from Comparative Example D1-1 was turbid in white and was therefore evaluated as “Δ” as to the coloring degree.

TABLE 30

| | Zn compound | | Added compound | | Ms compound | |
|---|---|---|---|---|---|---|
| | | Weight | | Weight | | Weight |
| | | parts | | parts | | parts |
| Example | Zinc | 156 | Urea | 0.1 | — | 0 |
| D1-1 | formate | | | | | |
| Example | Zinc acetate | 183 | Urea | 0.5 | — | 0 |
| D1-2 | | | | | | |
| Example | Zinc | 212 | Urea | 1.5 | — | 0 |
| D1-3 | propionate | | | | | |
| Example | Zinc acetate | 183 | Urea | 0.3 | Titanium | 0.5 |
| D1-4 | | | | | tetrabutoxide | |
| | | | | | tetramer | |
| Example | Zinc acetate | 183 | Urea | 1.0 | Tetramethoxysilane | 6.0 |
| D1-5 | | | | | | |

TABLE 31

| | X-ray | Hetero- | | Bonded | | | | Crystal grain | | |
|---|---|---|---|---|---|---|---|---|---|---|
| | diffraction | nonmetal | Atomic %/ | acyl | mol %/ | | Atomic %/ | diameter (nm) | | |
| | pattern | element | Zn | group | Zn | Ms | Zn | Dw | Ds (100) | Ds (002) |
| Example | Equal to | Nitrogen | 0.2 | Formyl | 6 | — | 0 | 17 | 16 | 17 |
| D1-1 | ZnO | | | group | | | | | | |
| Example | Equal to | Nitrogen | 1 | Ethanoyl | 3 | — | 0 | 15 | 14 | 16 |
| D1-2 | ZnO | | | group | | | | | | |
| Example | Equal to | Nitrogen | 3 | Propanoyl | 1 | — | 0 | 17 | 10 | 30 |
| D1-3 | ZnO | | | group | | | | | | |
| Example | Equal to | Nitrogen | 0.5 | Ethanoyl | 3 | Ti*1 | 0.2 | 17 | 15 | 20 |
| D1-4 | ZnO | | | group | | | | | | |
| Example | Equal to | Nitrogen | 2 | Ethanoyl | 0.2 | Si*2 | 4 | 14 | 12 | 18 |
| D1-5 | ZnO | | | group | | | | | | |
| Example | Equal to | Nitrogen | 0.7 | — | 0 | — | 0 | 15 | 14 | 16 |
| D1-6 | ZnO | Sulfur | 3 | | | | | | | |
| Example | Equal to | Nitrogen | 1 | Ethanoyl | 3 | — | 0 | 15 | 14 | 16 |
| D1-7 | ZnO | | | group | | | | | | |
| Comparative | Equal to | — | 0 | — | 0 | — | 0 | 16 | 15 | 17 |
| Example | ZnO | | | | | | | | | |
| D1-1 | | | | | | | | | | |
| Comparative | Equal to | Nitrogen | 2 | — | 0 | — | 0 | 18 | 11 | 31 |
| Example | ZnO | | | | | | | | | |
| D1-2 | | | | | | | | | | |

| | | | | | Visible-ray | Ultraviolet |
|---|---|---|---|---|---|---|
| | | Dispersion | | | transmission | absorption |
| | Hue of | particle | Coloring | Transparency | property (%) | property (%) |
| | powder | diameter | degree | degree | 600 nm | 400 nm |
| Example | Yellow | B | Δ | Δ | A | B |
| D1-1 | | | | | | |
| Example | Yellow | B | Δ | Δ | A | B |
| D1-2 | | | | | | |
| Example | Yellow | B | Δ | Δ | A | B |
| D1-3 | | | | | | |
| Example | Yellow | A | Δ | ◯ | A | B |
| D1-4 | | | | | | |
| Example | Yellow | A | Δ | ◯ | A | B |
| D1-5 | | | | | | |
| Example | Dark | C | X | X | A | A |
| D1-6 | yellow | | | | | |
| Example | Yellow | B | Δ | Δ | A | B |
| D1-7 | | | | | | |
| Comparative | White | C | Δ | X | A | C |
| Example | | | | | | |
| D1-1 | | | | | | |
| Comparative | Yellow | C | X | X | A | C |
| Example | | | | | | |
| D1-2 | | | | | | |

*1Being bonded as a (partially) hydrolyzed product of titanium tetrabutoxide tetramer.
*2Being bonded as a (partially) hydrolyzed product of tetramethoxysilane.

### EXAMPLE D2-1

A reaction liquid (21) was obtained in the same way as of Example D1-1 except that the raw material mixture was caused to further contain 1.8 parts of iron(II) acetate anhydride powder. Further in the same way, there was obtained a dispersion (21) in which the fine particles were contained in a concentration of 20 wt %.

The resultant dispersion and the metal oxide particles in this dispersion were evaluated in accordance with the aforementioned various evaluation methods. Their results are shown in Table 32.

### COMPARATIVE EXAMPLE D2-1

A reaction liquid (c21) was obtained in the same way as of Example D1-1 except that the urea was not used, but that the raw material mixture was caused to further contain 1.8 parts of iron(II) acetate anhydride powder. Further in the same way, there was obtained a dispersion (c21) in which the fine particles were contained in a concentration of 20 wt %.

The resultant dispersion and the metal oxide particles in this dispersion were evaluated in accordance with the aforementioned various evaluation methods. Their results are shown in Table 32.

As is shown in Table 32, the fine particles in the dispersion (21) having been obtained from Examples D2-1 were yellowish green, and were more inconspicuous in dispersion membrane coloring than the fine particles in the dispersion (11) having been obtained from Example D1-1, and were more excellent in ultraviolet absorption property than the fine particles having been obtained from Example D1-1 and Comparative Example D2-1.

### EXAMPLE D2-2

A reaction liquid (22) was obtained in the same way as of Example D1-3 except that the raw material mixture was caused to further contain 0.25 part of copper(I) acetate anhydride powder. Further in the same way, there was obtained a dispersion (22) in which the fine particles were contained in a concentration of 20 wt %.

The resultant dispersion and the metal oxide particles in this dispersion were evaluated in accordance with the aforementioned various evaluation methods. Their results are shown in Table 32.

### COMPARATIVE EXAMPLE D2-2

A reaction liquid (c22) was obtained in the same way as of Example D1-3 except that the urea was not used, but that the raw material mixture was caused to further contain 0.25 part of copper(I) acetate anhydride powder. Further in the same way, there was obtained a dispersion (c22) in which the fine particles were contained in a concentration of 20 wt %.

The resultant dispersion and the metal oxide particles in this dispersion were evaluated in accordance with the aforementioned various evaluation methods. Their results are shown in Table 32.

As is shown in Table 32, the fine particles in the dispersion (22) having been obtained from Example D2-2 were light gray, and were more inconspicuous in dispersion membrane coloring than the fine particles in the dispersion (13) having been obtained from Example D1-3, and were more excellent in ultraviolet absorption property than the fine particles having been obtained from Example D1-3 and Comparative Example D2-2.

### EXAMPLE D2-3

A reaction liquid (23) was obtained in the same way as of Example D1-2 except that the raw material mixture was caused to further contain 1.3 parts of nickel(II) acetate tetrahydrate powder. Further in the same way, there was obtained a dispersion (23) in which the fine particles were contained in a concentration of 20 wt %.

The resultant dispersion and the metal oxide particles in this dispersion were evaluated in accordance with the aforementioned various evaluation methods. Their results are shown in Table 32.

### EXAMPLE D2-4

A reaction liquid (24) was obtained in the same way as of Example D1-2 except that the raw material mixture was caused to further contain 1.8 parts of cobalt(II) acetate anhydride powder. Further in the same way, there was obtained a dispersion (24) in which the fine particles were contained in a concentration of 20 wt %.

The resultant dispersion and the metal oxide particles in this dispersion were evaluated in accordance with the aforementioned various evaluation methods. Their results are shown in Table 32.

As is shown in Table 32, the fine particles in the dispersions (23) and (24) having been obtained from Examples D2-3 and D2-4 were more inconspicuous in dispersion membrane coloring than the fine particles in the dispersion (12) having been obtained from Example D1-2, and were more excellent in ultraviolet absorption property than the fine particles having been obtained from Example D1-2.

TABLE 32

| | | | | | | | | | | | | | Visible- | Ultra- |
|---|---|---|---|---|---|---|---|---|---|---|---|---|---|---|
| | | | | | | | | | | | | | ray trans- | violet |
| | X-ray | | | | | | | Crystal grain | | | | | mission | absorption |
| | dif- | Hetero- | | | | Bonded | | diameter (nm) | | | | Color- | property | property |
| | fraction | nonmetal | Atomic | | Atomic | acyl | mol | | Ds | Ds | Hue of | ing | (%) | (%) |
| | pattern | element | %/Zn | M′ | %/Zn | group | %/Zn | Dw | (100) | (002) | powder | degree | 600 nm | 400 nm |
| Example | Equal to | Nitrogen | 0.2 | — | — | Formyl | 6 | 17 | 16 | 17 | Yellow | Δ | A | B |
| D1-1 | ZnO | | | | | group | | | | | | | | |
| Example | Equal to | Nitrogen | 0.2 | Fe (II) | 1 | Formyl | 5 | 15 | 15 | 15 | Yellowish | ◯ | A | A |
| D2-1 | ZnO | | | | | group | | | | | green | | | |
| Compar- | Equal to | — | — | Fe (II) | 1 | Formyl | 5 | 16 | 15 | 17 | Green | ◯ | A | B |
| ative | ZnO | | | | | group | | | | | | | | |
| Example | | | | | | | | | | | | | | |
| D2-1 | | | | | | | | | | | | | | |
| Example | Equal to | Nitrogen | 3 | — | — | Propanoyl | 1 | 17 | 10 | 30 | Yellow | Δ | A | B |
| D1-3 | ZnO | | | | | group | | | | | | | | |
| Example | Equal to | Nitrogen | 2.8 | Cu (I) | 0.2 | Propanoyl | 0.8 | 12 | 10 | 16 | Gray | ◯ | A | A |
| D2-2 | ZnO | | | | | group | | | | | | | | |
| Compar- | Equal to | — | — | Cu (I) | 0.2 | Propanoyl | 1.5 | 13 | 10 | 18 | Gray | ◯ | A | C |
| ative | ZnO | | | | | group | | | | | | | | |
| Example | | | | | | | | | | | | | | |
| D2-2 | | | | | | | | | | | | | | |
| Example | Equal to | Nitrogen | 1 | — | — | Ethanoyl | 3 | 15 | 14 | 16 | Yellow | Δ | A | B |
| D1-2 | ZnO | | | | | group | | | | | | | | |
| Example | Equal to | Nitrogen | 0.9 | Ni (II) | 0.5 | Ethanoyl | 2.5 | 15 | 14 | 16 | Green | ◯ | A | A |
| D2-3 | ZnO | | | | | group | | | | | | | | |
| Example | Equal to | Nitrogen | 1.1 | Co (II) | 1 | Ethanoyl | 2 | 14 | 13 | 15 | Blue | ◯ | A | A |
| D2-4 | ZnO | | | | | group | | | | | | | | |

### EXAMPLE D2-5

Into the same reactor as of Example D1-1, there was charged a mixture comprising 183 parts of zinc acetate anhydride powder, 0.05 part of zinc fluoride anhydride, 12 parts of bismuth(III) acetate oxide powder, 0.07 part of copper(I) acetate anhydride, and 3,880 parts of methanol, and then its gas phase portion was purged with nitrogen gas. Thereafter, under stirring, the temperature of the mixture was raised from 20° C. to 180° C. and then heat-retained at 180° C.±1° C. for 10 hours to thus carry out a reaction to form metal oxide particles and then cooled, thereby obtaining a reaction liquid (25) containing fine particles in a concentration of 2 wt %. The resultant reaction liquid (25) was heated under reduced pressure to thus partially distill off the solvent component from the reaction liquid, thereby obtaining a dispersion (25) in which the fine particles were contained in a concentration of 20 wt %.

The resultant dispersion and the metal oxide particles in this dispersion were evaluated in accordance with the aforementioned various evaluation methods. Their results are shown in Table 34.

### EXAMPLES D2-6 to D2-12

Reaction liquids (26) to (212) were obtained in the same way as of Example D2-5 except that the kinds and use amounts. of the raw materials being charged were changed as shown in Table 33. Further in the same way, there were obtained dispersions (26) to (212) in which the fine particles were contained in a concentration of 20 wt %.

The resultant dispersions and the metal oxide particles in these dispersions were evaluated in accordance with the aforementioned various evaluation methods. Their results are shown in Table 34.

TABLE 33

| | | |
|---|---|---|
| Zn compound | Added compound | M′ compound |

| | | Weight | | Weight | | Weight |
|---|---|---|---|---|---|---|
| | | parts | | parts | | parts |
| Example | Zinc | 183 | Zinc | 0.05 | Bismuth (III) | 12 |
| D2-5 | acetate | | fluoride | | acetate oxide | |
| | | | anhydride | | Copper (I) acetate | 0.07 |
| | | | | | anhydride | |
| Example | Zinc | 183 | Zinc | 0.18 | Manganese (II) | 12 |
| D2-6 | acetate | | fluoride | | acetate anhydride | |
| | | | anhydride | | Copper (II) acetate | 1 |
| | | | | | anhydride | |
| Example | Zinc | 183 | Sodium | 0.18 | Indium (III) | 19 |
| D2-7 | acetate | | sulfide | | acetate anhydride | |
| | | | anhydride | | | |
| Example | Zinc | 212 | Zinc | 0.35 | Aluminum | 5 |
| D2-8 | propionate | | fluoride | | tri-sec-butoxide | |
| | | | anhydride | | Magnesium (II) | 0.05 |
| | | | | | acetate | |
| | | | | | tetrahydrate | |
| Example | Zinc | 212 | Urea | 0.6 | Tin (IV) acetate | 8 |
| D2-9 | propionate | | | | anhydride | |
| Example | Zinc | 212 | Urea | 3 | Cerium (III) | 8 |
| D2-10 | propionate | | | | acetate | |
| | | | | | Copper (I) | 0.15 |
| | | | | | acetate | |
| | | | | | anhydride | |
| Example | Zinc | 156 | Zinc | 0.14 | Silver | 1.8 |
| D2-11 | formate | | chloride | | acetate | |
| | | | anhydride | | | |
| Example | Zinc | 156 | Lithium | 1.4 | — | — |
| D2-12 | formate | | iodide | | | |
| | | | anhydride | | | |

TABLE 34

| | | | | | | | | Crystal | | | | | Visible- | Ultra- |
|---|---|---|---|---|---|---|---|---|---|---|---|---|---|---|
| | | | | | | | | grain | | | | | ray trans- | violet |
| | X-ray | | | | | | | daimeter | | | | | mission | absorption |
| | dif- | Hetero- | | | | Bonded | | (nm) | | | | Color- | property | property |
| | fraction | nonmetal | Atomic | | Atomic | acyl | mol | | Ds | Ds | Hue of | ing | (%) | (%) |
| | pattern | element | %/Zn | M′ | %/Zn | group | %/Zn | Dw | (100) | (002) | powder | degree | 600 nm | 400 nm |
| Example | Equal to | Fluorine | 0.05 | Bi (III) | 4 | Ethanoyl | 0.5 | 17 | 17 | 18 | Gray | ◯ | A | A |
| D2-5 | ZnO | | | Cu (I) | 0.05 | group | | | | | | | | |
| Example | Equal to | Fluorine | 0.3 | Mn (II) | 6.3 | Ethanoyl | 0.8 | 15 | 16 | 13 | Yellow- | Δ | A | A |
| D2-6 | ZnO | | | Cu (I, | 0.5 | group | | | | | brownish | | | |
| | | | | II) | | | | | | | gray | | | |
| Example | Equal to | Sulfur | 0.2 | In (III) | 6 | Ethanoyl | 1.5 | 18 | 18 | 17 | Bluish | ◯ | A | A |
| D2-7 | ZnO | | | Na (I) | 0.4 | group | | | | | green | | | |
| Example | Equal to | Fluorine | 0.2 | Al (III) | 2 | Propanoyl | 1 | 15 | 11 | 23 | Gray | ◯ | A | B |
| D2-8 | ZnO | | | Mg (II) | 0.02 | group | | | | | | | | |
| Example | Equal to | Nitrogen | 1 | Sn (II, | 2 | Propanoyl | 4 | 14 | 11 | 20 | Gray | ◯ | A | B |
| D2-9 | ZnO | | | IV) | | group | | | | | | | | |
| Example | Equal to | Nitrogen | 4 | Ce (III) | 2 | Propanoyl | 8 | 12 | 11 | 14 | Gray | ◯ | A | A |
| D2-10 | ZnO | | | Cu (I) | 0.1 | group | | | | | | | | |
| Example | Equal to | Chlorine | 0.1 | Ag (0, | 1 | Formyl | 0.5 | 24 | 22 | 28 | Yellow | X | A | A |
| D2-11 | ZnO | | | I) | | group | | | | | | | | |
| Example | Equal to | Iodine | 1 | Li (I) | 1 | Formyl | 4 | 13 | 15 | 10 | White | ◯ | A | B |
| D2-12 | ZnO | | | | | group | | | | | | | | |

### EXAMPLE D3-1

Into the same reactor as of Example D1-1, there was charged a mixture comprising 340 parts of tetra-n-butoxytitanium, 300 parts of acetic acid, 1 part of urea, and 3,360 parts of 2-propanol, and then its gas phase portion was purged with nitrogen gas. Thereafter, under stirring, the temperature of the mixture was raised from 20° C. to 160° C. and then heat-retained at 160° C.±1° C. for 5 hours to thus carry out a reaction to form metal oxide particles and then cooled, thereby obtaining a yellow reaction liquid (31) of 2 wt % in fine particle concentration. Furthermore, from the resultant reaction liquid, there was obtained a 1-butanol dispersion (31) of 20 wt % in fine particle concentration.

The fine particles in the resultant dispersion were those to which the ethanoyl group was bonded in a molar ratio of 0.2% relative to Ti.

The resultant dispersion and the metal oxide particles in this dispersion were evaluated in accordance with the aforementioned various evaluation methods. Their results are shown in Table 36.

### EXAMPLES D3-2 to D3-6

Reaction liquids (32) to (36) were obtained in the same way as of Example D3-1 except that the kinds and use amounts of the raw materials being charged were changed as shown in Table 35. Further in the same way, there were obtained dispersions (32) to (36) in which the fine particles were contained in a concentration of 20wt %.

The resultant dispersions and the metal oxide particles in these dispersions were evaluated in accordance with the aforementioned various evaluation methods. Their results are shown in Table 36.

TABLE 35

| | |
|---|---|
| Added compound | M′ compound |

| | | Weight | | Weight |
|---|---|---|---|---|
| | | parts | | parts |
| Example D3-1 | Urea | 1 | — | — |
| Comparative | — | — | — | — |
| Example D3-1 | | | | |
| Example D3-2 | Urea | 1 | Iron (II) acetate | 4 |
| | | | anhydride | |
| Example D3-3 | Urea | 0.2 | Cobalt (II) acetate | 0.2 |
| | | | anhydride | |
| Example D3-4 | Urea | 4 | Nickel (II) acetate | 1 |
| | | | tetrahydrate | |
| Example D3-5 | Copper (I) iodide | 2 | — | — |
| | anhydride | | | |
| Example D3-6 | Lithium fluoride | 0.13 | Bismuth (III) | 12 |
| | | | acetate oxide | |

TABLE 36

| | | | | | | Crystal | | | Visible-ray | Ultra- |
|---|---|---|---|---|---|---|---|---|---|---|
| | X-ray | | | | | grain | | | trans- | violet |
| | dif- | Hetero- | | | | diameter | | | mission | absorption |
| | fraction | nonmetal | Atomic | | Atomic | (nm) | Hue of | Coloring | property | property |
| | pattern | element | %/Ti | M′ | %/Ti | Dw | powder | degree | (%) 600 nm | (%) 400 nm |
| Example | Equal to | Nitrogen | 1 | — | — | 14 | Yellow | Δ | B | B |
| D3-1 | TiO₂ | | | | | | | | | |
| Compar- | Equal to | — | — | — | — | 16 | Slight | ◯ | B | C |
| ative | TiO₂ | | | | | | yellow | | | |
| Example | | | | | | | | | | |
| D3-1 | | | | | | | | | | |
| Example | Equal to | Nitrogen | 1 | Fe (II) | 2 | 10 | Yellowish | ◯ | B | A |
| D3-2 | TiO₂ | | | | | | green | | | |
| Example | Equal to | Nitrogen | 0.2 | Co (II) | 0.1 | 14 | Blue | ◯ | B | B |
| D3-3 | TiO₂ | | | | | | | | | |
| Example | Equal to | Nitrogen | 3 | Ni (II) | 0.4 | 13 | Yellowish | ◯ | B | A |
| D3-4 | TiO₂ | | | | | | green | | | |
| Example | Equal to | Iodine | 0.9 | Cu (I) | 0.8 | 16 | Gray | ◯ | B | B |
| D3-5 | TiO₂ | | | | | | | | | |
| Example | Equal to | Fluorine | 0.4 | Bi (III) | 4 | 14 | Yellowish | Δ | B | A |
| D3-6 | TiO₂ | | | Li (I) | 0.5 | | brown | | | |

### EXAMPLE D4-1

An amount of 100 parts of the dispersion (21), having been obtained from Example D2-1, was mixed with 20 parts of a silicate binder (solid component content in terms of SiO₂: 51 wt %) and 0.2 part of a catalyst (n-butylamine) to thus prepare a paint.

The resultant paint was coated onto an alkali glass by use of a bar coater and then dried at normal temperature and then heated at 200° C. under a nitrogen atmosphere for 1 hour to thereby obtain a dispersion-membrane-coated glass, on the surface of which there was formed a metal-oxide-particles-dispersed membrane.

The resultant dispersion-membrane-coated glass was evaluated by the optical performances. As a result, this glass was an ultraviolet-interception glass which was excellent in the colorlessness and was 92% in transmittance at 600 nm and 25% in transmittance at 400 nm and 0.3% in haze.

Incidentally, the transmittance at each wavelength was determined by measuring a transmission spectrum with the same apparatus as used in the above evaluation method (4), and the haze was evaluated with the turbidimeter. In addition, the coloring degree was evaluated by observing the appearance with the eye.

### EXAMPLE D4-2

An amount of 100 parts of the dispersion (25), having been obtained from Example D2-5, was mixed with 50 parts of an acrylic resin binder (solid component content: 50 wt %) to thus prepare a paint.

The resultant paint was coated onto a polyester film by use of a bar coater and then dried at normal temperature and then heated at 100° C. for 10 minutes to thereby obtain a dispersion-membrane-coated film, on the surface of which there was formed a metal-oxide-particles-dispersed membrane.

The resultant dispersion-membrane-coated film was evaluated by the optical performances in the same way as of Example D4-1. As a result, this film was an ultraviolet-interception film which was excellent in the colorlessness and was 88% in transmittance at 600 nm and 25% in transmittance at 400 nm and 0.5% in haze.

### EXAMPLE D4-3

The dispersion (210), having been obtained from Example D2-10, was coated onto an alkali glass by use of a bar coater and then dried at normal temperature and then heated at 300° C. under a nitrogen atmosphere for 30 minutes to thereby obtain a membrane-coated glass, on the surface of which there was formed a metal oxide membrane.

The resultant membrane-coated glass was provided with a ZnO crystal membrane containing nitrogen, Ce, and Cu in ratios of 3.4 atomic %, 2 atomic %, and 0.1 atomic % respectively relative to Zn and was an ultraviolet-interception glass which was excellent in the colorlessness and was 90% in transmittance at 600 nm and 20% in transmittance at 400 nm and 0.2% in haze as a result of having been evaluated by the optical performances in the same way as of Example D4-1.

### EXAMPLE D4-4

The dispersion (35), having been obtained from Example D3-5, was coated onto an alkali glass by use of a bar coater and then dried at normal temperature and then heated at 400° C. under a nitrogen atmosphere to thereby obtain a membrane-coated glass, on the surface of which there was formed a metal oxide membrane.

The resultant membrane-coated glass was provided with a TiO₂ crystal membrane containing iodine and Cu in ratios of 0.8 atomic % and 0.8 atomic % respectively relative to Ti and was an ultraviolet-interception glass which was excellent in the colorlessness and was 90% in transmittance at 600 nm and 30% in transmittance at 400 nm and 0.3% in haze as a result of having been evaluated by the optical performances in the same way as of Example D4-1.

### INDUSTRIAL APPLICATION

The membrane according to the present invention is, for example, favorable for such as: window glass for buildings; window glass for cars (e.g. automobiles, electric trains), window glass for air transportation machines (e.g. airplanes, helicopters); films for agriculture; and various films for packing. However, the use is not limited to these. Various favorable uses are possible not only for the purpose of providing various functional films with the ultraviolet intercepting function, but also for the purpose of providing the various functional films with such as infrared intercepting function and electric conduction function.

The composition according to the present invention for membrane formation is, for example, favorable as a coating liquid for formation of an ultraviolet intercepting membrane or as an ultraviolet cutting paint, and besides, as a material for formation of the above membrane according to the present invention.

The metal oxide particle according to the present invention is, for example, favorable as a component for providing the ultraviolet intercepting ability in various uses for such as membranes or films, paints, and cosmetic materials, and besides, favorable as a constitutional component of the above membrane according to the present invention and of the above composition according to the present invention.

1. A metal oxide particle in the form of a fine particle, which is a metal oxide particle such that a component derived from a metal element (M′) other than a metal element (M) is contained in a particle comprising an oxide of the metal element (M),
wherein:
the metal element (M) is at least one member selected from the group consisting of Zn, Ti, Ce, In, Sn, Al, and Si; and
the metal element (M′) is at least one member selected from the group consisting of Cu, Ag, Mn, and Bi.

2. A metal oxide particle, which is a metal oxide particle such that a component derived from a metal element (M′) other than a metal element (M) is contained in a particle comprising an oxide of the metal element (M),
wherein:
the metal element (M) is at least one member selected from the group consisting of Zn, Ti, Ce, In, Sn, Al, and Si; and
the metal element (M′) includes at least, two members which are different from the metal element (M) and selected from the group consisting of Co, Cu, Fe, Bi, In, Al, Ga, Ti, Sn, Ce, Ni, Mn, and Ag.

3. A metal oxide particle according to claim 2, wherein the metal element (M′) includes the following essential components: at least one member selected from the group consisting of Co, Cu, and Fe; and at least one member selected from the group consisting of Bi, In, Al, Ga, Ti, Sn, and Ce.

4. A metal oxide particle according to claim 2, wherein the metal element (M′) includes, as an essential component, one member selected from the group consisting of Co, Cu, Fe, Ag, Mn, Ni, and Bi.

5. A metal oxide particle according to claim 2, wherein, in cases where the metal element (M′) is any of Fe, Co, and Ni, then at least a part thereof is 2 in valence.

6. A metal oxide particle, which is a metal oxide particle such that a component derived from a metal element (M′) other than a metal element (M) is contained in a particle comprising an oxide of the metal element (M),
wherein:
the metal element (M) is at least one member selected from the group consisting of Zn, Ti, Ce, In, Sn, Al, and Si; and
the metal element (M′) is at least one member selected from the group consisting of Co, Fe, and Ni, wherein at least a part of these Co, Fe, and Ni is 2 in valence.

7. A metal oxide particle, which is a metal oxide particle such that a component derived from a metal element (M′) other than a metal element (M) is contained in a particle comprising an oxide of the metal element (M),
wherein: the metal element (M) is Zn; the metal element (M′) is at least one member selected from the group consisting of Co, Fe, and Ni; and the metal oxide particle is not larger than 30 nm in crystal grain diameter in the vertical direction to the (002) plane and not smaller than 8 nm in crystal grain diameter in the vertical direction to the (100) plane.

8. A metal oxide particle, which is a metal oxide particle comprising an oxide of a metal element (M),
wherein:
the metal element (M) is at least one member selected from the group consisting of Zn, Ti, Ce, In, Sn, Al, and Si; and
at least one member selected from the group consisting of N, S, and group-17 (group-7B) elements, and further an acyl group, are contained in the oxide of the metal element (M).

9. A metal oxide particle, which is a metal oxide particle comprising an oxide of a metal element (M),
wherein:
the metal element (M) is at least one member selected from the group consisting of Zn, Ti, Ce, In, Sn, Al, and Si; and
at least two members selected from the group consisting of N, S, and group-17 (group-7B) elements are contained in the oxide of the metal element (M).

10. A metal oxide particle, which is a metal oxide particle comprising an oxide of a metal element (M),
wherein:
the metal element (M) is at least one member selected from the group consisting of Zn, Ti, Ce, In, Sn, Al, and Si;
at least one member selected from the group consisting of N, S, and group-17 (group-7B) elements is contained in the oxide of the metal element (M); and
a component derived from a metal element (M′) other than the metal element (M) is contained in the particle.

11. A metal oxide particle according to claim 10, wherein the metal element (M′) is at least one metal element which is different from the metal element (M) and selected from the group consisting of Co, Cu, Fe, Bi, In, Al, Ga, Ti, Sn, Ce, Ni, B, Mn, Ag, Au, platinum group metal elements, alkaline metal elements, and alkaline earth metal elements.

12. A metal oxide particle according to claim 2, which is in the range of 3 to 50 nm in primary particle diameter.

13. A metal oxide particle according to claim 2, wherein: the oxide of the metal element (M) is a crystal; and the metal oxide particle is not larger than 30 nm in crystal grain diameter (average value of values calculated in accordance with Scherrer equation as to three intense rays of XRD peaks).

14. A metal oxide particle according to claim 2, wherein: the oxide of the metal element (M) is a zinc oxide crystal; and the metal oxide particle is not larger than 30 nm in crystal grain diameter in the vertical direction to the lattice plane (002) and not smaller than 10 nm in crystal grain diameter in the vertical direction to the lattice plane (100) and/or lattice plane (110).

15. A metal oxide particle according to claim 2, wherein the particle comprising the oxide of the metal element (M) contains an acyl group of 0.1 to 14 mol % in molar ratio to the metal element (M).

16. A metal oxide particle according to claim 2, wherein the oxide of the metal element (M) is an oxide to surfaces of which there is bonded an alkoxide or its (partially) hydrolyzed product wherein the alkoxide includes at least one metal element different from the metal element (M′) contained in the oxide.

17. A metal oxide particle according to claim 2, in which, besides the metal element (M′), a component derived from an alkaline metal element and/or an alkaline earth metal element is also contained in the range of 0.001 to 5 atomic % relative to the metal element (M).

18. A composition, which comprises a metal oxide particle and a medium, wherein the metal oxide particle is dispersed in the medium and includes, as an essential component, the metal oxide particle as recited in claim 2.

19. A composition according to claim 18 for membrane formation, which comprises the following essential constitutional components: the metal oxide particle as recited in claim 2; and a dispersion solvent and/or a binder.

20. A composition according to claim 18, wherein the metal oxide particle is dispersed in the form of not larger than 1 μm in dispersion particle diameter.

21. A composition according to claim 18, which further comprises: a metal oxide particle including, as a metal element, at least one member selected from the group consisting of Cu, Fe, Ag, and Bi; and/or a superfine metal particle including, as a metal element, at least one member selected from the group consisting of Ag, Cu, Au, and platinum group metal elements.

22. A membrane, which comprises a metal oxide as an essential constitutional component, wherein the metal oxide includes the following essential components: the metal oxide particle as recited in claim 1; and/or a metal oxide crystal derived from this particle.

23. A membrane according to claim 22, which further comprises: a metal oxide particle including, as a metal element, at least one member selected from the group consisting of Cu, Fe, Ag, and Bi and/or a metal oxide crystal derived from this particle; and/or a superfine metal particle including, as a metal element, at least one member selected from the group consisting of Ag, Cu, Au, and platinum group metal elements and/or a crystal including a metal derived from this particle and/or a crystal including a metal oxide derived from this particle.

24. A metal-oxide-containing article, which is an article comprising a metal oxide particle and/or a metal oxide crystal derived from this particle, wherein the article includes, as essential components, a combination of the metal oxide particle as recited in claim 1 with: a metal oxide particle including, as a metal element, at least one member selected from the group consisting of Cu, Fe, Ag, and Bi; and/or a superfine metal particle including, as a metal element, at least one member selected from the group consisting of Cu, Ag, Au, and platinum group metal elements.

25. An ultraviolet absorbent material, which comprises the metal oxide particle as recited in claim 1.
